(19) United States
(12) Patent Application Publication
(10) Pub. No.: US 2011/0152243 A1
(43) Pub. Date: Jun. 23, 2011

(54) NOVEL THIENOPYRROLE COMPOUNDS

(75) Inventors: Neil Wishart, Jefferson, MA (US); Theresa Dunstan, Westborough, MA (US); Jeremy J. Edmunds, Acton, MA (US); Anna M. Ericsson, Shrewsbury, MA (US); Catherine R. Ferenz, Belchertown, MA (US); Adrian D. Hobson, Shrewsbury, MA (US); David C. Ihle, Worcester, MA (US); Kent D. Stewart, Gurnee, IL (US); Lei Wang, Acton, MA (US); Lu Wang, Westborough, MA (US)

(73) Assignee: ABBOTT LABORATORIES, Abbott Park, IL (US)

(21) Appl. No.: 12/974,311

(22) Filed: Dec. 21, 2010

Related U.S. Application Data
(60) Provisional application No. 61/289,595, filed on Dec. 23, 2009.

(51) Int. Cl. A61K 31/397; C07D 401/14; A61K 31/517; A61K 31/496; A61K 31/454; C07D 487/04; A61K 31/53; A61K 31/437; C07D 403/14; A61K 31/416; C07D 471/04; C07D 413/14; A61K 31/5377; A61P 3/10; A61P 17/06; A61P 1/04; A61P 19/02; A61P 35/00

(52) U.S. Cl. 514/210.21; 544/284; 514/266.22; 514/252.17; 546/199; 514/322; 514/249; 544/183; 514/243; 546/121; 514/300; 548/361.1; 514/406; 544/279; 514/264.1; 544/116; 514/234.8

(57) ABSTRACT

The invention provides a compound of Formula (I)

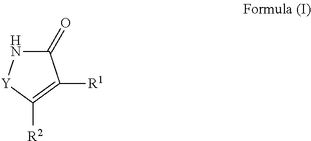

pharmaceutically acceptable salts, pro-drugs, biologically active metabolites, stereoisomers and isomers thereof wherein the variable are defined herein. The compounds of the invention are useful for treating immunological and oncological conditions.

### CROSS REFERENCE TO PRIOR APPLICATION

This application claims priority to U.S. Provisional Application Ser. No. 61/289,595 filed on Dec. 23, 2009, the contents of which are incorporated herein.

### BACKGROUND OF THE INVENTION

The invention provides a novel class of compounds, pharmaceutical compositions comprising such compounds and methods of using such compounds to treat or prevent diseases or disorders associated with abnormal or deregulated kinase activity, particularly diseases or disorders that involve abnormal activation of the PKC, Jak1, Jak2, Jak3, Tyk2, KDR, Flt-3, ROCK, CDK2, CDK4, TANK, Trk, FAK, Abl, Bcr-Abl, cMet, b-RAF, FGFR3, c-kit, PDGF-R, Syk, or Aurora kinases.

Protein kinases constitute a large family of structurally related enzymes that are responsible for the control of a variety of signal transduction processes within cells (see, e.g., Hardie and Hanks, The Protein Kinase Facts Book, I and II, Academic Press, San Diego, Calif., 1995). Protein kinases are thought to have evolved from a common ancestral gene due to the conservation of their structure and catalytic function. Almost all kinases contain a similar 25-300 amino acid catalytic domain. The kinases may be categorized into families by the substrate that they phosphorylate (e.g., protein-tyrosine, protein-serine/threonine, lipids etc.). Sequence motifs have been identified that generally correspond to each of these families (see e.g., Hanks and Hunter, (1995), FASEB J. 9:576-596; Knighton et al., (1991), Science 253:407-414; Hiles et al., (1992), Cell 70:419-429; Kunz et al., (1993), Cell 73:585-596; Garcia-Bustos et al., (1994), EMBO J 13:2352-2361).

The protein kinase C family is a group of serine/threonine kinases including at least ten related isoenzymes, including alpha, beta 1, beta 2, gamma, delta, epsilon, eta, lambda, iota, theta and zeta. The isoenzymes have been divided into three groups based on their different expression patterns and co-factor requirements. The classical PKC enzymes (cPKC), including alpha, beta 1, beta 2 and gamma isozymes require diacylglycerol (DAG), phosphatidylserine (PS) and calcium for activation. The novel PKC's (nPKC), including delta, epsilon, theta and eta isozymes, require DAG and PS but are calcium independent. The atypical PKC's (aPKC), including zeta, lambda/iota do not require calcium or DAG.

PKC isoforms have been shown to play key roles in cellular signaling, proliferation, differentiation, migration, survival, and death. In resting cells, PKCs are predominantly localized in the cytosol and are catalytically inactive due to autoinhibition by their pseudosubstrate domain. Upon cell activation, PKC isotype-specific signals trigger translocation from the cytosol to the membrane and induce conformational changes, which displace the pseudosubstrate moiety from the catalytic domain and enable PKC isotypes to phosphorylate specific protein substrates (Biochem. J. 370:361-371, 2003). Most isoforms are ubiquitously expressed, except PKCγ and PKCθ. While PKCγ is exclusively found in the brain, high protein levels of PKCθ are seen predominantly in hematopoietic cells and skeletal muscle. PKCα and PKCθ as well as PKCβ and PKGδ are functionally important for T and B cell signaling, respectively (Nat. Immunol. 5:785-790, 2004. Curr. Opin. Immunol. 16:367-373, 204. Nature. 416:860-865, 2002). PKCθ plays an essential role in T cell activation because it is the only isoform that is selectively translocated to the T cell/antigen-presenting cell contact site immediately after cell-cell interaction (Nature. 385:83-86, 1997). Furthermore, PKCθ is crucial for IL-2 production, a prerequisite for the proliferation of T cells (Eur. J. Immunol. 30:3645-3654, 2000). PKCθ-deficient mice are defective in NF-κB (Cell Mol. Immunol. 3:263-270, 2006), NFAT and AP-1 activation (Nature, 404 (96776), 402-407, 2000. Journal of Immunology 176:6004-6011, 2006) and are resistant to experimental autoimmune encephalomyelitis (J. Immunol. 176:2872-2879, 2006), collagen-induced arthritis (Journal of Immunology 177 (3), 1886-1893, 2006) and asthma (Journal of Immunology 173 (10), 6440-6447, 2004).

PKCα in T cells is required for proliferation and IFN-γ production (J. Immunol. 176:6004-6011, 2006).

B cells require PKCβ for proper antigen receptor function and PKGδ for the induction of tolerance (Nature. 416:860-865, 2002).

Thus, PKC isoforms in T and B cells are considered attractive therapeutic targets for autoimmune diseases and transplantation (Curr. Opin. Investig. Drugs. 7:432-437, 2006.).
Further, PKCε and PKCγ have been suggested to play a role in nociception and inflammatory pain (J. Pharm. Exp. Ther. Pain 110, 281-289, 2004) and PKCζ has been proposed as an intermediary in the activation of the NF-κB and IL-4/Stat6 pathway (Cell Death Differ. 13: 702, 2006). The NF-κB pathway is important for inflammatory and immune diseases, therefore a PKCζ inhibition may serve to reduce the severity of these type of diseases (Allergol. Int. 55: 245, 2006. J. Biol. Chem. 281: 24124, 2006. Arthritis Rheum. 56: 4074, 2007. J. Interferon Cytokine Res. 27: 622, 2007).

The novel compounds of this invention inhibit the activity of one or more protein kinases and are, therefore, expected to be useful in the treatment of kinase-mediated diseases.

### SUMMARY OF THE INVENTION

In a first embodiment the invention provides compound of Formula (I)

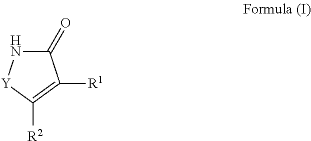

biologically active metabolites, pro-drugs, isomers, stereoisomers, solvates, hydrates and pharmaceutically acceptable salts thereof wherein

Y is —C(R^{c})₂—, —C(═O)—, —C(═S)—, —C(═NR^{e})—, —N(R^{e})—, —O—, —S—, —S(O)—, or —S(O)₂—;

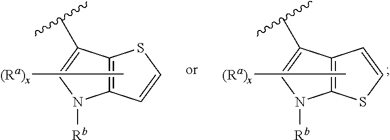

R¹ is

wherein

R^{a} is independently deuterium, halo, —OR^{d}, —CN, —(C₁-C₆)alkoxy, —N(R^{d})₂, —C(O)OR^{d}, —COR^{d}, —N(R^{d})S(O)₂R^{d}, —S(O)₂N(R^{d})₂, —C(O)N(R)₂, —N(R^{d})C(O)R^{d}, —SR^{d}, —S(O)R^{d}, —S(O)₂R^{d}, optionally substituted (C₂-C₆)alkenyl, optionally substituted (C₂-C₆)alkynyl, optionally substituted (C₁-C₆)alkyl, optionally substituted spirocyclic (C₅-C₁₄)cycloalkyl, optionally substituted spirocyclic (C₂-C₁₁)heterocyclyl, optionally substituted (C₃-C₆)cycloalkyl, optionally substituted (C₁-C₁₀)heterocyclyl, optionally substituted (C₆-C₁₀)aryl, optionally substituted (C₁-C₁₀)heteroaryl, optionally substituted bridged (C₅-C₁₂) cycloalkyl, or optionally substituted bridged (C₂-C₁₀)heterocyclyl; or

R^{a} is independently —(C(R^{d})₂)ₓ—B-E-G-J wherein
B is independently a bond, —N(R^{d})—, —O—, —C(O)—, —C(O)O—, —S—, —SO—, —SO₂—, —N(R^{d})S(O)₂—, —S(O)₂N(R^{d})—, —C(O)N(R^{d})—, —N(R^{d})C(O)— or —N(R^{d})C(O)N(R^{d})—;
E is independently a bond, N(R^{d}), optionally substituted (C₁-C₆)alkylene, optionally substituted (C₂-C₆)alkenylene, optionally substituted (C₂-C₆)alkynylene, optionally substituted spirocyclic (C₅-C₁₄)cycloalkylene, optionally substituted spirocyclic (C₃-C₁₁)heterocyclylene, optionally substituted bridged (C₅-C₁₂)cycloalkylene, optionally substituted bridged (C₂-C₁₀)heterocyclylene, optionally substituted (C₃-C₆)cycloalkylene, optionally substituted (C₁-C₁₀)heterocyclylene, optionally substituted (C₆-C₁₀)arylene, or optionally substituted (C₁-C₁₀)heteroarylene;
G is independently a bond, optionally substituted —(C₁-C₆)alkylene-, optionally substituted —(C₂-C₆)alkenylene-, optionally substituted —(C₂-C₆)alkynylene-, —O—, —S—, —S(O)ₚ—, —N(R^{c})—, —N(C(O)OR^{d})—, —N(C(O)R^{d})—, —N(S(O)ₚR^{d})—, —C(R^{d})₂O—, —O—(CR^{d})₂—, —C(R^{d})₂S—, —SC(R^{d})₂—, —C(R^{d})₂N(R^{d})—, —N(R^{d})C(R^{d})₂—, —C(R^{d})₂N(C(O)R^{d})—, —N(C(O)R^{d})C(R^{d})₂—, —C(R^{d})₂N(C(O)OR^{d})—, —N(C(O)OR^{d})C(R^{d})₂—, —C(R^{d})₂N(S(O)ₚR^{d})—, —N(S(O)ₚR^{d})C(R^{d})₂—, —C(R^{d})(N(R^{d})(OR^{d}))—, —C(R^{d})(ON(R^{d})₂)—, —C(R^{d})(N(R^{d})₂)—, —C(R^{d})(N(R^{d})S(O)ₚR^{d})—, —C(R^{d})(S(O)ₚN(R^{d})₂)—, —C(R^{d})(N(R^{d})C(O)OR^{d})—, —CR^{d}(OC(O)R^{d})—, —CR^{d}(C(O)OR^{d})—, —C(R^{d})(OC(O)N(R^{d})₂—, —C(═NOR^{d})—, —C(O)—, —C(O)O—, —C(R^{d})(OR^{d})—, —C(O)N(R^{d})—, —N(R^{d})C(O)—, —N(R^{d})S(O)ₚ—, —S(O)ₚN(R^{d})—, —N(R^{d})C(O)N(R^{d})—, —N(R^{d})S(O)ₚN(R^{d})—, —OC(O)N(R^{d})—, —N(R^{d})C(O)O—, —ON(R^{d})C(O)—, —C(O)N(R^{d})O—, —N(OR^{d})C(O)—, —C(O)N(OR^{d})—, —N(R^{d})—C(O)—(C(R^{d})₂)ₙ₊₁—N(R^{d})—, —N(R^{d})—(C(R^{d})₂)ₙ₊₁—C(O)—N(R^{d})—, —C(O)—N(R^{d})—(C(R^{d})₂)ₙ₊₂—N(R^{d})—, —N(R^{d})—(C(R^{d})₂)ₙ₊₂—N(R^{d})—C(O)—, —N(R^{d})—(C(R^{d})₂)ₙ₊₁—C(O)—, —C(O)—(C(R^{d})₂)ₙ₊₁—N(R^{d})—, —O—(CR^{d})ₙ₊₁—C(O)—, —C(O)—CR^{d})ₙ₊₁—O—, —O—(C(R^{d})₂)ₙ₊₂—O—, —N(R^{d})—C(O)—(CH₂)ₙ₊₁—O—, —O—(C(R^{d})₂)ₙ₊₁—C(O)—N(R^{d})—, —O—(C(R^{d})₂)ₙ₊₂ N(R^{d})—C(O)—, —C(O)—N(R^{d})—(C(R^{d})₂)ₙ₊₂—O—, —O—(C(R^{d})₂)ₙ₊₂N(R^{d})—, —N(R^{d})—(C(R^{d})₂)ₙ₊₂—O—, —N(R^{d})—(C(R^{d})₂)ₙ₊₂—N(R^{d})—, —C(O)N(R^{d})C(O)—, —S(O)ₚN(R^{d})C(O)—, —C(O)N(R^{d})S(O)ₚ—, —OS(O)ₚN(R^{d})—, —N(R^{d})S(O)ₚO—, —N(R^{d})S(O)ₚC(O)—, —C(O)S(O)ₚN(R^{d})—, —S(O)ₚN(C(O)R^{d})—, —N(C(O)R^{d})S(O)ₚ—, —N(S(O)ₚ(R^{d})C(O)—, —C(O)N(S(O)ₚ(R^{d}))—, —N(R^{d})P(O)(OR^{d})—, —N(R^{d})P(O)(OR^{d})O—, —N(C(O)R^{d})P(O)(OR^{d})—, or —N(C(O)R^{d})P(O)(OR^{d})O—;
wherein
n is 0 to 6;
p is 1 or 2;
J is independently H, N(R^{d})₂, optionally substituted (C₁-C₆)alkyl, optionally substituted (C₂-C₆)alkenyl, optionally substituted (C₂-C₆)alkynyl, optionally substituted spirocyclic (C₅-C₁₄)cycloalkyl, optionally substituted spirocyclic (C₃-C₁₄)heterocyclyl, optionally substituted bridged (C₅-C₁₂)cycloalkyl, optionally substituted bridged (C₂-C₁₀)heterocyclyl, optionally substituted (C₃-C₆)cycloalkyl, optionally substituted (C₁-C₁₀)heterocyclyl, optionally substituted (C₆-C₁₀)aryl, or optionally substituted (C₁-C₁₀)heteroaryl;
provided that —B-E-G-J does not form a three atom combination of oxygen atoms, nitrogen atoms or a combination of oxygen and nitrogen atoms directly bound to one another;

R^{b} is independently H, —C(O)R^{d}, —COOR^{d}, —S(O)₂N(R^{d})₂, —C(O)N(R^{d})₂, —S(O)R^{d}, —S(O)₂R^{d}, optionally substituted (C₁-C₆)alkyl, optionally substituted (C₂-C₆)alkenyl, optionally substituted (C₂-C₆)alkynyl, optionally substituted (C₂-C₆)alkoxy, optionally substituted spirocyclic (C₅-C₁₄)cycloalkyl, optionally substituted spirocyclic (C₂-C₁₀)heterocyclyl; optionally substituted (C₃-C₆)cycloalkyl, optionally substituted (C₁-C₁₀)heterocyclyl, optionally substituted (C₆-C₁₀)aryl, optionally substituted (C₁-C₁₀)heteroaryl, optionally substituted bridged (C₂-C₁₀)heterocyclyl, or optionally substituted bridged (C₂-C₁₀)cycloalkyl; or

R^{b} is independently —(C(R^{d})₂)ₓ—B-E-G-J;

R^{c} is independently H, OH, deuterium, F, —O-optionally substituted (C₃-C₆)cycloalkyl, optionally substituted —OC₁-C₆)alkyl, optionally substituted (C₁-C₆)alkyl or optionally substituted (C₃-C₆)cycloalkyl;

R^{d} is independently H, optionally substituted (C₁-C₆)alkyl, optionally substituted (C₂-C₆)alkenyl, optionally substituted (C₂-C₆)alkynyl, optionally substituted (C₃-C₆)cycloalkyl, optionally substituted (C₆-C₁₀)aryl, optionally substituted (C₁-C₁₀)heteroaryl or optionally substituted (C₁-C₁₀)heterocyclyl;

R^{e} is H, optionally substituted (C₁-C₆)alkyl or optionally substituted (C₃-C₆)cycloalkyl;

R² is optionally substituted (C₆-C₁₀)aryl, optionally substituted (C₃-C₆)cycloalkyl, optionally substituted (C₁-C₁₀)heterocyclyl or optionally substituted (C₁-C₁₀)heteroaryl; and

x is 0 to 3.

In a second embodiment the invention provides a compound according to the first embodiment wherein R² is

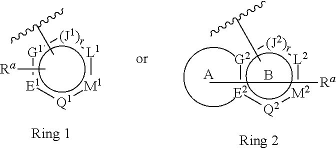

wherein

in Ring 1
r is 1 and E¹, G¹, J¹, L¹, M¹ and Q¹ are each independently C, CR^{a}, N; or
r is 0 and E¹, G¹, L¹, M¹ and Q¹ are each independently C, CR^{a}, NR^{b}, N, S or O; or in Ring 2
Ring A is a five to seven membered optionally substituted ring selected from aryl, heterocyclyl, heteroaryl and cycloalkyl fused to Ring B;
r is 1 and J², L², M² and Q² are each independently C, CR^{a}, or N; and E², and G² are independently C or N; or
r is 0 and L², M² and Q² are each independently C, CR^{a}, N, NR^{b}, S or O and E² and G², are independently C or N; or
when r is 0, L² and M² together or M² and Q² together optionally form a saturated or unsaturated four to seven membered carbocyclic or heterocylic ring, provided that none of L², M² or Q² is independently O or S and only one of L², M² or Q² is N; or
when r is 1, M² and Q² together optionally form a saturated or unsaturated four to seven membered carbocyclic or heterocylic ring, provided that neither M² or Q² is N; or
when r is 1, L² and M² together optionally form a saturated or unsaturated four to seven membered carbocyclic or heterocylic ring, provided that neither L² or M² is N.

In a third embodiment the invention provides a compound according to any of the foregoing embodiments wherein R² is Ring 1 and Ring 1 is

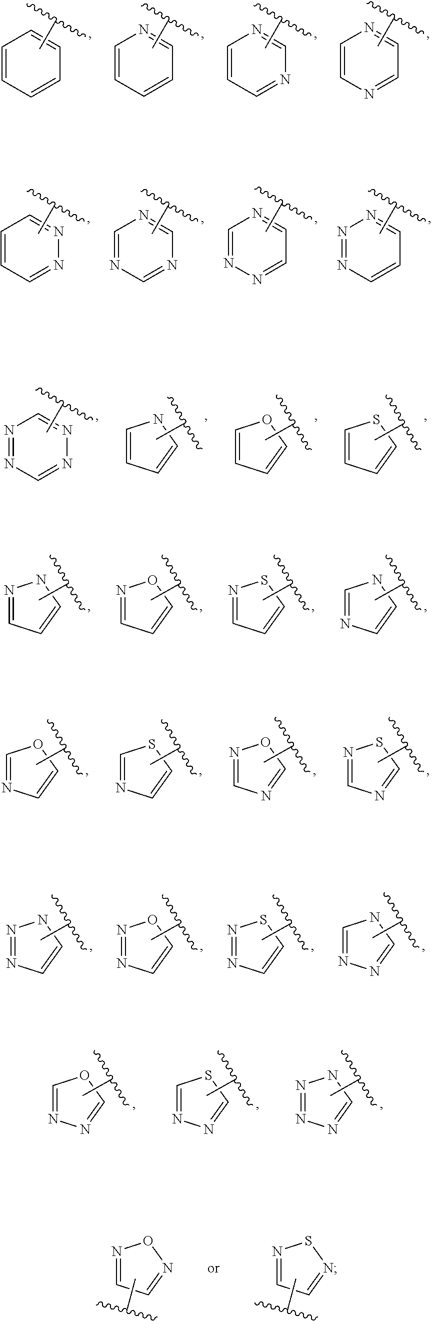

wherein carbon atoms in Ring 1 are optionally substituted by R^{a} and nitrogen atoms are optionally substituted by R^{b};

or R² is Ring 2 and Ring 2 is

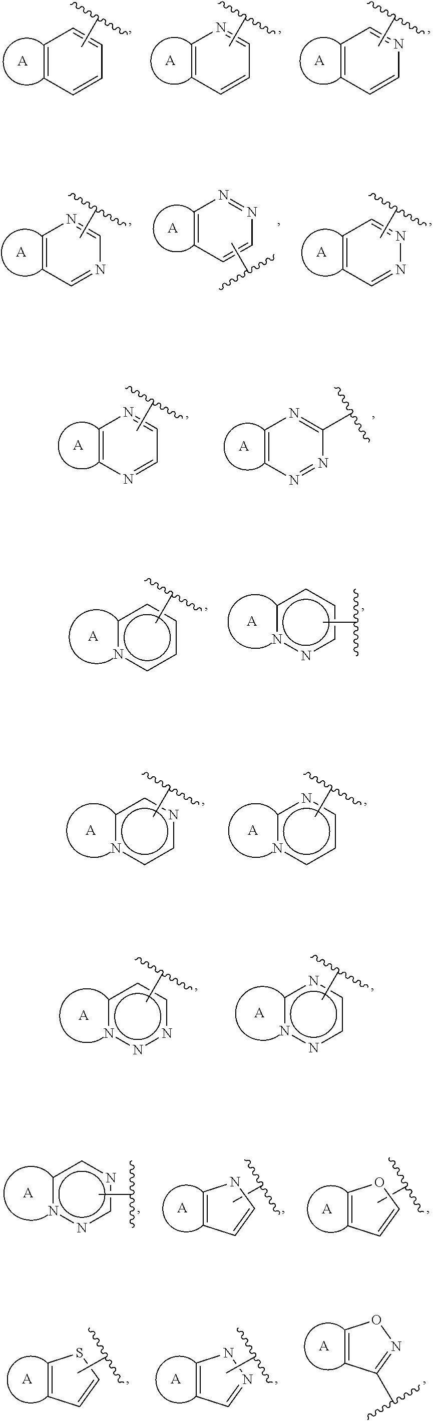

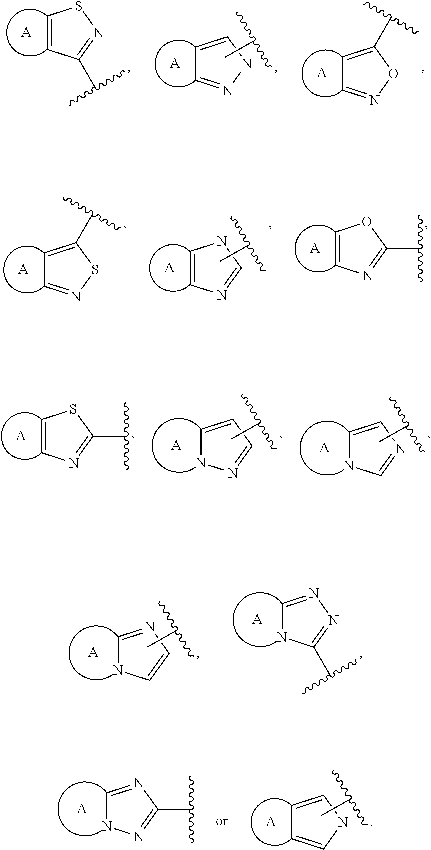

In a fourth embodiment the invention provides a compound according to any of the foregoing embodiments wherein Ring A is

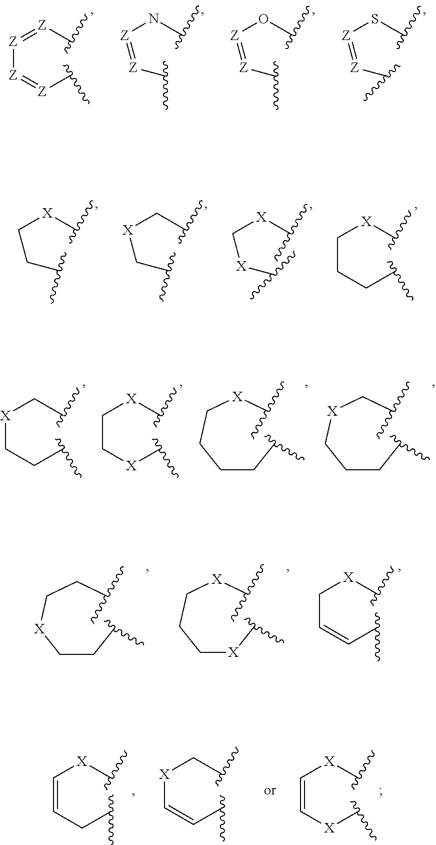

wherein X is independently —S—, —SO—, —SO₂—, —O—, —N(R^{b})—, or —C(R^{a})₂— and when X is N(R^{b}) then an adjacent carbon atom can be optionally substituted with oxo; and

Z is independently C, C(R^{a}) or N;

and carbon atoms in Ring A are optionally substituted by R^{a} and nitrogen atoms are optionally substituted by R^{b}.

In a fifth embodiment the invention provides a compound according to any of the foregoing embodiments wherein R² is

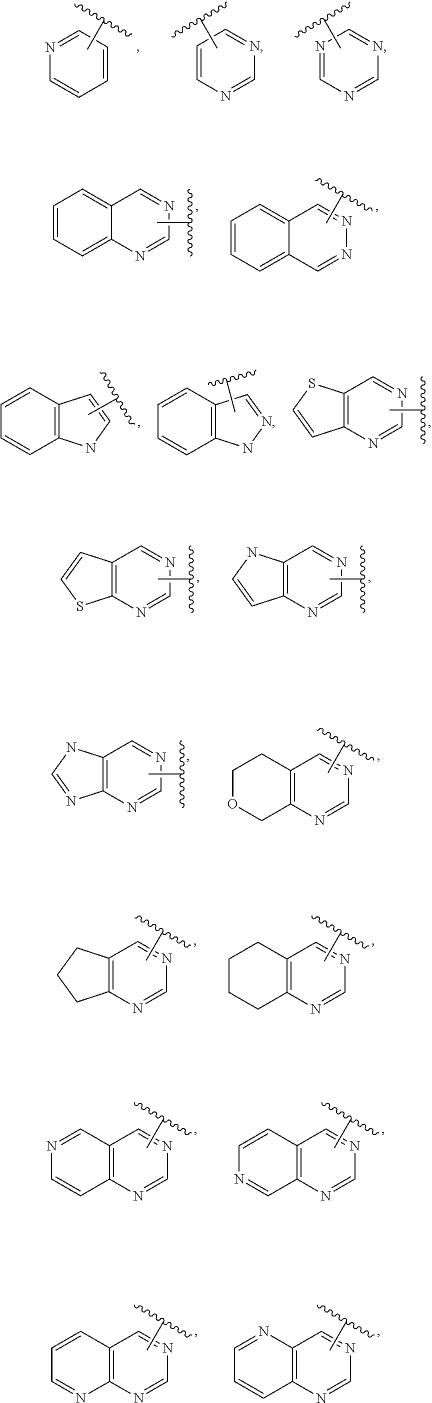

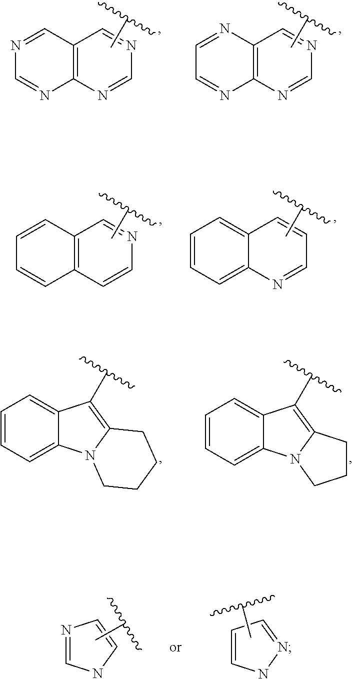

and carbon atoms in R² are independently optionally substituted by R^{a} and nitrogen atoms are optionally substituted by R^{b}.

In a sixth embodiment the invention provides a compound according to any of the foregoing embodiments wherein R^{a} is optionally substituted (C₁-C₆)alkyl or

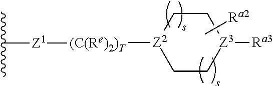

wherein

Z¹ is a bond or —N(R^{e});

Z² is CR^{a1} or N;

Z³ is CR^{a4} or N; or

Z³ is O and R^{a3} is not present;
R^{a1} is H or optionally substituted (C₁-C₆)alkyl;
R^{a2} and R^{a3} are each independently H, —CN, —CF₃, —OH, (C₁-C₆)alkoxy, optionally substituted (C₃-C₆) cycloalkyl, —C(O)—N(R^{e})(R^{f}), F, —N(R^{e})(R^{f}), optionally substituted (C₁-C₆)alkyl; optionally substituted (C₃-C₆)cycloalkyl, —(C(R^{e})₂)ₘ-optionally substituted heterocyclyl, —(C(R^{e})₂)ₘ-optionally substituted heteroaryl;
   provided that when Z³ is N then R^{a3} is not —CN or F;
   wherein R^{e} and R^{f} are independently H, optionally substituted (C₁-C₆)alkyl or optionally substituted (C₃-C₆)cycloalkyl; or
   —N(R^{e})(R^{f}) can form an optionally substituted 4-, 5- or 6-membered saturated or unsaturated heterocyclic ring;
R^{4a} is H, optionally substituted (C₁-C₆)alkyl or optionally substituted (C₃-C₆)cycloalkyl; or
R^{a1} and R^{a2} combine with the atoms to which they are attached to form a 4-, 5- or 6-membered optionally substituted saturated or unsaturated carbocyclic or optionally substituted saturated or unsaturated heterocylic ring; or
R^{a2} and R^{a3} combine with the atoms to which they are attached to form a 4-, 5- or 6-membered saturated or unsaturated carbocyclic or optionally substituted saturated or unsaturated heterocylic ring; or
R^{a3} and R^{a4} form a 4-, 5- or 6-membered optionally substituted saturated carbocyclic or heterocyclic ring to form a spirocyclic moiety;
m is 0, 1 or 2;
s is independently 0, 1, or 2; and

T is 0, 1, 2 or 3.

In a seventh embodiment the invention provides a compound according to any of the foregoing embodiments wherein R^{a} is

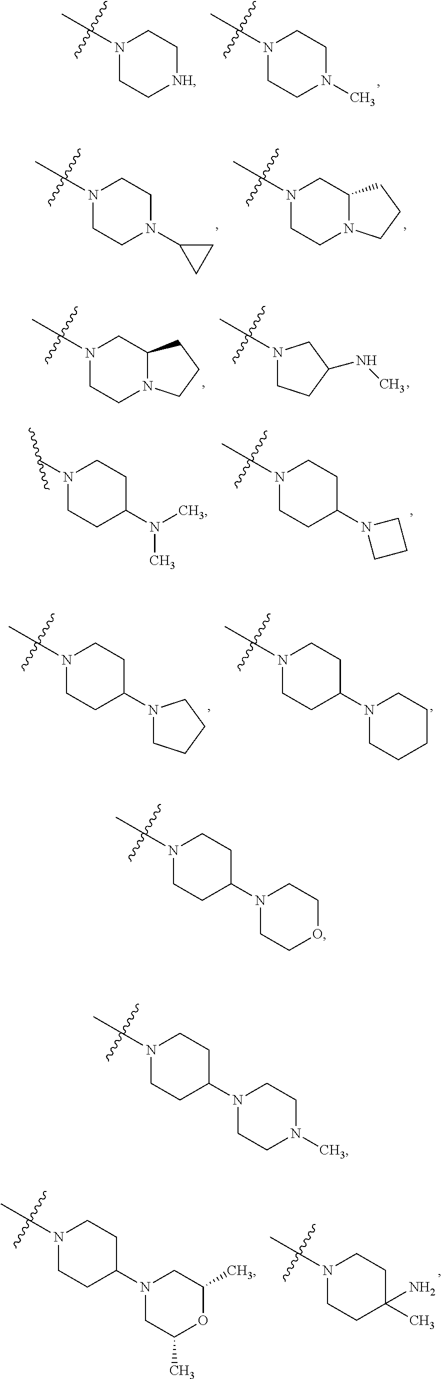

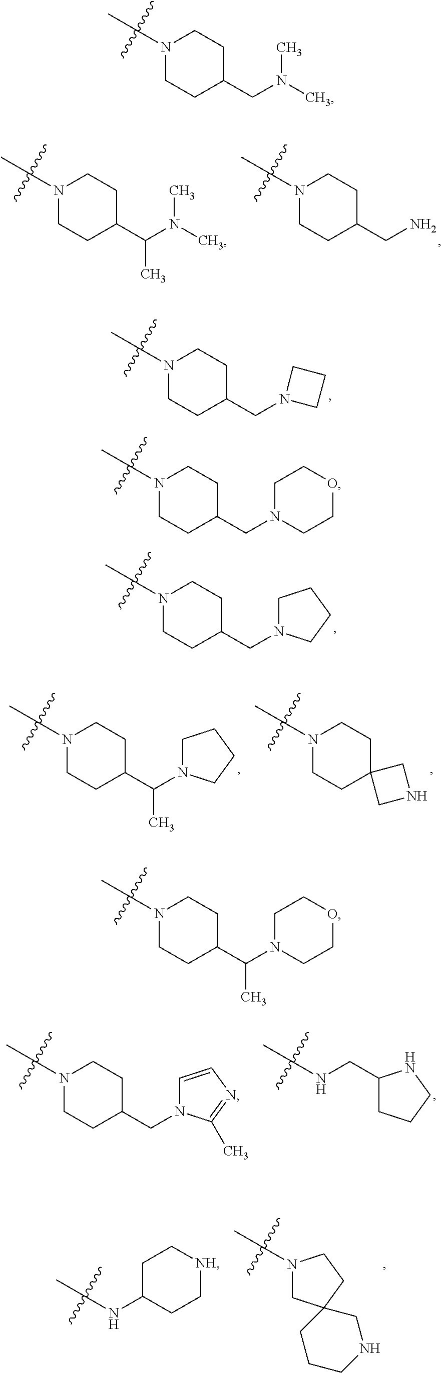

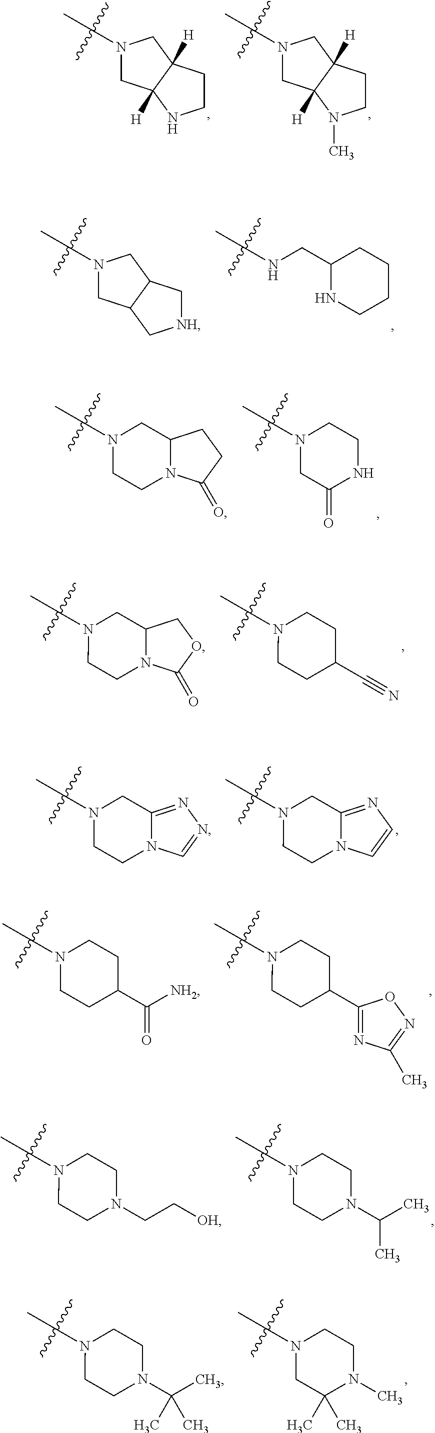

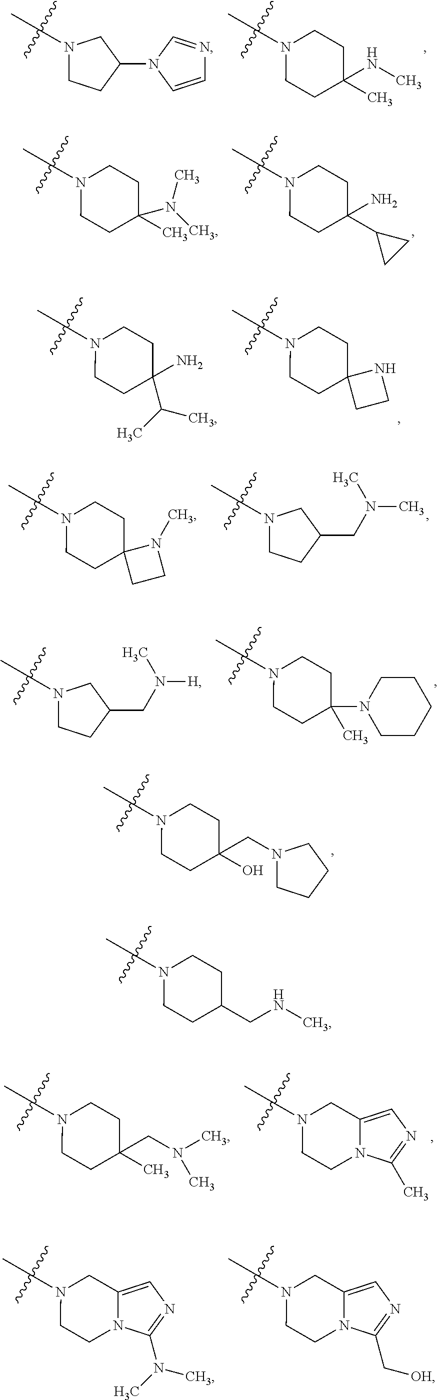

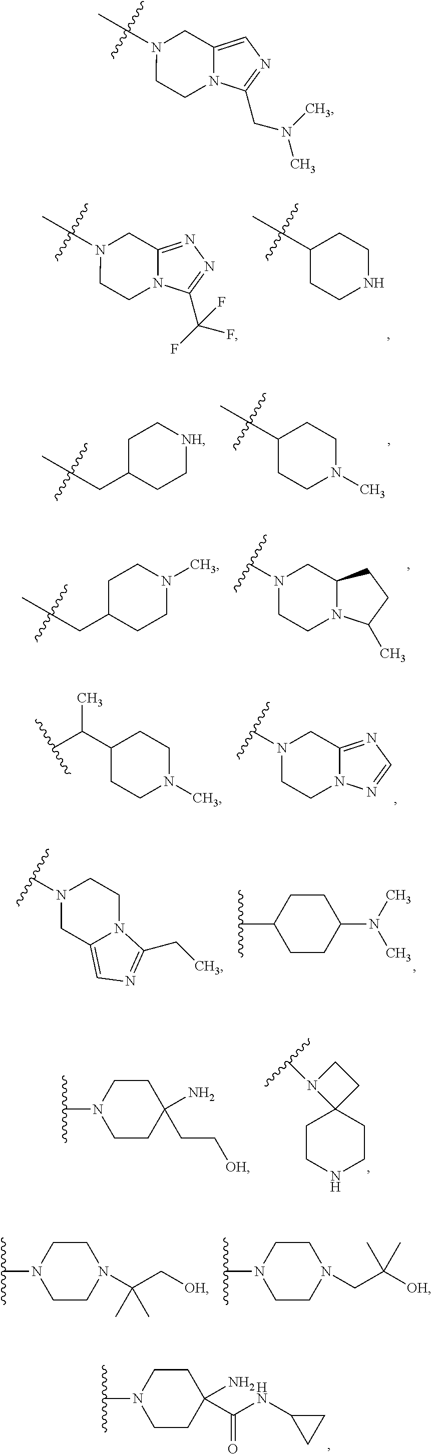

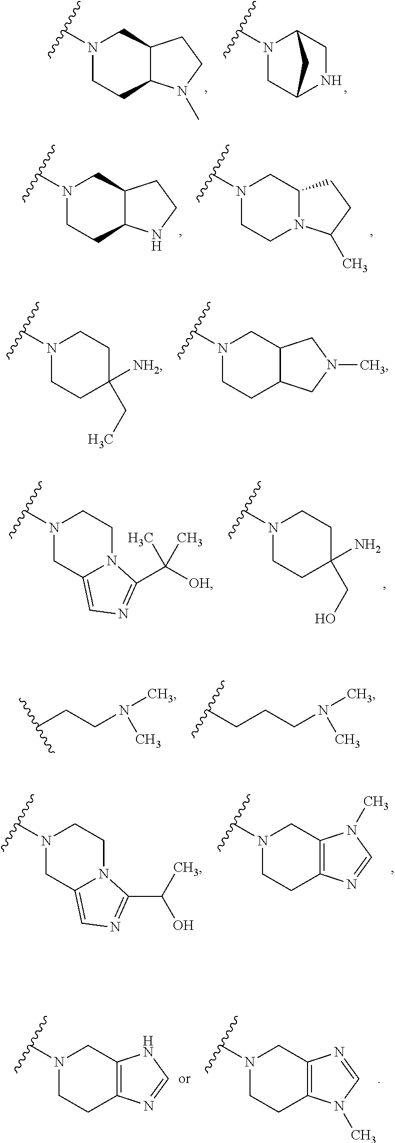

wherein R^{a} is optionally substituted by —(CH₂)_{T}CF₃, —(CH₂)_{T}CHF₂, —(CH₂)_{T}CH₂F, —F, —(CH₂)_{T}OH, —CH₂C(CH₃)₂OH, —C(CH₃)₂CH₂OH, —OCH₃, —OCF₃, —O(CH₂)_{T}CH₃, —(CH₂)_{T}CH₃, —(CH₂)_{T}OCH₃, —(CH₂)_{T}OC(CH₃)₃, —(CH₂)_{T}OCH(CH₃)₂, —(CH₂)_{T}OCH₂CH₃, —(CH₂)_{T}OCF₃, —(CF₂)_{T}CF₃, —(CF₂)_{T}CHF₂, —(CF₂)_{T}CH₂F, —(CHF)_{T}CF₃, —(CHF)_{T}CHF₂, —(CHF)_{T}CH₂F, —(CH₂)_{T}CH₃, —CH(CH₃)₂, —C(CH₃)₃, —(CH₂)_{T}CN, —(CH₂)_{T}NH₂, —(CH₂)_{T}NHCH₃, —(CH₂)_{T}N(CH₃)₂, —(CH₂)_{T}CONH₂, —(CH₂)_{T} CONHCH₃, —CON(CH₃)₂ or optionally substituted (C₃-C₆)cycloalkyl; and

T is 0, 1, 2 or 3

In an eighth embodiment the invention provides a compound according to any of the foregoing embodiments wherein R^{a} is (C₁-C₆)alkyl optionally substituted by one or more substituents selected from the group consisting of F, OH, (C₁-C₃)alkoxy, —NH₂, —N(H)CH₃, —N(CH₃)₂, —C(O)NH₂, —C(O)N(H)CH₃, —C(O)(C₃-C₆)cycloalkyl.

In a ninth embodiment the invention provides a compound according to any of the foregoing embodiments wherein R^{b} is

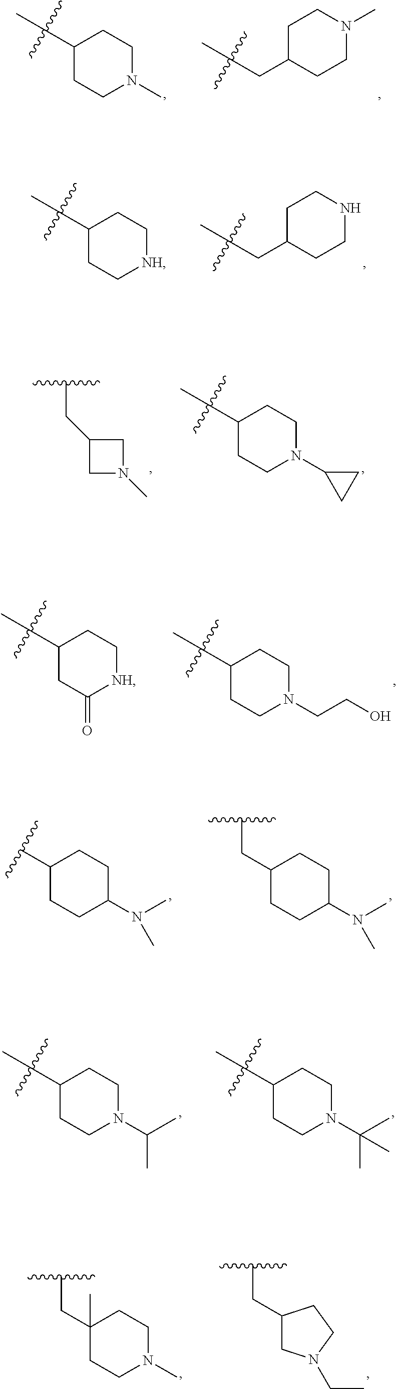

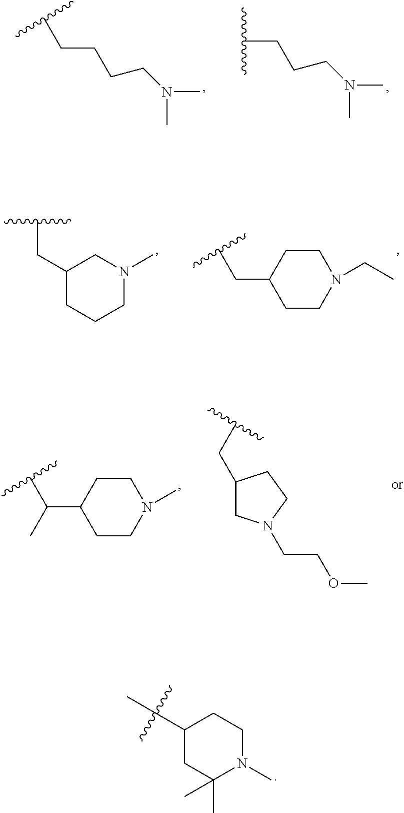

wherein R^{b} is optionally substituted by —(CH₂)_{T}CF₃, —(CH₂)_{T}CHF₂, —(CH₂)_{T}CH₂F, —F, —(CH₂)_{T}OH, —CH₂C(CH₃)₂OH, —C(CH₃)₂CH₂OH, —OCH₃, —OCF₃, —O(CH₂)_{T}CH₃, —(CH₂)_{T}CH₃, —(CH₂)_{T}OCH₃, —(CH₂)_{T}OC(CH₃)₃, —(CH₂)_{T}OCH(CH₃)₂, —(CH₂)_{T}OCH₂CH₃, —(CH₂)_{T}OCF₃, —(CF₂)_{T}CF₃, —(CF₂)_{T}CHF₂, —(CF₂)_{T}CH₂F, —(CHF)_{T}CF₃, —(CHF)_{T}CHF₂, —(CHF)_{T}CH₂F, —(CH₂)_{T}CH₃, CH(CH₃)₂, —C(CH₃)₃, —(CH₂)_{T}CN, —(CH₂)_{T}NH₂, —(CH₂)_{T}NHCH₃, —(CH₂)_{T}N(CH₃)₂, —(CH₂)_{T}CONH₂, —(CH₂)_{T}CONHCH₃, or —CON(CH₃)_{2 2} or optionally substituted (C₃-C₆)cycloalkyl.

In a tenth embodiment the invention provides a compound according to any of the foregoing embodiments wherein Y is —C(R^{c})₂—, —C(═O)—, —C(═S)—, —C(═NR^{e})—, or —S(O)—.

In an eleventh embodiment the invention provides a compound according to any of the foregoing embodiments compound of claim 9 wherein R² is

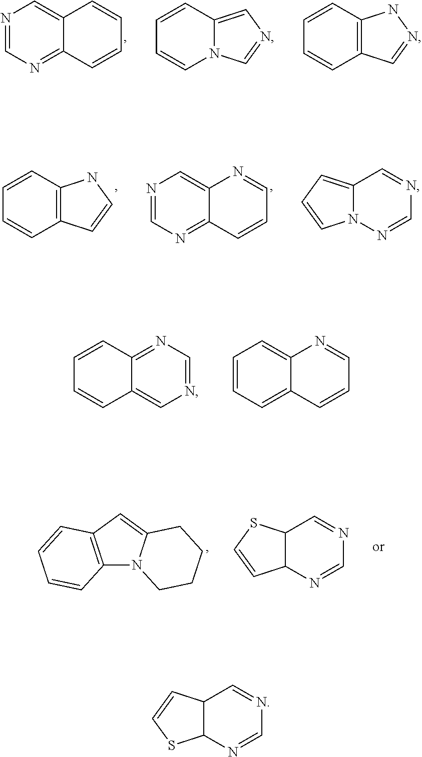

In a twelfth embodiment the invention provides a compound according to the first embodiment wherein the compound is
(R)-3-(2-(hexahydropyrrolo[1,2-a]pyrazin-2(1H)-yl)quinazolin-4-yl)-4-(6H-thieno[2,3-b]pyrrol-4-yl)-1H-pyrrole-2,5-dione;
3-(2-(4-(1-morpholinoethyl)piperidin-1-yl)quinazolin-4-yl)-4-(6H-thieno[2,3-b]pyrrol-4-yl)-1H-pyrrole-2,5-dione;
3-(2-(4-((2-methyl-1H-imidazol-1-yl)methyl)piperidin-1-yl)quinazolin-4-yl)-4-(6H-thieno[2,3-b]pyrrol-4-yl)-1H-pyrrole-2,5-dione;
3-(2-(4-(1-(pyrrolidin-1-yl)ethyl)piperidin-1-yl)quinazolin-4-yl)-4-(6H-thieno[2,3-b]pyrrol-4-yl)-1H-pyrrole-2,5-dione;
3-(2-(4-((dimethylamino)methyl)piperidin-1-yl)quinazolin-4-yl)-4-(6H-thieno[2,3-b]pyrrol-4-yl)-1H-pyrrole-2,5-dione;
3-(2-(4-((2R,6S)-2,6-dimethylmorpholino)piperidin-1-yl)quinazolin-4-yl)-4-(6H-thieno[2,3-b]pyrrol-4-yl)-1H-pyrrole-2,5-dione;
3-(2-(3-oxopiperazin-1-yl)quinazolin-4-yl)-4-(6H-thieno[2,3-b]pyrrol-4-yl)-1H-pyrrole-2,5-dione;
3-(2-(4-(4-methylpiperazin-1-yl)piperidin-1-yl)quinazolin-4-yl)-4-(6H-thieno[2,3-b]pyrrol-4-yl)-1H-pyrrole-2,5-dione;
1-(4-(2,5-dioxo-4-(6H-thieno[2,3-b]pyrrol-4-yl)-2,5-dihydro-1H-pyrrol-3-yl)quinazolin-2-yl)piperidine-4-carbonitrile;
3-(2-(4-(3-methyl-1,2,4-oxadiazol-5-yl)piperidin-1-yl)quinazolin-4-yl)-4-(6H-thieno[2,3-b]pyrrol-4-yl)-1H-pyrrole-2,5-dione;
3-(2-(4-(pyrrolidin-1-ylmethyl)piperidin-1-yl)quinazolin-4-yl)-4-(6H-thieno[2,3-b]pyrrol-4-yl)-1H-pyrrole-2,5-dione;
3-(2-(4-(1-(dimethylamino)ethyl)piperidin-1-yl)quinazolin-4-yl)-4-(6H-thieno[2,3-b]pyrrol-4-yl)-1H-pyrrole-2,5-dione;
3-(2-(4-(azetidin-1-yl)piperidin-1-yl)quinazolin-4-yl)-4-(6H-thieno[2,3-b]pyrrol-4-yl)-1H-pyrrole-2,5-dione;
1-(4-(2,5-dioxo-4-(6H-thieno[2,3-b]pyrrol-4-yl)-2,5-dihydro-1H-pyrrol-3-yl)quinazolin-2-yl)piperidine-4-carboxamide;
3-(2-(3-oxodihydro-1H-oxazolo[3,4-a]pyrazin-7(3H,8H,8aH)-yl)quinazolin-4-yl)-4-(6H-thieno[2,3-b]pyrrol-4-yl)-1H-pyrrole-2,5-dione;
3-(2-(4-morpholinopiperidin-1-yl)quinazolin-4-yl)-4-(6H-thieno[2,3-b]pyrrol-4-yl)-1H-pyrrole-2,5-dione;
3-(2-(4-(azetidin-1-ylmethyl)piperidin-1-yl)quinazolin-4-yl)-4-(6H-thieno[2,3-b]pyrrol-4-yl)-1H-pyrrole-2,5-dione;
3-(2-(4-cyclopropylpiperazin-1-yl)quinazolin-4-yl)-4-(6H-thieno[2,3-b]pyrrol-4-yl)-1H-pyrrole-2,5-dione;
3-(2-(4-(2-hydroxyethyl)piperazin-1-yl)quinazolin-4-yl)-4-(6H-thieno[2,3-b]pyrrol-4-yl)-1H-pyrrole-2,5-dione;
3-(6H-thieno[2,3-b]pyrrol-4-yl)-4-(2-(3-(trifluoromethyl)-5,6-dihydro-[1,2,4]triazolo[4,3-a]pyrazin-7(8H)-yl)quinazolin-4-yl)-1H-pyrrole-2,5-dione;
3-(2-(4-isopropylpiperazin-1-yl)quinazolin-4-yl)-4-(6H-thieno[2,3-b]pyrrol-4-yl)-1H-pyrrole-2,5-dione;
3-(2-(4-tert-butylpiperazin-1-yl)quinazolin-4-yl)-4-(6H-thieno[2,3-b]pyrrol-4-yl)-1H-pyrrole-2,5-dione;
3-(2-(3-(1H-imidazol-1-yl)pyrrolidin-1-yl)quinazolin-4-yl)-4-(6H-thieno[2,3-b]pyrrol-4-yl)-1H-pyrrole-2,5-dione;
3-(6H-thieno[2,3-b]pyrrol-4-yl)-4-(2-(3,3,4-trimethylpiperazin-1-yl)quinazolin-4-yl)-1H-pyrrole-2,5-dione;
3-(2-(4-hydroxy-4-(pyrrolidin-1-ylmethyl)piperidin-1-yl)quinazolin-4-yl)-4-(6H-thieno[2,3-b]pyrrol-4-yl)-1H-pyrrole-2,5-dione;
3-(2-(3-methyl-5,6-dihydroimidazo[1,5-a]pyrazin-7(8H)-yl)quinazolin-4-yl)-4-(6H-thieno[2,3-b]pyrrol-4-yl)-1H-pyrrole-2,5-dione;
3-(2-(6,7-dihydro-3H-imidazo[4,5-c]pyridin-5 (4H)-yl)quinazolin-4-yl)-4-(6H-thieno[2,3-b]pyrrol-4-yl)-1H-pyrrole-2,5-dione;
3-(2-(3-((dimethylamino)methyl)pyrrolidin-1-yl)quinazolin-4-yl)-4-(6H-thieno[2,3-b]pyrrol-4-yl)-1H-pyrrole-2,5-dione;
3-(2-(1,4′-bipiperidin-1′-yl)quinazolin-4-yl)-4-(4H-thieno[3,2-b]pyrrol-6-yl)-1H-pyrrole-2,5-dione;
3-(2-(4-(dimethylamino)piperidin-1-yl)quinazolin-4-yl)-4-(4H-thieno[3,2-b]pyrrol-6-yl)-1H-pyrrole-2,5-dione;
(R)-3-(2-(hexahydropyrrolo[1,2-a]pyrazin-2(1H)-yl)quinazolin-4-yl)-4-(4H-thieno[3,2-b]pyrrol-6-yl)-1H-pyrrole-2,5-dione;
(S)-3-(2-(hexahydropyrrolo[1,2-a]pyrazin-2(1H)-yl)quinazolin-4-yl)-4-(4H-thieno[3,2-b]pyrrol-6-yl)-1H-pyrrole-2,5-dione;
3-(2-(4-morpholinopiperidin-1-yl)quinazolin-4-yl)-4-(4H-thieno[3,2-b]pyrrol-6-yl)-1H-pyrrole-2,5-dione;
3-(2-(4-(pyrrolidin-1-yl)piperidin-1-yl)quinazolin-4-yl)-4-(4H-thieno[3,2-b]pyrrol-6-yl)-1H-pyrrole-2,5-dione;
3-(2-(5,6-dihydroimidazo[1,2-a]pyrazin-7(8H)-yl)quinazolin-4-yl)-4-(4H-thieno[3,2-b]pyrrol-6-yl)-1H-pyrrole-2,5-dione;
3-(2-(5,6-dihydro-[1,2,4]triazolo[4,3-a]pyrazin-7(8H)-yl)quinazolin-4-yl)-4-(4H-thieno[3,2-b]pyrrol-6-yl)-1H-pyrrole-2,5-dione;
3-(2-(6-oxohexahydropyrrolo[1,2-a]pyrazin-2(1H)-yl)quinazolin-4-yl)-4-(4H-thieno[3,2-b]pyrrol-6-yl)-1H-pyrrole-2,5-dione;
3-(2-((3aS,6aS)-1-methylhexahydropyrrolo[3,4-b]pyrrol-5(1H)-yl)quinazolin-4-yl)-4-(4H-thieno[3,2-b]pyrrol-6-yl)-1H-pyrrole-2,5-dione;
3-(2-(4-methylpiperazin-1-yl)quinazolin-4-yl)-4-(6H-thieno[2,3-b]pyrrol-4-yl)-1H-pyrrole-2,5-dione;
3-(2-(4-methylpiperazin-1-yl)quinazolin-4-yl)-4-(4H-thieno[3,2-b]pyrrol-6-yl)-1H-pyrrole-2,5-dione;
3-(2-methyl-6H-thieno[2,3-b]pyrrol-4-yl)-4-(2-(4-methylpiperazin-1-yl)quinazolin-4-yl)-1H-pyrrole-2,5-dione;
3-(2-(4-methylpiperazin-1-yl)pyrimidin-4-yl)-4-(6H-thieno[2,3-b]pyrrol-4-yl)-1H-pyrrole-2,5-dione;
3-(2-(hexahydropyrrolo[1,2-a]pyrazin-2(1H)-yl)quinazolin-4-yl)-4-(6H-thieno[2,3-b]pyrrol-4-yl)-1H-pyrrole-2,5-dione;
3-(1-(1-methylpiperidin-4-yl)-1H-indazol-3-yl)-4-(6H-thieno[2,3-b]pyrrol-4-yl)-1H-pyrrole-2,5-dione;
3-(1-((1-methylpiperidin-4-yl)methyl)-1H-indazol-3-yl)-4-(6H-thieno[2,3-b]pyrrol-4-yl)-1H-pyrrole-2,5-dione;
3-(2-(piperazin-1-yl)quinazolin-4-yl)-4-(4H-thieno[3,2-b]pyrrol-6-yl)-1H-pyrrole-2,5-dione;
3-(2-((1S,4S)-2,5-diazabicyclo[2.2.1]heptan-2-yl)quinazolin-4-yl)-4-(6H-thieno[2,3-b]pyrrol-4-yl)-1H-pyrrole-2,5-dione;
3-(2-(hexahydropyrrolo[3,4-c]pyrrol-2(1H)-yl)quinazolin-4-yl)-4-(4H-thieno[3,2-b]pyrrol-6-yl)-1H-pyrrole-2,5-dione;
3-(2-(4-(aminomethyl)piperidin-1-yl)quinazolin-4-yl)-4-(4H-thieno[3,2-b]pyrrol-6-yl)-1H-pyrrole-2,5-dione;
3-(2-(piperazin-1-yl)quinazolin-4-yl)-4-(6H-thieno[2,3-b]pyrrol-4-yl)-1H-pyrrole-2,5-dione;
3-(2-(2,7-diazaspiro[4.5]decan-2-yl)quinazolin-4-yl)-4-(4H-thieno[3,2-b]pyrrol-6-yl)-1H-pyrrole-2,5-dione;
3-(2-(3-(methylamino)pyrrolidin-1-yl)quinazolin-4-yl)-4-(6H-thieno[2,3-b]pyrrol-4-yl)-1H-pyrrole-2,5-dione;
3-(2-((3aS,6aS)-hexahydropyrrolo[3,4-b]pyrrol-5(1H)-yl)quinazolin-4-yl)-4-(4H-thieno[3,2-b]pyrrol-6-yl)-1H-pyrrole-2,5-dione;
3-(2-(piperidin-4-ylamino)quinazolin-4-yl)-4-(4H-thieno[3,2-b]pyrrol-6-yl)-1H-pyrrole-2,5-dione;
3-(2-(piperidin-2-ylmethylamino)quinazolin-4-yl)-4-(4H-thieno[3,2-b]pyrrol-6-yl)-1H-pyrrole-2,5-dione;
3-(2-(4-amino-4-methylpiperidin-1-yl)quinazolin-4-yl)-4-(4H-thieno[3,2-b]pyrrol-6-yl)-1H-pyrrole-2,5-dione;
3-(1-(piperidin-4-yl)-1H-indazol-3-yl)-4-(6H-thieno[2,3-b]pyrrol-4-yl)-1H-pyrrole-2,5-dione;
3-(1-(piperidin-4-ylmethyl)-1H-indazol-3-yl)-4-(6H-thieno[2,3-b]pyrrol-4-yl)-1H-pyrrole-2,5-dione;
3-(2-(pyrrolidin-2-ylmethylamino)quinazolin-4-yl)-4-(6H-thieno[2,3-b]pyrrol-4-yl)-1H-pyrrole-2,5-dione;
3-(2-(2,7-diazaspiro[3.5]nonan-7-yl)quinazolin-4-yl)-4-(6H-thieno[2,3-b]pyrrol-4-yl)-1H-pyrrole-2,5-dione;
3-(2-(3-((methylamino)methyl)pyrrolidin-1-yl)quinazolin-4-yl)-4-(6H-thieno[2,3-b]pyrrol-4-yl)-1H-pyrrole-2,5-dione;
3-(2-(4-((methylamino)methyl)piperidin-1-yl)quinazolin-4-yl)-4-(6H-thieno[2,3-b]pyrrol-4-yl)-1H-pyrrole-2,5-dione;
3-(6H-thieno[2,3-b]pyrrol-4-yl)-4-(1-((1-(2,2,2-trifluoroethyl)piperidin-4-yl)methyl)-1H-indol-3-yl)-1H-pyrrole-2,5-dione;
3-(1-((1-(2,2-difluoroethyl)piperidin-4-yl)methyl)-1H-indol-3-yl)-4-(6H-thieno[2,3-b]pyrrol-4-yl)-1H-pyrrole-2,5-dione;
3-(1-((1-(2,2-difluoroethyl)piperidin-4-yl)methyl)-1H-indazol-3-yl)-4-(6H-thieno[2,3-b]pyrrol-4-yl)-1H-pyrrole-2,5-dione;
3-(6H-thieno[2,3-b]pyrrol-4-yl)-4-(1-((1-(2,2,2-trifluoroethyl)piperidin-4-yl)methyl)-1H-indazol-3-yl)-1H-pyrrole-2,5-dione;
3-(1-((1-(2-fluoroethyl)piperidin-4-yl)methyl)-1H-indazol-3-yl)-4-(6H-thieno[2,3-b]pyrrol-4-yl)-1H-pyrrole-2,5-dione;
3-(1-((1-(2-fluoroethyl)piperidin-4-yl)methyl)-1H-indol-3-yl)-4-(6H-thieno[2,3-b]pyrrol-4-yl)-1H-pyrrole-2,5-dione;
3-(1-((1-(2-methoxyethyl)piperidin-4-yl)methyl)-1H-indol-3-yl)-4-(6H-thieno[2,3-b]pyrrol-4-yl)-1H-pyrrole-2,5-dione;
3-(1-((1-(2-methoxyethyl)piperidin-4-yl)methyl)-1H-indazol-3-yl)-4-(6H-thieno[2,3-b]pyrrol-4-yl)-1H-pyrrole-2,5-dione;
3-(1-((4-fluoro-1-methylpiperidin-4-yl)methyl)-1H-indazol-3-yl)-4-(6H-thieno[2,3-b]pyrrol-4-yl)-1H-pyrrole-2,5-dione;
3-(1-((4-fluoro-1-methylpiperidin-4-yl)methyl)-1H-indol-3-yl)-4-(6H-thieno[2,3-b]pyrrol-4-yl)-1H-pyrrole-2,5-dione;
3-(1-((3-fluoro-1-methylpiperidin-4-yl)methyl)-1H-indol-3-yl)-4-(6H-thieno[2,3-b]pyrrol-4-yl)-1H-pyrrole-2,5-dione;
3-(1-((3-fluoro-1-methylpiperidin-4-yl)methyl)-1H-indazol-3-yl)-4-(6H-thieno[2,3-b]pyrrol-4-yl)-1H-pyrrole-2,5-dione;
3-(1-((3-fluoropiperidin-4-yl)methyl)-1H-indazol-3-yl)-4-(6H-thieno[2,3-b]pyrrol-4-yl)-1H-pyrrole-2,5-dione;
3-(1-((3-fluoropiperidin-4-yl)methyl)-1H-indol-3-yl)-4-(6H-thieno[2,3-b]pyrrol-4-yl)-1H-pyrrole-2,5-dione;
3-(1-((2-(hydroxymethyl)piperidin-4-yl)methyl)-1H-indol-3-yl)-4-(6H-thieno[2,3-b]pyrrol-4-yl)-1H-pyrrole-2,5-dione;
3-(1-((2-(hydroxymethyl)piperidin-4-yl)methyl)-1H-indazol-3-yl-4-(6H-thieno[2,3-b]pyrrol-4-yl)-1H-pyrrole-2,5-dione;
3-(1-((2-(hydroxymethyl)-1-methylpiperidin-4-yl)methyl)-1H-indazol-3-yl)-4-(6H-thieno[2,3-b]pyrrol-4-yl)-1H-pyrrole-2,5-dione;
3-(1-((2-(hydroxymethyl)-1-methylpiperidin-4-yl)methyl)-1H-indol-3-yl)-4-(6H-thieno[2,3-b]pyrrol-4-yl)-1H-pyrrole-2,5-dione;
3-(6H-thieno[2,3-b]pyrrol-4-yl)-4-(1-((2-((trifluoromethoxy)methyl)piperidin-4-yl)methyl)-1H-indazol-3-yl)-1H-pyrrole-2,5-dione;
3-(6H-thieno[2,3-b]pyrrol-4-yl)-4-(1-((2-((trifluoromethoxy)methyl)piperidin-4-yl)methyl)-1H-indol-3-yl)-1H-pyrrole-2,5-dione;
3-(1-((1-methyl-2-((trifluoromethoxy)methyl)piperidin-4-yl)methyl)-1H-indol-3-yl)-4-(6H-thieno[2,3-b]pyrrol-4-yl)-1H-pyrrole-2,5-dione;
3-(1-((1-methyl-2-((trifluoromethoxy)methyl)piperidin-4-yl)methyl)-1H-indazol-3-yl)-4-(6H-thieno[2,3-b]pyrrol-4-yl)-1H-pyrrole-2,5-dione;
3-(2-(4-ethyl-3-hydroxy-4-(methylamino)piperidin-1-yl)quinazolin-4-yl)-4-(6H-thieno[2,3-b]pyrrol-4-yl)-1H-pyrrole-2,5-dione;
3-(2-(4-ethyl-3-methoxy-4-(methylamino)piperidin-1-yl)quinazolin-4-yl)-4-(6H-thieno[2,3-b]pyrrol-4-yl)-1H-pyrrole-2,5-dione;
3-(2-(4-ethyl-4-(methylamino)-3-(trifluoromethoxy)piperidin-1-yl)quinazolin-4-yl)-4-(6H-thieno[2,3-b]pyrrol-4-yl)-1H-pyrrole-2,5-dione;
3-(2-(4-amino-3-hydroxy-4-methylpiperidin-1-yl)quinazolin-4-yl)-4-(6H-thieno[2,3-b]pyrrol-4-yl)-1H-pyrrole-2,5-dione;
3-(2-(4-amino-3-methoxy-4-methylpiperidin-1-yl)quinazolin-4-yl)-4-(6H-thieno[2,3-b]pyrrol-4-yl)-1H-pyrrole-2,5-dione;
3-(2-(4-amino-4-methyl-3-(trifluoromethoxy)piperidin-1-yl)quinazolin-4-yl)-4-(6H-thieno[2,3-b]pyrrol-4-yl)-1H-pyrrole-2,5-dione;
3-(2-(3-hydroxy-4-methyl-4-(methylamino)piperidin-1-yl)quinazolin-4-yl)-4-(6H-thieno[2,3-b]pyrrol-4-yl)-1H-pyrrole-2,5-dione;
3-(2-(3-methoxy-4-methyl-4-(methylamino)piperidin-1-yl)quinazolin-4-yl)-4-(6H-thieno[2,3-b]pyrrol-4-yl)-1H-pyrrole-2,5-dione;
3-(2-(4-methyl-4-(methylamino)-3-(trifluoromethoxy)piperidin-1-yl)quinazolin-4-yl)-4-(6H-thieno[2,3-b]pyrrol-4-yl)-1H-pyrrole-2,5-dione;
3-(2-(3-ethyl-5,6-dihydroimidazo[1,5-a]pyrazin-7(8H)-yl)quinazolin-4-yl)-4-(6H-thieno[2,3-b]pyrrol-4-yl)-1H-pyrrole-2,5-dione;
3-(2-(3-isopropyl-5,6-dihydroimidazo[1,5-a]pyrazin-7(8H)-yl)quinazolin-4-yl)-4-(6H-thieno[2,3-b]pyrrol-4-yl)-1H-pyrrole-2,5-dione;
3-(2-(3-cyclopropyl-5,6-dihydroimidazo[1,5-a]pyrazin-7(8H)-yl)quinazolin-4-yl)-4-(6H-thieno[2,3-b]pyrrol-4-yl)-1H-pyrrole-2,5-dione;
3-(1-(morpholin-2-ylmethyl)-1H-indazol-3-yl)-4-(6H-thieno[2,3-b]pyrrol-4-yl)-1H-pyrrole-2,5-dione;
3-(1-(morpholin-2-ylmethyl)-1H-indol-3-yl)-4-(6H-thieno[2,3-b]pyrrol-4-yl)-1H-pyrrole-2,5-dione;
3-(1-((4-methylmorpholin-2-yl)methyl)-1H-indazol-3-yl)-4-(6H-thieno[2,3-b]pyrrol-4-yl)-1H-pyrrole-2,5-dione;
3-(1-((4-methylmorpholin-2-yl)methyl)-1H-indol-3-yl)-4-(6H-thieno[2,3-b]pyrrol-4-yl)-1H-pyrrole-2,5-dione;
3-(2-(3-tert-butyl-5,6-dihydroimidazo[1,5-a]pyrazin-7(8H)-yl)quinazolin-4-yl)-4-(6H-thieno[2,3-b]pyrrol-4-yl)-1H-pyrrole-2,5-dione;
3-(1-((4-fluoropiperidin-4-yl)methyl)-1H-indol-3-yl)-4-(6H-thieno[2,3-b]pyrrol-4-yl)-1H-pyrrole-2,5-dione;
3-(1-((4-fluoropiperidin-4-yl)methyl)-1H-indazol-3-yl)-4-(6H-thieno[2,3-b]pyrrol-4-yl)-1H-pyrrole-2,5-dione;
3-(8-methyl-2-(4-methylpiperazin-1-yl)quinazolin-4-yl)-4-(6H-thieno[2,3-b]pyrrol-4-yl)-1H-pyrrole-2,5-dione;
3-(7-methyl-2-(4-methylpiperazin-1-yl)quinazolin-4-yl)-4-(6H-thieno[2,3-b]pyrrol-4-yl)-1H-pyrrole-2,5-dione;
3-(6-methyl-2-(4-methylpiperazin-1-yl)quinazolin-4-yl)-4-(6H-thieno[2,3-b]pyrrol-4-yl)-1H-pyrrole-2,5-dione;
3-(5-methyl-2-(4-methylpiperazin-1-yl)quinazolin-4-yl)-4-(6H-thieno[2,3-b]pyrrol-4-yl)-1H-pyrrole-2,5-dione;
3-(2-(4-methylpiperazin-1-yl)thieno[3,2-d]pyrimidin-4-yl)-4-(6H-thieno[2,3-b]pyrrol-4-yl)-1H-pyrrole-2,5-dione;
3-(2-(4-methylpiperazin-1-yl)thieno[2,3-d]pyrimidin-4-yl)-4-(6H-thieno[2,3-b]pyrrol-4-yl)-1H-pyrrole-2,5-dione;
3-(1-((1-methylpiperidin-3-yl)methyl)-1H-indazol-3-yl)-4-(6H-thieno[2,3-b]pyrrol-4-yl)-1H-pyrrole-2,5-dione;
3-(1-((1-(2-methoxyethyl)piperidin-3-yl)methyl)-1H-indazol-3-yl)-4-(6H-thieno[2,3-b]pyrrol-4-yl)-1H-pyrrole-2,5-dione;
3-(1-((1-(2-methoxyethyl)pyrrolidin-3-yl)methyl)-1H-indazol-3-yl)-4-(6H-thieno[2,3-b]pyrrol-4-yl)-1H-pyrrole-2,5-dione;
3-(1-(2-(dimethylamino)ethyl)-1H-indazol-3-yl)-4-(6H-thieno[2,3-b]pyrrol-4-yl)-1H-pyrrole-2,5-dione;
3-(1-(2-(dimethylamino)ethyl)-1H-indol-3-yl)-4-(6H-thieno[2,3-b]pyrrol-4-yl)-1H-pyrrole-2,5-dione;
3-(1-(4-(dimethylamino)butyl)-1H-indazol-3-yl)-4-(6H-thieno[2,3-b]pyrrol-4-yl)-1H-pyrrole-2,5-dione;
3-(1-(4-(dimethylamino)butyl)-1H-indol-3-yl)-4-(6H-thieno[2,3-b]pyrrol-4-yl)-1H-pyrrole-2,5-dione;
3-(1-(3-(dimethylamino)propyl)-1H-indazol-3-yl)-4-(6H-thieno[2,3-b]pyrrol-4-yl)-1H-pyrrole-2,5-dione;
3-(1-((2-(methoxymethyl)-1-methylpiperidin-4-yl)methyl)-1H-indol-3-yl)-4-(6H-thieno[2,3-b]pyrrol-4-yl)-1H-pyrrole-2,5-dione;
3-(1-((2-(methoxymethyl)-1-methylpiperidin-4-yl)methyl)-1H-indazol-3-yl)-4-(6H-thieno[2,3-b]pyrrol-4-yl)-1H-pyrrole-2,5-dione;
3-(1-((2-(methoxymethyl)piperidin-4-yl)methyl)-1H-indazol-3-yl)-4-(6H-thieno[2,3-b]pyrrol-4-yl)-1H-pyrrole-2,5-dione;
3-(1-((2-(methoxymethyl)piperidin-4-yl)methyl)-1H-indol-3-yl)-4-(6H-thieno[2,3-b]pyrrol-4-yl)-1H-pyrrole-2,5-dione;
3-(6H-thieno[2,3-b]pyrrol-4-yl)-4-(1-((2-((trifluoromethoxy)methyl)piperidin-4-yl)methyl)-1H-indol-3-yl)-1H-pyrrole-2,5-dione;
3-(1-((1-methyl-2-((trifluoromethoxy)methyl)piperidin-4-yl)methyl)-1H-indazol-3-yl)-4-(6H-thieno[2,3-b]pyrrol-4-yl)-1H-pyrrole-2,5-dione;
3-(2-(4-amino-3-fluoro-4-methylpiperidin-1-yl)quinazolin-4-yl)-4-(6H-thieno[2,3-b]pyrrol-4-yl)-1H-pyrrole-2,5-dione;
3-(2-(3-fluoro-4-methyl-4-(methylamino)piperidin-1-yl)quinazolin-4-yl)-4-(6H-thieno[2,3-b]pyrrol-4-yl)-1H-pyrrole-2,5-dione;
3-(2-(4-ethyl-3-fluoro-4-(methylamino)piperidin-1-yl)quinazolin-4-yl)-4-(6H-thieno[2,3-b]pyrrol-4-yl)-1H-pyrrole-2,5-dione;
3-(2-(4-amino-4-ethyl-3-fluoropiperidin-1-yl)quinazolin-4-yl)-4-(6H-thieno[2,3-b]pyrrol-4-yl)-1H-pyrrole-2,5-dione;
3-(2-(4-amino-3-fluoro-4-isopropylpiperidin-1-yl)quinazolin-4-yl)-4-(6H-thieno[2,3-b]pyrrol-4-yl)-1H-pyrrole-2,5-dione;
3-(2-(3-fluoro-4-isopropyl-4-(methylamino)piperidin-1-yl)quinazolin-4-yl)-4-(6H-thieno[2,3-b]pyrrol-4-yl)-1H-pyrrole-2,5-dione;
3-(2-(4-amino-4-(trifluoromethyl)piperidin-1-yl)quinazolin-4-yl)-4-(6H-thieno[2,3-b]pyrrol-4-yl)-1H-pyrrole-2,5-dione;
3-(2-(4-(methylamino)-4-(trifluoromethyl)piperidin-1-yl)quinazolin-4-yl)-4-(6H-thieno[2,3-b]pyrrol-4-yl)-1H-pyrrole-2,5-dione;
3-(2-(4-amino-4-(hydroxymethyl)piperidin-1-yl)quinazolin-4-yl)-4-(6H-thieno[2,3-b]pyrrol-4-yl)-1H-pyrrole-2,5-dione;
3-(2-(4-(hydroxymethyl)-4-(methylamino)piperidin-1-yl)quinazolin-4-yl)-4-(6H-thieno[2,3-b]pyrrol-4-yl)-1H-pyrrole-2,5-dione;
3-(2-(4-(2-hydroxyethylamino)-4-methylpiperidin-1-yl)quinazolin-4-yl)-4-(6H-thieno[2,3-b]pyrrol-4-yl)-1H-pyrrole-2,5-dione;
3-(2-(4-amino-4-(2-hydroxyethyl)piperidin-1-yl)quinazolin-4-yl)-4-(6H-thieno[2,3-b]pyrrol-4-yl)-1H-pyrrole-2,5-dione;
3-(2-(4-(2-hydroxyethyl)-4-(methylamino)piperidin-1-yl)quinazolin-4-yl)-4-(6H-thieno[2,3-b]pyrrol-4-yl)-1H-pyrrole-2,5-dione;
4-amino-1-(4-(2,5-dioxo-4-(6H-thieno[2,3-b]pyrrol-4-yl)-2,5-dihydro-1H-pyrrol-3-yl)quinazolin-2-yl)piperidine-4-carbonitrile;
1-(4-(2,5-dioxo-4-(6H-thieno[2,3-b]pyrrol-4-yl)-2,5-dihydro-1H-pyrrol-3-yl)quinazolin-2-yl)-4-(methylamino)piperidine-4-carbonitrile;
3-(2-(4-(difluoromethyl)-4-(methylamino)piperidin-1-yl)quinazolin-4-yl)-4-(6H-thieno[2,3-b]pyrrol-4-yl)-1H-pyrrole-2,5-dione;
3-(2-(4-(fluoromethyl)-4-(methylamino)piperidin-1-yl)quinazolin-4-yl)-4-(6H-thieno[2,3-b]pyrrol-4-yl)-1H-pyrrole-2,5-dione;
3-(2-(4-methyl-4-(methylamino)piperidin-1-yl)quinazolin-4-yl)-4-(6H-thieno[2,3-b]pyrrol-4-yl)-1H-pyrrole-2,5-dione;
3-(2-(4-ethyl-4-(methylamino)piperidin-1-yl)quinazolin-4-yl)-4-(6H-thieno[2,3-b]pyrrol-4-yl)-1H-pyrrole-2,5-dione;
3-(2-(4-(2-hydroxy-2-methylpropylamino)-4-methylpiperidin-1-yl)quinazolin-4-yl)-4-(6H-thieno[2,3-b]pyrrol-4-yl)-1H-pyrrole-2,5-dione;
3-(2-(4-(2-hydroxypropylamino)-4-methylpiperidin-1-yl)quinazolin-4-yl)-4-(6H-thieno[2,3-b]pyrrol-4-yl)-1H-pyrrole-2,5-dione;
3-(2-(4-amino-4-isopropyl-3-(trifluoromethoxy)piperidin-1-yl)quinazolin-4-yl)-4-(6H-thieno[2,3-b]pyrrol-4-yl)-1H-pyrrole-2,5-dione;
3-(6H-thieno[2,3-b]pyrrol-4-yl)-4-(2-(4-((2,2,2-trifluoroethylamino)methyl)piperidin-1-yl)quinazolin-4-yl)-1H-pyrrole-2,5-dione;
3-(2-(4-((2,2-difluoroethylamino)methyl)piperidin-1-yl)quinazolin-4-yl)-4-(6H-thieno[2,3-b]pyrrol-4-yl)-1H-pyrrole-2,5-dione;
3-(2-(4-((2-fluoroethylamino)methyl)piperidin-1-yl)quinazolin-4-yl)-4-(6H-thieno[2,3-b]pyrrol-4-yl)-1H-pyrrole-2,5-dione;
3-(2-(4-((2-hydroxyethylamino)methyl)piperidin-1-yl)quinazolin-4-yl)-4-(6H-thieno[2,3-b]pyrrol-4-yl)-1H-pyrrole-2,5-dione;
3-(2-(4-((2-hydroxypropylamino)methyl)piperidin-1-yl)quinazolin-4-yl)-4-(6H-thieno[2,3-b]pyrrol-4-yl)-1H-pyrrole-2,5-dione;
3-(2-(4-((2-hydroxy-2-methylpropylamino)methyl)piperidin-1-yl)quinazolin-4-yl)-4-(6H-thieno[2,3-b]pyrrol-4-yl)-1H-pyrrole-2,5-dione;
3-(2-(4-hydroxy-4-((methylamino)methyl)piperidin-1-yl)quinazolin-4-yl)-4-(6H-thieno[2,3-b]pyrrol-4-yl)-1H-pyrrole-2,5-dione
3-(2-(4-fluoro-4-((methylamino)methyl)piperidin-1-yl)quinazolin-4-yl)-4-(6H-thieno[2,3-b]pyrrol-4-yl)-1H-pyrrole-2,5-dione;
3-(2-(4-((methylamino)methyl)-4-(trifluoromethyl)piperidin-1-yl)quinazolin-4-yl)-4-(6H-thieno[2,3-b]pyrrol-4-yl)-1H-pyrrole-2,5-dione;
3-(2-(2-(hydroxymethyl)piperazin-1-yl)quinazolin-4-yl)-4-(6H-thieno[2,3-b]pyrrol-4-yl)-1H-pyrrole-2,5-dione;
3-(2-(2-(hydroxymethyl)-4-methylpiperazin-1-yl)quinazolin-4-yl)-4-(6H-thieno[2,3-b]pyrrol-4-yl)-1H-pyrrole-2,5-dione;
3-(2-(3-(hydroxymethyl)-4-methylpiperazin-1-yl)quinazolin-4-yl)-4-(6H-thieno[2,3-b]pyrrol-4-yl)-1H-pyrrole-2,5-dione;
3-(2-(3-(hydroxymethyl)piperazin-1-yl)quinazolin-4-yl)-4-(6H-thieno[2,3-b]pyrrol-4-yl)-1H-pyrrole-2,5-dione;
3-(2-(4-(2-hydroxy-2-methylpropyl)piperazin-1-yl)quinazolin-4-yl)-4-(6H-thieno[2,3-b]pyrrol-4-yl)-1H-pyrrole-2,5-dione;
3-(2-(4-(1-hydroxy-2-methylpropan-2-yl)piperazin-1-yl)quinazolin-4-yl)-4-(6H-thieno[2,3-b]pyrrol-4-yl)-1H-pyrrole-2,5-dione;
3-(2-(7,7-difluorohexahydropyrrolo[1,2-a]pyrazin-2(1H)-yl)quinazolin-4-yl)-4-(6H-thieno[2,3-b]pyrrol-4-yl)-1H-pyrrole-2,5-dione;
3-(2-(3-(dimethylamino)-5,6-dihydroimidazo[1,5-a]pyrazin-7(8H)-yl)quinazolin-4-yl)-4-(6H-thieno[2,3-b]pyrrol-4-yl)-1H-pyrrole-2,5-dione;
3-(1-(3,5-difluoro-1-methylpiperidin-4-yl)-1H-indol-3-yl)-4-(6H-thieno[2,3-b]pyrrol-4-yl)-1H-pyrrole-2,5-dione;
3-(1-(3,5-difluoro-1-methylpiperidin-4-yl)-1H-indazol-3-yl)-4-(6H-thieno[2,3-b]pyrrol-4-yl)-1H-pyrrole-2,5-dione;
3-(1-(3,3-difluoro-1-methylpiperidin-4-yl)-1H-indol-3-yl)-4-(6H-thieno[2,3-b]pyrrol-4-yl)-1H-pyrrole-2,5-dione;
3-(1-(3,3-difluoro-1-methylpiperidin-4-yl)-1H-indazol-3-yl)-4-(6H-thieno[2,3-b]pyrrol-4-yl)-1H-pyrrole-2,5-dione;
3-(1-(3-fluoro-1-methylpiperidin-4-yl)-1H-indol-3-yl)-4-(6H-thieno[2,3-b]pyrrol-4-yl)-1H-pyrrole-2,5-dione;
3-(1-(3-fluoro-1-methylpiperidin-4-yl)-1H-indazol-3-yl)-4-(6H-thieno[2,3-b]pyrrol-4-yl)-1H-pyrrole-2,5-dione;
3-(1-((3,3-difluoro-1-methylpiperidin-4-yl)methyl)-1H-indol-3-yl)-4-(6H-thieno[2,3-b]pyrrol-4-yl)-1H-pyrrole-2,5-dione;
3-(1-((3,3-difluoro-1-methylpiperidin-4-yl)methyl)-1H-indazol-3-yl)-4-(6H-thieno[2,3-b]pyrrol-4-yl)-1H-pyrrole-2,5-dione;
3-(1-(1-(2-(dimethylamino)ethyl)piperidin-4-yl)-1H-indol-3-yl)-4-(6H-thieno[2,3-b]pyrrol-4-yl)-1H-pyrrole-2,5-dione;
3-(1-(1-(2-(dimethylamino)ethyl)piperidin-4-yl)-1H-indazol-3-yl)-4-(6H-thieno[2,3-b]pyrrol-4-yl)-1H-pyrrole-2,5-dione;
3-(1-(2-methyloctahydro-1H-pyrido[1,2-a]pyrazin-8-yl)-1H-indol-3-yl)-4-(6H-thieno[2,3-b]pyrrol-4-yl)-1H-pyrrole-2,5-dione;
3-(1-(2-methyloctahydro-1H-pyrido[1,2-a]pyrazin-8-yl)-1H-indazol-3-yl)-4-(6H-thieno[2,3-b]pyrrol-4-yl)-1H-pyrrole-2,5-dione;
3-(1-(octahydro-1H-pyrido[1,2-a]pyrazin-8-yl)-1H-indol-3-yl)-4-(6H-thieno[2,3-b]pyrrol-4-yl)-1H-pyrrole-2,5-dione;
3-(1-(octahydro-1H-pyrido[1,2-a]pyrazin-8-yl)-1H-indazol-3-yl)-4-(6H-thieno[2,3-b]pyrrol-4-yl)-1H-pyrrole-2,5-dione;
3-(1-(octahydropyrido[2,1-c][1,4]oxazin-8-yl)-1H-indol-3-yl)-4-(6H-thieno[2,3-b]pyrrol-4-yl)-1H-pyrrole-2,5-dione;
3-(1-(octahydropyrido[2,1-c][1,4]oxazin-8-yl)-1H-indazol-3-yl)-4-(6H-thieno[2,3-b]pyrrol-4-yl)-1H-pyrrole-2,5-dione;
3-(1-(1-(2-hydroxy-2-methylpropyl)piperidin-4-yl)-1H-indol-3-yl)-4-(6H-thieno[2,3-b]pyrrol-4-yl)-1H-pyrrole-2,5-dione;
3-(1-(1-(2-hydroxy-2-methylpropyl)piperidin-4-yl)-1H-indazol-3-yl)-4-(6H-thieno[2,3-b]pyrrol-4-yl)-1H-pyrrole-2,5-dione;
3-(1-(1-(2-methoxy-2-methylpropyl)piperidin-4-yl)-1H-indol-3-yl)-4-(6H-thieno[2,3-b]pyrrol-4-yl)-1H-pyrrole-2,5-dione;
3-(1-(1-(2-methoxy-2-methylpropyl)piperidin-4-yl)-1H-indazol-3-yl)-4-(6H-thieno[2,3-b]pyrrol-4-yl)-1H-pyrrole-2,5-dione;
3-(2-(4-(3-fluoropyrrolidin-1-yl)piperidin-1-yl)quinazolin-4-yl)-4-(6H-thieno[2,3-b]pyrrol-4-yl)-1H-pyrrole-2,5-dione;
3-(1-(1-(2-fluoroethyl)piperidin-4-yl)-1H-indol-3-yl)-4-(6H-thieno[2,3-b]pyrrol-4-yl)-1H-pyrrole-2,5-dione;
3-(1-(1-(2-fluoroethyl)piperidin-4-yl)-1H-indazol-3-yl)-4-(6H-thieno[2,3-b]pyrrol-4-yl)-1H-pyrrole-2,5-dione;
3-(1-(1-(2,2-difluoroethyl)piperidin-4-yl)-1H-indol-3-yl)-4-(6H-thieno[2,3-b]pyrrol-4-yl)-1H-pyrrole-2,5-dione;
3-(1-(1-(2,2-difluoroethyl)piperidin-4-yl)-1H-indazol-3-yl)-4-(6H-thieno[2,3-b]pyrrol-4-yl)-1H-pyrrole-2,5-dione;
3-(6H-thieno[2,3-b]pyrrol-4-yl)-4-(1-(1-(2,2,2-trifluoroethyl)piperidin-4-yl)-1H-indol-3-yl)-1H-pyrrole-2,5-dione;
3-(6H-thieno[2,3-b]pyrrol-4-yl)-4-(1-(1-(2,2,2-trifluoroethyl)piperidin-4-yl)-1H-indazol-3-yl)-1H-pyrrole-2,5-dione;
3-(1-(1-(2-methoxyethyl)piperidin-4-yl)-1H-indol-3-yl)-4-(6H-thieno[2,3-b]pyrrol-4-yl)-1H-pyrrole-2,5-dione;
3-(1-(1-(2-methoxyethyl)piperidin-4-yl)-1H-indazol-3-yl)-4-(6H-thieno[2,3-b]pyrrol-4-yl)-1H-pyrrole-2,5-dione;
3-(1-((1-ethylpiperidin-4-yl)methyl)-1H-indol-3-yl)-4-(6H-thieno[2,3-b]pyrrol-4-yl)-1H-pyrrole-2,5-dione;
3-(1-((1-ethylpiperidin-4-yl)methyl)-1H-indazol-3-yl)-4-(6H-thieno[2,3-b]pyrrol-4-yl)-1H-pyrrole-2,5-dione;
3-(1-((1-isopropylpiperidin-4-yl)methyl)-1H-indol-3-yl)-4-(6H-thieno[2,3-b]pyrrol-4-yl)-1H-pyrrole-2,5-dione;
3-(1-((1-isopropylpiperidin-4-yl)methyl)-1H-indazol-3-yl)-4-(6H-thieno[2,3-b]pyrrol-4-yl)-1H-pyrrole-2,5-dione;
3-(2-(4-(dimethylamino)-3-fluoropiperidin-1-yl)quinazolin-4-yl)-4-(6H-thieno[2,3-b]pyrrol-4-yl)-1H-pyrrole-2,5-dione;
3-(2-(3-fluoro-4-(pyrrolidin-1-yl)piperidin-1-yl)quinazolin-4-yl)-4-(6H-thieno[2,3-b]pyrrol-4-yl)-1H-pyrrole-2,5-dione;
3-(2-(4-(aziridin-1-yl)-3-fluoropiperidin-1-yl)quinazolin-4-yl)-4-(6H-thieno[2,3-b]pyrrol-4-yl)-1H-pyrrole-2,5-dione;
3-(2-(4-(azetidin-1-yl)-3-fluoropiperidin-1-yl)quinazolin-4-yl)-4-(6H-thieno[2,3-b]pyrrol-4-yl)-1H-pyrrole-2,5-dione;
3-(2-(4-amino-4-(2-hydroxy-2-methylpropyl)piperidin-1-yl)quinazolin-4-yl)-4-(6H-thieno[2,3-b]pyrrol-4-yl)-1H-pyrrole-2,5-dione;
3-(2-(4-amino-4-(fluoromethyl)piperidin-1-yl)quinazolin-4-yl)-4-(6H-thieno[2,3-b]pyrrol-4-yl)-1H-pyrrole-2,5-dione;
3-(2-(2-methyloctahydro-1H-pyrido[1,2-a]pyrazin-8-yl)quinazolin-4-yl)-4-(6H-thieno[2,3-b]pyrrol-4-yl)-1H-pyrrole-2,5-dione;
3-(2-(octahydropyrido[2,1-c][1,4]oxazin-8-yl)quinazolin-4-yl)-4-(6H-thieno[2,3-b]pyrrol-4-yl)-1H-pyrrole-2,5-dione;
3-(2-(1-(2-methoxy-2-methylpropyl)piperidin-4-yl)quinazolin-4-yl)-4-(6H-thieno[2,3-b]pyrrol-4-yl)-1H-pyrrole-2,5-dione;
3-(2-(1-(2-hydroxy-2-methylpropyl)piperidin-4-yl)quinazolin-4-yl)-4-(6H-thieno[2,3-b]pyrrol-4-yl)-1H-pyrrole-2,5-dione;
3-(2-(octahydro-1H-pyrido[1,2-a]pyrazin-8-yl)quinazolin-4-yl)-4-(6H-thieno[2,3-b]pyrrol-4-yl)-1H-pyrrole-2,5-dione;
3-(2-(3-fluoropiperidin-4-yl)quinazoin-4-yl)-4-(6H-thieno[2,3-b]pyrrol-4-yl)-1H-pyrrole-2,5-dione;
3-(2-(3-fluoro-1-methylpiperidin-4-yl)quinazolin-4-yl)-4-(6H-thieno[2,3-b]pyrrol-4-yl)-1H-pyrrole-2,5-dione;
3-(2-(2-methyloctahydro-1H-pyrido[1,2-a]pyrazin-8-yl)quinazolin-4-yl)-4-(6H-thieno[2,3-b]pyrrol-4-yl)-1H-pyrrole-2,5-dione;
3-(4H-thieno[3,2-b]pyrrol-6-yl)-4-(1-((1-(2,2,2-trifluoroethyl)piperidin-4-yl)methyl)-1H-indol-3-yl)-1H-pyrrole-2,5-dione;
3-(1-((1-(2,2-difluoroethyl)piperidin-4-yl)methyl)-1H-indol-3-yl)-4-(4H-thieno[3,2-b]pyrrol-6-yl)-1H-pyrrole-2,5-dione;
3-(1-((1-(2,2-difluoroethyl)piperidin-4-yl)methyl)-1H-indazol-3-yl)-4-(4H-thieno[3,2-b]pyrrol-6-yl)-1H-pyrrole-2,5-dione;
3-(4H-thieno[3,2-b]pyrrol-6-yl)-4-(1-((1-(2,2,2-trifluoroethyl)piperidin-4-yl)methyl)-1H-indazol-3-yl)-1H-pyrrole-2,5-dione;
3-(1-((1-(2-fluoroethyl)piperidin-4-yl)methyl)-1H-indazol-3-yl)-4-(4H-thieno[3,2-b]pyrrol-6-yl)-1H-pyrrole-2,5-dione;
3-(1-((1-(2-fluoroethyl)piperidin-4-yl)methyl)-1H-indol-3-yl)-4-(4H-thieno[3,2-b]pyrrol-6-yl)-1H-pyrrole-2,5-dione;
3-(1-((1-(2-methoxyethyl)piperidin-4-yl)methyl)-1H-indol-3-yl)-4-(4H-thieno[3,2-b]pyrrol-6-yl)-1H-pyrrole-2,5-dione;
3-(1-((1-(2-methoxyethyl)piperidin-4-yl)methyl)-1H-indazol-3-yl)-4-(4H-thieno[3,2-b]pyrrol-6-yl)-1H-pyrrole-2,5-dione;
3-(1-((4-fluoro-1-methylpiperidin-4-yl)methyl)-1H-indazol-3-yl)-4-(4H-thieno[3,2-b]pyrrol-6-yl)-1H-pyrrole-2,5-dione;
3-(1-((4-fluoro-1-methylpiperidin-4-yl)methyl)-1H-indol-3-yl)-4-(4H-thieno[3,2-b]pyrrol-6-yl)-1H-pyrrole-2,5-dione;
3-(1-((3-fluoro-1-methylpiperidin-4-yl)methyl)-1H-indol-3-yl)-4-(4H-thieno[3,2-b]pyrrol-6-yl)-1H-pyrrole-2,5-dione;
3-(1-((3-fluoro-1-methylpiperidin-4-yl)methyl)-1H-indazol-3-yl)-4-(4H-thieno[3,2-b]pyrrol-6-yl)-1H-pyrrole-2,5-dione;
3-(1-((3-fluoropiperidin-4-yl)methyl)-1H-indazol-3-yl)-4-(4H-thieno[3,2-b]pyrrol-6-yl)-1H-pyrrole-2,5-dione;
3-(1-((3-fluoropiperidin-4-yl)methyl)-1H-indol-3-yl)-4-(4H-thieno[3,2-b]pyrrol-6-yl)-1H-pyrrole-2,5-dione;
3-(1-((2-(hydroxymethyl)piperidin-4-yl)methyl)-1H-indol-3-yl)-4-(4H-thieno[3,2-b]pyrrol-6-yl)-1H-pyrrole-2,5-dione;
3-(1-((2-(hydroxymethyl)piperidin-4-yl)methyl)-1H-indazol-3-yl)-4-(4H-thieno[3,2-b]pyrrol-6-yl)-1H-pyrrole-2,5-dione;
3-(1-((2-(hydroxymethyl)-1-methylpiperidin-4-yl)methyl)-1H-indazol-3-yl)-4-(4H-thieno[3,2-b]pyrrol-6-yl)-1H-pyrrole-2,5-dione;
3-(1-((2-(hydroxymethyl)-1-methylpiperidin-4-yl)methyl)-1H-indol-3-yl)-4-(4H-thieno[3,2-b]pyrrol-6-yl)-1H-pyrrole-2,5-dione;
3-(4H-thieno[3,2-b]pyrrol-6-yl)-4-(1-((2-((trifluoromethoxy)methyl)piperidin-4-yl)methyl)-1H-indazol-3-yl)-1H-pyrrole-2,5-dione;
3-(4H-thieno[3,2-b]pyrrol-6-yl)-4-(1-((2-((trifluoromethoxy)methyl)piperidin-4-yl)methyl)-1H-indol-3-yl)-1H-pyrrole-2,5-dione;
3-(1-((1-methyl-2-((trifluoromethoxy)methyl)piperidin-4-yl)methyl)-1H-indol-3-yl)-4-(4H-thieno[3,2-b]pyrrol-6-yl)-1H-pyrrole-2,5-dione;
3-(1-((1-methyl-2-((trifluoromethoxy)methyl)piperidin-4-yl)methyl)-1H-indazol-3-yl)-4-(4H-thieno[3,2-b]pyrrol-6-yl)-1H-pyrrole-2,5-dione;
3-(2-(4-ethyl-3-hydroxy-4-(methylamino)piperidin-1-yl)quinazolin-4-yl)-4-(4H-thieno[3,2-b]pyrrol-6-yl)-1H-pyrrole-2,5-dione;
3-(2-(4-ethyl-3-methoxy-4-(methylamino)piperidin-1-yl)quinazolin-4-yl)-4-(4H-thieno[3,2-b]pyrrol-6-yl)-1H-pyrrole-2,5-dione;
3-(2-(4-ethyl-4-(methylamino)-3-(trifluoromethoxy)piperidin-1-yl)quinazolin-4-yl)-4-(4H-thieno[3,2-b]pyrrol-6-yl)-1H-pyrrole-2,5-dione;
3-(2-(4-amino-3-hydroxy-4-methylpiperidin-1-yl)quinazolin-4-yl)-4-(4H-thieno[3,2-b]pyrrol-6-yl)-1H-pyrrole-2,5-dione;
3-(2-(4-amino-3-methoxy-4-methylpiperidin-1-yl)quinazolin-4-yl)-4-(4H-thieno[3,2-b]pyrrol-6-yl)-1H-pyrrole-2,5-dione;
3-(2-(4-amino-4-methyl-3-(trifluoromethoxy)piperidin-1-yl)quinazolin-4-yl)-4-(4H-thieno[3,2-b]pyrrol-6-yl)-1H-pyrrole-2,5-dione;
3-(2-(3-hydroxy-4-methyl-4-(methylamino)piperidin-1-yl)quinazolin-4-yl)-4-(4H-thieno[3,2-b]pyrrol-6-yl)-1H-pyrrole-2,5-dione;
3-(2-(3-methoxy-4-methyl-4-(methylamino)piperidin-1-yl)quinazolin-4-yl)-4-(4H-thieno[3,2-b]pyrrol-6-yl)-1H-pyrrole-2,5-dione;
3-(2-(4-methyl-4-(methylamino)-3-(trifluoromethoxy)piperidin-1-yl)quinazolin-4-yl)-4-(4H-thieno[3,2-b]pyrrol-6-yl)-1H-pyrrole-2,5-dione;
3-(2-(3-ethyl-5,6-dihydroimidazo[1,5-a]pyrazin-7(8H)-yl)quinazolin-4-yl)-4-(4H-thieno[3,2-b]pyrrol-6-yl)-1H-pyrrole-2,5-dione;
3-(2-(3-isopropyl-5,6-dihydroimidazo[1,5-a]pyrazin-7(8H)-yl)quinazolin-4-yl)-4-(4H-thieno[3,2-b]pyrrol-6-yl)-1H-pyrrole-2,5-dione;
3-(2-(3-cyclopropyl-5,6-dihydroimidazo[1,5-a]pyrazin-7(8H)-yl)quinazolin-4-yl)-4-(4H-thieno[3,2-b]pyrrol-6-yl)-1H-pyrrole-2,5-dione;
3-(1-(morpholin-2-ylmethyl)-1H-indazol-3-yl)-4-(4H-thieno[3,2-b]pyrrol-6-yl)-1H-pyrrole-2,5-dione;
3-(1-(morpholin-2-ylmethyl)-1H-indol-3-yl)-4-(4H-thieno[3,2-b]pyrrol-6-yl)-1H-pyrrole-2,5-dione;
3-(1-((4-methylmorpholin-2-yl)methyl)-1H-indazol-3-yl)-4-(4H-thieno[3,2-b]pyrrol-6-yl)-1H-pyrrole-2,5-dione;
3-(1-((4-methylmorpholin-2-yl)methyl)-1H-indol-3-yl)-4-(4H-thieno[3,2-b]pyrrol-6-yl)-1H-pyrrole-2,5-dione;
3-(2-(3-tert-butyl-5,6-dihydroimidazo[1,5-a]pyrazin-7(8H)-yl)quinazolin-4-yl)-4-(4H-thieno[3,2-b]pyrrol-6-yl)-1H-pyrrole-2,5-dione;
3-(1-((4-fluoropiperidin-4-yl)methyl)-1H-indol-3-yl)-4-(4H-thieno[3,2-b]pyrrol-6-yl)-1H-pyrrole-2,5-dione;
3-(1-((4-fluoropiperidin-4-yl)methyl)-1H-indazol-3-yl)-4-(4H-thieno[3,2-b]pyrrol-6-yl)-1H-pyrrole-2,5-dione;
3-(8-methyl-2-(4-methylpiperazin-1-yl)quinazolin-4-yl)-4-(4H-thieno[3,2-b]pyrrol-6-yl)-1H-pyrrole-2,5-dione;
3-(7-methyl-2-(4-methylpiperazin-1-yl)quinazolin-4-yl)-4-(4H-thieno[3,2-b]pyrrol-6-yl)-1H-pyrrole-2,5-dione;
3-(6-methyl-2-(4-methylpiperazin-1-yl)quinazolin-4-yl)-4-(4H-thieno[3,2-b]pyrrol-6-yl)-1H-pyrrole-2,5-dione;
3-(5-methyl-2-(4-methylpiperazin-1-yl)quinazolin-4-yl)-4-(4H-thieno[3,2-b]pyrrol-6-yl)-1H-pyrrole-2,5-dione;
3-(2-(4-methylpiperazin-1-yl)thieno[3,2-d]pyrimidin-4-yl)-4-(4H-thieno[3,2-b]pyrrol-6-yl)-1H-pyrrole-2,5-dione;
3-(2-(4-methylpiperazin-1-yl)thieno[2,3-d]pyrimidin-4-yl)-4-(4H-thieno[3,2-b]pyrrol-6-yl)-1H-pyrrole-2,5-dione;
3-(1-((1-methylpiperidin-3-yl)methyl)-1H-indazol-3-yl)-4-(4H-thieno[3,2-b]pyrrol-6-yl)-1H-pyrrole-2,5-dione;
3-(1-((1-(2-methoxyethyl)piperidin-3-yl)methyl)-1H-indazol-3-yl)-4-(4H-thieno[3,2-b]pyrrol-6-yl)-1H-pyrrole-2,5-dione;
3-(1-((1-(2-methoxyethyl)pyrrolidin-3-yl)methyl)-1H-indazol-3-yl)-4-(4H-thieno[3,2-b]pyrrol-6-yl)-1H-pyrrole-2,5-dione;
3-(1-(2-(dimethylamino)ethyl)-1H-indazol-3-yl)-4-(4H-thieno[3,2-b]pyrrol-6-yl)-1H-pyrrole-2,5-dione;
3-(1-(2-(dimethylamino)ethyl)-1H-indol-3-yl)-4-(4H-thieno[3,2-b]pyrrol-6-yl)-1H-pyrrole-2,5-dione;
3-(1-(4-(dimethylamino)butyl)-1H-indazol-3-yl)-4-(4H-thieno[3,2-b]pyrrol-6-yl)-1H-pyrrole-2,5-dione;
3-(1-(4-(dimethylamino)butyl)-1H-indol-3-yl)-4-(4H-thieno[3,2-b]pyrrol-6-yl)-1H-pyrrole-2,5-dione;
3-(1-(3-(dimethylamino)propyl)-1H-indazol-3-yl)-4-(4H-thieno[3,2-b]pyrrol-6-yl)-1H-pyrrole-2,5-dione;
3-(1-((2-(methoxymethyl)-1-methylpiperidin-4-yl)methyl)-1H-indol-3-yl)-4-(4H-thieno[3,2-b]pyrrol-6-yl)-1H-pyrrole-2,5-dione;
3-(1-((2-(methoxymethyl)-1-methylpiperidin-4-yl)methyl)-1H-indazol-3-yl)-4-(4H-thieno[3,2-b]pyrrol-6-yl)-1H-pyrrole-2,5-dione;
3-(1-((2-(methoxymethyl)piperidin-4-yl)methyl)-1H-indazol-3-yl)-4-(4H-thieno[3,2-b]pyrrol-6-yl)-1H-pyrrole-2,5-dione;
3-(1-((2-(methoxymethyl)piperidin-4-yl)methyl)-1H-indol-3-yl)-4-(4H-thieno[3,2-b]pyrrol-6-yl)-1H-pyrrole-2,5-dione;
3-(4H-thieno[3,2-b]pyrrol-6-yl)-4-(1-((2-((trifluoromethoxy)methyl)piperidin-4-yl)methyl)-1H-indol-3-yl)-1H-pyrrole-2,5-dione;
3-(1-((1-methyl-2-((trifluoromethoxy)methyl)piperidin-4-yl)methyl)-1H-indazol-3-yl)-4-(4H-thieno[3,2-b]pyrrol-6-yl)-1H-pyrrole-2,5-dione;
3-(2-(4-amino-3-fluoro-4-methylpiperidin-1-yl)quinazolin-4-yl)-4-(4H-thieno[3,2-b]pyrrol-6-yl)-1H-pyrrole-2,5-dione;
3-(2-(3-fluoro-4-methyl-4-(methylamino)piperidin-1-yl)quinazolin-4-yl)-4-(4H-thieno[3,2-b]pyrrol-6-yl)-1H-pyrrole-2,5-dione;
3-(2-(4-ethyl-3-fluoro-4-(methylamino)piperidin-1-yl)quinazolin-4-yl)-4-(4H-thieno[3,2-b]pyrrol-6-yl)-1H-pyrrole-2,5-dione;
3-(2-(4-amino-4-ethyl-3-fluoropiperidin-1-yl)quinazolin-4-yl)-4-(4H-thieno[3,2-b]pyrrol-6-yl)-1H-pyrrole-2,5-dione;
3-(2-(4-amino-3-fluoro-4-isopropylpiperidin-1-yl)quinazolin-4-yl)-4-(4H-thieno[3,2-b]pyrrol-6-yl)-1H-pyrrole-2,5-dione;
3-(2-(3-fluoro-4-isopropyl-4-(methylamino)piperidin-1-yl)quinazolin-4-yl)-4-(4H-thieno[3,2-b]pyrrol-6-yl)-1H-pyrrole-2,5-dione;
3-(2-(4-amino-4-(trifluoromethyl)piperidin-1-yl)quinazolin-4-yl)-4-(4H-thieno[3,2-b]pyrrol-6-yl)-1H-pyrrole-2,5-dione;
3-(2-(4-(methylamino)-4-(trifluoromethyl)piperidin-1-yl)quinazolin-4-yl)-4-(4H-thieno[3,2-b]pyrrol-6-yl)-1H-pyrrole-2,5-dione;
3-(2-(4-amino-4-(hydroxymethyl)piperidin-1-yl)quinazolin-4-yl)-4-(4H-thieno[3,2-b]pyrrol-6-yl)-1H-pyrrole-2,5-dione;
3-(2-(4-(hydroxymethyl)-4-(methylamino)piperidin-1-yl)quinazolin-4-yl)-4-(4H-thieno[3,2-b]pyrrol-6-yl)-1H-pyrrole-2,5-dione;
3-(2-(4-(2-hydroxyethylamino)-4-methylpiperidin-1-yl)quinazolin-4-yl)-4-(4H-thieno[3,2-b]pyrrol-6-yl)-1H-pyrrole-2,5-dione;
3-(2-(4-amino-4-(2-hydroxyethyl)piperidin-1-yl)quinazolin-4-yl)-4-(4H-thieno[3,2-b]pyrrol-6-yl)-1H-pyrrole-2,5-dione;
3-(2-(4-(2-hydroxyethyl)-4-(methylamino)piperidin-1-yl)quinazolin-4-yl)-4-(4H-thieno[3,2-b]pyrrol-6-yl)-1H-pyrrole-2,5-dione;
4-amino-1-(4-(2,5-dioxo-4-(4H-thieno[3,2-b]pyrrol-6-yl)-2,5-dihydro-1H-pyrrol-3-yl)quinazolin-2-yl)piperidine-4-carbonitrile;
1-(4-(2,5-dioxo-4-(4H-thieno[3,2-b]pyrrol-6-yl)-2,5-dihydro-1H-pyrrol-3-yl)quinazolin-2-yl)-4-(methylamino)piperidine-4-carbonitrile;
3-(2-(4-(difluoromethyl)-4-(methylamino)piperidin-1-yl)quinazolin-4-yl)-4-(4H-thieno[3,2-b]pyrrol-6-yl)-1H-pyrrole-2,5-dione;
3-(2-(4-(fluoromethyl)-4-(methylamino)piperidin-1-yl)quinazolin-4-yl)-4-(4H-thieno[3,2-b]pyrrol-6-yl)-1H-pyrrole-2,5-dione;
3-(2-(4-methyl-4-(methylamino)piperidin-1-yl)quinazolin-4-yl)-4-(4H-thieno[3,2-b]pyrrol-6-yl)-1H-pyrrole-2,5-dione;
3-(2-(4-ethyl-4-(methylamino)piperidin-1-yl)quinazolin-4-yl)-4-(4H-thieno[3,2-b]pyrrol-6-yl)-1H-pyrrole-2,5-dione;
3-(2-(4-(2-hydroxy-2-methylpropylamino)-4-methylpiperidin-1-yl)quinazolin-4-yl)-4-(4H-thieno[3,2-b]pyrrol-6-yl)-1H-pyrrole-2,5-dione;
3-(2-(4-(2-hydroxypropylamino)-4-methylpiperidin-1-yl)quinazolin-4-yl)-4-(4H-thieno[3,2-b]pyrrol-6-yl)-1H-pyrrole-2,5-dione;
3-(2-(4-amino-4-isopropyl-3-(trifluoromethoxy)piperidin-1-yl)quinazolin-4-yl)-4-(4H-thieno[3,2-b]pyrrol-6-yl)-1H-pyrrole-2,5-dione;
3-(4H-thieno[3,2-b]pyrrol-6-yl)-4-(2-(4-((2,2,2-trifluoroethylamino)methyl)piperidin-1-yl)quinazolin-4-yl)-1H-pyrrole-2,5-dione;
3-(2-(4-((2,2-difluoroethylamino)methyl)piperidin-1-yl)quinazolin-4-yl)-4-(4H-thieno[3,2-b]pyrrol-6-yl)-1H-pyrrole-2,5-dione;
3-(2-(4-((2-fluoroethylamino)methyl)piperidin-1-yl)quinazolin-4-yl)-4-(4H-thieno[3,2-b]pyrrol-6-yl)-1H-pyrrole-2,5-dione;
3-(2-(4-((2-hydroxyethylamino)methyl)piperidin-1-yl)quinazolin-4-yl)-4-(4H-thieno[3,2-b]pyrrol-6-yl)-1H-pyrrole-2,5-dione;
3-(2-(4-((2-hydroxypropylamino)methyl)piperidin-1-yl)quinazolin-4-yl)-4-(4H-thieno[3,2-b]pyrrol-6-yl)-1H-pyrrole-2,5-dione;
3-(2-(4-((2-hydroxy-2-methylpropylamino)methyl)piperidin-1-yl)quinazolin-4-yl)-4-(4H-thieno[3,2-b]pyrrol-6-yl)-1H-pyrrole-2,5-dione;
3-(2-(4-hydroxy-4-((methylamino)methyl)piperidin-1-yl)quinazolin-4-yl)-4-(4H-thieno[3,2-b]pyrrol-6-yl)-1H-pyrrole-2,5-dione;
3-(2-(4-fluoro-4-((methylamino)methyl)piperidin-1-yl)quinazolin-4-yl)-4-(4H-thieno[3,2-b]pyrrol-6-yl)-1H-pyrrole-2,5-dione;
3-(2-(4-((methylamino)methyl)-4-(trifluoromethyl)piperidin-1-yl)quinazolin-4-yl)-4-(4H-thieno[3,2-b]pyrrol-6-yl)-1H-pyrrole-2,5-dione;
3-(2-(2-(hydroxymethyl)piperazin-1-yl)quinazolin-4-yl)-4-(4H-thieno[3,2-b]pyrrol-6-yl)-1H-pyrrole-2,5-dione;
3-(2-(2-(hydroxymethyl)-4-methylpiperazin-1-yl)quinazolin-4-yl)-4-(4H-thieno[3,2-b]pyrrol-6-yl)-1H-pyrrole-2,5-dione;
3-(2-(3-(hydroxymethyl)-4-methylpiperazin-1-yl)quinazolin-4-yl)-4-(4H-thieno[3,2-b]pyrrol-6-yl)-1H-pyrrole-2,5-dione;
3-(2-(3-(hydroxymethyl)piperazin-1-yl)quinazolin-4-yl)-4-(4H-thieno[3,2-b]pyrrol-6-yl)-1H-pyrrole-2,5-dione;
3-(2-(4-(2-hydroxy-2-methylpropyl)piperazin-1-yl)quinazolin-4-yl)-4-(4H-thieno[3,2-b]pyrrol-6-yl)-1H-pyrrole-2,5-dione;
3-(2-(4-(1-hydroxy-2-methylpropan-2-yl)piperazin-1-yl)quinazolin-4-yl)-4-(4H-thieno[3,2-b]pyrrol-6-yl)-1H-pyrrole-2,5-dione;
3-(2-(7,7-difluorohexahydropyrrolo[1,2-a]pyrazin-2(1H)-yl)quinazolin-4-yl)-4-(4H-thieno[3,2-b]pyrrol-6-yl)-1H-pyrrole-2,5-dione;
3-(2-(3-(dimethylamino)-5,6-dihydroimidazo[1,5-a]pyrazin-7(8H)-yl)quinazolin-4-yl)-4-(4H-thieno[3,2-b]pyrrol-6-yl)-1H-pyrrole-2,5-dione;
3-(1-(3,5-difluoro-1-methylpiperidin-4-yl)-1H-indol-3-yl)-4-(4H-thieno[3,2-b]pyrrol-6-yl)-1H-pyrrole-2,5-dione;
3-(1-(3,5-difluoro-1-methylpiperidin-4-yl)-1H-indazol-3-yl)-4-(4H-thieno[3,2-b]pyrrol-6-yl)-1H-pyrrole-2,5-dione;
3-(1-(3,3-difluoro-1-methylpiperidin-4-yl)-1H-indol-3-yl)-4-(4H-thieno[3,2-b]pyrrol-6-yl)-1H-pyrrole-2,5-dione;
3-(1-(3,3-difluoro-1-methylpiperidin-4-yl)-1H-indazol-3-yl)-4-(4H-thieno[3,2-b]pyrrol-6-yl)-1H-pyrrole-2,5-dione;
3-(1-(3-fluoro-1-methylpiperidin-4-yl)-1H-indol-3-yl)-4-(4H-thieno[3,2-b]pyrrol-6-yl)-1H-pyrrole-2,5-dione;
3-(1-(3-fluoro-1-methylpiperidin-4-yl)-1H-indazol-3-yl)-4-(4H-thieno[3,2-b]pyrrol-6-yl)-1H-pyrrole-2,5-dione;
3-(1-((3,3-difluoro-1-methylpiperidin-4-yl)methyl)-1H-indol-3-yl)-4-(4H-thieno[3,2-b]pyrrol-6-yl)-1H-pyrrole-2,5-dione;
3-(1-((3,3-difluoro-1-methylpiperidin-4-yl)methyl)-1H-indazol-3-yl)-4-(4H-thieno[3,2-b]pyrrol-6-yl)-1H-pyrrole-2,5-dione;
3-(1-(1-(2-(dimethylamino)ethyl)piperidin-4-yl)-1H-indol-3-yl)-4-(4H-thieno[3,2-b]pyrrol-6-yl)-1H-pyrrole-2,5-dione;
3-(1-(1-(2-(dimethylamino)ethyl)piperidin-4-yl)-1H-indazol-3-yl)-4-(4H-thieno[3,2-b]pyrrol-6-yl)-1H-pyrrole-2,5-dione;
3-(1-(2-methyloctahydro-1H-pyrido[1,2-a]pyrazin-8-yl)-1H-indol-3-yl)-4-(4H-thieno[3,2-b]pyrrol-6-yl)-1H-pyrrole-2,5-dione;
3-(1-(2-methyloctahydro-1H-pyrido[1,2-a]pyrazin-8-yl)-1H-indazol-3-yl)-4-(4H-thieno[3,2-b]pyrrol-6-yl)-1H-pyrrole-2,5-dione;
3-(1-(octahydro-1H-pyrido[1,2-a]pyrazin-8-yl)-1H-indol-3-yl)-4-(4H-thieno[3,2-b]pyrrol-6-yl)-1H-pyrrole-2,5-dione;
3-(1-(octahydro-1H-pyrido[1,2-a]pyrazin-8-yl)-1H-indazol-3-yl)-4-(4H-thieno[3,2-b]pyrrol-6-yl)-1H-pyrrole-2,5-dione;
3-(1-(octahydropyrido[2,1-c][1,4]oxazin-8-yl)-1H-indol-3-yl)-4-(4H-thieno[3,2-b]pyrrol-6-yl)-1H-pyrrole-2,5-dione;
3-(1-(octahydropyrido[2,1-c][1,4]oxazin-8-yl)-1H-indazol-3-yl)-4-(4H-thieno[3,2-b]pyrrol-6-yl)-1H-pyrrole-2,5-dione;
3-(1-(1-(2-hydroxy-2-methylpropyl)piperidin-4-yl)-1H-indol-3-yl)-4-(4H-thieno[3,2-b]pyrrol-6-yl)-1H-pyrrole-2,5-dione;
3-(1-(1-(2-hydroxy-2-methylpropyl)piperidin-4-yl)-1H-indazol-3-yl)-4-(4H-thieno[3,2-b]pyrrol-6-yl)-1H-pyrrole-2,5-dione;
3-(1-(1-(2-methoxy-2-methylpropyl)piperidin-4-yl)-1H-indol-3-yl)-4-(4H-thieno[3,2-b]pyrrol-6-yl)-1H-pyrrole-2,5-dione;
3-(1-(1-(2-methoxy-2-methylpropyl)piperidin-4-yl)-1H-indazol-3-yl)-4-(4H-thieno[3,2-b]pyrrol-6-yl)-1H-pyrrole-2,5-dione;
3-(2-(4-(3-fluoropyrrolidin-1-yl)piperidin-1-yl)quinazolin-4-yl)-4-(4H-thieno[3,2-b]pyrrol-6-yl)-1H-pyrrole-2,5-dione;
3-(1-(1-(2-fluoroethyl)piperidin-4-yl)-1H-indol-3-yl)-4-(4H-thieno[3,2-b]pyrrol-6-yl)-1H-pyrrole-2,5-dione;
3-(1-(1-(2-fluoroethyl)piperidin-4-yl)-1H-indazol-3-yl)-4-(4H-thieno[3,2-b]pyrrol-6-yl)-1H-pyrrole-2,5-dione;
3-(1-(1-(2,2-difluoroethyl)piperidin-4-yl)-1H-indol-3-yl)-4-(4H-thieno[3,2-b]pyrrol-6-yl)-1H-pyrrole-2,5-dione;
3-(1-(1-(2,2-difluoroethyl)piperidin-4-yl)-1H-indazol-3-yl)-4-(4H-thieno[3,2-b]pyrrol-6-yl)-1H-pyrrole-2,5-dione;
3-(4H-thieno[3,2-b]pyrrol-6-yl)-4-(1-(1-(2,2,2-trifluoroethyl)piperidin-4-yl)-1H-indol-3-yl)-1H-pyrrole-2,5-dione;
3-(4H-thieno[3,2-b]pyrrol-6-yl)-4-(1-(1-(2,2,2-trifluoroethyl)piperidin-4-yl)-1H-indazol-3-yl)-1H-pyrrole-2,5-dione;
3-(1-(1-(2-methoxyethyl)piperidin-4-yl)-1H-indol-3-yl)-4-(4H-thieno[3,2-b]pyrrol-6-yl)-1H-pyrrole-2,5-dione;
3-(1-(1-(2-methoxyethyl)piperidin-4-yl)-1H-indazol-3-yl)-4-(4H-thieno[3,2-b]pyrrol-6-yl)-1H-pyrrole-2,5-dione;
3-(1-((1-ethylpiperidin-4-yl)methyl)-1H-indol-3-yl)-4-(4H-thieno[3,2-b]pyrrol-6-yl)-1H-pyrrole-2,5-dione;
3-(1-((1-ethylpiperidin-4-yl)methyl)-1H-indazol-3-yl)-4-(4H-thieno[3,2-b]pyrrol-6-yl)-1H-pyrrole-2,5-dione;
3-(1-((1-isopropylpiperidin-4-yl)methyl)-1H-indol-3-yl)-4-(4H-thieno[3,2-b]pyrrol-6-yl)-1H-pyrrole-2,5-dione;
3-(1-((1-isopropylpiperidin-4-yl)methyl)-1H-indazol-3-yl)-4-(4H-thieno[3,2-b]pyrrol-6-yl)-1H-pyrrole-2,5-dione;
3-(2-(4-(dimethylamino)-3-fluoropiperidin-1-yl)quinazolin-4-yl)-4-(4H-thieno[3,2-b]pyrrol-6-yl)-1H-pyrrole-2,5-dione;
3-(2-(3-fluoro-4-(pyrrolidin-1-yl)piperidin-1-yl)quinazolin-4-yl)-4-(4H-thieno[3,2-b]pyrrol-6-yl)-1H-pyrrole-2,5-dione;
3-(2-(4-(aziridin-1-yl)-3-fluoropiperidin-1-yl)quinazolin-4-yl)-4-(4H-thieno[3,2-b]pyrrol-6-yl)-1H-pyrrole-2,5-dione;
3-(2-(4-(azetidin-1-yl)-3-fluoropiperidin-1-yl)quinazolin-4-yl)-4-(4H-thieno[3,2-b]pyrrol-6-yl)-1H-pyrrole-2,5-dione;
3-(2-(4-amino-4-(2-hydroxy-2-methylpropyl)piperidin-1-yl)quinazolin-4-yl)-4-(4H-thieno[3,2-b]pyrrol-6-yl)-1H-pyrrole-2,5-dione;
3-(2-(4-amino-4-(fluoromethyl)piperidin-1-yl)quinazolin-4-yl)-4-(4H-thieno[3,2-b]pyrrol-6-yl)-1H-pyrrole-2,5-dione;
3-(2-(2-methyloctahydro-1H-pyrido[1,2-a]pyrazin-8-yl)quinazolin-4-yl)-4-(4H-thieno[3,2-b]pyrrol-6-yl)-1H-pyrrole-2,5-dione;
3-(2-(octahydropyrido[2,1-c][1,4]oxazin-8-yl)quinazolin-4-yl)-4-(4H-thieno[3,2-b]pyrrol-6-yl)-1H-pyrrole-2,5-dione;
3-(2-(1-(2-methoxy-2-methylpropyl)piperidin-4-yl)quinazolin-4-yl)-4-(4H-thieno[3,2-b]pyrrol-6-yl)-1H-pyrrole-2,5-dione;
3-(2-(1-(2-hydroxy-2-methylpropyl)piperidin-4-yl)quinazolin-4-yl)-4-(4H-thieno[3,2-b]pyrrol-6-yl)-1H-pyrrole-2,5-dione;
3-(2-(octahydro-1H-pyrido[1,2-a]pyrazin-8-yl)quinazolin-4-yl)-4-(4H-thieno[3,2-b]pyrrol-6-yl)-1H-pyrrole-2,5-dione;
3-(2-(3-fluoropiperidin-4-yl)quinazolin-4-yl)-4-(4H-thieno[3,2-b]pyrrol-6-yl)-1H-pyrrole-2,5-dione;
3-(2-(3-fluoro-1-methylpiperidin-4-yl)quinazolin-4-yl)-4-(4H-thieno[3,2-b]pyrrol-6-yl)-1H-pyrrole-2,5-dione;
3-(2-(2-methyloctahydro-1H-pyrido[1,2-a]pyrazin-8-yl)quinazolin-4-yl)-4-(4H-thieno[3,2-b]pyrrol-6-yl)-1H-pyrrole-2,5-dione;
3-[2-(5,6-dihydro-8H-[1,2,4]triazolo[1,5-a]pyrazin-7-yl)-quinazolin-4-yl]-4-(6H-thieno[2,3-b]pyrrol-4-yl)-pyrrole-2,5-dione;
3-[2-(4-methyl-piperazin-1-yl)-5,6,7,8-tetrahydro-quinazolin-4-yl]-4-(6H-thieno[2,3-b]pyrrol-4-yl)-pyrrole-2,5-dione;
3-[1-(1-methyl-piperidin-4-ylmethyl)-1H-indol-3-yl]-4-(6H-thieno[2,3-b]pyrrol-4-yl)-pyrrole-2,5-dione;
3-[2-(4-methyl-piperazin-1-yl)-thieno[3,2-d]pyrimidin-4-yl]-4-(6H-thieno[2,3-b]pyrrol-4-yl)-pyrrole-2,5-dione;
3-[2-(4-methyl-piperazin-1-yl)-6,7-dihydro-5H-cyclopentapyrimidin-4-yl]-4-(6H-thieno[2,3-b]pyrrol-4-yl)-pyrrole-2,5-dione;
3-[2-(2-methyl-octahydro-pyrrolo[3,4-c]pyridin-5-yl)-quinazolin-4-yl]-4-(6H-thieno[2,3-b]pyrrol-4-yl)-pyrrole-2,5-dione;
3-[2-(3-methyl-5,6-dihydro-8H-[1,2,4]triazolo[4,3-a]pyrazin-7-yl)-quinazolin-4-yl]-4-(6H-thieno[2,3-b]pyrrol-4-yl)-pyrrole-2,5-dione;
3-[2-(3-hydroxymethyl-5,6-dihydro-8H-imidazo[1,5-a]pyrazin-7-yl)-quinazolin-4-yl]-4-(6H-thieno[2,3-b]pyrrol-4-yl)-pyrrole-2,5-dione;
3-[2-(3-dimethylaminomethyl-5,6-dihydro-8H-imidazo[1,5-a]pyrazin-7-yl)-quinazolin-4-yl]-4-(6H-thieno[2,3-b]pyrrol-4-yl)-pyrrole-2,5-dione;
3-{2-[3-(1-hydroxy-1-methyl-ethyl)-5,6-dihydro-8H-imidazo[1,5-a]pyrazin-7-yl]-quinazolin-4-yl}-4-(6H-thieno[2,3-b]pyrrol-4-yl)-pyrrole-2,5-dione;
3-[2-(5,6-Dihydro-8H-imidazo[1,5-a]pyrazin-7-yl)-quinazolin-4-yl]-4-(6H-thieno[2,3-b]pyrrol-4-yl)-pyrrole-2,5-dione;
3-[2-(1-methyl-1,4,6,7-tetrahydro-imidazo[4,5-c]pyridin-5-yl)-quinazolin-4-yl]-4-(6H-thieno[2,3-b]pyrrol-4-yl)-pyrrole-2,5-dione;
3-[2-(3-methyl-3,4,6,7-tetrahydro-imidazo[4,5-c]pyridin-5-yl)-quinazolin-4-yl]-4-(6H-thieno[2,3-b]pyrrol-4-yl)-pyrrole-2,5-dione;
3-[2-(4-dimethylamino-4-methyl-piperidin-1-yl)-quinazolin-4-yl]-4-(6H-thieno[2,3-b]pyrrol-4-yl)-pyrrole-2,5-dione;
3-[2-(3-hydroxymethyl-4-methyl-piperazin-1-yl)-quinazolin-4-yl]-4-(6H-thieno[2,3-b]pyrrol-4-yl)-pyrrole-2,5-dione;
3-[2-(4-methyl-piperazin-1-yl)-pyrido[3,4-d]pyrimidin-4-yl]-4-(6H-thieno[2,3-b]pyrrol-4-yl)-pyrrole-2,5-dione;
3-[2-(4-methyl-piperazin-1-yl)-pyrido[3,2-d]pyrimidin-4-yl]-4-(6H-thieno[2,3-b]pyrrol-4-yl)-pyrrole-2,5-dione;
3-[2-((3R,4R)-4-dimethylamino-3-hydroxymethyl-piperidin-1-yl)-quinazolin-4-yl]-4-(6H-thieno[2,3-b]pyrrol-4-yl)-pyrrole-2,5-dione;
3-{2-[3-(1-hydroxy-ethyl)-5,6-dihydro-8H-imidazo[1,5-a]pyrazin-7-yl]-quinazolin-4-yl}-4-(6H-thieno[2,3-b]pyrrol-4-yl)-pyrrole-2,5-dione;
3-(2-piperidin-4-ylmethyl-2H-indazol-3-yl)-4-(6H-thieno[2,3-b]pyrrol-4-yl)-pyrrole-2,5-dione;
3-{1-[1-(2-fluoro-ethyl)-piperidin-4-ylmethyl]-1H-indazol-3-yl}-4-(6H-thieno[2,3-b]pyrrol-4-yl)-pyrrole-2,5-dione;
3-{1-[1-(2-methoxy-ethyl)-piperidin-4-ylmethyl]-1H-indazol-3-yl}-4-(6H-thieno[2,3-b]pyrrol-4-yl)-pyrrole-2,5-dione;
3-[1-(3-dimethylamino-propyl)-1H-indazol-3-yl]-4-(6H-thieno[2,3-b]pyrrol-4-yl)-pyrrole-2,5-dione;
3-[1-(1-methyl-piperidin-3-ylmethyl)-1H-indazol-3-yl]-4-(6H-thieno[2,3-b]pyrrol-4-yl)-pyrrole-2,5-dione;
3-[5-methyl-2-(4-methyl-piperazin-1-yl)-quinazolin-4-yl]-4-(6H-thieno[2,3-b]pyrrol-4-yl)-pyrrole-2,5-dione;
3-[2-(4-methyl-4-methylamino-piperidin-1-yl)-quinazolin-4-yl]-4-(6H-thieno[2,3-b]pyrrol-4-yl)-pyrrole-2,5-dione;
3-[1-(1-ethyl-piperidin-4-ylmethyl)-1H-indazol-3-yl]-4-(6H-thieno[2,3-b]pyrrol-4-yl)-pyrrole-2,5-dione;
3-{1-[1-(2-methoxy-ethyl)-pyrrolidin-3-ylmethyl]-1H-indazol-3-yl}-4-(6H-thieno[2,3-b]pyrrol-4-yl)-pyrrole-2,5-dione;
3-[2-((3S,4S)-3-fluoro-4-pyrrolidin-1-yl-piperidin-1-yl)-quinazolin-4-yl]-4-(6H-thieno[2,3-b]pyrrol-4-yl)-pyrrole-2,5-dione;
3-[2-((3R,4S)-3-fluoro-4-pyrrolidin-1-yl-piperidin-1-yl)-quinazolin-4-yl]-4-(6H-thieno[2,3-b]pyrrol-4-yl)-pyrrole-2,5-dione;
3-{2-[4-amino-4-(2-hydroxy-ethyl)-piperidin-1-yl]-quinazolin-4-yl}-4-(6H-thieno[2,3-b]pyrrol-4-yl)-pyrrole-2,5-dione;
3-[2-(3-ethyl-5,6-dihydro-8H-imidazo[1,5-a]pyrazin-7-yl)-quinazolin-4-yl]-4-(6H-thieno[2,3-b]pyrrol-4-yl)-pyrrole-2,5-dione;
3-[6-methyl-2-(4-methyl-piperazin-1-yl)-quinazolin-4-yl]-4-(6H-thieno[2,3-b]pyrrol-4-yl)-pyrrole-2,5-dione;
3-[8-methyl-2-(4-methyl-piperazin-1-yl)-quinazolin-4-yl]-4-(6H-thieno[2,3-b]pyrrol-4-yl)-pyrrole-2,5-dione;
3-[1-(4-dimethylamino-butyl)-1H-indazol-3-yl]-4-(6H-thieno[2,3-b]pyrrol-4-yl)-pyrrole-2,5-dione;
3-[2-(4-methyl-piperazin-1-yl)-thieno[2,3-d]pyrimidin-4-yl]-4-(6H-thieno[2,3-b]pyrrol-4-yl)-pyrrole-2,5-dione;
3-[2-((3R,4S)-4-dimethylamino-3-fluoro-piperidin-1-yl)-quinazolin-4-yl]-4-(6H-thieno[2,3-b]pyrrol-4-yl)-pyrrole-2,5-dione;
4-amino-1-{4-[2,5-dioxo-4-(6H-thieno[2,3-b]pyrrol-4-yl)-2,5-dihydro-1H-pyrrol-3-yl]-quinazolin-2-yl}-piperidine-4-carboxylic acid cyclopropylamide;
3-[1-(1-methyl-azetidin-3-ylmethyl)-1H-indazol-3-yl]-4-(6H-thieno[2,3-b]pyrrol-4-yl)-pyrrole-2,5-dione;
3-{2-[4-(2-hydroxy-1,1-dimethyl-ethyl)-piperazin-1-yl]-quinazolin-4-yl}-4-(6H-thieno[2,3-b]pyrrol-4-yl)-pyrrole-2,5-dione;
3-{2-[4-(2-hydroxy-1,1-dimethyl-ethyl)-piperazin-1-yl]-quinazolin-4-yl}-4-(6H-thieno[2,3-b]pyrrol-4-yl)-pyrrole-2,5-dione;
3-{2-[4-(2-hydroxy-1,1-dimethyl-ethyl)-piperazin-1-yl]-quinazolin-4-yl}-4-(6H-thieno[2,3-b]pyrrol-4-yl)-pyrrole-2,5-dione;
3-{2-[4-(2-hydroxy-2-methyl-propyl)-piperazin-1-yl]-quinazolin-4-yl}-4-(6H-thieno[2,3-b]pyrrol-4-yl)-pyrrole-2,5-dione;
3-[2-(4-amino-4-hydroxymethyl-piperidin-1-yl)-quinazolin-4-yl]-4-(6H-thieno[2,3-b]pyrrol-4-yl)-pyrrole-2,5-dione;
3-[2-(4-amino-4-ethyl-piperidin-1-yl)-quinazolin-4-yl]-4-(6H-thieno[2,3-b]pyrrol-4-yl)-pyrrole-2,5-dione;
3-[2-(4-amino-4-ethyl-piperidin-1-yl)-quinazolin-4-yl]-4-(6H-thieno[2,3-b]pyrrol-4-yl)-pyrrole-2,5-dione;
3-[2-(1-hydroxymethyl-5,6-dihydro-8H-imidazo[1,5-a]pyrazin-7-yl)-quinazolin-4-yl]-4-(6H-thieno[2,3-b]pyrrol-4-yl)-pyrrole-2,5-dione;
3-[2-(1-hydroxymethyl-5,6-dihydro-8H-imidazo[1,5-a]pyrazin-7-yl)-quinazolin-4-yl]-4-(6H-thieno[2,3-b]pyrrol-4-yl)-pyrrole-2,5-dione;
3-[(S)-2-(hexahydro-pyrrolo[1,2-a]pyrazin-2-yl)-quinazolin-4-yl]-4-(6H-thieno[2,3-b]pyrrol-4-yl)-pyrrole-2,5-dione;
3-[(S)-2-(hexahydro-pyrrolo[1,2-a]pyrazin-2-yl)-quinazolin-4-yl]-4-(6H-thieno[2,3-b]pyrrol-4-yl)-pyrrole-2,5-dione;
3-[2-(4-methyl-piperazin-1-yl)-pyrrolo[2,1-f][1,2,4]triazin-4-yl]-4-(6H-thieno[2,3-b]pyrrol-4-yl)-pyrrole-2,5-dione;
3-[2-(4-methyl-piperazin-1-yl)-pyrrolo[2,1-f][1,2,4]triazin-4-yl]-4-(6H-thieno[2,3-b]pyrrol-4-yl)-pyrrole-2,5-dione;
3-[2-(4-methyl-piperazin-1-yl)-pyrrolo[2,1-f][1,2,4]triazin-4-yl]-4-(6H-thieno[2,3-b]pyrrol-4-yl)-pyrrole-2,5-dione;
3-[1-(4-dimethylamino-cyclohexyl)-1H-indazol-3-yl]-4-(6H-thieno[2,3-b]pyrrol-4-yl)-pyrrole-2,5-dione;
3-[1-(4-dimethylamino-cyclohexyl)-1H-indazol-3-yl]-4-(6H-thieno[2,3-b]pyrrol-4-yl)-pyrrole-2,5-dione;
3-[1-(1,4-dimethyl-piperidin-4-ylmethyl)-1H-indazol-3-yl]-4-(6H-thieno[2,3-b]pyrrol-4-yl)-pyrrole-2,5-dione;
3-[1-(1,4-dimethyl-piperidin-4-ylmethyl)-1H-indazol-3-yl]-4-(6H-thieno[2,3-b]pyrrol-4-yl)-pyrrole-2,5-dione;
3-[1-((3S,4R)-3-fluoro-piperidin-4-ylmethyl)-1H-indazol-3-yl]-4-(6H-thieno[2,3-b]pyrrol-4-yl)-pyrrole-2,5-dione;
3-[1-((3S,4R)-3-fluoro-piperidin-4-ylmethyl)-1H-indazol-3-yl]-4-(6H-thieno[2,3-b]pyrrol-4-yl)-pyrrole-2,5-dione;
3-[1-((3S,4R)-3-fluoro-1-methyl-piperidin-4-ylmethyl)-1H-indazol-3-yl]-4-(6H-thieno[2,3-b]pyrrol-4-yl)-pyrrole-2,5-dione;
3-[2-(1,7-diaza-spiro[3.5]non-7-yl)-quinazolin-4-yl]-4-(6H-thieno[2,3-b]pyrrol-4-yl)-pyrrole-2,5-dione;
3-[2-(1,7-diaza-spiro[3.5]non-1-yl)-quinazolin-4-yl]-4-(6H-thieno[2,3-b]pyrrol-4-yl)-pyrrole-2,5-dione;
3-[3-(1-methyl-piperidin-4-ylmethyl)-indol-1-yl]-4-(6H-thieno[2,3-b]pyrrol-4-yl)-pyrrole-2,5-dione;
3-[5-methoxy-2-(4-methyl-piperazin-1-yl)-quinazolin-4-yl]-4-(6H-thieno[2,3-b]pyrrol-4-yl)-pyrrole-2,5-dione;
3-[5-methoxy-2-(4-methyl-piperazin-1-yl)-quinazolin-4-yl]-4-(6H-thieno[2,3-b]pyrrol-4-yl)-pyrrole-2,5-dione;
3-{1-[1-(1-methyl-piperidin-4-yl)-ethyl]-1H-indazol-3-yl}-4-(6H-thieno[2,3-b]pyrrol-4-yl)-pyrrole-2,5-dione;
3-[5-hydroxy-2-(4-methyl-piperazin-1-yl)-quinazolin-4-yl]-4-(6H-thieno[2,3-b]pyrrol-4-yl)-pyrrole-2,5-dione;
3-[2-((3S,4S)-3-fluoro-4-pyrrolidin-1-yl-piperidin-1-yl)-quinazolin-4-yl]-4-(6H-thieno[2,3-b]pyrrol-4-yl)-pyrrole-2,5-dione;
3-[2-((3R,4R)-3-fluoro-4-pyrrolidin-1-yl-piperidin-1-yl)-quinazolin-4-yl]-4-(6H-thieno[2,3-b]pyrrol-4-yl)-pyrrole-2,5-dione;
3-[6-chloro-2-(4-methyl-piperazin-1-yl)-quinazolin-4-yl]-4-(6H-thieno[2,3-b]pyrrol-4-yl)-pyrrole-2,5-dione;
3-[6-methoxy-2-(4-methyl-piperazin-1-yl)-quinazolin-4-yl]-4-(6H-thieno[2,3-b]pyrrol-4-yl)-pyrrole-2,5-dione;
3-[6-methoxy-2-(4-methyl-piperazin-1-yl)-quinazolin-4-yl]-4-(6H-thieno[2,3-b]pyrrol-4-yl)-pyrrole-2,5-dione;
3-[6-methoxy-2-(4-methyl-piperazin-1-yl)-quinazolin-4-yl]-4-(6H-thieno[2,3-b]pyrrol-4-yl)-pyrrole-2,5-dione;
3-[(S)-2-(hexahydro-pyrrolo[1,2-a]pyrazin-2-yl)-thieno[2,3-d]pyrimidin-4-yl]-4-(6H-thieno[2,3-b]pyrrol-4-yl)-pyrrole-2,5-dione;
3-[(3aR,7aS)-2-(octahydro-pyrrolo[3,2-c]pyridin-5-yl)-quinazolin-4-yl]-4-(6H-thieno[2,3-b]pyrrol-4-yl)-pyrrole-2,5-dione;
3-[2-((3aR,7aS)-1-methyl-octahydro-pyrrolo[3,2-c]pyridin-5-yl)-quinazolin-4-yl]-4-(6H-thieno[2,3-b]pyrrol-4-yl)-pyrrole-2,5-dione;
3-[(R)-2-(hexahydro-pyrrolo[1,2-a]pyrazin-2-yl)-quinazolin-4-yl]-4-(6H-thieno[2,3-b]pyrrol-4-yl)-pyrrole-2,5-dione;
3-[(R)-2-(hexahydro-pyrrolo[1,2-a]pyrazin-2-yl)-quinazolin-4-yl]-4-(4H-thieno[3,2-b]pyrrol-6-yl)-pyrrole-2,5-dione;
3-[(R)-2-(hexahydro-pyrrolo[1,2-a]pyrazin-2-yl)-quinazolin-4-yl]-4-(4H-thieno[3,2-b]pyrrol-6-yl)-pyrrole-2,5-dione;
3-(2-piperazin-1-yl-thieno[2,3-d]pyrimidin-4-yl)-4-(6H-thieno[2,3-b]pyrrol-4-yl)-pyrrole-2,5-dione;
3-[7-fluoro-3-(4-methyl-piperazin-1-yl)-isoquinolin-1-yl]-4-(6H-thieno[2,3-b]pyrrol-4-yl)-pyrrole-2,5-dione;
3-[7-fluoro-3-(4-methyl-piperazin-1-yl)-isoquinolin-1-yl]-4-(6H-thieno[2,3-b]pyrrol-4-yl)-pyrrole-2,5-dione;
3-[6-fluoro-2-(4-methyl-piperazin-1-yl)-quinazolin-4-yl]-4-(6H-thieno[2,3-b]pyrrol-4-yl)-pyrrole-2,5-dione;
3-[6-fluoro-2-(4-methyl-piperazin-1-yl)-quinazolin-4-yl]-4-(6H-thieno[2,3-b]pyrrol-4-yl)-pyrrole-2,5-dione;
3-(1H-indol-3-yl)-4-[6-(1-methyl-piperidin-4-ylmethyl)-6H-thieno[2,3-b]pyrrol-4-yl]-pyrrole-2,5-dione;
3-(1H-indol-3-yl)-4-[6-(1-methyl-piperidin-4-ylmethyl)-6H-thieno[2,3-b]pyrrol-4-yl]-pyrrole-2,5-dione;
3-[1-(4-dimethylamino-cyclohexylmethyl)-1H-indazol-3-yl]-4-(6H-thieno[2,3-b]pyrrol-4-yl)-pyrrole-2,5-dione;
3-[1-(4-dimethylamino-cyclohexylmethyl)-1H-indazol-3-yl]-4-(6H-thieno[2,3-b]pyrrol-4-yl)-pyrrole-2,5-dione;
3-[2-(4-methyl-piperazin-1-yl)-thieno[2,3-d]pyrimidin-4-yl]-4-(4H-thieno[3,2-b]pyrrol-6-yl)-pyrrole-2,5-dione;
3-[5-fluoro-2-(4-methyl-piperazin-1-yl)-quinazolin-4-yl]-4-(6H-thieno[2,3-b]pyrrol-4-yl)-pyrrole-2,5-dione;
3-[5-fluoro-2-(4-methyl-piperazin-1-yl)-quinazolin-4-yl]-4-(6H-thieno[2,3-b]pyrrol-4-yl)-pyrrole-2,5-dione;
3-[3-(1-methyl-piperidin-4-ylmethyl)-imidazo[1,5-a]pyridin-1-yl]-4-(6H-thieno[2,3-b]pyrrol-4-yl)-pyrrole-2,5-dione;
3-[2-(4-methyl-piperazin-1-yl)-quinolin-4-yl]-4-(4H-thieno[3,2-b]pyrrol-6-yl)-pyrrole-2,5-dione;
3-(8-hydroxymethyl-6,7,8,9-tetrahydro-pyrido[1,2-a]indol-10-yl)-4-(6H-thieno[2,3-b]pyrrol-4-yl)-pyrrole-2,5-dione;
3-[5-fluoro-2-(4-methyl-piperazin-1-yl)-quinazolin-4-yl]-4-(4H-thieno[3,2-b]pyrrol-6-yl)-pyrrole-2,5-dione; or
3-(8-dimethylaminomethyl-6,7,8,9-tetrahydro-pyrido[1,2-a]indol-10-4-(6H-thieno[2,3-b]pyrrol-4-yl)-pyrrole-2,5-dione.

In a thirteenth embodiment the invention provides a method of inhibiting one or more protein kinase activity in a patient comprising administering a therapeutically effective amount of a compound of any of the foregoing embodiments or a physiologically acceptable salt, pro-drug or biologically active metabolites thereof to said patient.

In a fourteenth embodiment the invention provides a method according to any of the foregoing embodiments wherein said protein kinase is selected from the group consisting of PKC, Jak1, Jak2, Jak3, Tyk2, KDR, Flt-3, ROCK, CDK2, CDK4, TANK, Trk, FAK, Abl, Bcr-Abl, cMet, b-RAF, FGFR3, c-kit, PDGF-R, Syk, or Aurora kinases.

In a fifteenth embodiment the invention provides a method of treating a condition in a patient comprising administering a therapeutically effective amount of a compound according to any of the foregoing embodiments or a physiologically acceptable salt, pro-drug or biologically active metabolites thereof to said patient, wherein said condition is an immunological disorder, an oncological disorder, a diabetic disorder or organ transplant.

In a sixteenth embodiment the invention provides a method according to any of the foregoing embodiments wherein the immunological disorder is rheumatoid arthritis, ankylosing spondylitis, juvenile rheumatoid arthritis, Crohn's Disease, psoriatic arthritis, juvenile idiopathic arthritis, plaque psoriasis, multiple sclerosis, psosiasis, ulcerative colitis or inflammatory bowel disease or uveitis.

In a seventeenth embodiment the invention provides a method according to any of the foregoing embodiments wherein the oncological disorder is cancer, lymphoma, myeloma, leukaemia, malignant ascites, hematopoietic cancers, lung cancer, breast cancer, colon cancer or bladder cancer.

In an eighteenth embodiment the invention provides a method according to any of the foregoing embodiments wherein the diabetic disorder is diabetes, insulin-dependent diabetes mellitus glaucoma, diabetic retinopathy, macular edema, diabetic neuropathy or microangiopathy,

In a nineteenth embodiment the invention provides a method according to any of the foregoing embodiments wherein the organ transplant is liver, heart, lung or kidney transplant.

In a twentieth embodiment the invention provides a pharmaceutical composition comprising a compound according to any of the foregoing embodiments and a pharmaceutically acceptable carrier or diluent.

### DETAILED DESCRIPTION OF THE INVENTION

Protein kinases are a broad and diverse class, of over 500 enzymes, that include oncogenes, growth factors receptors, signal transduction intermediates, apoptosis related kinases and cyclin dependent kinases. They are responsible for the transfer of a phosphate group to specific tyrosine, serine or threonine amino acid residues, and are broadly classified as tyrosine and serine/threonine kinases as a result of their substrate specificity.

The protein kinase C family is a group of serine/threonine kinases that comprises twelve related isoenzymes. Its members are encoded by different genes and are sub-classified according to their requirements for activation. The classical enzymes (cPKC) require diacylglycerol (DAG), phosphatidylserine (PS) and calcium for activation. The novel PKC's (nPKC) require DAG and PS but are calcium independent. The atypical PKC's (aPKC) do not require calcium or DAG.

PKCtheta is a member of the nPKC sub-family (Baier, G., et al., J. Biol. Chem., 1993, 268, 4997). It has a restricted expression pattern, found predominantly in T cells and skeletal muscle (Mischak, H. et al., FEBS Lett., 1993, 326, p. 51), with some expression reported in mast cells (Liu, Y. et al., J. Leukoc. Biol., 2001, 69, p. 831) and endothelial cells (Mattila, P. et al., Life Sci., 1994, 55, p. 1253).

Upon T cell activation, a supramolecular activation complex (SMAC) forms at the site of contact between the T cell and the antigen presenting cell (APC). PKCtheta is the only PKC isoform found to localize at the SMAC (Monks, C. et al., Nature, 1997, 385, 83), placing it in proximity with other signaling enzymes that mediate T cell activation processes.

In another study (Baier-Bitterlich, G. et al., Mol. Cell. Biol., 1996, 16, 842) the role of PKCtheta in the activation of AP-1, a transcription factor important in the activation of the IL-2 gene, was confirmed. In unstimulated T cells, constitutively active PKCtheta stimulated AP-1 activity while in cells with dominant negative PKCtheta, AP-1 activity was not induced upon activation by PMA.

Other studies showed that PKCtheta, via activation of IκB kinase beta, mediates activation of NF-κB induced by T cell receptor/CD28 co-stimulation (N. Coudronniere et al., Proc. Nat. Acad. Sci. U.S.A., 2000, 97, p. 3394; and Lin, X. et al., Mol. Cell. Biol., 2000, 20, p. 2933).

Proliferation of peripheral T cells from PKCtheta knockout mice, in response to T cell receptor (TCR)/CD28 stimulation was greatly diminished compared to T cells from wild type mice. In addition, the amount of IL-2 released from the T cells was also greatly reduced (Sun, Z. et al., Nature, 2000, 404, p. 402). It has also been shown that PKCtheta-deficient mice show impaired pulmonary inflammation and airway hyperresponsiveness (AHR) in a Th2-dependent murine asthma model, with no defects in viral clearance and Th1-dependent cytotoxic T cell function (Berg-Brown, N. N. et al., J. Exp. Med., 2004, 199, p. 743; Marsland, B. J. et al., J. Exp. Med., 2004, 200, p. 181). The impaired Th2 cell response results in reduced levels of IL-4 and immunoglobulin E (IgE), contributing to the AHR and inflammatory pathophysiology. Otherwise, the PKCtheta knockout mice seemed normal and fertile.

Evidence also exists that PKCtheta participates in the IgE receptor (FcεRI)-mediated response of mast cells (Liu, Y. et al., J. Leukoc. Biol., 2001, 69, p. 831). In human-cultured mast cells (HCMC), it has been demonstrated that PKC kinase activity rapidly localizes to the membrane following FcεRI cross-linking (Kimata, M. et al., Biochem. Biophys. Res. Commun., 1999, 257(3), p. 895). A recent study examining in vitro activity of bone marrow mast cells (BMMC) derived from wild-type and PKCtheta-deficient mice shows that upon FceRI cross linking, BMMCs from PKCtheta-deficient mice reduced levels of IL-6, tumor necrosis factor-alpha (TNFα) and IL-13 in comparison with BMMCs from wild-type mice, suggesting a potential role for PKCtheta in mast cell cytokine production in addition to T cell activation (Ciarletta, A. B. et al., poster presentation at the 2005 American Thoracic Society International Conference).

The studies cited above and others studies confirm the critical role of PKCtheta in T cells activation and in mast cell (MC) signaling. Thus an inhibitor of PKCtheta would be of therapeutic benefit in treating immunological disorders and other diseases mediated by the inappropriate activation of T cells and MC signaling.

Many of the kinases, whether a receptor or non-receptor tyrosine kinase or a S/T kinase have been found to be involved in cellular signaling pathways involved in numerous pathogenic conditions, including immunomodulation, inflammation, or proliferative disorders such as cancer.

Many autoimmune diseases and disease associated with chronic inflammation, as well as acute responses, have been linked to excessive or unregulated production or activity of one or more cytokines.

The compounds of the invention are also useful in the treatment of cardiovascular disorders, such as acute myocardial infarction, acute coronary syndrome, chronic heart failure, myocardial infarction, atherosclerosis, viral myocarditis, cardiac allograft rejection, and sepsis-associated cardiac dysfunction. Furthermore, the compounds of the present invention are also useful for the treatment of central nervous system disorders such as meningococcal meningitis, Alzheimer's disease and Parkinson's disease.

The compounds of the invention are also useful in the treatment of an ocular condition, a cancer, rheumatoid arthritis, ankylosing spondilitis, a solid tumor, a sarcoma, fibrosarcoma, osteoma, melanoma, retinoblastoma, a rhabdomyosarcoma, glioblastoma, neuroblastoma, teratocarcinoma, hypersensitivity reactions, hyperkinetic movement disorders, hypersensitivity pneumonitis, hypertension, hypokinetic movement disorders, aordic and peripheral aneuryisms, hypothalamic-pituitary-adrenal axis evaluation, aortic dissection, arterial hypertension, arteriosclerosis, arteriovenous fistula, ataxia, spinocerebellar degenerations, streptococcal myositis, structural lesions of the cerebellum, subacute sclerosing panencephalitis, Syncope, syphilis of the cardiovascular system, systemic anaphalaxis, systemic inflammatory response syndrome, systemic onset juvenile rheumatoid arthritis, T-cell or FAB ALL, telangiectasia, thromboangitis obliterans, transplants, trauma/hemorrhage, type III hypersensitivity reactions, type IV hypersensitivity, unstable angina, uremia, urosepsis, urticaria, valvular heart diseases, varicose veins, vasculitis, venous diseases, venous thrombosis, ventricular fibrillation, viral and fungal infections, vital encephalitis/aseptic meningitis, vital-associated hemaphagocytic syndrome, Wernicke-Korsakoff syndrome, Wilson's disease, xenograft rejection of any organ or tissue, heart transplant rejection, hemachromatosis, hemodialysis, hemolytic uremic syndrome/thrombolytic thrombocytopenic purpura, hemorrhage, idiopathic pulmonary fibrosis, antibody mediated cytotoxicity, Asthenia, infantile spinal muscular atrophy, inflammation of the aorta, influenza A, ionizing radiation exposure, iridocyclitis/uveitis/optic neuritis, juvenile spinal muscular atrophy, lymphoma, myeloma, leukaemia, malignant ascites, hematopoietic cancers, a diabetic condition such as insulin-dependent diabetes mellitus glaucoma, diabetic retinopathy or microangiopathy, sickle cell anaemia, chronic inflammation, glomerulonephritis, graft rejection, Lyme disease, von Hippel Lindau disease, pemphigoid, Paget's disease, fibrosis, sarcoidosis, cirrhosis, thyroiditis, hyperviscosity syndrome, Osler-Weber-Rendu disease, chronic occlusive pulmonary disease, asthma or edema following burns, trauma, radiation, stroke, hypoxia, ischemia, ovarian hyperstimulation syndrome, post perfusion syndrome, post pump syndrome, post-MI cardiotomy syndrome, preeclampsia, menometrorrhagia, endometriosis, pulmonary hypertension, infantile hemangioma, or infection by Herpes simplex, Herpes Zoster, human immunodeficiency virus, parapoxvirus, protozoa or toxoplasmosis, progressive supranucleo palsy, primary pulmonary hypertension, radiation therapy, Raynaud's phenomenon, Raynaud's disease, Refsum's disease, regular narrow QRS tachycardia, renovascular hypertension, restrictive cardiomyopathy, sarcoma, senile chorea, senile dementia of Lewy body type, shock, skin allograft, skin changes syndrome, ocular or macular edema, ocular neovascular disease, scleritis, radial keratotomy, uveitis, vitritis, myopia, optic pits, chronic retinal detachment, post-laser treatment complications, conjunctivitis, Stargardt's disease, Eales disease, retinopathy, macular degeneration, restenosis, ischemia/reperfusion injury, ischemic stroke, vascular occlusion, carotid obstructive disease, ulcerative colitis, inflammatory bowel disease, diabetes, diabetes mellitus, insulin dependent diabetes mellitus, allergic diseases, dermatitis scleroderma, graft versus host disease, organ transplant rejection (including but not limited to bone marrow and solid organ rejection), acute or chronic immune disease associated with organ transplantation, sarcoidosis, disseminated intravascular coagulation, Kawasaki's disease, nephrotic syndrome, chronic fatigue syndrome, Wegener's granulomatosis, Henoch-Schoenlein purpurea, microscopic vasculitis of the kidneys, chronic active hepatitis, septic shock, toxic shock syndrome, sepsis syndrome, cachexia, infectious diseases, parasitic diseases, acquired immunodeficiency syndrome, acute transverse myelitis, Huntington's chorea, stroke, primary biliary cirrhosis, hemolytic anemia, malignancies, Addison's disease, idiopathic Addison's disease, sporadic, polyglandular deficiency type I and polyglandular deficiency type II, Schmidt's syndrome, adult (acute) respiratory distress syndrome, alopecia, alopecia greata, seronegative arthopathy, arthropathy, Reiter's disease, psoriatic arthropathy, ulcerative colitic arthropathy, enteropathic synovitis, chlamydia, yersinia and salmonella associated arthropathy, atheromatous disease/arteriosclerosis, atopic allergy, autoimmune bullous disease, pemphigus vulgaris, pemphigus foliaceus, pemphigoid, linear IgA disease, autoimmune haemolytic anaemia, Coombs positive haemolytic anaemia, acquired pernicious anaemia, juvenile pernicious anaemia, peripheral vascular disorders, peritonitis, pernicious anemia, myalgic encephalitis/Royal Free Disease, chronic mucocutaneous candidiasis, giant cell arteritis, primary sclerosing hepatitis, cryptogenic autoimmune hepatitis, Acquired Immunodeficiency Disease Syndrome, Acquired Immunodeficiency Related Diseases, Hepatitis A, Hepatitis B, Hepatitis C, His bundle arrythmias, HIV infection/HIV neuropathy, common varied immunodeficiency (common variable hypogammaglobulinemia), dilated cardiomyopathy, female infertility, ovarian failure, premature ovarian failure, fibrotic lung disease, chronic wound healing, cryptogenic fibrosing alveolitis, post-inflammatory interstitial lung disease, interstitial pneumonitis, pneumocystis carinii pneumonia, pneumonia, connective tissue disease associated interstitial lung disease, mixed connective tissue disease, associated lung disease, systemic sclerosis associated interstitial lung disease, rheumatoid arthritis associated interstitial lung disease, systemic lupus erythematosus associated lung disease, dermatomyositis/polymyositis associated lung disease, Sjögren's disease associated lung disease, ankylosing spondylitis associated lung disease, vasculitic diffuse lung disease, haemosiderosis associated lung disease, drug-induced interstitial lung disease, radiation fibrosis, bronchiolitis obliterans, chronic eosinophilic pneumonia, lymphocytic infiltrative lung disease, postinfectious interstitial lung disease, gouty arthritis, autoimmune hepatitis, type-1 autoimmune hepatitis (classical autoimmune or lupoid hepatitis), type-2 autoimmune hepatitis (anti-LKM antibody hepatitis), autoimmune mediated hypoglycaemia, type B insulin resistance with acanthosis nigricans, hypoparathyroidism, acute immune disease associated with organ transplantation, chronic immune disease associated with organ transplantation, osteoarthritis, primary sclerosing cholangitis, psoriasis type 1, psoriasis type 2, idiopathic leucopaenia, autoimmune neutropaenia, renal disease NOS, glomerulonephritides, microscopic vasulitis of the kidneys, Lyme disease, discoid lupus erythematosus, male infertility idiopathic or NOS, sperm autoimmunity, multiple sclerosis (all subtypes), sympathetic ophthalmia, pulmonary hypertension secondary to connective tissue disease, acute and chronic pain (different forms of pain), Goodpasture's syndrome, pulmonary manifestation of polyarteritis nodosa, acute rheumatic fever, rheumatoid spondylitis, Still's disease, systemic sclerosis, Sjögren's syndrome, Takayasu's disease/arteritis, autoimmune thrombocytopenia, toxicity, transplants, and diseases involving inappropriate vascularization for example diabetic retinopathy, retinopathy of prematurity, choroidal neovascularization due to age-related macular degeneration, and infantile hemangiomas in human beings. In addition, such compounds may be useful in the treatment of disorders such as ascites, effusions, and exudates, including for example macular edema, cerebral edema, acute lung injury, adult respiratory distress syndrome (ARDS), proliferative disorders such as restenosis, fibrotic disorders such as hepatic cirrhosis and atherosclerosis, mesangial cell proliferative disorders such as diabetic nephropathy, malignant nephrosclerosis, thrombotic microangiopathy syndromes, and glomerulopathies, myocardial angiogenesis, coronary and cerebral collaterals, ischemic limb angiogenesis, ischemia/reperfusion injury, peptic ulcer Helicobacter related diseases, virally-induced angiogenic disorders, preeclampsia, menometrorrhagia, cat scratch fever, rubeosis, neovascular glaucoma and retinopathies such as those associated with diabetic retinopathy, retinopathy of prematurity, or age-related macular degeneration. In addition, these compounds can be used as active agents against hyperproliferative disorders such as thyroid hyperplasia (especially Grave's disease), and cysts (such as hypervascularity of ovarian stroma characteristic of polycystic ovarian syndrome (Stein-Leventhal syndrome) and polycystic kidney disease since such diseases require a proliferation of blood vessel cells for growth and/or metastasis.

Compounds of Formula (I) of the invention can be used alone or in combination with an additional agent, e.g., a therapeutic agent, said additional agent being selected by the skilled artisan for its intended purpose. For example, the additional agent can be a therapeutic agent art-recognized as being useful to treat the disease or condition being treated by the compound of the present invention. The additional agent also can be an agent that imparts a beneficial attribute to the therapeutic composition e.g., an agent that affects the viscosity of the composition.

It should further be understood that the combinations which are to be included within this invention are those combinations useful for their intended purpose. The agents set forth below are illustrative for purposes and not intended to be limited. The combinations, which are part of this invention, can be the compounds of the present invention and at least one additional agent selected from the lists below. The combination can also include more than one additional agent, e.g., two or three additional agents if the combination is such that the formed composition can perform its intended function.

Preferred combinations are non-steroidal anti-inflammatory drug(s) also referred to as NSAIDS which include drugs like ibuprofen. Other preferred combinations are corticosteroids including prednisolone; the well known side-effects of steroid use can be reduced or even eliminated by tapering the steroid dose required when treating patients in combination with the compounds of this invention. Non-limiting examples of therapeutic agents for rheumatoid arthritis with which a compound of Formula (I) of the invention can be combined include the following: cytokine suppressive anti-inflammatory drug(s) (CSAIDs); antibodies to or antagonists of other human cytokines or growth factors, for example, TNF, LT, IL-1, IL-2, IL-3, IL-4, IL-5, IL-6, IL-7, IL-8, IL-12, IL-15, IL-16, IL-21, IL-23, interferons, EMAP-II, GM-CSF, FGF, and PDGF. Compounds of the invention can be combined with antibodies to cell surface molecules such as CD2, CD3, CD4, CD8, CD25, CD28, CD30, CD40, CD45, CD69, CD80 (B7.1), CD86 (B7.2), CD90, CTLA or their ligands including CD154 (gp39 or CD40L).

Preferred combinations of therapeutic agents may interfere at different points in the autoimmune and subsequent inflammatory cascade; preferred examples include TNF antagonists like chimeric, humanized or human TNF antibodies, D2E7 (U.S. Pat. No. 6,090,382, HUMIRA™), CA2 (REMICADE™), SIMPONI™ (golimumab), CIMZIA™, ACTEMRA™, CDP 571, and soluble p55 or p75 TNF receptors, derivatives, thereof, p75TNFR1gG (ENBREL™) or p55TNFR1gG (Lenercept), and also TNFα converting enzyme (TACE) inhibitors; similarly IL-1 inhibitors (Interleukin-1-converting enzyme inhibitors, IL-1RA etc.) may be effective for the same reason. Other preferred combinations include Interleukin 11. Yet other preferred combinations are the other key players of the autoimmune response which may act parallel to, dependent on or in concert with IL-18 function; especially preferred are IL-12 antagonists including IL-12 antibodies or soluble IL-12 receptors, or IL-12 binding proteins. It has been shown that IL-12 and IL-18 have overlapping but distinct functions and a combination of antagonists to both may be most effective. Yet another preferred combination is non-depleting anti-CD4 inhibitors. Yet other preferred combinations include antagonists of the co-stimulatory pathway CD80 (B7.1) or CD86 (B7.2) including antibodies, soluble receptors or antagonistic ligands.

A compound of Formula (I) of the invention may also be combined with agents, such as methotrexate, 6-mercaptopurine, azathioprine sulphasalazine, mesalazine, olsalazine chloroquinine/hydroxychloroquine, pencillamine, aurothiomalate (intramuscular and oral), azathioprine, cochicine, corticosteroids (oral, inhaled and local injection), beta-2 adrenoreceptor agonists (salbutamol, terbutaline, salmeteral), xanthines (theophylline, aminophylline), cromoglycate, nedocromil, ketotifen, ipratropium and oxitropium, cyclosporin, FK506, rapamycin, mycophenolate mofetil, leflunomide, NSAIDs, for example, ibuprofen, corticosteroids such as prednisolone, phosphodiesterase inhibitors, adensosine agonists, antithrombotic agents, complement inhibitors, adrenergic agents, agents which interfere with signaling by proinflammatory cytokines such as TNFα or IL-1 (e.g., NIK, IKK, p38 or MAP kinase inhibitors), IL-1β converting enzyme inhibitors, T-cell signaling inhibitors such as kinase inhibitors, metalloproteinase inhibitors, sulfasalazine, 6-mercaptopurines, angiotensin converting enzyme inhibitors, soluble cytokine receptors and derivatives thereof (e.g. soluble p55 or p75 TNF receptors and the derivatives p75TNFRIgG (Enbrel™) and p55TNFRIgG (Lenercept), sIL-1RI, sIL-1RII, sIL-6R), antiinflammatory cytokines (e.g. IL-4, IL-10, IL-11, IL-13 and TGFβ), celecoxib, folic acid, hydroxychloroquine sulfate, rofecoxib, etanercept, infliximab, naproxen, valdecoxib, sulfasalazine, methylprednisolone, meloxicam, methylprednisolone acetate, gold sodium thiomalate, aspirin, triamcinolone acetonide, propoxyphene napsylate/apap, folate, nabumetone, diclofenac, piroxicam, etodolac, diclofenac sodium, oxaprozin, oxycodone HCl, hydrocodone bitartrate/apap, diclofenac sodium/misoprostol, fentanyl, anakinra, tramadol HCl, salsalate, sulindac, cyanocobalamin/fa/pyridoxine, acetaminophen, alendronate sodium, prednisolone, morphine sulfate, lidocaine hydrochloride, indomethacin, glucosamine sulf/chondroitin, amitriptyline HCl, sulfadiazine, oxycodone HCl/acetaminophen, olopatadine HCl misoprostol, naproxen sodium, omeprazole, cyclophosphamide, rituximab, IL-1 TRAP, MRA, CTLA4-IG, IL-18 BP, anti-IL-12, Anti-IL15, BIRB-796, SCIO-469, VX-702, AMG-548, VX-740, Roflumilast, IC-485, CDC-801, S1P1 agonists (such as FTY720), and Mesopram. Preferred combinations include methotrexate or leflunomide and in moderate or severe rheumatoid arthritis cases, cyclosporin and anti-TNF antibodies as noted above.

Non-limiting examples of therapeutic agents for inflammatory bowel disease with which a compound of Formula (I) of the invention can be combined include the following: budenoside; epidermal growth factor; corticosteroids; cyclosporin, sulfasalazine; aminosalicylates; 6-mercaptopurine; azathioprine; metronidazole; lipoxygenase inhibitors; mesalamine; olsalazine; balsalazide; antioxidants; thromboxane inhibitors; IL-1 receptor antagonists; anti-IL-1β monoclonal antibodies; anti-IL-6 monoclonal antibodies; growth factors; elastase inhibitors; pyridinyl-imidazole compounds; antibodies to or antagonists of other human cytokines or growth factors, for example, TNF, LT, IL-1, IL-2, IL-6, IL-7, IL-8, IL-12, IL-15, IL-16, IL-23, EMAP-II, GM-CSF, FGF, and PDGF; cell surface molecules such as CD2, CD3, CD4, CD8, CD25, CD28, CD30, CD40, CD45, CD69, CD90 or their ligands; methotrexate; cyclosporine; FK506; rapamycin; mycophenolate mofetil; leflunomide; NSAIDs, for example, ibuprofen; corticosteroids such as prednisolone; phosphodiesterase inhibitors; adenosine agonists; antithrombotic agents; complement inhibitors; adrenergic agents; agents which interfere with signaling by proinflammatory cytokines such as TNFα or IL-1 (e.g. NIK, IKK, or MAP kinase inhibitors); IL-1β converting enzyme inhibitors; TNFα converting enzyme inhibitors; T-cell signaling inhibitors such as kinase inhibitors; metalloproteinase inhibitors; sulfasalazine; azathioprine; 6-mercaptopurines; angiotensin converting enzyme inhibitors; soluble cytokine receptors and derivatives thereof (e.g. soluble p55 or p75 TNF receptors, sIL-1RI, sIL-1RII, sIL-6R) and antiinflammatory cytokines (e.g. IL-4, IL-10, IL-11, IL-13 and TGFβ). Preferred examples of therapeutic agents for Crohn's disease with which a compound of Formula (I) can be combined include the following: TNF antagonists, for example, anti-TNF antibodies, D2E7 (U.S. Pat. No. 6,090,382, HUMIRA™), CA2 (REMICADE™), CDP 571, TNFR-Ig constructs, (p75TNFRIgG (ENBREL™) and p55TNFRIgG (LENERCEPT™) inhibitors and PDE4 inhibitors. A compound of Formula (I) can be combined with corticosteroids, for example, budenoside and dexamethasone; sulfasalazine, 5-aminosalicylic acid; olsalazine; and agents which interfere with synthesis or action of proinflammatory cytokines such as IL-1, for example, IL-1β converting enzyme inhibitors and IL-1ra; T cell signaling inhibitors, for example, tyrosine kinase inhibitors; 6-mercaptopurine; IL-11; mesalamine; prednisone; azathioprine; mercaptopurine; infliximab; methylprednisolone sodium succinate; diphenoxylate/atrop sulfate; loperamide hydrochloride; methotrexate; omeprazole; folate; ciprofloxacin/dextrose-water; hydrocodone bitartrate/apap; tetracycline hydrochloride; fluocinonide; metronidazole; thimerosal/boric acid; cholestyramine/sucrose; ciprofloxacin hydrochloride; hyoscyamine sulfate; meperidine hydrochloride; midazolam hydrochloride; oxycodone HCl/acetaminophen; promethazine hydrochloride; sodium phosphate; sulfamethoxazole/trimethoprim; celecoxib; polycarbophil; propoxyphene napsylate; hydrocortisone; multivitamins; balsalazide disodium; codeine phosphate/apap; colesevelam HCl; cyanocobalamin; folic acid; levofloxacin; methylprednisolone; natalizumab and interferon-gamma.

Non-limiting examples of therapeutic agents for multiple sclerosis with which a compound of Formula (I) can be combined include the following: corticosteroids; prednisolone; methylprednisolone; azathioprine; cyclophosphamide; cyclosporine; methotrexate; 4-aminopyridine; tizanidine; interferon-β1a (AVONEX®; Biogen); interferon-β1b (BETASERON®; Chiron/Berlex); interferon α-n3) (Interferon Sciences/Fujimoto), interferon-α (Alfa Wassermann/J&J), interferon β1A-IF (Serono/Inhale Therapeutics), Peginterferon α 2b (Enzon/Schering-Plough), Copolymer 1 (Cop-1; COPAXONE®; Teva Pharmaceutical Industries, Inc.); hyperbaric oxygen; intravenous immunoglobulin; cladribine; antibodies to or antagonists of other human cytokines or growth factors and their receptors, for example, TNF, LT, IL-1, IL-2, IL-6, IL-7, IL-8, IL-12, IL-23, IL-15, IL-16, EMAP-II, GM-CSF, FGF, and PDGF. A compound of Formula (I) can be combined with antibodies to cell surface molecules such as CD2, CD3, CD4, CD8, CD19, CD20, CD25, CD28, CD30, CD40, CD45, CD69, CD80, CD86, CD90 or their ligands. A compound of Formula (I) may also be combined with agents such as methotrexate, cyclosporine, FK506, rapamycin, mycophenolate mofetil, leflunomide, an S1P1 agonist, NSAIDs, for example, ibuprofen, corticosteroids such as prednisolone, phosphodiesterase inhibitors, adensosine agonists, antithrombotic agents, complement inhibitors, adrenergic agents, agents which interfere with signaling by proinflammatory cytokines such as TNFα or IL-1 (e.g., NIK, IKK, p38 or MAP kinase inhibitors), IL-1β converting enzyme inhibitors, TACE inhibitors, T-cell signaling inhibitors such as kinase inhibitors, metalloproteinase inhibitors, sulfasalazine, azathioprine, 6-mercaptopurines, angiotensin converting enzyme inhibitors, soluble cytokine receptors and derivatives thereof (e.g. soluble p55 or p75 TNF receptors, sIL-1RI, sIL-1RII, sIL-6R) and antiinflammatory cytokines (e.g. IL-4, IL-10, IL-13 and TGFβ).

Preferred examples of therapeutic agents for multiple sclerosis in which a compound of Formula (I) can be combined to include interferon-β, for example, IFNβ1a and IFNβ1b; copaxone, corticosteroids, caspase inhibitors, for example inhibitors of caspase-1, IL-1 inhibitors, TNF inhibitors, and antibodies to CD40 ligand and CD80.

A compound of Formula (I) may also be combined with agents, such as alemtuzumab, dronabinol, daclizumab, mitoxantrone, xaliproden hydrochloride, fampridine, glatiramer acetate, natalizumab, sinnabidol, α-immunokine NNSO3, ABR-215062, AnergiX.MS, chemokine receptor antagonists, BBR-2778, calagualine, CPI-1189, LEM (liposome encapsulated mitoxantrone), THC.CBD (cannabinoid agonist), MBP-8298, mesopram (PDE4 inhibitor), MNA-715, anti-IL-6 receptor antibody, neurovax, pirfenidone allotrap 1258 (RDP-1258), sTNF-R1, talampanel, teriflunomide, TGF-beta2, tiplimotide, VLA-4 antagonists (for example, TR-14035, VLA4 Ultrahaler, Antegran-ELAN/Biogen), interferon gamma antagonists and IL-4 agonists.

Non-limiting examples of therapeutic agents for ankylosing spondylitis with which a compound of Formula (I) can be combined include the following: ibuprofen, diclofenac, misoprostol, naproxen, meloxicam, indomethacin, diclofenac, celecoxib, rofecoxib, sulfasalazine, methotrexate, azathioprine, minocyclin, prednisone, and anti-TNF antibodies, D2E7 (U.S. Pat. No. 6,090,382; HUMIRA™), CA2 (REMICADE™), CDP 571, TNFR-Ig constructs, (p75TNFRIgG (ENBREL™) and p55TNFRIgG (LENERCEPT™)

Non-limiting examples of therapeutic agents for asthma with which a compound of Formula (I) can be combined include the following: albuterol, salmeterol/fluticasone, montelukast sodium, fluticasone propionate, budesonide, prednisone, salmeterol xinafoate, levalbuterol HCl, albuterol sulfate/ipratropium, prednisolone sodium phosphate, triamcinolone acetonide, beclomethasone dipropionate, ipratropium bromide, azithromycin, pirbuterol acetate, prednisolone, theophylline anhydrous, methylprednisolone sodium succinate, clarithromycin, zafirlukast, formoterol fumarate, influenza virus vaccine, amoxicillin trihydrate, flunisolide, allergy injection, cromolyn sodium, fexofenadine hydrochloride, flunisolide/menthol, amoxicillin/clavulanate, levofloxacin, inhaler assist device, guaifenesin, dexamethasone sodium phosphate, moxifloxacin HCl, doxycycline hyclate, guaifenesin/d-methorphan, p-ephedrine/cod/chlorphenir, gatifloxacin, cetirizine hydrochloride, mometasone furoate, salmeterol xinafoate, benzonatate, cephalexin, pe/hydrocodone/chlorphenir, cetirizine HCl/pseudoephed, phenylephrine/cod/promethazine, codeine/promethazine, cefprozil, dexamethasone, guaifenesin/pseudoephedrine, chlorpheniramine/hydrocodone, nedocromil sodium, terbutaline sulfate, epinephrine, methylprednisolone, anti-IL-13 antibody, and metaproterenol sulfate.

Non-limiting examples of therapeutic agents for COPD with which a compound of Formula (I) can be combined include the following: albuterol sulfate/ipratropium, ipratropium bromide, salmeterol/fluticasone, albuterol, salmeterol xinafoate, fluticasone propionate, prednisone, theophylline anhydrous, methylprednisolone sodium succinate, montelukast sodium, budesonide, formoterol fumarate, triamcinolone acetonide, levofloxacin, guaifenesin, azithromycin, beclomethasone dipropionate, levalbuterol HCl, flunisolide, ceftriaxone sodium, amoxicillin trihydrate, gatifloxacin, zafirlukast, amoxicillin/clavulanate, flunisolide/menthol, chlorpheniramine/hydrocodone, metaproterenol sulfate, methylprednisolone, mometasone furoate, p-ephedrine/cod/chlorphenir, pirbuterol acetate, p-ephedrine/loratadine, terbutaline sulfate, tiotropium bromide, (R,R)-formoterol, TgAAT, cilomilast and roflumilast.

Non-limiting examples of therapeutic agents for HCV with which a compound of Formula (I) can be combined include the following: Interferon-alpha-2α, Interferon-alpha-2β, Interferon-alpha con1, Interferon-alpha-n1, pegylated interferon-alpha-2α, pegylated interferon-alpha-2β, ribavirin, peginterferon alfa-2b+ribavirin, ursodeoxycholic acid, glycyrrhizic acid, thymalfasin, Maxamine, VX-497 and any compounds that are used to treat HCV through intervention with the following targets: HCV polymerase, HCV protease, HCV helicase, and HCV IRES (internal ribosome entry site).

Non-limiting examples of therapeutic agents for Idiopathic Pulmonary Fibrosis with which a compound of Formula (I) can be combined include the following: prednisone, azathioprine, albuterol, colchicine, albuterol sulfate, digoxin, gamma interferon, methylprednisolone sodium succinate, lorazepam, furosemide, lisinopril, nitroglycerin, spironolactone, cyclophosphamide, ipratropium bromide, actinomycin d, alteplase, fluticasone propionate, levofloxacin, metaproterenol sulfate, morphine sulfate, oxycodone HCl, potassium chloride, triamcinolone acetonide, tacrolimus anhydrous, calcium, interferon-alpha, methotrexate, mycophenolate mofetil and interferon-gamma-1β.

Non-limiting examples of therapeutic agents for myocardial infarction with which a compound of Formula (I) can be combined include the following: aspirin, nitroglycerin, metoprolol tartrate, enoxaparin sodium, heparin sodium, clopidogrel bisulfate, carvedilol, atenolol, morphine sulfate, metoprolol succinate, warfarin sodium, lisinopril, isosorbide mononitrate, digoxin, furosemide, simvastatin, ramipril, tenecteplase, enalapril maleate, torsemide, retavase, losartan potassium, quinapril hydrochloride/magnesium carbonate, bumetanide, alteplase, enalaprilat, amiodarone hydrochloride, tirofiban HCl m-hydrate, diltiazem hydrochloride, captopril, irbesartan, valsartan, propranolol hydrochloride, fosinopril sodium, lidocaine hydrochloride, eptifibatide, cefazolin sodium, atropine sulfate, aminocaproic acid, spironolactone, interferon, sotalol hydrochloride, potassium chloride, docusate sodium, dobutamine HCl, alprazolam, pravastatin sodium, atorvastatin calcium, midazolam hydrochloride, meperidine hydrochloride, isosorbide dinitrate, epinephrine, dopamine hydrochloride, bivalirudin, rosuvastatin, ezetimibe/simvastatin, avasimibe, and cariporide.

Non-limiting examples of therapeutic agents for psoriasis with which a compound of Formula (I) can be combined include the following: calcipotriene, clobetasol propionate, triamcinolone acetonide, halobetasol propionate, tazarotene, methotrexate, fluocinonide, betamethasone diprop augmented, fluocinolone acetonide, acitretin, tar shampoo, betamethasone valerate, mometasone furoate, ketoconazole, pramoxine/fluocinolone, hydrocortisone valerate, flurandrenolide, urea, betamethasone, clobetasol propionate/emoll, fluticasone propionate, azithromycin, hydrocortisone, moisturizing formula, folic acid, desonide, pimecrolimus, coal tar, diflorasone diacetate, etanercept folate, lactic acid, methoxsalen, hc/bismuth subgal/znox/resor, methylprednisolone acetate, prednisone, sunscreen, halcinonide, salicylic acid, anthralin, clocortolone pivalate, coal extract, coal tar/salicylic acid, coal tar/salicylic acid/sulfur, desoximetasone, diazepam, emollient, fluocinonide/emollient, mineral oil/castor oil/na lact, mineral oil/peanut oil, petroleum/isopropyl myristate, psoralen, salicylic acid, soap/tribromsalan, thimerosal/boric acid, celecoxib, infliximab, cyclosporine, alefacept, efalizumab, tacrolimus, pimecrolimus, PUVA, UVB, sulfasalazine, ABT-874 and ustekinamab.

Non-limiting examples of therapeutic agents for psoriatic arthritis with which a compound of Formula (I) can be combined include the following: methotrexate, etanercept, rofecoxib, celecoxib, folic acid, sulfasalazine, naproxen, leflunomide, methylprednisolone acetate, indomethacin, hydroxychloroquine sulfate, prednisone, sulindac, betamethasone diprop augmented, infliximab, methotrexate, folate, triamcinolone acetonide, diclofenac, dimethylsulfoxide, piroxicam, diclofenac sodium, ketoprofen, meloxicam, methylprednisolone, nabumetone, tolmetin sodium, calcipotriene, cyclosporine, diclofenac sodium/misoprostol, fluocinonide, glucosamine sulfate, gold sodium thiomalate, hydrocodone bitartrate/apap, ibuprofen, risedronate sodium, sulfadiazine, thioguanine, valdecoxib, alefacept, D2E7 (U.S. Pat. No. 6,090,382, HUMIRA™), and efalizumab.

Non-limiting examples of therapeutic agents for restenosis with which a compound of Formula (I) can be combined include the following: sirolimus, paclitaxel, everolimus, tacrolimus, ABT-578, and acetaminophen.

Non-limiting examples of therapeutic agents for sciatica with which a compound of Formula (I) can be combined include the following: hydrocodone bitartrate/apap, rofecoxib, cyclobenzaprine HCl, methylprednisolone, naproxen, ibuprofen, oxycodone HCl/acetaminophen, celecoxib, valdecoxib, methylprednisolone acetate, prednisone, codeine phosphate/apap, tramadol HCl/acetaminophen, metaxalone, meloxicam, methocarbamol, lidocaine hydrochloride, diclofenac sodium, gabapentin, dexamethasone, carisoprodol, ketorolac tromethamine, indomethacin, acetaminophen, diazepam, nabumetone, oxycodone HCl, tizanidine HCl, diclofenac sodium/misoprostol, propoxyphene n-pap, asa/oxycod/oxycodone ter, ibuprofen/hydrocodone bit, tramadol HCl, etodolac, propoxyphene HCl, amitriptyline HCl, carisoprodol/codeine phos/asa, morphine sulfate, multivitamins, naproxen sodium, orphenadrine citrate, and temazepam.

Preferred examples of therapeutic agents for SLE (Lupus) with which a compound of Formula (I) can be combined include the following: NSAIDS, for example, diclofenac, naproxen, ibuprofen, piroxicam, indomethacin; COX2 inhibitors, for example, celecoxib, rofecoxib, valdecoxib; anti-malarials, for example, hydroxychloroquine; steroids, for example, prednisone, prednisolone, budenoside, dexamethasone; cytotoxics, for example, azathioprine, cyclophosphamide, mycophenolate mofetil, methotrexate; inhibitors of PDE4 or purine synthesis inhibitor, for example Cellcept®. A compound of Formula (I) may also be combined with agents such as sulfasalazine, 5-aminosalicylic acid, olsalazine, Imuran® and agents which interfere with synthesis, production or action of proinflammatory cytokines such as IL-1, for example, caspase inhibitors like IL-1β converting enzyme inhibitors and IL-1ra. A compound of Formula (I) may also be used with T cell signaling inhibitors, for example, tyrosine kinase inhibitors; or molecules that target T cell activation molecules, for example, CTLA-4-IgG or anti-B7 family antibodies, anti-PD-1 family antibodies. A compound of Formula (I) can be combined with IL-11 or anti-cytokine antibodies, for example, fonotolizumab (anti-IFNg antibody), or anti-receptor receptor antibodies, for example, anti-IL-6 receptor antibody and antibodies to B-cell surface molecules. A compound of Formula (I) may also be used with LJP 394 (abetimus), agents that deplete or inactivate B-cells, for example, Rituximab (anti-CD20 antibody), lymphostat-B (anti-BlyS antibody), TNF antagonists, for example, anti-TNF antibodies, D2E7 (U.S. Pat. No. 6,090,382; HUMIRA™), CA2 (REMICADE™), CDP 571, TNFR-Ig constructs, (p75TNFRIgG (ENBREL™) and p55TNFRIgG (LENERCEPT™).

In this invention, the following definitions are applicable:

A “therapeutically effective amount” is an amount of a compound of Formula (I) or a combination of two or more such compounds, which inhibits, totally or partially, the progression of the condition or alleviates, at least partially, one or more symptoms of the condition. A therapeutically effective amount can also be an amount which is prophylactically effective. The amount which is therapeutically effective will depend upon the patient's size and gender, the condition to be treated, the severity of the condition and the result sought. For a given patient, a therapeutically effective amount can be determined by methods known to those of skill in the art.

“Pharmaceutically acceptable salts” refers to those salts which retain the biological effectiveness and properties of the free bases and which are obtained by reaction with inorganic acids, for example, hydrochloric acid, hydrobromic acid, sulfuric acid, nitric acid, and phosphoric acid or organic acids such as sulfonic acid, carboxylic acid, organic phosphoric acid, methanesulfonic acid, ethanesulfonic acid, p-toluenesulfonic acid, citric acid, fumaric acid, maleic acid, succinic acid, benzoic acid, salicylic acid, lactic acid, tartaric acid (e.g. (+) or (−)-tartaric acid or mixtures thereof), amino acids (e.g. (+) or (−)-amino acids or mixtures thereof), and the like. These salts can be prepared by methods known to those skilled in the art.

Certain compounds of Formula (I) which have acidic substituents may exist as salts with pharmaceutically acceptable bases. The present invention includes such salts. Examples of such salts include sodium salts, potassium salts, lysine salts and arginine salts. These salts may be prepared by methods known to those skilled in the art.

Certain compounds of Formula (I) and their salts may exist in more than one crystal form and the present invention includes each crystal form and mixtures thereof.

Certain compounds of Formula (I) and their salts may also exist in the form of solvates, for example hydrates, and the present invention includes each solvate and mixtures thereof.

Certain compounds of Formula (I) may contain one or more chiral centers, and exist in different optically active forms. When compounds of Formula (I) contain one chiral center, the compounds exist in two enantiomeric forms and the present invention includes both enantiomers and mixtures of enantiomers, such as racemic mixtures. The enantiomers may be resolved by methods known to those skilled in the art, for example by formation of diastereoisomeric salts which may be separated, for example, by crystallization; formation of diastereoisomeric derivatives or complexes which may be separated, for example, by crystallization, gas-liquid or liquid chromatography; selective reaction of one enantiomer with an enantiomer-specific reagent, for example enzymatic esterification; or gas-liquid or liquid chromatography in a chiral environment, for example on a chiral support for example silica with a bound chiral ligand or in the presence of a chiral solvent. It will be appreciated that where the desired enantiomer is converted into another chemical entity by one of the separation procedures described above, a further step is required to liberate the desired enantiomeric form. Alternatively, specific enantiomers may be synthesized by asymmetric synthesis using optically active reagents, substrates, catalysts or solvents, or by converting one enantiomer into the other by asymmetric transformation.

When a compound of Formula (I) contains more than one chiral center, it may exist in diastereoisomeric forms. The diastereoisomeric compounds may be separated by methods known to those skilled in the art, for example chromatography or crystallization and the individual enantiomers may be separated as described above. The present invention includes each diastereoisomer of compounds of Formula (I), and mixtures thereof.

Certain compounds of Formula (I) may exist in different tautomeric forms or as different geometric isomers, and the present invention includes each tautomer and/or geometric isomer of compounds of Formula (I) and mixtures thereof.

Certain compounds of Formula (I) may exist in different stable conformational forms which may be separable. Torsional asymmetry due to restricted rotation about an asymmetric single bond, for example because of steric hindrance or ring strain, may permit separation of different conformers. The present invention includes each conformational isomer of compounds of Formula (I) and mixtures thereof.

Certain compounds of Formula (I) may exist in zwitterionic form and the present invention includes each zwitterionic form of compounds of Formula (I) and mixtures thereof.

As used herein the term “pro-drug” refers to an agent which is converted into the parent drug in vivo by some physiological chemical process (e.g., a pro-drug on being brought to the physiological pH is converted to the desired drug form). Pro-drugs are often useful because, in some situations, they may be easier to administer than the parent drug. They may, for instance, be bioavailable by oral administration whereas the parent drug is not. The pro-drug may also have improved solubility in pharmacological compositions over the parent drug. An example, without limitation, of a pro-drug would be a compound of the present invention wherein it is administered as an ester (the “pro-drug”) to facilitate transmittal across a cell membrane where water solubility is not beneficial, but then it is metabolically hydrolyzed to the carboxylic acid once inside the cell where water solubility is beneficial.

Pro-drugs have many useful properties. For example, a pro-drug may be more water soluble than the ultimate drug, thereby facilitating intravenous administration of the drug. A pro-drug may also have a higher level of oral bioavailability than the ultimate drug. After administration, the pro-drug is enzymatically or chemically cleaved to deliver the ultimate drug in the blood or tissue.

Exemplary pro-drugs upon cleavage release the corresponding free acid, and such hydrolyzable ester-forming residues of the compounds of this invention include but are not limited to carboxylic acid substituents wherein the free hydrogen is replaced by (C₁-C₄)alkyl, (C₁-C₁₂)alkanoyloxymethyl, (C₄-C₉)1-(alkanoyloxy)ethyl, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl (such as β-dimethylaminoethyl), carbamoyl-(C₁-C₂)alkyl, N,N-di(C₁-C₂)-alkylcarbamoyl-(C₁-C₂)alkyl and piperidino-, pyrrolidino- or morpholino(C₂-C₃)alkyl.

Other exemplary pro-drugs release an alcohol of Formula (I) wherein the free hydrogen of the hydroxyl substituent is replaced by (C₁-C₆)alkanoyloxymethyl, 1-((C₁-C₆)alkanoyloxy)ethyl, 1-methyl-1((C₁-C₆)alkanoyloxy)ethyl, (C₁-C₁₂)alkoxycarbonyloxymethyl, N—(C₁-C₆)alkoxycarbonylamino-methyl, succinoyl, (C₁-C₆)alkanoyl, α-amino(C₁-C₄)alkanoyl, arylacetyl and α-aminoacyl, or α-aminoacyl-α-aminoacyl wherein said α-aminoacyl moieties are independently any of the naturally occurring L-amino acids found in proteins, P(O)(OH)₂, —P(O)(O(C₁-C₆)alkyl)₂ or glycosyl (the radical resulting from detachment of the hydroxyl of the hemiacetal of a carbohydrate).

Other exemplary pro-drugs release an amine of Formula (I) wherein the free hydrogen of the amine group is replaced by —C(O)alkyl, —C(O)O-alkyl, N-phosphonoxyalkyl, alkyl, cycloalkyl, aryl, heteroaryl or heterocyclyl, wherein the alkyl, cycloalkyl, aryl, heteroaryl or heterocyclyl can be optionally substituted with, for example, halogen and hydroxyl.

As used herein “solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like.

As used herein, “hydrate” is a solvate wherein the solvent molecule is water.

As used herein, the term “bridged (C₅-C₁₂) cycloalkyl group” means a saturated or unsaturated, bicyclic or polycyclic bridged hydrocarbon group having two or three C₃-C₁₀ cycloalkyl rings. Non bridged cycloalkyls are excluded. For purposes of exemplification, which should not be construed as limiting the scope of this invention, bridged cyclic hydrocarbon may include bicyclo[2.1.1]hexyl, bicyclo[2.2.1]heptyl, bicyclo[2.2.2]octyl, bicyclo[3.2.1]octyl, bicyclo[4.3.1]decyl, bicyclo[3.3.1]nonyl, bornyl, bornenyl, norbornyl, norbonenyl, 6,6-dimethylbicyclo[3.1.1]heptyl, tricyclobutyl, and adamantyl.

As used herein the term “bridged (C₂-C₁₀) heterocyclyl” means bicyclic or polycyclic bridged hydrocarbon groups containing one or more heteroatoms such as nitrogen, oxygen and sulfur. For purposes of exemplification, which should not be construed as limiting the scope of this invention, bridged (C₂-C₁₀) heterocyclyl may include azanorbornyl, quinuclidinyl, isoquinuclidinyl, tropanyl, azabicyclo[3.2.1]octanyl, azabicyclo[2.2.1]heptanyl, 2-azabicyclo[3.2.1]octanyl, azabicyclo[3.2.1]octanyl, azabicyclo[3.2.2]nonanyl, azabicyclo[3.3.0]nonanyl, and azabicyclo[3.3.1]nonanyl.

As used herein, “spirocyclic (C₂-C₁₀) heterocyclyl” means bicyclic or polycyclic hydrocarbon group having two or three (C₃-C₁₀) rings at least one of which contains a heteroatom such as nitrogen, oxygen or sulfur. For purposes of exemplification, which should not be construed as limiting the scope of this invention, spirocyclic (C₂-C₁₀) heterocyclyl may include diazaspiro[3.5]nonane and diazaspiro[4.5]decane.

As used herein, “spirocyclic (C₅-C₁₁) carbocyclyl” means a saturated or unsaturated, bicyclic or polycyclic hydrocarbon group having two or three (C₃-C₁₀) cycloalkyl rings. For purposes of exemplification, which should not be construed as limiting the scope of this invention, spirocyclic (C₅-C₁₁) carbocyclyl includes spiro[5.5]undecane, spiro[4.5]decane and spiro[4.4]nonane.

The term “heterocyclic”, “heterocyclyl” or “heterocyclylene”, as used herein, include non-aromatic ring systems, including, but not limited to, monocyclic, bicyclic, and tricyclic rings, which can be completely saturated or which can contain one or more units of unsaturation. (for the avoidance of doubt, the degree of unsaturation does not result in an aromatic ring system) and have 5 to 12 atoms including at least one heteroatom, such as nitrogen, oxygen, or sulfur. For purposes of exemplification, which should not be construed as limiting the scope of this invention, the following are examples of heterocyclic rings: azepinyl, azetidinyl, indolinyl, isoindolinyl, morpholinyl, piperazinyl, piperidinyl, pyrrolidinyl, quinucludinyl, thiomorpholinyl, tetrahydropyranyl, tetrahydrofuranyl, tetrahydroindolyl, thiomorpholinyl and tropanyl.

The term “heteroaryl” or “heteroarylene” as used herein, include aromatic ring systems, including, but not limited to, monocyclic, bicyclic and tricyclic rings, and have 5 to 12 atoms including at least one heteroatom, such as nitrogen, oxygen, or sulfur. For purposes of exemplification, which should not be construed as limiting the scope of this invention: azaindolyl, benzo(b)thienyl, benzimidazolyl, benzofuranyl, benzoxazolyl, benzothiazolyl, benzothiadiazolyl, benzoxadiazolyl, 6,7-dihydro-5H-cyclopentapyrimidinyl, furanyl, imidazolyl, imidazopyridinyl, indolyl, indazolyl, isoxazolyl, isothiazolyl, octahydro-pyrrolopyrrolyl, oxadiazolyl, oxazolyl, phthalazinyl, pteridinyl, purinyl, pyranyl, 5,8-dihydro-6H-pyrano[3,4-d]pyridinyl, pyrazinyl, pyrazolyl, pyridinyl, pyrido[2,3-d]pyrimidinyl, pyrido[4,3-d]pyrimidinyl, pyrido[3,4-d]pyrimidinyl, pyrimidinyl, pyrimido[4,5-d]pyrimidinyl, pyrrolyl, pyrrolo[2,3-d]pyrimidinyl, pyrazolo[3,4-d]pyrimidinyl, quinolinyl, quinazolinyl, 5,6,7,8-tetrahydroquinazolinyl, triazolyl, thiazolyl, thieno[2,3-d]pyrimidinyl, thieno[3,2-d]pyrimidinyl, thiophenyl, tetrazolyl, thiadiazolyl, thienyl, [1,3,5]triazinyl, 5,6,7,8-tetrahydro-imidazo[1,5-a]pyrazinyl, and 5,6,7,8-tetrahydro-triazolo[1,2,4]pyrazinyl.

As used herein, “alkyl” and “alkylene” include straight chained or branched hydrocarbons which are completely saturated. For purposes of exemplification, which should not be construed as limiting the scope of this invention, examples of alkyls are methyl, ethyl, propyl, isopropyl, butyl, pentyl, hexyl and isomers thereof.

As used herein, “alkenyl”, “alkenylene”, “alkynylene” and “alkynyl” mean hydrocarbon moieties containing two to eight carbons and include straight chained or branched hydrocarbons which contain one or more units of unsaturation, one or more double bonds for alkenyl and one or more triple bonds for alkynyl. For purposes of exemplification, which should not be construed as limiting the scope of this invention, examples of alkenyl are ethenyl, propenyl and butenyl, and examples of alkynyl are ethynyl, propynyl and butynyl.

As used herein, “aryl” or “arylene” groups include aromatic carbocyclic ring systems (e.g. phenyl) and fused polycyclic aromatic ring systems. For purposes of exemplification, which should not be construed as limiting the scope of this invention, aryl groups include naphthyl, biphenyl and 1,2,3,4-tetrahydronaphthyl.

As used herein, “cycloalkyl” or “cycloalkylene” means C₃-C₁₂ monocyclic or multicyclic (e.g., bicyclic, tricyclic, etc.) hydrocarbons that are completely saturated or have one or more unsaturated bonds but do not amount to an aromatic group. For purposes of exemplification, which should not be construed as limiting the scope of this invention, examples of a cycloalkyl group are cyclopropyl, cyclobutyl, cyclopentyl, cyclopentenyl, cyclohexyl and cyclohexenyl.

As used herein, many moieties or substituents are termed as being either “substituted” or “optionally substituted”. When a moiety is modified by one of these terms, unless otherwise noted, it denotes that any portion of the moiety that is known to one skilled in the art as being available for substitution can be substituted, which includes one or more substituents, where if more than one substituent then each substituent is independently selected. Such means for substitution are well-known in the art and/or taught by the instant disclosure. For purposes of exemplification, which should not be construed as limiting the scope of this invention, some examples of groups that are substituents are: deuterium, optionally substituted (C₁-C₈)alkyl groups, optionally substituted (C₂-C₈)alkenyl groups, (C₂-C₈)alkynyl groups, optionally substituted (C₃-C₁₀)cycloalkyl groups, halogen (F, Cl, Br or I), halogenated (C₁-C₈)alkyl groups (for example but not limited to —CF₃), —O—(C₁-C₈)alkyl groups, —OH, —S—(C₁-C₈)alkyl groups, —SH, —NH(C₁-C₈)alkyl groups, —N((C₁-C₈)alkyl)₂ groups, —NH₂, —NH—(C₁-C₆)alkyl-optionally substituted heterocycle, —NH-heterocycle, —C(O)NH₂, —C(O)NH(C₁-C₈)alkyl groups, —C(O)N((C₁-C₈)alkyl)₂, —NHC(O)H, —NHC(O)(C₁-C₈)alkyl groups, —NHC(O)(C₃-C₈)cycloalkyl groups, —N((C₁-C₈)alkyl)C(O)H, —N((C₁-C₈)alkyl)C(O)(C₁-C₈)alkyl groups, —NHC(O)NH₂, —NHC(O)NH(C₁-C₈)alkyl groups, —N((C₁-C₈)alkyl)C(O)NH₂ groups, —NHC(O)N((C₁-C₈)alkyl)₂ groups, —N((C₁-C₈)alkyl)C(O)N((C₁-C₈)alkyl)₂ groups, —N((C₁-C₈)alkyl)C(O)NH((C₁-C₈)alkyl), —C(O)H, —C(O)(C₁-C₈)alkyl groups, —CN, —NO₂, —S(O)(C₁-C₈)alkyl groups, —S(O)₂(C₁-C₈)alkyl groups, —S(O)₂N((C₁-C₈)alkyl)₂ groups, —S(O)₂NH(C₁-C₈)alkyl groups, —S(O)₂NH(C₃-C₈)cycloalkyl groups, —S(O)₂NH₂ groups, —NHS(O)₂(C₁-C₈)alkyl groups, —N((C₁-C₈)alkyl)S(O)₂(C₁-C₈)alkyl groups, —(C₁-C₈)alkyl-O—(C₁-C₈)alkyl groups, —O—(C₁-C₈)alkyl-O—(C₁-C₈)alkyl groups, —C(O)OH, —C(O)O(C₁-C₈)alkyl groups, —NHOH, —NHO(C₁-C₈)alkyl groups, —O-halogenated (C₁-C₈)alkyl groups (for example but not limited to —OCF₃), —S(O)₂-halogenated (C₁-C₈)alkyl groups (for example but not limited to —S(O)₂CF₃), —S-halogenated (C₁-C₈)alkyl groups (for example but not limited to —SCF₃), —(C₁-C₆)alkyl-optionally substituted heterocycle (for example but not limited to azetidine, piperidine, piperazine, pyrrolidine, tetrahydrofuran, pyran or morpholine), —(C₁-C₆)alkyl-heteroaryl (for example but not limited to tetrazole, imidazole, furan, pyrazine or pyrazole), optionally substituted phenyl, —NHC(O)O—(C₁-C₆)alkyl groups, —N((C₁-C₆)alkyl)C(O)O—(C₁-C₆)alkyl groups, —C(═NH)—(C₁-C₆)alkyl groups, —C(═NOH)—(C₁-C₆)alkyl groups, or —C(═N—O—(C₁-C₆)alkyl)-(C₁-C₆)alkyl groups.

As described herein, a bond drawn from a substituent to the center of one right within a multiple-ring system (as shown below) represents substitution of the substituent at any substitutable position in any of the rings within the multiple ring system. For example, Figure a represents possible substitution in any of the positions shown in Figure b.

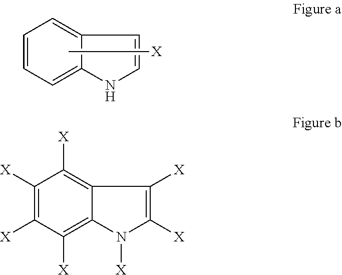

If, however, two rings in a multiple ring system each have different substituents drawn form the center of each ring, then, unless otherwise specified, each substituent only represents substitution on the ring to which it is attached. For example, in Figure c, Y is an optional substituent for Ring A only, and X is an optional substituent for Ring B only.

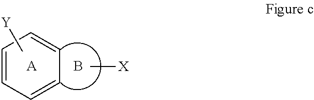

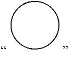

represents an aromatic ring, for example

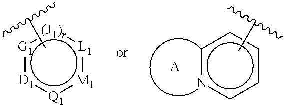

One or more compounds of this invention can be administered to a human patient by themselves or in pharmaceutical compositions where they are mixed with biologically suitable carriers or excipient(s) at doses to treat or ameliorate a disease or condition as described herein. Mixtures of these compounds can also be administered to the patient as a simple mixture or in suitable formulated pharmaceutical compositions. A therapeutically effective dose refers to that amount of the compound or compounds sufficient to result in the prevention or attenuation of a disease or condition as described herein. Techniques for formulation and administration of the compounds of the instant application may be found in references well known to one of ordinary skill in the art, such as “Remington's Pharmaceutical Sciences,” Mack Publishing Co., Easton, Pa., latest edition.

Suitable routes of administration may, for example, include oral, eyedrop, rectal, transmucosal, topical, or intestinal administration; parenteral delivery, including intramuscular, subcutaneous, intramedullary injections, as well as intrathecal, direct intraventricular, intravenous, intraperitoneal, intranasal, or intraocular injections.

Alternatively, one may administer the compound in a local rather than a systemic manner, for example, via injection of the compound directly into an edematous site, often in a depot or sustained release formulation.

Furthermore, one may administer the drug in a targeted drug delivery system, for example, in a liposome coated with endothelial cell-specific antibody.

The pharmaceutical compositions of the present invention may be manufactured in a manner that is itself known, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in a conventional manner using one or more physiologically acceptable carriers comprising excipients and auxiliaries which facilitate processing of the active compounds into preparations which can be used pharmaceutically. Proper formulation is dependent upon the route of administration chosen.

For injection, the agents of the invention may be formulated in aqueous solutions, preferably in physiologically compatible buffers such as Hanks' solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained by combining the active compound with a solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations that can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

The compounds can be formulated for parenteral administration by injection, e.g. bolus injection or continuous infusion. Formulations for injection may be presented in unit dosage form, e.g. in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly or by intramuscular injection). Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

An example of a pharmaceutical carrier for the hydrophobic compounds of the invention is a cosolvent system comprising benzyl alcohol, a nonpolar surfactant, a water-miscible organic polymer, and an aqueous phase. The cosolvent system may be the VPD co-solvent system. VPD is a solution of 3% w/v benzyl alcohol, 8% w/v of the nonpolar surfactant polysorbate 80, and 65% w/v polyethylene glycol 300, made up to volume in absolute ethanol. The VPD co-solvent system (VPD:5W) consists of VPD diluted 1:1 with a 5% dextrose in water solution. This co-solvent system dissolves hydrophobic compounds well, and itself produces low toxicity upon systemic administration. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of polysorbate 80; the fraction size of polyethylene glycol may be varied; other biocompatible polymers may replace polyethylene glycol, e.g. polyvinyl pyrrolidone; and other sugars or polysaccharides may substitute for dextrose.

Alternatively, other delivery systems for hydrophobic pharmaceutical compounds may be employed. Liposomes and emulsions are well known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds for a few hours up to over several days. Depending on the chemical nature and the biological stability of the therapeutic reagent, additional strategies for protein stabilization may be employed.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Many of the compounds of the invention may be provided as salts with pharmaceutically compatible counterions. Pharmaceutically compatible salts may be formed with many acids, including but not limited to hydrochloric, sulfuric, acetic, lactic, tartaric, malic, succinic, etc. Salts tend to be more soluble in aqueous or other protonic solvents than are the corresponding free base forms.

Pharmaceutical compositions suitable for use in the present invention include compositions wherein the active ingredients are contained in an effective amount to achieve its intended purpose. More specifically, a therapeutically effective amount means an amount effective to prevent development of or to alleviate the existing symptoms of the subject being treated. Determination of the effective amounts is well within the capability of those skilled in the art.

For any compound used in a method of the present invention, the therapeutically effective dose can be estimated initially from cellular assays. For example, a dose can be formulated in cellular and animal models to achieve a circulating concentration range that includes the IC₅₀ as determined in cellular assays (i.e., the concentration of the test compound which achieves a half-maximal inhibition of a given protein kinase activity). In some cases it is appropriate to determine the IC₅₀ in the presence of 3 to 5% serum albumin since such a determination approximates the binding effects of plasma protein on the compound. Such information can be used to more accurately determine useful doses in humans. Further, the most preferred compounds for systemic administration effectively inhibit protein kinase signaling in intact cells at levels that are safely achievable in plasma.

A therapeutically effective dose refers to that amount of the compound that results in amelioration of symptoms in a patient. Toxicity and therapeutic efficacy of such compounds can be determined by standard pharmaceutical procedures in cell cultures or experimental animals, e.g., for determining the maximum tolerated dose (MTD) and the ED₅₀ (effective dose for 50% maximal response). The dose ratio between toxic and therapeutic effects is the therapeutic index and it can be expressed as the ratio between MTD and ED₅₀. Compounds which exhibit high therapeutic indices are preferred. The data obtained from these cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. The exact formulation, route of administration and dosage can be chosen by the individual physician in view of the patient's condition (see e.g. Fingl et al., 1975, in “The Pharmacological Basis of Therapeutics”, Ch. 1 p. 1). In the treatment of crises, the administration of an acute bolus or an infusion approaching the MTD may be required to obtain a rapid response.

Dosage amount and interval may be adjusted individually to provide plasma levels of the active moiety which are sufficient to maintain the kinase modulating effects, or minimal effective concentration (MEC). The MEC will vary for each compound but can be estimated from in vitro data; e.g. the concentration necessary to achieve 50-90% inhibition of protein kinase using the assays described herein. Dosages necessary to achieve the MEC will depend on individual characteristics and route of administration. However, HPLC assays or bioassays can be used to determine plasma concentrations.

Dosage intervals can also be determined using the MEC value. Compounds should be administered using a regimen which maintains plasma levels above the MEC for 10-90% of the time, preferably between 30-90% and most preferably between 50-90% until the desired amelioration of symptoms is achieved. In cases of local administration or selective uptake, the effective local concentration of the drug may not be related to plasma concentration.

The amount of composition administered will, of course, be dependent on the subject being treated, on the subject's weight, the severity of the affliction, the manner of administration and the judgment of the prescribing physician.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. Compositions comprising a compound of the invention formulated in a compatible pharmaceutical carrier may also be prepared, placed in an appropriate container, and labelled for treatment of an indicated condition.

In some formulations it may be beneficial to use the compounds of the present invention in the form of particles of very small size, for example as obtained by fluid energy milling.

The use of compounds of the present invention in the manufacture of pharmaceutical compositions is illustrated by the following description. In this description the term “active compound” denotes any compound of the invention but particularly any compound which is the final product of one of the following Examples.

### a) Capsules

In the preparation of capsules, 10 parts by weight of active compound and 240 parts by weight of lactose can be de-aggregated and blended. The mixture can be filled into hard gelatin capsules, each capsule containing a unit dose or part of a unit dose of active compound.

### b) Tablets

Tablets can be prepared, for example, from the following ingredients.

Parts by weight

| | |
|---|---|
| Active compound | 10 |
| Lactose | 190 |
| Maize starch | 22 |
| Polyvinylpyrrolidone | 10 |
| Magnesium stearate | 3 |

The active compound, the lactose and some of the starch can be de-aggregated, blended and the resulting mixture can be granulated with a solution of the polyvinylpyrrolidone in ethanol. The dry granulate can be blended with the magnesium stearate and the rest of the starch. The mixture is then compressed in a tabletting machine to give tablets each containing a unit dose or a part of a unit dose of active compound.

### c) Enteric Coated Tablets

Tablets can be prepared by the method described in (b) above. The tablets can be enteric coated in a conventional manner using a solution of 20% cellulose acetate phthalate and 3% diethyl phthalate in ethanol:dichloromethane (1:1).

### d) Suppositories

In the preparation of suppositories, for example, 100 parts by weight of active compound can be incorporated in 1300 parts by weight of triglyceride suppository base and the mixture formed into suppositories each containing a therapeutically effective amount of active ingredient.

In the compositions of the present invention the active compound may, if desired, be associated with other compatible pharmacologically active ingredients. For example, the compounds of this invention can be administered in combination with another therapeutic agent that is known to treat a disease or condition described herein. For example, with one or more additional pharmaceutical agents that inhibit or prevent the production of VEGF or angiopoietins, attenuate intracellular responses to VEGF or angiopoietins, block intracellular signal transduction, inhibit vascular hyperpermeability, reduce inflammation, or inhibit or prevent the formation of edema or neovascularization. The compounds of the invention can be administered prior to, subsequent to or simultaneously with the additional pharmaceutical agent, whichever course of administration is appropriate. The additional pharmaceutical agents include, but are not limited to, anti-edemic steroids, NSAIDS, ras inhibitors, anti-TNF agents, anti-IL1 agents, antihistamines, PAF-antagonists, COX-1 inhibitors, COX-2 inhibitors, NO synthase inhibitors, Akt/PTB inhibitors, IGF-1R inhibitors, PI3 kinase inhibitors, calcineurin inhibitors and immunosuppressants. The compounds of the invention and the additional pharmaceutical agents act either additively or synergistically. Thus, the administration of such a combination of substances that inhibit angiogenesis, vascular hyperpermeability and/or inhibit the formation of edema can provide greater relief from the deletrious effects of a hyperproliferative disorder, angiogenesis, vascular hyperpermeability or edema than the administration of either substance alone. In the treatment of malignant disorders combinations with antiproliferative or cytotoxic chemotherapies or radiation are included in the scope of the present invention.

The present invention also comprises the use of a compound of Formula (I) as a medicament.

### ABBREVIATIONS

Ac Acetyl
ATP Adenosine triphosphate
Boc t-Butoxycarbonyl
Boc₂O Di-tert-butyl dicarbonate
BOP-Cl Bis(2-oxo-3-oxazolidinyl)phosphonic chloride
BSA Bovine serum albumin
n-BuLi n-Butyllithium
t-BuLi t-Butyllithium
CaCl₂ Calcium chloride
CO₂ Carbon dioxide
CT Computed tomography
Cs₂CO₃ Cesium carbonate
d Doublet
DCE Dichloroethane
DCM Dichloromethane (methylene chloride)
dd Doublet of doublets
ddd Doublet of doublets of doublets
DIEA N,N-Diisopropylethylamine
DMEM Dulbecco's Modified Eagle Medium
DMF N,N-Dimethylformamide
DMSO Dimethyl sulfoxide
DTT Dithiothreitol
EDC.HCl N-(3-Dimethylaminopropyl)-N′-ethylcarbodiimide hydrochloride
EDTA Ethylene diamine tetraacetic acid
EGTA Ethylene glycol-O,O′-bis(2-aminoethyl)-N,N,N′,N′-tetraacetic acid
equiv Equivalent(s)
EtOAc Ethyl acetate
Et₂O Diethyl ether
EtOH Ethanol
FBS Fetal bovine serum
g Gram(s)
h Hour(s)
HATU O-(7-Azabenzotriazol-1-yl)-N,N,N′,N′-tetramethyluronium hexafluorophosphate
HCl Hydrochloric acid
HEPES N-2-Hydroxyethylpiperazine-N′-2-ethanesulfonic acid
HOBt Hydroxybenzotriazole
HPLC High-pressure liquid chromatography
IBCF Isobutylchloroformate
i.d. Intradermal
KF Potassium fluoride
K₂CO₃ Potassium carbonate
KOt-Bu Potassium tert-butoxide
LC/MS Liquid chromatography/mass spectrometry
LDA Lithium diisoproylamide
LiAlH₄ Lithium aluminum hydride
LiBH₄ Lithium borohydride
LiBr Lithium bromide
LiHMDS Lithium bis(trimethylsilyl)amide
m Multiplet
M Molar
MgCl₂ Magnesium chloride
MeCN Acetonitrile
MeOH Methyl alcohol
MgSO₄ Magnesium sulfate
min Minute(s)
mmol Millimole
MOPS 3-(N-Morpholino)-2-hydroxypropanesulfonic acid
MS Mass spectrometry
MsCl Methanesulfonyl chloride
MTT 3-(4,5-Dimethylthiazol-2-yl)-2,5-diphenyltetrazolium bromide
N Normal
Na(AcO)₃BH Sodium triacetoxyborohydride
NaCl Sodium chloride
NaF Sodium fluoride
NaH Sodium hydride
NaHCO₃ Sodium bicarbonate
NaBH₃CN Sodium cyanoborohydride
Na₂CO₃ Sodium carbonate
NaOH Sodium hydroxide
Na₂SO₄ Sodium sulfate
Na₃VO₄ Sodium orthovanadate
NH₃ Ammonia
NH₄Cl Ammonium chloride
NH₄OAc Ammonium acetate
NH₄OH Ammonium hydroxide
NMP N-Methylpyrrolidinone
NMR Nuclear magnetic resonance
p para
PBS Phosphate buffered saline
Pd/C Palladium on carbon
Pd₂(dba)₃ Bis(dibenzylideneacetone)palladium(0)
Pd(OAc)₂ Palladium(II) acetate
Pd(PH₃)₄ Tetrakis(triphenylphosphine)palladium(0)
pH -log[H⁺]
PHA Phytohaemagglutinin
ppm Parts per million
PrOH Propanol
PVDF Polyvinylidene fluoride
RB Reaction buffer
RP-HPLC Reverse-phase high-pressure liquid chromatography
RPMI Roswell park memorial institute
rpm Revolutions per minute
Rₜ Retention time
s Singlet
SDS-PAGE Sodium dodecyl sulfate polyacrylamide gel electrophoresis
sec Second
SOCl₂ Thionyl chloride
t Triplet
t- Tertiary
TBTU 2-(1H-Benzo[d][1,2,3]triazol-1-yl)-1,1,3,3-tetramethyluronium tetrafluoroborate
td Triplet of doublets
TEA Triethylamine
TFA Trifluoroacetic acid
tert- Tertiary
THF Tetrahydrofuran

General Synthetic Schemes

Compounds of the invention may be prepared using the synthetic transformations illustrated in Schemes I-IV. Starting materials are commercially available or may be prepared by the procedures described herein, by literature procedures, or by procedures that would be well known to one skilled in the art of organic chemistry.

Methods for preparing thienopyrroles such as 3 and 12 of the invention are illustrated in Scheme I and II. Thienopyrrole esters such as 3 and 12 may be respectively prepared from tert-butyl 2-iodothiophen-3-ylcarbamate and tert-butyl 3-iodothiophen-2-ylcarbamate as described by D. Wensbo, U. Annby and S. Gronowitz in Tetrahedron, 1995, 51(37), 10323 or from tert-butyl 2-bromothiophen-3-ylcarbamate and tert-butyl 3-bromothiophen-2-ylcarbamate as shown herein. Thienopyrroles with additional substitution may be constructed as described by D. Bonafoux and W. Xiaoyun in WO 2009102462 A1. Thienopyrrole esters such as 3 and 12 are synthesized from tert-butyl halothiophenylcarbamates such as 2 and 11, via a “one-pot two step sequence” involving alkylation with a crotonate followed by an intramolecular Heck coupling using conditions described in General Procedure B. The tert-butyl bromothiophenylcarbamates 2 and 11 can be prepared from commercially available bromothiophenecarboxylates 1 and 10 respectively utilizing a Curtius rearrangement using methods known to one skilled in the art (see, for example, Y. Yang, A.-B. Hörnfeldt, S. Gronowitz Chemica Scripta, 1998, 28, 275 or General Procedure A). Alternatively, a thiophenecarboxylate may be converted to a tert-butyl thiophenylcarbamate using the Curtius rearrangement. Halogenation using methods known to one skilled in the art (see, for example, Larock, R. C. “Comprehensive Organic Transformations: A Guide to Functional Group Preparations, 2^{nd} edition”, 1999, Wiley-VCH) and in particular bromination, with bromine, can give the tert-butyl bromothiophenylcarbamates as described in Preparation #1.

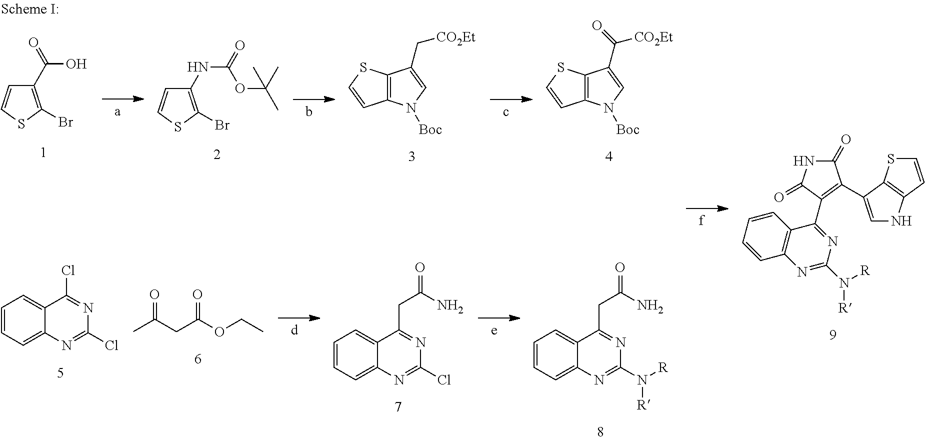

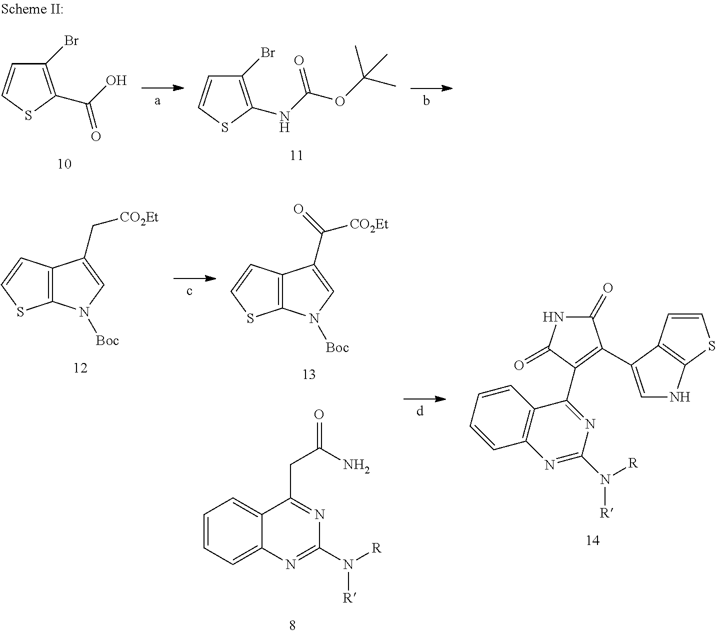

Esters such as 3 and 12 may be oxidized to α-keto esters using methods known to one skilled in the art (see, for example March, J. “Advanced Organic Chemistry, 4^{th} edition”, 1992, John Wiley & Sons, Inc. or General Procedure C).

A variety of methods are known for replacement of an aryl halide such as the chloride in the 4-position of quinazoline 5 with an alkyl acetate group (see for example, (a) S. Gregson and G. Shaw Journal of the Chemical Society, Perkin Transactions 1, 1985, 187-90. (b) W. M. Odijk and G. J. Koomen Tetrahedron, 1985, 41, 1893-1904. (c) N. Hamamichi and T. Miyasaka Journal of Heterocyclic Chemistry, 1990, 27, 2011-2015. (d) T. Hama and J. F. Hartwig Organic Letters, 2008, 10, 1549-1552.). Conversion to a primary amide by amino-de-alkoxylation of an ester using methods known to one skilled in the art (see, for example, Larock, R. C. “Comprehensive Organic Transformations: A Guide to Functional Group Preparations, 2^{nd} edition”, 1999, Wiley-VCH) may be used to prepare 2-(2-chloroquinazolin-4-yl)acetamide, 7, as demonstrated in Preparation #D.1.

Intermediate 7 may be converted to a 2-aminoquinazoline such as 8 using an aromatic nucleophilic substitution of an appropriate leaving group such as the chloride in 7 with a nucleophile such as an amine using methods known to one skilled in the art (see, for example, March, J. “Advanced Organic Chemistry”, 4^{th} edition, 1992, John Wiley & Sons, Inc. or General Procedure E).

Formation of maleimides such as 9 or 14 using a condensation of an α-keto ester such as 4 or 13 with an acetamide such as 8 may be accomplished as demonstrated in General Procedure F. Alternatively, the maleimide core may be formed prior to incorporation of the amine by the condensation of an α-keto ester such as 4 or 13 with an acetamide such as 7 as illustrated in Schemes III and IV. Intermediates such as 15 and 16 may then undergo an aromatic nucleophilic substitution with a nucleophile such as an amine to prepare compounds such as 9 and 14 as demonstrated in General Procedure E.

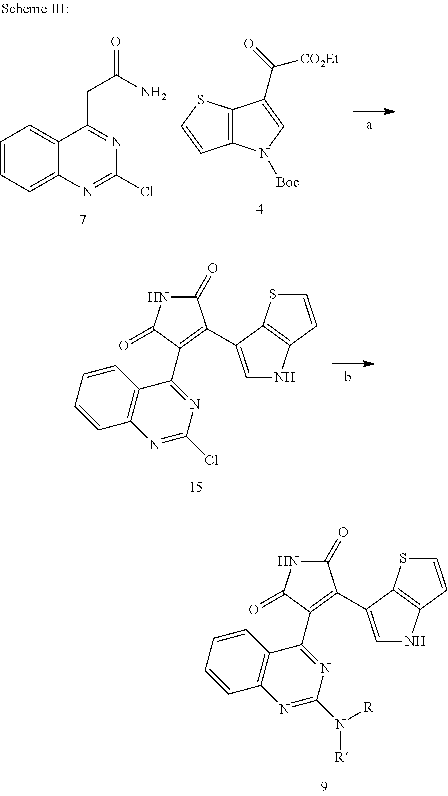

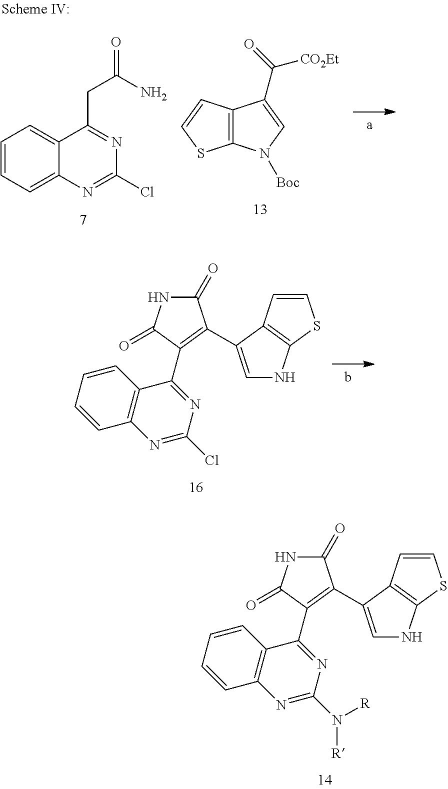

### GENERAL PROCEDURES AND EXAMPLES

The general synthetic schemes that were utilized to construct the majority of compounds disclosed in this application are described below in Schemes 1-22. These schemes are provided for illustrative purposes only and are not to be construed as limiting the scope of the invention.

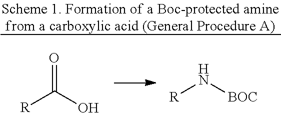

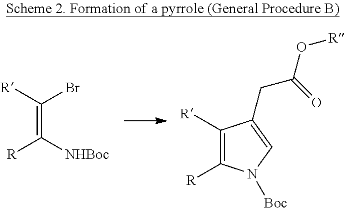

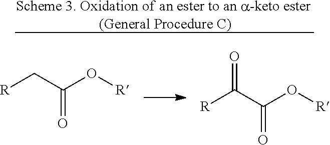

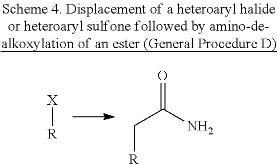

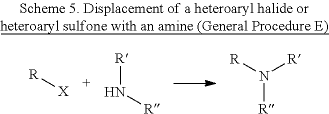

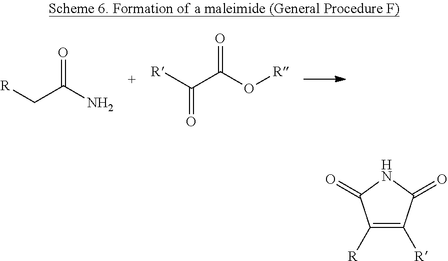

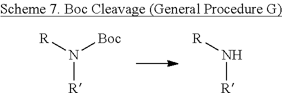

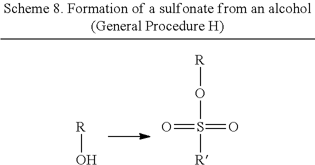

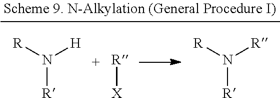

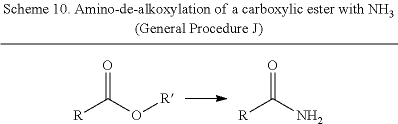

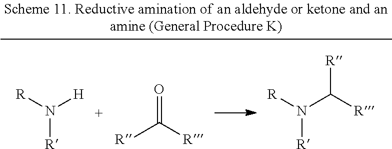

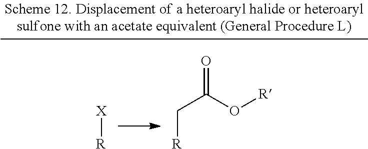

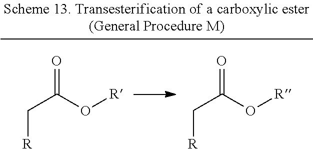

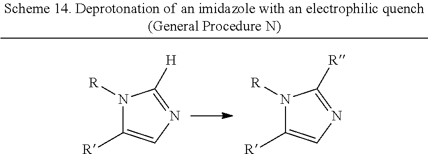

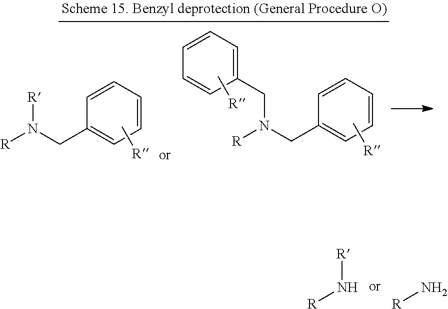

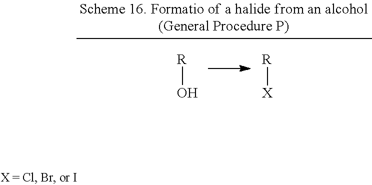

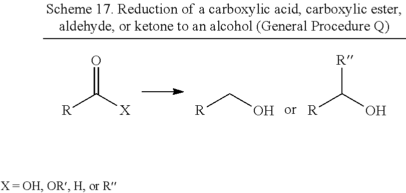

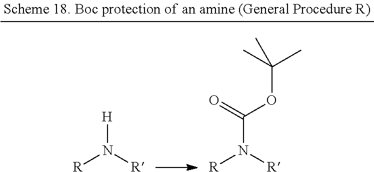

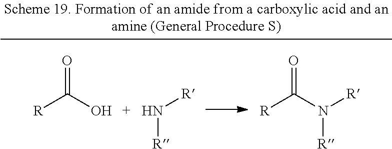

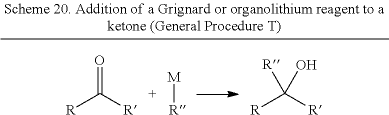

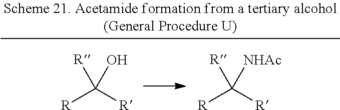

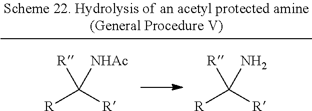

### LIST OF GENERAL PROCEDURES

General Procedure A: Formation of a Boc-protected amine from a carboxylic acid
General Procedure B: Formation of a pyrrole
General Procedure C: Oxidation of an ester to an α-keto ester
General Procedure D: Displacement of a heteroaryl halide or heteroaryl sulfone followed by amino-de-alkoxylation of an ester
General Procedure E: Displacement of a heteroaryl halide or heteroaryl sulfone with an amine
General Procedure F: Formation of a maleimide
General Procedure G: Boc cleavage
General Procedure H: Formation of a sulfonate from an alcohol
General Procedure I: N-Alkylation
General Procedure J: Amino-de-alkoxylation of a carboxylic ester with NH₃
General Procedure K: Reductive amination of an aldehyde or ketone and an amine
General Procedure L: Displacement of a heteroaryl halide or heteroaryl sulfone with an acetate equivalent
General Procedure M: Transesterification of a carboxylic ester
General Procedure N: Deprotonation of an imidazole with an electrophilic quench
General Procedure O: Benzyl deprotection
General Procedure P: Formation of a halide from an alcohol
General Procedure Q: Reduction of a carboxylic acid, carboxylic ester, aldehyde, or ketone to an alcohol
General Procedure R: Boc protection of an amine
General Procedure S: Formation of an amide from a carboxylic acid and an amine
General Procedure T: Addition of a Grignard or organolithium reagent to a ketone
General Procedure U: Acetamide formation from a tertiary alcohol
General Procedure V: Hydrolysis of an acetyl protected amine

### Analytical Methods

Analytical data is included within the procedures below, in the illustrations of the general procedures, or in the tables of examples. Unless otherwise stated, all ¹H NMR data were collected on a Varian Mercury Plus 400 MHz or a Varian Inova 600 MHz instrument and chemical shifts are quoted in parts per million (ppm). Mass-Triggered purification was performed using a Waters 2667 binary LC pump and two Waters 515 LC pump for at-column dilution and makeup flow. The fraction collector was a Waters 2767 Collection Manager. Detection methods included a Waters 2996 photodiode array and a Waters 2000 ZQ mass spectrometer, a 1:1000 splitter flow (by LC Packings) was used to split flow to detectors and fraction collection. LC/MS and HPLC data is referenced to LC/MS and HPLC conditions using the lower case method letter provided in Table 1.

TABLE 1
LC/MS and HPLC methods

| Method | Conditions |
|---|---|
| a | LC/MS: The gradient was 5-60% B in 1.5 min then 60-95% B to 2.5 min with a |
| | hold at 95% B for 1.2 min (1.3 mL/min flow rate). Mobile phase A was 10 mM |
| | NH₄OAc, mobile phase B was HPLC grade MeCN. The column used for the |
| | chromatography is a 4.6 × 50 mm MAC-MOD Halo C8 column (2.7 μm particles). |
| | Detection methods are diode array (DAD) and evaporative light scattering (ELSD) |
| | detection as well as positive/negative electrospray ionization. |
| b | LC/MS: The gradient was 5-60% B in 0.75 min then 60-95% B to 1.15 min with a |
| | hold at 95% B for 0.75 min (1.3 mL/min flow rate). Mobile phase A was 10 mM |
| | NH₄OAc, mobile phase B was HPLC grade MeCN. The column used for the |
| | chromatography is a 4.6 × 50 mm MAC-MOD Halo C8 column (2.7 μm particles). |
| | Detection methods are diode array (DAD) and evaporative light scattering (ELSD) |
| | detection as well as positive/negative electrospray ionization. |
| c | LC/MS: The gradient was 5-60% B in 1.5 min then 60-95% B to 2.5 min with a |
| | hold at 95% B for 1.2 min (1.3 mL/min flow rate). Mobile phase A was 10 mM |
| | NH₄OAc, mobile phase B was HPLC grade MeCN. The column used for the |
| | chromatography is a 4.6 × 50 mm MAC-MOD Halo C18 column (2.7 μm particles). |
| | Detection methods are diode array (DAD) and evaporative light scattering (ELSD) |
| | detection as well as positive/negative electrospray ionization. |
| d | HPLC: The gradient was 10% B for 2.5 min then 10-15% B in 0.5 min then 15-75% |
| | B in 6 min then 75% B for 0.1 min then 75-95% in 1.5 min (22.5 mL/min flow rate). |
| | Mobile phase A: 50 mM NH₄OAc in water, mobile phase B was HPLC grade |
| | MeCN. The column used for chromatography is 19 × 50 mm Waters Atlantis T3 |
| | OBD C18 column (5.0 μm particles). Detection methods are photodiode array |
| | (DAD) and Waters ZQ 2000 mass spectrometer. |
| e | LC/MS: The gradient was 5-60% B in 0.60 min then 60-95% B in 1.0 min with a |
| | hold at 95% B for 0.30 (1.25 mL/min flow rate). Mobile phase A was 10 mM |
| | NH₄OAc, mobile phase B was HPLC grade MeCN. The column used for the |
| | chromatography is 2.1 × 30 mm Acquity UPLC HSS T3 column (1.8 μm particles). |
| | Detection methods are diode array (DAD) and evaporative light scattering (ELSD) |
| | detection as well as pos/neg electrospray ionization. |
| f | LC/MS: The gradient was 5% B for 0.2 min then 5-95% B in 1.7 min with a hold at |
| | 95% B for 1.3 min (2.3 mL/min flow rate). Mobile phase A was 0.05% TFA in |
| | water, mobile phase B was 0.05% TFA in HPLC grade MeCN. The column used for |
| | the chromatography is a 4.6 × 50 mm XBridge C18 column (3.5 μm particles). |
| | Detection methods are diode array (DAD) and evaporative light scattering (ELSD) |
| | detection as well as positive/negative electrospray ionization. |
| g | LC/MS: The gradient was 5% B for 0.2 min then 5-95% B in 1.7 min with a hold at |
| | 95% B for 1.3 min (2.3 mL/min flow rate). Mobile phase A was 10 mM ammonium |
| | formate in water, mobile phase B was HPLC grade MeCN. The column used for the |
| | chromatography is a 4.6 × 50 mm XBridge C18 column (3.5 μm particles). Detection |
| | methods are diode array (DAD) and evaporative light scattering (ELSD) detection as |
| | well as positive/negative electrospray ionization. |
| h | HPLC: The gradient was 12% B for 2.5 min then 12-27% B over 6.5 min then 27- |
| | 98% B over 0.1 min then 98% for 1.5 min (25 mL/min flow rate). Mobile phase A |
| | was 50 mM NH₄OAc (pH 4.5) and mobile phase B was HPLC grade MeCN, the |
| | column used for the chromatography was a 19 × 50 mm Waters Atlantis T3 OBD |
| | C18 column (5 μm particles). Detection methods are photodiode array (DAD) and |
| | Waters ZQ 2000 mass spectrometer. |
| i | HPLC: The gradient was 12% B for 2.5 min then 12-36% B over 7.5 min then 36- |
| | 98% B over 2.0 min then 98% for 1.2 min (25 mL/min flow rate). Mobile phase A |
| | was 50 mM NH₄OAc (pH 4.5) and mobile phase B was HPLC grade MeCN, the |
| | column used for the chromatography was a 21 × 100 mm Phenomenex MAX-RP |
| | column (4 μm particles), detection methods are photodiode array (DAD) and Waters |
| | ZQ 2000 mass spectrometer. |

### Chiral Preparative HPLC Purification

Chiral purification is performed using Varian 218 LC pumps, a Varian CVM 500 with switching valves and heaters for automatic solvent, column and temperature control and a Varian 701 Fraction collector. Detection methods include a Varian 210 variable wavelength detector, an in-line polarimeter (PDR-chiral advanced laser polarimeter, model ALP2002) used to measure qualitative optical rotation (+/−) and an evaporative light scattering detector (ELSD) (a PS-ELS 2100 (Polymer Laboratories)) using a 100:1 split flow. ELSD settings are as follows: evaporator: 46° C., nebulizer: 24° C. and gas flow: 1.1 SLM.

TABLE 2
Chiral HPLC methods

| Method | Conditions |
|---|---|
| 1 | Isocratic 30% A for 30 min (20 mL/min flow rate). Mobile phase A was ethanol |
| | (200 proof), mobile phase B was HPLC grade heptane with 0.12% diethylamine |
| | added. The column used for the chromatography was a (S,S) Whelk-O1, 21 × 250 |
| | mm column (5 μm particles). Detection methods were evaporative light scattering |
| | (ELSD) detection, and/or UV (variable wavelength) as well as optical rotation. |

### Purification Methods

For the general procedures, intermediate and final compounds may be purified by any technique or combination of techniques known to one skilled in the art. Some examples that are not limiting include flash chromatography with a solid phase (i.e. silica gel, alumina, etc.) and a solvent (or combination of solvents, i.e. heptane, EtOAc, DCM, MeOH, MeCN, water, etc.) that elutes the desired compounds; preparatory TLC with a solid phase (i.e. silica gel, alumina etc.) and a solvent (or combination of solvents, i.e. heptane, EtOAc, DCM, MeOH, MeCN, water, etc.) that elutes the desired compounds; reverse phase HPLC (see Table 1 for some non-limiting conditions); recrystallization from an appropriate solvent (i.e. MeOH, EtOH, i-PrOH, EtOAc, toluene, etc.) or combination of solvents (i.e. EtOAc/heptane, EtOAc/MeOH, etc.); chiral chromatography with a solid phase and an appropriate solvent (i.e. EtOH/heptane, MeOH/heptane, i-PrOH/heptane, etc. with or without a modifier such as diethylamine, TFA, etc.) to elute the desired compound; precipitation from a combination of solvents (i.e. DMF/water, DMSO/DCM, EtOAc/heptane, etc.); trituration with an appropriate solvent (i.e. EtOAc, DCM, MeCN, MeOH, EtOH, i-PrOH, n-PrOH, etc.); extractions by dissolving a compound in a liquid and washing with an appropriately immiscible liquid (i.e. DCM/water, EtOAc/water, DCM/saturated NaHCO₃, EtOAc/saturated NaHCO₃, DCM/10% aqueous HCl, EtOAc/10% aqueous HCl, etc.); distillation (i.e. simple, fractional, Kugelrohr, etc.); gas chromatography using an appropriate temperature, carrier gas and flow rate; sublimation at an appropriate temperature and pressure; filtration through a media (i.e. Florosil®, alumina, Celite®, silica gel, etc.) with a solvent (i.e. heptane, hexanes, EtOAc, DCM, MeOH, etc.) or combination of solvents; salt formation with solid support (resin based, i.e. ion exchange) or without. Descriptions of these techniques can be found in the following references: Gordon, A. J. and Ford, R. A. “The Chemist's Companion”, 1972; Palleros, D. R. “Experimental Organic Chemistry”, 2000; Still, W. C., Kahn and M. Mitra, A. J. Org. Chem. 1978, 43, 2923; Yan, B. “Analysis and Purification Methods in Combinatorial Chemistry” 2003; Harwood, L. M., Moody, C. J. and Percy, J. M. “Experimental Organic Chemistry: Standard and Microscale, 2^{nd} Edition”, 1999; Stichlmair, J. G. and Fair, J. R. “Distillation; Principles and Practices” 1998; Beesley T. E. and Scott, R. P. W. “Chiral Chromatography”, 1999; Landgrebe, J. A. “Theory and Practice in the Organic Laboratory, 4^{th} Ed.”, 1993; Skoog, D. A. and Leary, J. J. “Principles of Instrumental Analysis, 4^{th} Ed.” 1992; G. Subramanian, “Chiral Separation Techniques 3^{rd} Edition” 2007; Y. Kazakevich, R. Lobrutto, “HPLC for Pharmaceutical Scientists” 2007.

### Degassing Methods

Preparations of intermediate and final compounds obtained via the General Procedures can be optionally degassed using one or more of the Degassing Methods described below. The reaction mixtures may be degassed by a single or multiple applications of any technique or combination of techniques known to one skilled in the art. Some examples that are not limiting include bubbling a continuous stream of an inert gas (e.g. nitrogen, argon, etc.) through a mixture of reagents and a solvent suitable for the transformation (e.g. THF, 1,4-dioxane, EtOAc, DCM, toluene, MeOH, EtOH, DMF, MeCN, water, etc.); freeze-thawing of a mixture of reagents in a solvent (e.g. THF, 1,4-dioxane, EtOAc, DCM, toluene, MeOH, EtOH, DMF, MeCN, water, etc.) where the resulting solution is cooled below its freezing point and evacuated under reduced pressure, then allowed to warm above the freezing point and purged with an atmosphere of inert gas (e.g. nitrogen, argon, etc.); evacuation under reduced pressure of a mixture of reagents with or without a suitable solvent for the transformation (e.g. THF, 1,4-dioxane, EtOAc, DCM, toluene, MeOH, EtOH, DMF, MeCN, water, etc.) followed by purging of the mixture with an inert gas (e.g. nitrogen, argon, etc.); evacuation under reduced pressure of a mixture of reagents in a suitable solvent for the transformation (e.g. THF, 1,4-dioxane, EtOAc, DCM, toluene, MeOH, EtOH, DMF, MeCN, water, etc.) with the aid of mechanical agitation (e.g. stirring, shaking, sonication, etc.) followed by purging of the mixture with an inert gas (e.g. nitrogen, argon, etc.). Some descriptions of these techniques can be found in the following references, Gordon, A. J. and Ford, R. A. “The Chemist's Companion”, 1972; Palleros, D. R. “Experimental Organic Chemistry”, 2000; Harwood, L. M., Moody, C. J. and Percy, J. M. “Experimental Organic Chemistry: Standard and Microscale, 2^{nd} Edition”, 1999; Landgrebe, J. A. “Theory and Practice in the Organic Laboratory, 4^{th} Edition”, 1993; Leonard, J., Lygo, B. and Procter, G. “Advanced Practical Organic Chemistry, 2^{nd} Edition”, 1998; Meyers, A. G.; Dragovich, P. S. Organic Syntheses, 1995, 72, 104; Hajos, Z. G., Parrish, D. R. Organic Syntheses, 1985, 63, 26.

### PREPARATIONS AND EXAMPLES

The general synthetic methods used in each of the General Procedures follow and include an illustration of a compound that was synthesized using the designated General Procedure. None of the specific conditions and reagents noted herein are to be construed as limiting the scope of the invention and are provided for illustrative purposes only. All starting materials are commercially available from Sigma-Aldrich (including Fluka and Discovery CPR) unless otherwise noted after the chemical name. Reagent/reactant names given are as named on the commercial bottle or as generated by IUPAC conventions, CambridgeSoft® Chemdraw Ultra 9.0.7 or AutoNom 2000. Compounds designated as salts (e.g. hydrochloride, acetate) may contain more than one molar equivalent of the salt.

### Preparation #1

### tert-Butyl 3-bromo-5-methylthiophen-2-ylcarbamate

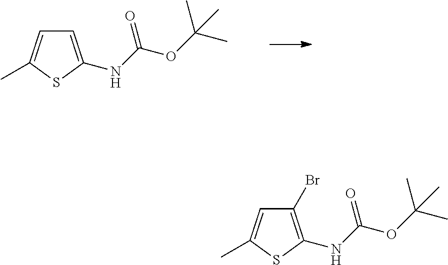

To a solution of tert-butyl 5-methylthiophen-2-ylcarbamate (13.0 g, 60.9 mmol, prepared using A from 5-methylthiophene-2-carboxylic acid) in MeOH (500 mL) at about 0° C. was added a solution of bromine (9.74 g, 60.9 mmol) in MeOH (500 mL) dropwise over about 2.5 h. The reaction mixture was warmed to ambient temperature and stirred for about 2 h. Water (400 mL) was added and the pH of the reaction was adjusted to about 7 using 2 N aqueous NaOH. MeOH was removed under reduced pressure and the remaining aqueous layer was extracted with DCM. The combined organics were dried over MgSO₄, filtered, and concentrated under reduced pressure. The remaining oil was purified by flash column chromatography on silica gel eluting with a gradient of 0-20% EtOAc in heptane. The solvent was removed under reduced pressure to give tert-butyl 3-bromo-5-methylthiophen-2-ylcarbamate (11.5 g, 65% yield): LC/MS (Table 1, Method a) Rₜ=2.57 min; MS m/z: 291 (M+H)⁺.

### Preparation #2

### (R)-Octahydropyrrolo[1,2-a]pyrazine (R)-2-hydroxy-2-phenylacetate

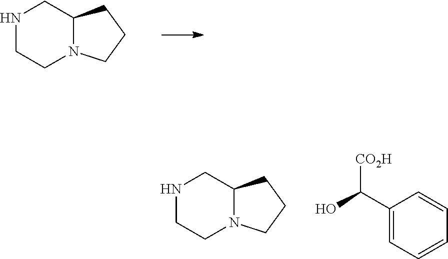

To R-mandelic acid (261 g, 1.72 mol) and MeCN (3.5 L) at about 45° C. was slowly added (R)-octahydropyrrolo[1,2-a]pyrazine (217 g, 1.72 mol, Focus Synthesis) over about 45 min. The slurry was mixed at about 50° C. for about 30 min and then cooled down slowly to about 20° C. over about 6 h. The resulting slurry was mixed about 20° C. for about 2 h. The product was filtered and rinsed with MeCN (600 mL). The wet cake was dried under vacuum at about 50° C. for about 16 h to afford (R)-octahydropyrrolo[1,2-a]pyrazine (R)-2-hydroxy-2-phenylacetate (366 g, 91% yield): LC/MS (Table 1, Method a) Rₜ=0.41 min; MS m/z: 127 (M+H)⁺.

### Preparation #3

### 1,7-Diazaspiro[3.5]nonane dihydrochloride

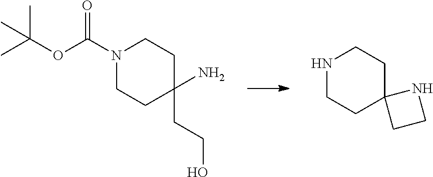

To a solution of tert-buty 4-amino-4-(2-hydroxyethyl)piperidine-1-carboxylate (0.233 g, 0.954 mmol, prepared using Q with LiBH₄ and 4-amino-4-methoxycarbonylpiperidine-1-carboxylic acid tert-butyl ester (AstaTech)), pyridine (0.386 mL, 4.77 mmol), and DCM (1.0 mL) under a nitrogen atmosphere at about 0° C. was added in one portion p-toluenesulfonyl chloride (0.364 g, 1.91 mmol). After about 1 h, the ice bath was removed and the solution was left to stir at ambient temperature for about 41 h. p-Toluenesulfonyl chloride (0.182 g, 0.954 mmol) was added. After stirring for about 30 h, saturated aqueous NH₄Cl (2 mL) and water (8 mL) were added. The mixture was extracted with DCM (3×10 mL). The combined organics were dried over Na₂SO₄, filtered, and concentrated. The residue was dissolved in 1,4-dioxane (20 mL) under a nitrogen atmosphere. KOt-Bu (1 M solution in THF, 1.24 mL, 1.24 mmol) was added. A reflux condenser was attached and the solution was warmed to about 80° C. After about 15 h, the mixture was allowed to cool to ambient temperature. Water (20 mL) and EtOAc (20 mL) were added. The layers were separated and the organics were dried over Na₂SO₄, filtered, and concentrated under reduced pressure. The residue was purified by flash column chromatography on silica gel eluting with a gradient of 20-60% EtOAc in heptane. The volatiles were removed under reduced pressure. 1-Pentanol (2.0 mL) was added under a nitrogen atmosphere and then sodium (0.164 g, 7.15 mmol) was added portionwise over about 1 h. The mixture was warmed to about 50° C. for about 13 h. The mixture was slowly warmed to about 140° C. over about 4 h. After about 17 h, the mixture was allowed to cool to ambient temperature. Water (3 mL) and Et₂O (5 mL) were added. The layers were separated and the organics were extracted with water (3×2 mL). The aqueous phase was acidified with 2 N aqueous HCl and then concentrated under reduced pressure to afford a yellow-white solid. The material was slurried in MeOH (10 mL) and then filtered. The volatiles were removed under reduced pressure to afford a yellow-white solid. The residue was slurried in 1% (7 N NH₃ in MeOH) in 10% MeOH/DCM (5 mL) and then filtered. The volatiles were removed under reduced pressure. MeOH (2.6 mL) was added under a nitrogen atmosphere. Magnesium powder (0.051 g, 2.1 mmol) was added. The mixture was sonicated for about 2 h. MeOH (1.5 mL) and magnesium powder (0.051 g, 2.1 mmol) were added. The mixture was sonicated for 3 h. The volatiles were removed under reduced pressure. The residue was slurried in Et₂O (10 mL). Sodium sulfate decahydrate (1.5 g) was added. The mixture was stirred for about 17 h. The mixture was filtered through Celite® with Et₂O rinses. The solution was acidified with 1 M HCl in Et₂O. The volatiles were removed under reduced pressure to afford 1,7-diazaspiro[3.5]nonane dihydrochloride (0.038 g, 21% yield): LC/MS (Table 1, Method e) Rₜ=0.14 min; MS m/z: 127 (M+H)⁺.

### Preparation #4

### tert-Butyl 6,7-dihydro-1H-imidazo[4,5-c]pyridine-5(4H)-carboxylate

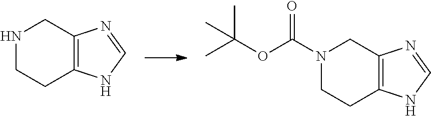

To a slurry of 4,5,6,7-tetrahydro-3H-imidazo[4,5-c]pyridine hydrochloride (0.911 g, 5.71 mmol, D-L Chiral Chemicals) in DCM (28.5 mL) under a nitrogen atmosphere was added TEA (3.18 mL, 22.8 mmol). Di-tert-butyldicarbonate (2.62 g, 12.0 mmol) was added. After stirring for about 20 h, the solution was washed with saturated aqueous NaHCO₃ (50 mL) and brine (25 mL). The organics were dried over Na₂SO₄, filtered, and concentrated. Ammonia (7 N in MeOH, 14.3 mL, 28.5 mmol) was added. After stirring for about 15 h, the volatiles were removed under reduced pressure. The residue was purified by flash column chromatography on silica gel eluting with a gradient of 50-100% (1% (7 N NH₃ in MeOH) in 10% MeOH/DCM) in DCM. The volatiles were removed under reduced pressure to afford tert-butyl 6,7-dihydro-1H-imidazo[4,5-c]pyridine-5(4H)-carboxylate (0.880 g, 69% yield): LC/MS (Table 1, Method a) Rₜ=1.20 min; MS m/z: 224 (M+H)⁺.

### Preparation #5

### Ethyl 2-(3-((1-methylpiperidin-4-yl)methyl)imidazo[1,5-a]pyridin-1-yl)acetate

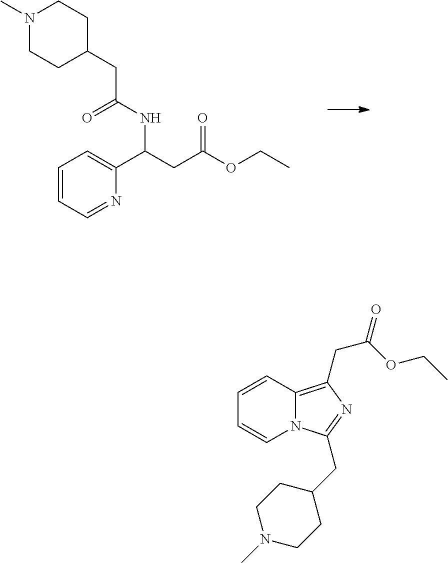

Phosphorous oxychloride (10.6 mL, 114 mmol, Sinopharm Chemical Reagent Co. Ltd.) was added to a mixture of ethyl 3-(2-(1-methylpiperidin-4-yl)acetamido)-3-(pyridin-2-yl)propanoate (2.64 g, 7.92 mmol, prepared using K with formaldehyde (Sinopharm Chemical Reagent Co. Ltd.), Na(AcO)₃BH (Sinopharm Chemical Reagent Co. Ltd.), and ethyl 3-(2-(piperidin-4-yl)acetamido)-3-(pyridin-2-yl)propanoate (prepared using G with TFA (Sinopharm Chemical Reagent Co. Ltd.) and Preparation #S.1)) in DCE (80 mL). The mixture was stirred at about 80° C. for about 5 h. After cooling to ambient temperature, the mixture was diluted with DCE (20 mL), and then concentrated under reduced pressure. The resulting residue was partitioned between EtOAc (200 mL) and saturated aqueous NaHCO₃. The organic layer was separated and the aqueous layer was extracted with EtOAc (5×300 mL). The combined organic layers were dried over Na₂SO₄. The filtrate was concentrated to give a residue which was directly used in the next step, ethyl 2-(3-((1-methylpiperidin-4-yl)methyl)imidazo[1,5-a]pyridin-1-yl)acetate: (1.57 g, 63% yield): LC/MS (Table 1, Method g) Rₜ=1.60 min; MS m/z: 316 (M+H)⁺.

### Preparation #6

### 2-(7-Fluoro-3-(4-methylpiperazin-1-yl)isoquinolin-1-yl)acetamide

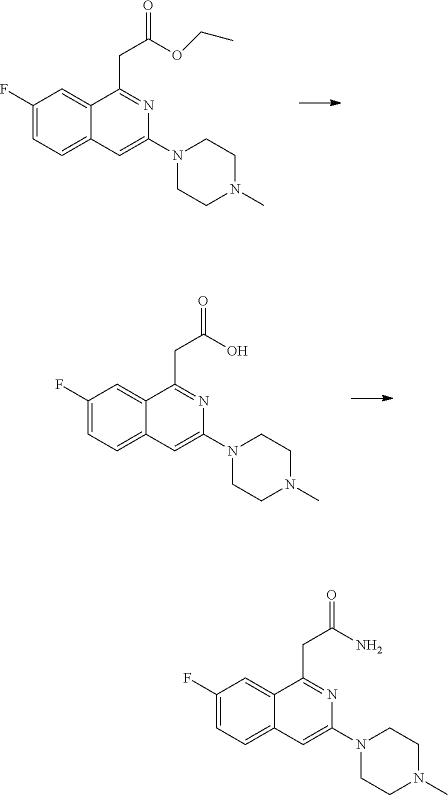

Sodium hydroxide (1 M aqueous solution, 0.97 mL, 0.97 mmol) was added to a solution of ethyl 2-(7-fluoro-3-(4-methylpiperazin-1-yl)isoquinolin-1-yl)acetate (0.20 g, 0.60 mmol, prepared using E with 1-methylpiperazine and ethyl 2-(3-chloro-7-fluoroisoquinolin-1-yl)acetate (prepared using M with HCl gas, EtOH, and tert-butyl 2-(3-chloro-7-fluoroisoquinolin-1-yl)acetate (Preparation #L.3))) in EtOH (2 mL). The reaction mixture was stirred at ambient temperature for about 1 h, then cooled to about 0° C., and acidified with HCl (37% wt). The volatiles were removed using a flow of nitrogen and the residue was triturated with a solution of 10% MeOH in Et₂O. The resulting solid was collected by filtration and then dissolved in DMF (2 mL). [Dimethylamino-([1,2,3]triazolo[4,5-b]pyridin-3-yloxy)-methylene]dimethylammonium hexafluorophosphate (0.27 g, 0.72 mmol), TEA (0.50 mL, 3.62 mmol), and NH₃ (0.5 M solution in 1,4-dioxane, 12 mL, 6.0 mmol) were added and the resulting mixture was stirred at ambient temperature for about 5 min. The mixture was filtered. The filtrate was collected and partitioned between water and EtOAc. The organic layer was dried over MgSO₄, filtered, and evaporated under reduced pressure. The residue was purified by flash column chromatography on silica gel eluting with a gradient of 0-20% MeOH in DCM to give 2-(7-fluoro-3-(4-methylpiperazin-1-yl)isoquinolin-1-yl)acetamide (0.050 g, 31% yield): LC/MS (Table 1, Method b) Rₜ=1.04 min; MS m/z: 303 (M+H)⁺.

### Preparation #7

### 1-Methyloctahydro-1H-pyrrolo[3,2-c]pyridine

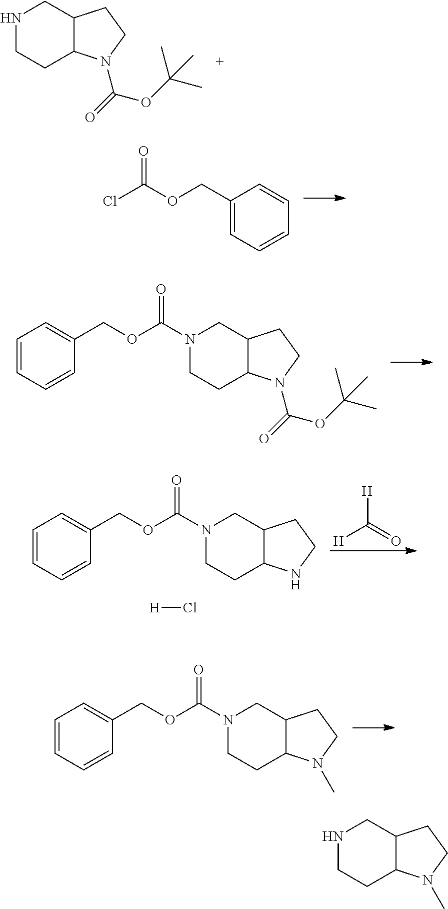

To a solution of tert-butyl octahydro-1H-pyrrolo[3,2-c]pyridine-1-carboxylate (1.05 g, 4.64 mmol, Shah, S. K et. al, Bioorg. Med. Chem. Lett. 2005, 15, 977-982) in DCM (20 mL) was added TEA (1.29 mL, 9.29 mmol) followed by dropwise addition of benzyl chloroformate (0.795 mL, 5.57 mmol) at ambient temperature. After about 4 h, the mixture was diluted with DCM and then washed with water and brine. The organics were dried over Na₂SO₄, filtered and concentrated under reduced pressure. The residue was purified by flash column chromatography on silica gel eluting with a gradient of 0-75% EtOAc in heptane to give 5-benzyl 1-tert-butyl hexahydro-1H-pyrrolo[3,2-c]pyridine-1,5(6H)-dicarboxylate (1.05 g, 63% yield): LC/MS (Table 1, Method a) Rₜ=2.59 min; MS m/z: 361 (M+H)⁺. A solution of 5-benzyl 1-tert-butyl hexahydro-1H-pyrrolo[3,2-c]pyridine-1,5(6H)-dicarboxylate (1.05 g, 2.92 mmol) in HCl (4.0 M in 1,4-dioxane, 7.31 mL, 29.2 mmol) was stirred at ambient temperature for about 1 h. The volatiles were removed under reduced pressure and the residue was dried under high vacuum for about 18 h to give benzyl hexahydro-1H-pyrrolo[3,2-c]pyridine-5(6H)-carboxylate hydrochloride (0.867 g, 100% yield): LC/MS (Table 1, Method a) Rₜ=1.41 min; MS m/z: 261 (M+H)⁺. To a solution of benzyl hexahydro-1H-pyrrolo[3,2-c]pyridine-5(6H)-carboxylate hydrochloride (0.867 g, 2.92 mmol) in MeOH (15 mL) was added formaldehyde (37 wt. % in water, 1.09 mL, 14.6 mmol). The mixture was stirred at ambient temperature for about 1 h. NaBH₃CN (0.404 g, 6.43 mmol) was added in one portion. The mixture was stirred at ambient temperature for about 20 h. The volatiles were removed under reduced pressure. The residue was dissolved with water. The pH was adjusted to about 11 with 2 N aqueous NaOH. The mixture was extracted with DCM (2×15 mL). The combined organic layers were dried over MgSO₄, filtered, and concentrated under reduced pressure to give benzyl 1-methylhexahydro-1H-pyrrolo[3,2-c]pyridine-5(6H)-carboxylate (0.750 g, 94% yield): LC/MS (Table 1, Method a) Rₜ=1.54 min; MS m/z: 275 (M+H)⁺. A solution of benzyl 1-methylhexahydro-1H-pyrrolo[3,2-c]pyridine-5(6H)-carboxylate (0.126 g, 0.459 mmol) in MeOH (20 mL) was added to 10% Pd/C (0.020 g, 0.019 mmol). The mixture was shaken at about 60 psi of hydrogen for about 16 h, and then filtered through a pad of Celite® washing with EtOAc. The filtrate was concentrated under reduced pressure to give 1-methyloctahydro-1H-pyrrolo[3,2-c]pyridine (0.057 g, 89% yield): LC/MS (Table 1, Method a) Rₜ=1.45 min; MS m/z: 227 (M+H)⁺.

### Preparation #8

### tert-Butyl 4-((1-(2-amino-2-oxoethyl)-1H-indol-3-yl)methyl)piperidine-1-carboxylate

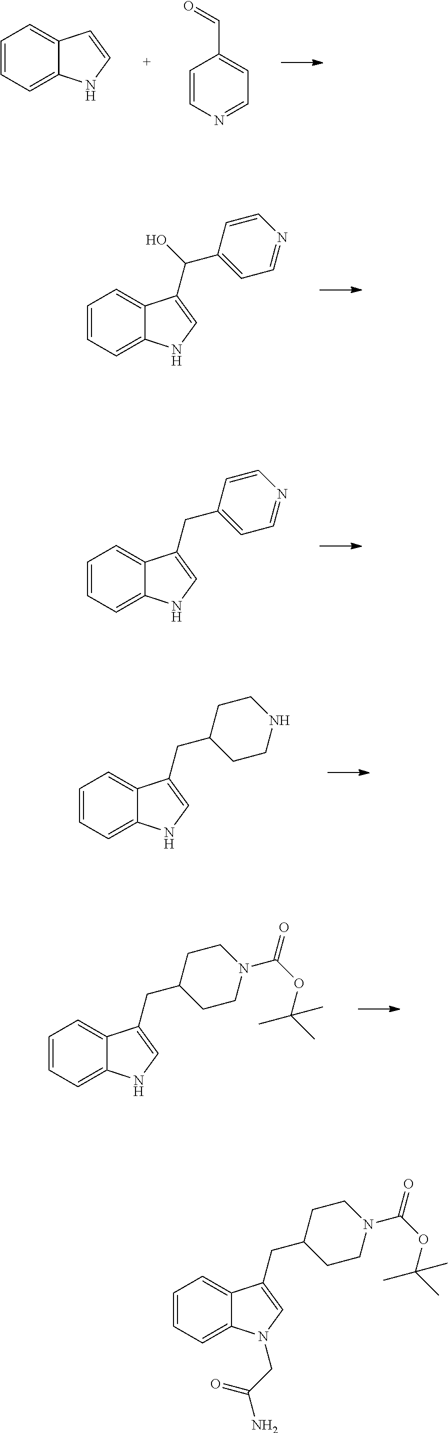

To a solution of 1H-indole (15.0 g, 128 mmol) in MeOH (100 mL) was added isonicotinaldehyde (12.5 mL, 128 mmol). The mixture was cooled to about 0° C. with an ice-water bath. NaOH (50% in water, 14.2 mL, 128 mmol) was added dropwise. The mixture was stirred at about 0° C. for about 1 h and then warmed to ambient temperature. After about 5 h, the mixture was diluted with water. The precipitate was collected by filtration, washed with water, and dried under vacuum at about 60° C. for about 16 h to give (1H-indol-3-yl)(pyridin-4-yl)methanol (27.4 g, 96% yield): LC/MS (Table 1, Method a) Rₜ=1.25 min; MS m/z: 225 (M+H)⁺. To a suspension of (1H-indol-3-yl)(pyridin-4-yl)methanol (27.4 g, 122 mmol) in DCM (300 mL) was added triethylsilane (21.8 mL, 136 mmol) followed by TFA (105 mL, 1360 mmol) at ambient temperature. The mixture was stirred for about 2 h before the volatiles were removed under reduced pressure. The residue was adjusted to about pH 9 with saturated aqueous Na₂CO₃. The mixture was extracted with EtOAc. The organic layer was concentrated under reduced pressure and the residue was purified by flash column chromatography on silica gel eluting with a gradient of 0-100% EtOAc in heptane followed by trituration with heptane to give 3-(pyridin-4-ylmethyl)-1H-indole (5.80 g, 23% yield): LC/MS (Table 1, Method a) Rₜ=1.32 min; MS m/z: 209 (M+H)⁺. 3-(Pyridin-4-ylmethyl)-1H-indole (5.00 g, 24.0 mmol) and acetic acid (25 mL) were added to platinum(IV) oxide (1.00 g, 4.40 mmol, Johnson Matthey) in a 250 mL stainless steel pressure bottle and stirred for about 2 hr under about 30 psi hydrogen at ambient temperature. The mixture was filtered and the filtrate was concentrated under reduced pressure. The residue was dissolved with EtOAc and then washed with saturated aqueous Na₂CO₃. The organic layer became turbid. A small amount of MeOH was added to form a clear solution. The organic layer was washed with 2 N aqueous NaOH and brine, dried over MgSO₄, filtered, and concentrated under reduced pressure. The resulting solid was triturated with Et₂O and then dried to give 3-(piperidin-4-ylmethyl)-1H-indole (4.35 g, 73% yield): LC/MS (Table 1, Method a) Rₜ=1.51 min; MS m/z: 215.12 (M+H)⁺. To a solution of 3-(piperidin-4-ylmethyl)-1H-indole (0.500 g, 2.33 mmol) in 1,4-dioxane (5 mL) was added NaOH (1.0 M aqueous solution, 5.00 mL, 5.00 mmol). A solution of Boc₂O (0.509 g, 2.33 mmol) in 1,4-dioxane (5.00 mL) was added dropwise. The mixture was stirred at ambient temperature for about 16 h. The organic phase was separated, washed with water and brine, dried over MgSO₄, filtered, and concentrated under reduced pressure. The residue was purified by flash column chromatography on silica gel eluting with a gradient of 0-25% EtOAc in heptane to give tert-butyl 4-((1H-indol-3-yl)-methyl)piperidine-1-carboxylate (0.661 g, 90% yield): LC/MS (Table 1, Method a) Rₜ=2.80 min; no ionization. To a solution of tert-butyl 4-((1H-indol-3-yl)methyl)piperidine-1-carboxylate (0.661 g, 2.10 mmol) in DMF (6 mL) at about 0° C. under a nitrogen atmosphere was added NaH (60% dispersion in mineral oil, 0.109 g, 2.73 mmol) in one portion. The mixture was stirred at about 0° C. for about 30 min. A solution of 2-bromoacetamide (0.305 g, 2.21 mmol) in DMF (6.00 mL) was added dropwise. The mixture was stirred at about 0° C. for about 2 h and then at ambient temperature for about 16 h. The mixture was partitioned between EtOAc and water. The aqueous layer was further extracted with EtOAc (3×25 mL). The combined organic layers were concentrated under reduced pressure. The residue was purified by flash column chromatography on silica gel eluting with a gradient of 0-100% EtOAc in heptane to give tert-butyl 4-((1-(2-amino-2-oxoethyl)-1H-indol-3-yl)methyl)piperidine-1-carboxylate (0.387 g, 50% yield): LC/MS (Table 1, Method a) Rₜ=2.35 min; MS m/z: 372 (M+H)⁺.

### Preparation #9

### Methyl 2-(8-((tert-butyldimethylsilyloxy)methyl)-6,7,8,9-tetrahydropyrido[1,2-a]indol-10-yl)-2-oxoacetate

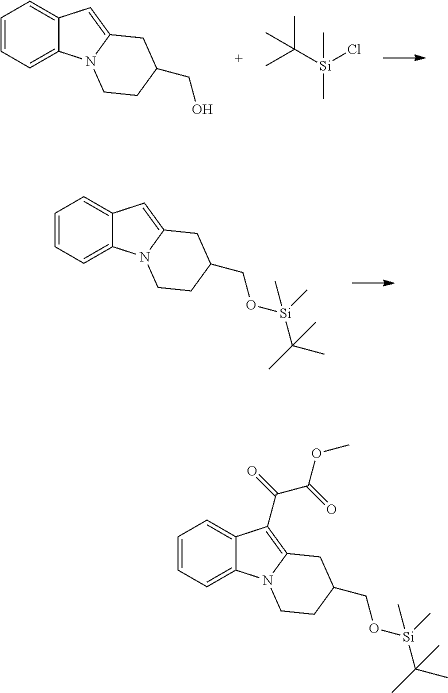

To a solution of (6,7,8,9-tetrahydropyrido[1,2-a]indol-8-yl)methanol (3.30 g, 16.4 mmol, U.S. Pat. No. 5,721,245A1) and imidazole (2.68 g, 39.4 mmol) in DMF (41.0 mL) was added tert-butylchlorodimethylsilane (2.97 g, 19.7 mmol) in one portion. The mixture was stirred at ambient temperature for about 5 h. Water (80 mL) was added and the mixture was extracted with EtOAc (3×30 mL). The combined organic layers were washed with brine, dried over MgSO₄, filtered and concentrated under reduced pressure. The residue was purified by flash column chromatography on silica gel eluting with a gradient of 0-10% EtOAc in heptane to give 8-((tert-butyldimethylsilyloxy)methyl)-6,7,8,9-tetrahydropyrido[1,2-a]indole (5.17 g, 100% yield): LC/MS (Table 1, Method a) Rₜ=1.84 min; MS m/z: 316 (M+H)⁺. To a colorless solution of 8-((tert-butyldimethylsilyloxy)methyl)-6,7,8,9-tetrahydropyrido[1,2-a]indole (0.100 g, 0.317 mmol) in DCM (4.0 mL) at about 0° C. was added oxalyl chloride (0.033 mL, 0.380 mmol) dropwise. The mixture was stirred at about 0° C. for about 1 h. A mixture of DIEA (0.166 mL, 0.951 mmol) and anhydrous MeOH (0.490 mL, 12.0 mmol) was added. The mixture was stirred at about 0° C. for about 30 min and then at ambient temperature for about 30 min. The volatiles were removed under reduced pressure. The residue was purified by flash column chromatography on silica gel eluting with a gradient of 0-40% EtOAc in heptane to give methyl 2-(8-((tert-butyldimethylsilyloxy)methyl)-6,7,8,9-tetrahydropyrido[1,2-a]indol-10-yl)-2-oxoacetate (0.106 g, 83% yield): LC/MS (Table 1, Method a) Rₜ=3.21 min; MS m/z: 402.

### Preparation #10

### tert-Butyl 4-(2-amino-2-oxoethyl)-6H-thieno[2,3-b]pyrrole-6-carboxylate

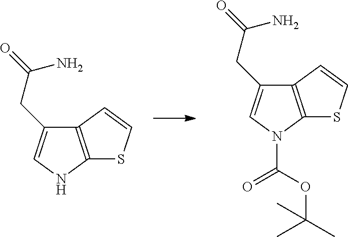

To a solution of 2-(6H-thieno[2,3-b]pyrrol-4-yl)acetamide (0.200 g, 1.11 mmol, prepared using J with tert-butyl 4-(2-ethoxy-2-oxoethyl)-6H-thieno[2,3-b]pyrrole-6-carboxylate (Preparation #B.1)) and 4-(dimethylamino)pyridine (0.020 g, 0.17 mmol) in THF (6 mL) was added dropwise a solution of Boc₂O (0.254 g, 1.17 mmol) in THF (1.00 mL). The mixture was stirred at ambient temperature for about 3 h. The volatiles were removed under reduced pressure. The residue was purified by flash column chromatography on silica gel eluting with a gradient of 0-5% MeOH in DCM to give tert-butyl 4-(2-amino-2-oxoethyl)-6H-thieno[2,3-b]pyrrole-6-carboxylate (0.311 g, 100% yield): LC/MS (Table 1, Method a) Rₜ=2.10 min; MS m/z: 281 (M+H)⁺.

### Example #1

### 3-(8-(Hydroxymethyl)-6,7,8,9-tetrahydropyrido[1,2-a]indol-10-yl)-4-(6H-thieno[2,3-b]pyrrol-4-yl)-1H-pyrrole-2,5-dione

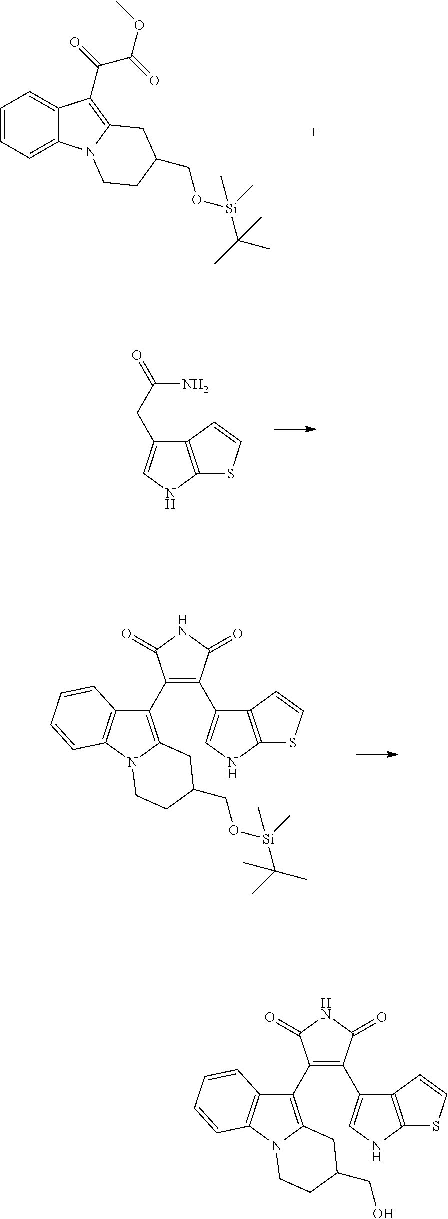

To a suspension of methyl 2-(8-((tert-butyldimethylsilyloxy)methyl)-6,7,8,9-tetrahydropyrido[1,2-a]indol-10-yl)-2-oxoacetate (0.106 g, 0.264 mmol, Preparation #9) and 2-(6H-thieno[2,3-b]pyrrol-4-yl)acetamide (0.095 g, 0.53 mmol, prepared using J with tert-butyl 4-(2-ethoxy-2-oxoethyl)-6H-thieno[2,3-b]pyrrole-6-carboxylate (Preparation #B.1)) in THF (5 mL) at about −10° C. was added KOt-Bu (1.0 M in THF, 1.85 mL, 1.85 mmol) dropwise. The mixture was stirred at about −10° C. for about 1 h and then at ambient temperature for about 1 h. The mixture was warmed to about 50° C. for about 1 h. After cooling to ambient temperature, the reaction mixture was partitioned between EtOAc and water. The organic layer was separated and the aqueous layer was extracted with EtOAc (2×15 mL). The combined organics were concentrated under reduced pressure. The residue was dissolved with THF (2 mL). HCl (1.0 M in water, 2.0 mL, 2.0 mmol) was added. The mixture was stirred at ambient temperature for about 15 min. The reaction mixture was partitioned between EtOAc and water. The organic layer was separated and the aqueous layer was extracted with EtOAc (2×15 mL). The combined organic layers were dried over MgSO₄, filtered and concentrated. The residue was purified by flash column chromatography on silica gel eluting with a gradient of 50-75% EtOAc in heptane to give 3-(8-(hydroxymethyl)-6,7,8,9-tetrahydropyrido[1,2-a]indol-10-yl)-4-(6H-thieno[2,3-b]pyrrol-4-yl)-1H-pyrrole-2,5-dione (0.0048 g, 4% yield): LC/MS (Table 1, Method c) Rₜ=2.05 min; MS m/z: 418 (M+H)⁺.

### Example #2

### 3-(8-((Dimethylamino)methyl)-6,7,8,9-tetrahydropyrido[1,2-a]indol-10-yl)-4-(6H-thieno[2,3-b]pyrrol-4-yl)-1H-pyrrole-2,5-dione

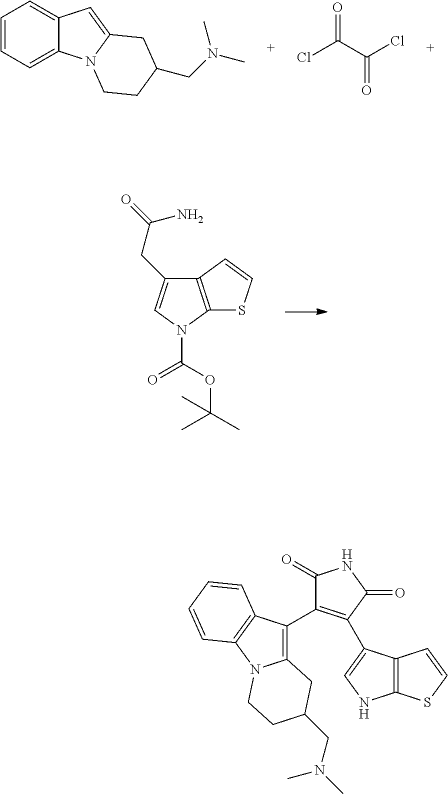

A solution of N,N-dimethyl-1-(6,7,8,9-tetrahydropyrido[1,2-a]indol-8-yl)methanamine (0.050 g, 0.220 mmol, prepared using I with dimethylamine and (6,7,8,9-tetrahydropyrido[1,2-a]indol-8-yl)methyl methanesulfonate (prepared using H with MsCl and (6,7,8,9-tetrahydropyrido[1,2-a]indol-8-yl)methanol (U.S. Pat. No. 5,721,245A1))) and TEA (0.037 mL, 0.26 mmol) in DCM (2.00 mL) was cooled to about 0° C. Oxalyl chloride (0.023 mL, 0.26 mmol) was added dropwise. The mixture was stirred at about 0° C. for about 1 h. The volatiles were removed under reduced pressure. tert-Butyl 4-(2-amino-2-oxoethyl)-6H-thieno[2,3-b]pyrrole-6-carboxylate (0.123 g, 0.438 mmol, Preparation #10) and THF were added. The brown suspension was cooled to about −10° C. KOt-Bu (1.0 M in THF, 1.53 mL, 1.53 mmol) was added dropwise. The mixture was stirred at about −10° C. for about 30 min, at ambient temperature for about 16 h, and then at about 50° C. for about 1 h. The reaction mixture was partitioned between EtOAc and water. The organic layer was separated and the aqueous layer was extracted with EtOAc (2×15 mL). The combined organic layers were dried over MgSO₄, filtered, and concentrated under reduced pressure. The residue was purified by flash column chromatography on silica gel eluting with a gradient of (100:0:0-100:5:0.5 DCM/MeOH/(7 M NH₃ in MeOH) and then hold 100:5:0.5 DCM/MeOH/(7 M NH₃ in MeOH) to give 3-(8-((dimethylamino)methyl)-6,7,8,9-tetrahydropyrido[1,2-a]indol-10-yl)-4-(6H-thieno[2,3-b]pyrrol-4-yl)-1H-pyrrole-2,5-dione (0.009 g, 9% yield): LC/MS (Table 1, Method c) Rₜ=1.77 min; MS m/z: 445 (M+H)⁺.

### General Procedure A

### Formation of a Boc-Protected Amine from a Carboxylic Acid

TEA (1-2 equiv, preferably 1 equiv) is added to a suspension of the carboxylic acid (1 equiv) in an organic solvent such as tert-butanol under a nitrogen atmosphere. Diphenylphosphoryl azide (0.8-1.3 equiv, preferably 0.9-1.1 equiv) is added and the resulting mixture is stirred at about 40-80° C. (preferably 65° C.) for about 1-24 h (preferably 4 h). The reaction mixture is allowed to cool to ambient temperature and the volatiles are removed under reduced pressure.

### Illustrations of General Procedure A

### Preparation #A.1

### tert-Butyl 3-bromothiophen-2-ylcarbamate

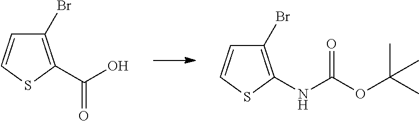

A round bottomed flask was charged with 3-bromothiophene-2-carboxylic acid (25.0 g, 117 mmol) and tert-butanol (234 mL). The suspension was placed under a nitrogen atmosphere and TEA (16.3 mL, 117 mmol) was added followed by diphenyl phosphoryl azide (27.8 mL, 129 mmol). The resulting mixture was heated to about 65° C. for about 4 h. The reaction mixture was cooled to ambient temperature and concentrated under reduced pressure. The remaining residue was purified by flash column chromatography on silica gel eluting with a gradient of 0-5% EtOAc in heptane. The solvent was removed under reduced pressure to give tert-butyl 3-bromothiophen-2-ylcarbamate (26.3 g, 81% yield): LC/MS (Table 1, Method a) Rₜ=2.48 min; MS m/z: 278 (M+H)⁺.

### Preparation #A.2

### tert-Butyl 2-bromothiophen-3-ylcarbamate

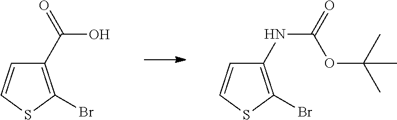

A round bottomed flask was charged with 2-bromothiophene-3-carboxylic acid (15.0 g, 72.4 mmol) and tert-butanol (188 mL). The suspension was placed under a nitrogen atmosphere and TEA (10.1 mL, 72.4 mmol) was added followed by diphenyl phosphoryl azide (14.2 mL, 65.9 mmol). The resulting mixture was heated to about 65° C. for about 4 h. The reaction mixture was cooled to ambient temperature and the solvent was removed under reduced pressure. The remaining residue was purified by flash column chromatography on silica gel eluting with a gradient of 1-5% EtOAc in heptane. The solvent was removed under reduced pressure to give tert-butyl 2-bromothiophen-3-ylcarbamate (12.4 g, 68% yield): LC/MS (Table 1, Method a) Rₜ=2.46 min; MS m/z: 278 (M+H)⁺.

### General Procedure B

### Formation of a Pyrrole

A base such as K₂CO₃ or Na₂CO₃ (2-10 equiv, preferably K₂CO₃, 4 equiv) is added to a solution of the carbamate (1 equiv) and an organic solvent such as DMF or THF (preferably DMF) at ambient temperature under a nitrogen atmosphere. The halocrotonate (1-3 equiv, preferably 1.5 equiv) is added and the mixture is stirred for about 14-24 h (preferably 16 h). Triphenylphosphine (0.04-0.20 equiv, preferably 0.1 equiv) and Pd(OAc)₂ (0.02-0.10 equiv, preferably 0.05 equiv) are added. The reaction mixture is heated to about 40-100° C. (preferably 75° C.) for about 3-24 h (preferably 6 h). The volatiles are removed under reduced pressure. The residue is partitioned between an organic solvent such as EtOAc or DCM (preferably EtOAc) and water and/or brine. The aqueous layer is optionally extracted additional times. The resulting organic layer is optionally washed with brine, dried over Na₂SO₄ or MgSO₄, filtered, and concentrated under reduced pressure. Intermediates and final compounds prepared via this General Procedure can be optionally purified using one or more of the Purification Methods described above.

### Illustrations of General Procedure B

### Preparation #B.1

### tert-Butyl 4-(2-ethoxy-2-oxoethyl)-6H-thieno[2,3-b]pyrrole-6-carboxylate

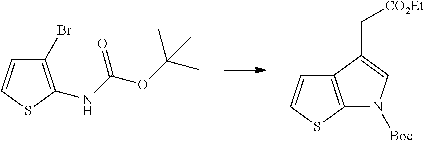

To a solution of tert-butyl 3-bromothiophen-2-ylcarbamate (26.3 g, 94.0 mmol, Preparation #A.1) in DMF (189 mL) was added K₂CO₃ (52.2 g, 378 mmol) and ethyl 4-bromocrotonate (26.0 mL, 142 mmol). The mixture was stirred at ambient temperature for about 16 h and triphenylphosphine (2.48 g, 9.44 mmol) and Pd(OAc)₂ (1.06 g, 4.72 mmol) were added. The reaction mixture was heated to about 75° C. for about 6 hours then cooled to ambient temperature and concentrated under reduced pressure. The remaining residue was partitioned between EtOAc (300 mL) and water (300 mL), the layers were separated and the aqueous phase was extracted with EtOAc (2×50 mL). The combined organics were washed with brine, dried over Na₂SO₄, filtered and concentrated under reduced pressure. The residue was purified by flash column chromatography on silica gel eluting with a gradient of 0-10% EtOAc in heptane to give tert-butyl 4-(2-ethoxy-2-oxoethyl)-6H-thieno[2,3-b]pyrrole-6-carboxylate (19.9 g, 68% yield): LC/MS (Table 1, Method a) Rₜ=2.85 min; MS m/z: 310 (M+H)⁺.

### Preparation #B.2

### tert-Butyl 6-(2-ethoxy-2-oxoethyl)-4H-thieno[3,2-b]pyrrole-4-carboxylate

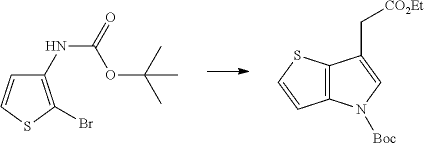

To a solution of tert-butyl 2-bromothiophen-3-ylcarbamate (12.4 g, 44.7 mmol, Preparation #A.2) in DMF (89 mL) was added K₂CO₃ (27.7 g, 179 mmol) and ethyl 4-bromocrotonate (12.3 mL, 67.1 mmol). The mixture was stirred at ambient temperature for about 16 h and triphenylphosphine (1.17 g, 4.47 mmol) and Pd(OAc)₂ (0.502 g, 2.24 mmol) were added. The reaction mixture was heated to about 75° C. for about 6 h then cooled to ambient temperature and concentrated under reduced pressure. The remaining residue was partitioned between EtOAc (200 mL) and water (200 mL), the layers were separated and the aqueous phase was extracted with EtOAc (2×50 mL). The combined organics were washed with brine, dried over MgSO₄, filtered and concentrated under reduced pressure. The residue was purified using flash column chromatography on silica gel eluting with a gradient of 0-5% EtOAc in heptane to give tert-butyl 6-(2-ethoxy-2-oxoethyl)-4H-thieno[3,2-b]pyrrole-4-carboxylate (9.51 g, 69% yield): LC/MS (Table 1, Method a) Rₜ=2.71 min; MS m/z: 310 (M+H)⁺.

### General Procedure C

### Oxidation of an Ester to an α-Keto Ester

Selenium dioxide (1.8-3 equiv, preferably 2 equiv) is added to a solution of the ester (1 equiv), an organic solvent such as THF or 1,4-dioxane (preferably THF), and water under a nitrogen atmosphere. The mixture is heated to about 40-100° C. (preferably 65° C.) for about 1-24 h (preferably 6 h) and then is cooled to ambient temperature. The reaction mixture is optionally filtered through Celite®, silica, or MgSO₄ (preferably silica) washing with an organic solvent such as DCM and then partitioned with brine. The organics are concentrated under reduced pressure.

### Illustrations of General Procedure C

### Preparation #C.1

### tert-Butyl 4-(2-ethoxy-2-oxoacetyl)-6H-thieno[2,3-b]pyrrole-6-carboxylate

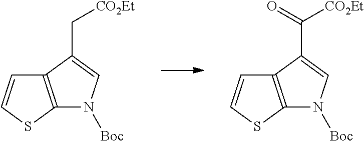

A round bottomed flask was charged with tert-butyl 4-(2-ethoxy-2-oxoethyl)-6H-thieno[2,3-b]pyrrole-6-carboxylate (26.3 g, 85.0 mmol, Preparation #B.1), selenium dioxide (18.9 g, 170 mmol), THF (270 mL) and water (30 mL). The suspension was heated at about 65° C. for about 7 h and the reaction was cooled to ambient temperature. The reaction mixture was filtered and the filtrate was washed with brine (2×15 mL). The organics were dried over MgSO₄, filtered, and concentrated under reduced pressure. A solution of 10% MeOH in DCM (300 mL) was added, the precipitate was filtered off, and silica gel was added to the filtrate. The filtrate was concentrated under reduced pressure and the remaining silica mixture was purified by flash column chromatography on silica gel eluting with a gradient of 5-35% EtOAc in heptane to give tert-butyl 4-(2-ethoxy-2-oxoacetyl)-6H-thieno[2,3-b]pyrrole-6-carboxylate (8.90 g, 31% yield): LC/MS (Table 1, Method c) Rₜ=2.80 min; MS m/z: 324 (M+H)⁺.

### Preparation #C.2

### tert-Butyl 6-(2-ethoxy-2-oxoacetyl)-4H-thieno[3,2-b]pyrrole-4-carboxylate

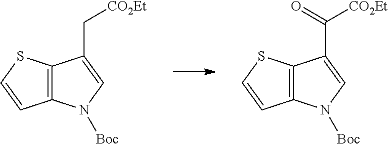

To a solution of tert-butyl 6-(2-ethoxy-2-oxoethyl)-4H-thieno[3,2-b]pyrrole-4-carboxylate (4.87 g, 15.7 mmol, Preparation #B.2) in THF (28.3 mL) and water (3.15 mL) was added selenium dioxide (3.49 g, 31.5 mmol). The suspension was heated at about 85° C. for about 4 h. The reaction mixture was cooled to ambient temperature and filtered through a pad of silica gel washing with DCM (200 mL). The filtrate was concentrated under reduced pressure and the remaining residue was dissolved in EtOAc (100 mL). The solids were filtered off and the filtrate was concentrated under reduced pressure. The residue was dissolved in DCM (200 mL) filtered through a pad of silica gel and the filtrate was concentrated under reduced pressure to give tert-butyl 6-(2-ethoxy-2-oxoacetyl)-4H-thieno[3,2-b]pyrrole-4-carboxylate (3.57 g, 70% yield): LC/MS (Table 1, Method c) Rₜ=2.92 min; MS m/z: 324 (M+H)⁺.

### General Procedure D

### Displacement of a Heteroaryl Halide or Heteroaryl Sulfone Followed by Amino-De-Alkoxylation of an Ester

A solution of ethyl acetoacetate (preferably 2 equiv) and an organic solvent such as THF or 1,4-dioxane (preferably THF) is added dropwise to a mixture of NaH (1-2 equiv, preferably 1.3-1.5 equiv) optionally as a dispersion in mineral oil in an organic solvent such as THF or 1,4-dioxane (preferably THF) at about −10-25° C. (preferably 0° C.) under a nitrogen atmosphere. After about 5-60 min (preferably 5-10 min), the volatiles are removed under reduced pressure. The heteroaryl halide or heteroaryl sulfonate (1 equiv) and an organic solvent such as toluene are added. The resulting slurry is warmed to 40-110° C. (preferably 110° C.) for about 0.5-24 h (preferably 1-2 h). The volatiles are removed under reduced pressure. The residue is treated with NH₄OH (10-200 equiv, preferably 25-130 equiv) at ambient temperature. Optionally, an organic solvent such as EtOH is added. The reaction mixture is then stirred at ambient temperature to about 40-80° C. (preferably 45-50° C.) for about 1 to 48 h (preferably 1-16 h). The volatiles are removed under reduced pressure

### Illustration of General Procedure D

### Preparation #D.1

### 2-(2-Chloroquinazolin-4-yl)acetamide

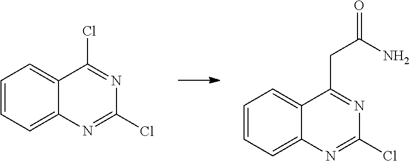

To a suspension of NaH (60% dispersion in mineral oil, 6.01 g, 150 mmol) in THF (130 mL) at about 0° C. under a nitrogen atmosphere was added ethyl acetoacetate (29.3 mL, 231 mmol, Oakwood) dropwise over about 10 min. The solvent was removed under reduced pressure and toluene (579 mL) was added. 2,4-Dichloroquinazoline (23.0 g, 116 mmol, AstaTech) was added and the reaction mixture was stirred at about 110° C. for about 1 h. The reaction was cooled to ambient temperature and concentrated under reduced pressure. NH₄OH (434 mL, 3120 mmol) was added and the mixture was stirred at ambient temperature for about 1.5 h. EtOH (180 mL) was added and the reaction mixture was heated to about 50° C. for about 22 h. The reaction was cooled to ambient temperature and the volume was reduced by about half under reduced pressure forming a heterogeneous mixture. After about 2 h at ambient temperature, the mixture was cooled to about 0° C. The precipitate was collected by filtration and the solids were washed with water (2×60 mL) to give 2-(2-chloroquinazolin-4-yl)acetamide (14.3 g, 56% yield): LC/MS (Table 1, Method b) Rₜ=1.04 min; MS m/z: 222 (M+H)⁺.

### Preparation #D.2

### 2-(2-Chlorothieno[2,3-d]pyrimidin-4-yl)acetamide

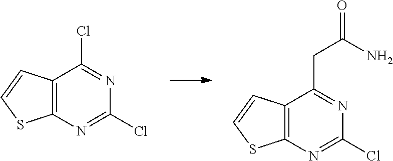

To a suspension of NaH (60% dispersion in mineral oil, 1.42 g, 35.5 mmol) in THF (28.3 mL) at about 0° C. under a nitrogen atmosphere was added ethyl acetoacetate (5.99 mL, 47.4 mmol) dropwise. Upon completion of addition, the reaction solution was stirred for about 5 min at about 0° C. The solvent was removed under reduced pressure and toluene (118 mL) was added. 2,4-Dichlorothieno[2,3-d]pyrimidine (4.86 g, 23.7 mmol, ArkPharm) was added and the reaction mixture was stirred at reflux for about 2 h. The reaction was cooled to ambient temperature and concentrated under reduced pressure. NH₄OH (118 mL, 3030 mmol) was added and the mixture was stirred at about 45° C. for about 16 h. The mixture was cooled to about 0° C. and filtered. The precipitate was rinsed with 5% MeOH in DCM (2×20 mL), followed by DCM (2×10 mL) and MeOH (2×20 mL) to give 2-(2-chlorothieno[2,3-d]pyrimidin-4-yl)acetamide (2.74 g, 46% yield): LC/MS (Table 1, method c) Rₜ=1.29 min; MS m/z: 228 (M+H)⁺.

### General Procedure E

### Displacement of a Heteroaryl Halide or Heteroaryl Sulfonate with an Amine

To a solution of heteroaryl halide or heteroaryl sulfonate (preferably 1 equiv) optionally in an organic solvent such as DMF or NMP (preferably DMF) is added an amine or an amine salt (1-12 equiv, preferably 2-5 equiv) with or without a base such as K₂CO₃, TEA or DIEA (3-10 equiv, preferably TEA, 2-3 equiv) under a nitrogen atmosphere. The reaction mixture is stirred for about 0.5-72 h (preferably 0.5-24 h) at 20-120° C. (preferably 20-60° C.). Alternatively, a degassed mixture of heteroaryl halide or heteroaryl sulfonate (preferably 1 equiv) optionally in an organic solvent such as THF, 1,4-dioxane, toluene, or DMF (preferably 1,4-dioxane), an amine or an amine salt (1-12 equiv, preferably 1 equiv), a base such as sodium tert-butoxide or Cs₂CO₃ (1-10 equiv, preferably sodium tert-butoxide, 1.1 equiv), a palladium source such as Pd(OAc)₂, PdCl₂, or Pd₂(dba)₃ (0.02-0.2 equiv, preferably Pd(OAc)₂, 0.12 equiv), and a ligand such as 2,2′-bis(diphenylphosphino)-1,1′-binaphthalene, di(tert-butyl)(1,1′-biphenyl-2-yl)phosphine, bis(adamant-1-yl)(butyl)phosphine (0.02-0.2 equiv, preferably 2,2′-bis(diphenylphosphino)-1,1′-binaphthalene, 0.06 equiv) is warmed, after degassing as described in Degassing Methods, either thermally or using microwave irradiation to about 60-140° C. (preferably 85° C.) for about 0.5-48 h (preferably 0.5 h). An appropriate solvent such as water or EtOAc is added to induce precipitation and the material is collected by filtration or the volatiles are removed under reduced pressure. Alternatively, the reaction mixture is diluted with water, saturated aqueous NaHCO₃, and/or brine and extracted with an appropriate organic solvent such as DCM or EtOAc affording an organic layer which is then optionally dried over Na₂SO₄ or MgSO₄, filtered, and concentrated under reduced pressure.

### Illustrations of General Procedure E

### Preparation #E.1

### 2-(2-(4-Methylpiperazin-1-yl)quinazolin-4-yl)acetamide

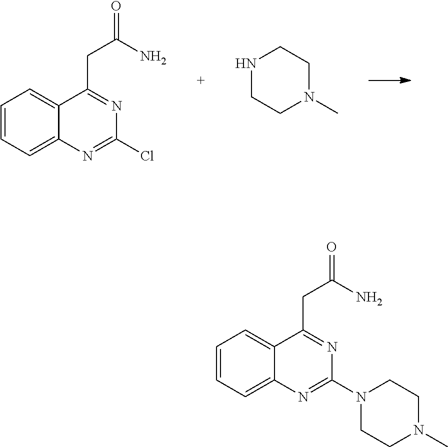

To solution of 2-(2-chloroquinazolin-4-yl) acetamide (8.20 g, 37.0 mmol, Preparation #D.1) in NMP (74 mL) was added 1-methylpiperazine (20.5 mL, 185 mmol). The reaction mixture was heated to about 60° C. and stirred for about 30 min. The reaction was cooled to ambient temperature, EtOAc (90 mL) was added and the mixture was stirred at ambient temperature for about 2 h. The reaction was cooled to about 0° C. and the solids were collected by filtration washing with EtOAc to give 2-(2-(4-methylpiperazin-1-yl)quinazolin-4-yl)acetamide (7.37 g, 70% yield): LC/MS (Table 1, Method c) Rₜ=1.41 min; MS m/z: 286 (M+H)⁺.

### Preparation #E.2

### tert-Butyl 2-(5-fluoro-2-(4-methylpiperazin-1-yl)quinazolin-4-yl)acetate

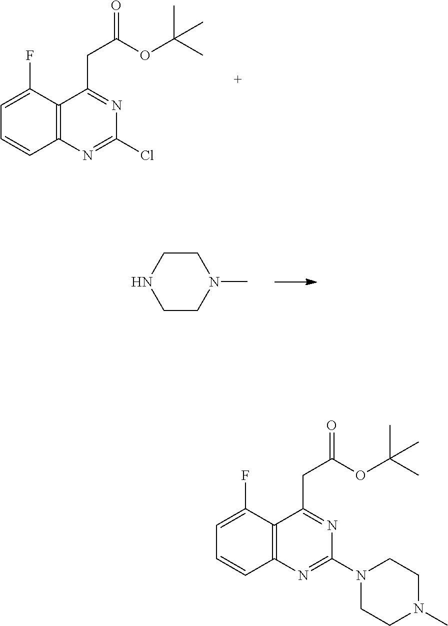

To a solution of tert-butyl 2-(2-chloro-5-fluoroquinazolin-4-yl) acetate (2.59 g, 8.73 mmol, Preparation #L.2) in DMF (17.5 mL) under a nitrogen atmosphere was added 1-methylpiperazine (2.91 mL, 26.2 mmol). The reaction was stirred at ambient temperature for about 16 h. Brine (60 mL) and EtOAc (90 mL) were added, the layers were separated and the aqueous layer was extracted with EtOAc (90 mL). The combined organics were dried over MgSO₄, filtered, and concentrated under reduced pressure. EtOAc (40 mL) was added and the mixture was filtered. The organic solution was concentrated under reduced pressure. The residue was purified by flash column chromatography on silica gel eluting with a gradient of 0-5% MeOH in DCM. The product containing fractions were concentrated under reduced pressure. Brine (40 mL) and EtOAc (70 mL) were added to the residue, the layers were separated, and the organic layer was dried over MgSO₄, filtered, and concentrated. Brine (40 mL) and EtOAc (70 mL) were added to the residue, the layers were separated, and the organic layer was dried over MgSO₄, filtered, and concentrated to afford tert-butyl 2-(5-fluoro-2-(4-methylpiperazin-1-yl)quinazolin-4-yl)acetate (2.00 g, 64% yield): LC/MS (Table 1, method c) Rₜ=1.90 min; MS m/z: 361 (M+H)⁺.

### Preparation #E.3

### 2-(2-(4-Methylpiperazin-1-yl)thieno[2,3-d]pyrimidin-4-yl)acetamide

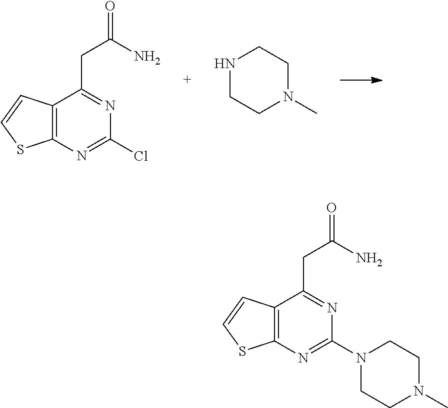

To a solution of 2-(2-chlorothieno[2,3-d]pyrimidin-4-yl)acetamide (2.19 g, 8.64 mmol, Preparation #D.2) in DMF (17.3 mL) under a nitrogen atmosphere was added 1-methylpiperazine (4.80 mL, 43.2 mmol). The reaction was stirred at about 35° C. for about 1 h, and then at ambient temperature for about 16 h. Brine (20 mL) was added to the reaction mixture. The solid was collected by filtration rinsing with water (30 mL) and EtOAc (40 mL). The solid was suspended in a biphasic mixture of DCM (40 mL) and saturated aqueous NaHCO₃ (40 mL). The precipitate was collected by filtration. The solid was dried in a vacuum oven at about 60° C. The organics were separated from the biphasic solution and the aqueous layer was extracted with DCM (40 mL). The combined organics were dried over MgSO₄, filtered, and concentrated under reduced pressure. The residue was triturated with EtOAc (30 mL). The solid was collected by filtration and dried in a vacuum oven at about 60° C. The two crops of solid were combined to give 2-(2-(4-methylpiperazin-1-yl)thieno[2,3-d]pyrimidin-4-yl)acetamide (2.22 g, 88% yield): LC/MS (Table 1, method c) Rₜ=1.01 min; MS m/z: 292 (M+H)⁺.

### Preparation #E.4

### (R)-tert-Butyl 2-(5-fluoro-2-(hexahydropyrrolo[1,2-a]pyrazin-2(1H)-yl)quinazolin-4-yl)acetate

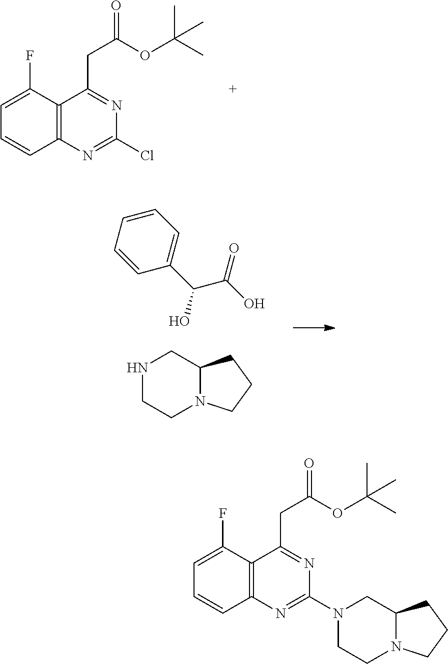

To a solution of tert-butyl 2-(2-chloro-5-fluoroquinazolin-4-yl)acetate (1.14 g, 3.84 mmol, Preparation #L.2) and (R)-octahydropyrrolo[1,2-a]pyrazine (R)-2-hydroxy-2-phenylacetate (2.14 g, 7.68 mmol, Preparation #2) in DMF (7.68 mL) under a nitrogen atmosphere was added TEA (1.07 mL, 7.68 mmol). The reaction was stirred at ambient temperature for about 2 h. Brine (30 mL) was added and the mixture was extracted with EtOAc (2×60 mL). The combined organics were dried over MgSO₄, filtered, and concentrated under reduced pressure. The residue was dissolved in EtOAc (60 mL) and washed with brine (20 mL). The organic layer was dried over MgSO₄, filtered, and concentrated under reduced pressure. The residue was purified by flash column chromatography on silica gel eluting with a gradient of 0-7% MeOH in DCM. The fractions containing product were combined and concentrated under reduced pressure to afford (R)-tert-butyl 2-(5-fluoro-2-(hexahydropyrrolo[1,2-a]pyrazin-2(1H)-yl)quinazolin-4-yl)acetate (0.947 g, 64% yield): LC/MS (Table 1, Method c) Rₜ=1.97 min; MS m/z: 387 (M+H)⁺.

### Example #E.1.1

### (R)-3-(2-(Hexahydropyrrolo[1,2-a]pyrazin-2(1H)-yl)quinazolin-4-yl)-4-(6H-thieno[2,3-b]pyrrol-4-yl)-1H-pyrrole-2,5-dione

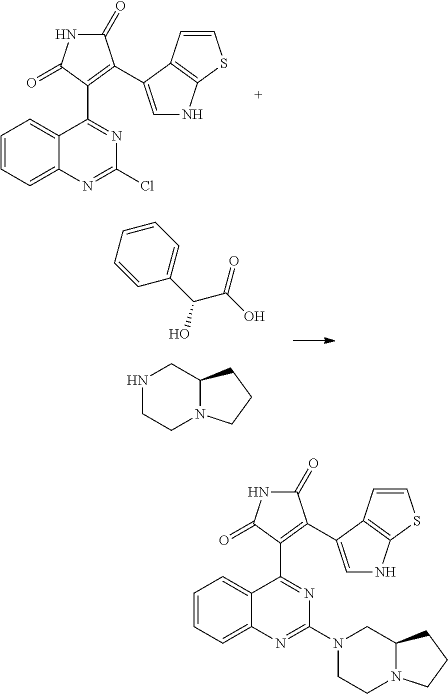

A solution of 3-(2-chloroquinazolin-4-yl)-4-(6H-thieno[2, 3-1)]pyrrol-4-yl)-1H-pyrrole-2,5-dione (0.200 g, 0.525 mmol, Preparation #F.1), (R)-octahydropyrrolo[1,2-a]pyrazine (R)-2-hydroxy-2-phenylacetate (0.292 g, 1.05 mmol, Preparation #2), TEA (0.220 mL, 1.58 mmol), and DMF (1.0 mL) was stirred under a nitrogen atmosphere for about 18 h. Saturated aqueous NaHCO₃ (5 mL) was added and the resulting slurry filtered. The filter cake was washed with water (10 mL). The solid was dissolved in 10% MeOH in DCM (40 mL). The solution was dried over MgSO₄, filtered, and concentrated under reduced pressure. The residue was purified by flash column chromatography on silica gel eluting with a gradient of 1-7.5% MeOH in DCM then 7.5% MeOH in DCM, concentrated from EtOH and dried in a vacuum oven at about 60° C. for about 18 h to give (R)-3-(2-(hexahydropyrrolo[1,2-a]pyrazin-2(1H)-yl)quinazolin-4-yl)-4-(6H-thieno[2,3-b]pyrrol-4-yl)-1H-pyrrole-2,5-dione (0.160 g, 64% yield): LC/MS (Table 1, Method c) Rₜ=1.60 min; MS m/z: 471 (M+H)⁺.

TABLE E.1
Examples prepared from 3-(2-chloroquinazolin-4-yl)-4-(6H-thieno[2,3-b]pyrrol-4-
yl)-1H-pyrrole-2,5-dione (Preparation #F.1) using General Procedure E:

| | | | Rₜ min | m/z |
|---|---|---|---|---|
| | | Example | (Table 1, | ESI + |
| Amine | Product | # | Method) | (M + H)⁺ |
| 4-(1-(Piperidin-4- yl)ethyl)morpholine | 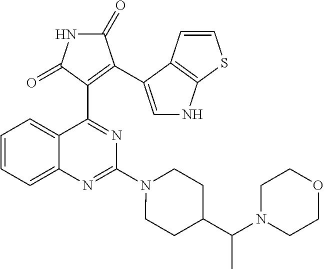 | E.1.2 | 1.85 (c) | 544 |
| 4-((2-Methyl-1H-imidazol- 1-yl)methyl)piperidine | 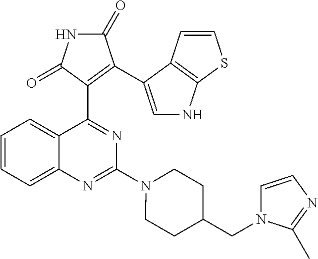 | E.1.3 | 1.78 (c) | 524 |
| 4-(1-(Pyrrolidin-1- yl)ethyl)piperidine | 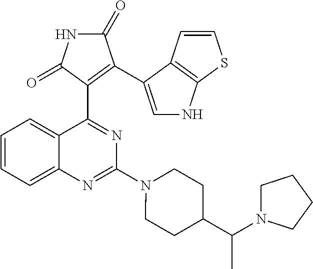 | E.1.4 | 1.78 (c) | 527 |
| N,N-Dimethyl-1-(piperidin- 4-yl)methanamine | 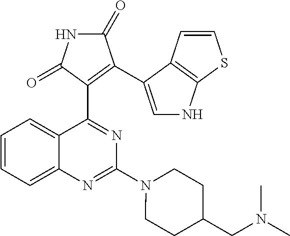 | E.1.5 | 1.78 (c) | 487 |
| (2R,6S)-2,6-Dimethyl-4- (piperidin-4-yl)morpholine | 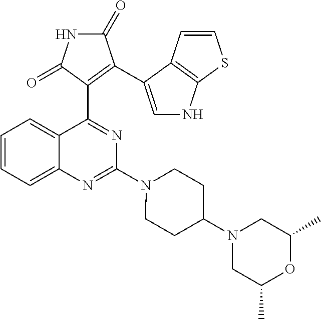 | E.1.6 | 1.87 (c) | 543 |
| Piperizinone (Tyger) | 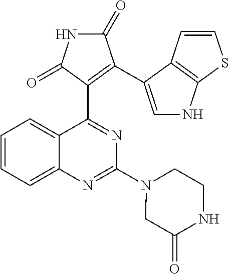 | E.1.7 | 1.64 (c) | 445 |
| 1-Methyl-4-(piperidin-4- yl)piperazine (Oakwood) | 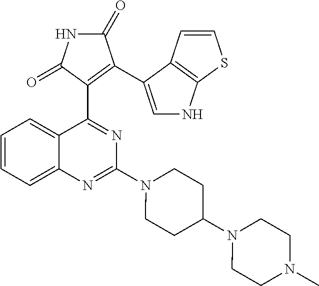 | E.1.8 | 1.73 (c) | 528 |
| 4-Cyanopiperidine (Oakwood) | 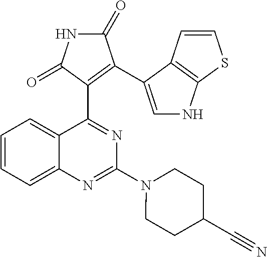 | E.1.9 | 2.38 (c) | 455 |
| 4-(3-Methyl-1,2,4- oxadiazol-5-yl)piperidine (Alfa-Aesar) | 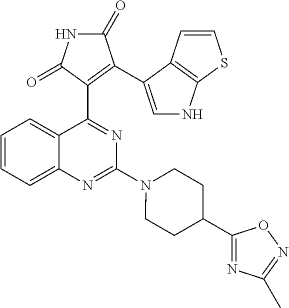 | E.1.10 | 2.30 (c) | 512 |
| 4-(Pyrrolidin-1- ylmethyl)piperidine | 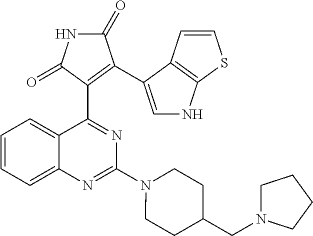 | E.1.11 | 1.90 (c) | 513 |
| N,N-Dimethyl-1-piperidin- 4-ylethanamine | 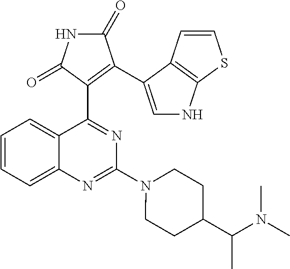 | E.1.12 | 1.88 (c) | 501 |
| 4-(Azetidin-1-yl)piperidine dihydrochloride (prepared using G with tert-butyl 4- (azetidin-1-yl)piperidine-1- carboxylate (prepared using K with 1-Boc-4- piperidinone, azetidine, NaBH₃CN, and titanium tetraisopropoxide)) | 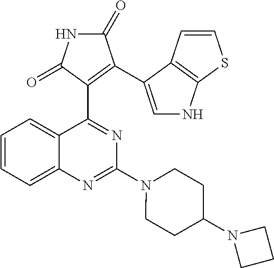 | E.1.13 | 1.86 (c) | 485 |
| 4-Piperidinecarboxamide | 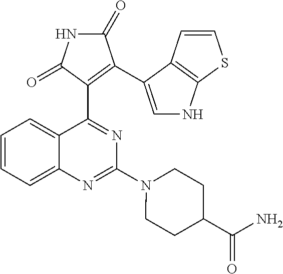 | E.1.14 | 1.89 (c) | 473 |
| Hexahydro-oxazolo[3,4- a]pyrazin-3-one hydrochloride (Tyger) | 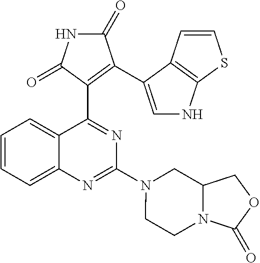 | E.1.15 | 1.88 (c) | 487 |
| 4-(Piperidin-4- ylmethyl)morpholine | 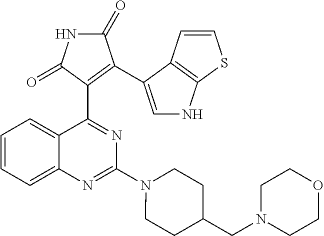 | E.1.16 | 1.82 (c) | 529 |
| 4-(Azetidin-1- ylmethyl)piperidine dihydrochloride (prepared using G with tert-butyl 4- (azetidin-1- ylmethyl)piperidine-1- carboxylate (prepared using I with azetidine, Cs₂CO₃, and N-Boc-4-(4- toluenesulfonyloxymethyl) piperidine (AstaTech))) | 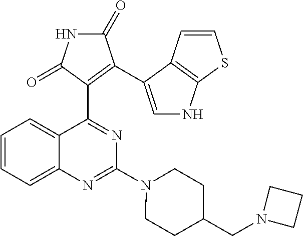 | E.1.17 | 1.85 (c) | 499 |
| 1-Cyclopropylpiperazine dihydrochloride (CNH Technologies) | 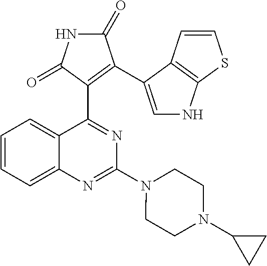 | E.1.18 | 1.93 (c) | 471 |
| 1-(2- Hydroxyethyl)piperazine | 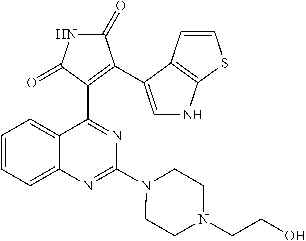 | E.1.19 | 1.58 (c) | 475 |
| 3-(Trifluoromethyl)-1,2,4- triazolo[4,3-a]pyrazine hydrochloride (Accela ChemBio) | 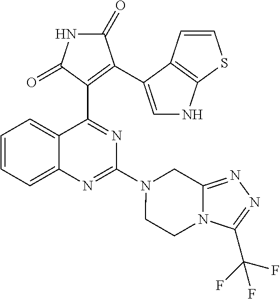 | E.1.20 | 2.02 (c) | 537 |
| 1-Isopropylpiperazine | 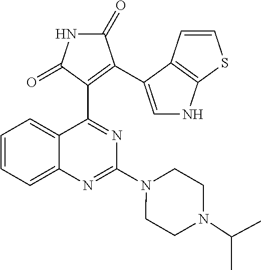 | E.1.21 | 1.71 (c) | 473 |
| 1-t-Butylpiperazine (Wako Pure Chemical Industries) | 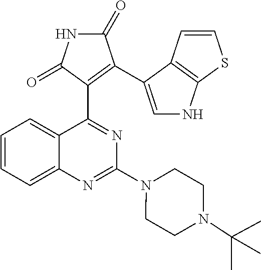 | E.1.22 | 1.76 (c) | 487 |
| 1-(Pyrrolidin-3-yl)-1H- imidazole dihydrochloride (ChemBridge) | 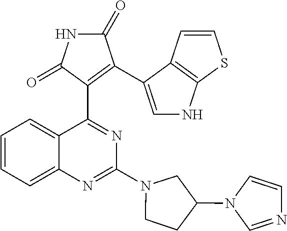 | E.1.23 | 1.77 (c) | 482 |
| 1,2,2-Trimethylpiperazine hydrochloride (ChemBridge) | 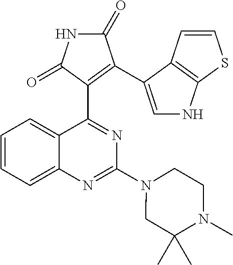 | E.1.24 | 1.75 (c) | 473 |
| 4-(1-Pyrrolidinylmethyl)-4- piperidinol (ChemBridge) | 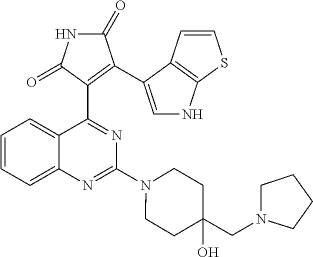 | E.1.25 | 1.74 (c) | 529 |
| 3-Methyl-5,6,7,8- tetrahydroimidazo[1,5- a]pyrazine (WO 2003076427A1) | 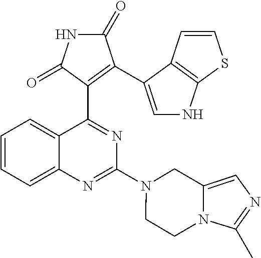 | E.1.26 | 1.63 (c) | 482 |
| 4,5,6,7-Tetrahydro-3H- imidazo [4,5-c]pyridine hydrochloride (D-L Chiral Chemicals) | 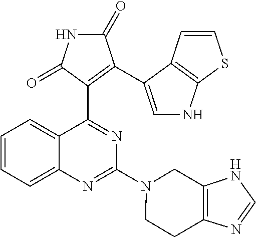 | E.1.27 | 1.61 (c) | 468 |
| N,N-Dimethyl-1- (pyrrolidin-3- yl)methanamine dihydrochloride (prepared using G with tert-butyl 3- ((dimethylamino)methyl) pyrrolidine-1-carboxylate (prepared using K with 3- (aminoethyl)-1-Boc- pyrrolidine (Beta Pharma), formaldehyde, NaBH₃CN)) | 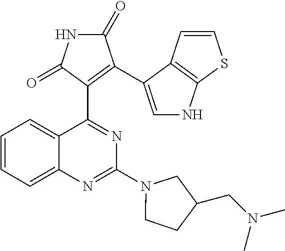 | E.1.28 | 1.57 (c) | 473 |
| cis-3-Fluoro-4-(pyrrolidin- 1-yl)piperidine, dihydrochloride (prepared using G with cis-tert-butyl 3-fluoro-4-(pyrrolidin-1- yl)piperidine-1-carboxylate (prepared using K with pyrrolidine, titanium tetraisopropoxide, NaBH₃CN, and tert-butyl 3- fluoro-4-oxopiperidine-1- carboxylate (Bioorg. Med. Che., m. Lett. 2010, 20, 1735-1739.) | 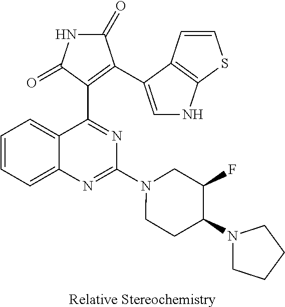 | E.1.29 | 1.65 (c) | 517 |
| trans-3-Fluoro-4- (pyrrolidin-1-yl)piperidine, dihydrochloride (prepared using G with trans-tert- butyl 3-fluoro-4- (pyrrolidin-1-yl)piperidine- 1-carboxylate (prepared using K with pyrrolidine, titanium tetraisopropoxide, NaBH₃CN, and tert-butyl 3- fluoro-4-oxopiperidine-1- carboxylate (Bioorg. Med. Chem. Lett. 2010, 20, 1735- 1739.) | 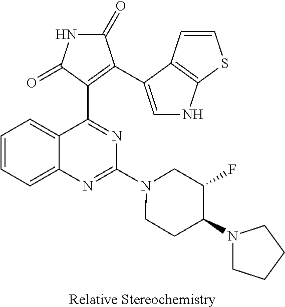 | E.1.30 | 1.72 (c) | 517 |
| trans-3-Fluoro-4- (pyrrolidin-1-yl)piperidine, dihydrochloride (prepared using G with trans-tert- butyl 3-fluoro-4- (pyrrolidin-1-yl)piperidine- 1-carboxylate (prepared using K with pyrrolidine, titanium tetraisopropoxide, NaBH₃CN, and tert-butyl 3-fluoro-4-oxopiperidine-1- carboxylate (Bioorg. Med. Chem. Lett. 2010, 20, 1735- 1739.))) | 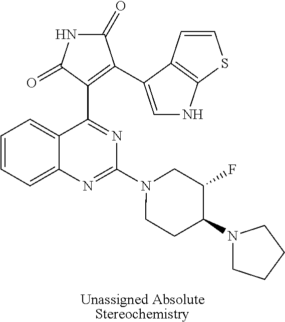 | E.1.31 | 1.81 (c) | 517 |
| trans-3-Fluoro-4- (pyrrolidin-1-yl)piperidine, dihydrochloride (prepared using G with trans-tert- butyl 3-fluoro-4- (pyrrolidin-1-yl)piperidine- 1-carboxylate (prepared using K with pyrrolidine, titanium tetraisopropoxide, NaBH₃CN, and tert-butyl 3-fluoro-4-oxopiperidine-1- carboxylate (Planty, B.; Pujol, C.; Lamothe, M.; Maraval, C.; Horn, C.; Le Grand, B.; Perez, M. Bioorg. Med. Chem. Lett. 2010, 20, 1735-1739.))) | 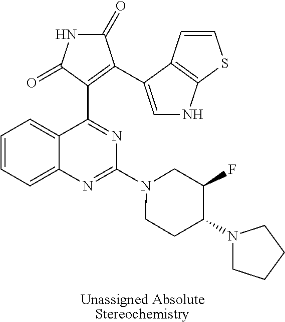 | E.1.32 | 1.79 (c) | 517 |
| cis-3-Fluoro-N,N- dimethylpiperidin-4-amine dihydrochloride (prepared using G wilh tert-butyl 4- (dimethylamino)-3- fluoropiperidine-1- carboxylate (prepared using K with dimethylamine, titanium tetraisopropoxide, NaBH₃CN, and tert-butyl 3-fluoro-4-oxopiperidine-1- carboxylate (Bioorg. Med. Chem. Lett. 2010, 20, 1735- 1739) | 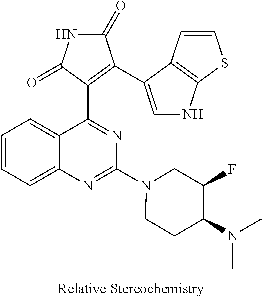 | E.1.33 | 1.66 (c) | 491 |
| 2-(4-Ammopiperidin-4- yl)ethanol dihydrochloride (prepared using G with tert- butyl 4-amino-4-(2- hydroxyethyl)piperidine-1- carboxylate (prepared using Q with LiBH₄ and 4-amino-4- methoxycarbonylpiperidine- 1-carboxylic acid tert-butyl ester (AstaTech))) | 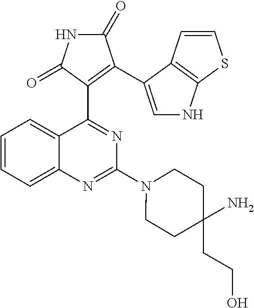 | E.1.34 | 1.56 (c) | 489 |
| 1,7-Diazaspiro[3.5]nonane dihydrochloride (Preparation #3) | 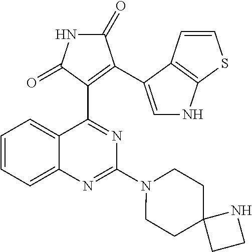 | E.1.35 | 1.74 (c) | 471 |
| 1,7-Diazaspiro[3.5]nonane dihydrochloride (Preparation #3) | 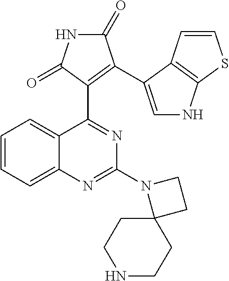 | E.1.36 | 1.65 (c) | 471 |
| trans-(4- (Dimethylamino)piperidin- 3-yl)methanol hydrochloride (prepared using O with palladium hydroxide on carbon, hydrogen, and trans-(1- benzyl-4- (dimethylamino)piperidin- 3-yl)methanol (prepared using K with formaldehyde, NaBH₃CN, and trans-4- amino-1-benzylpiperidin-3- yl)methanol (Astatech))) | 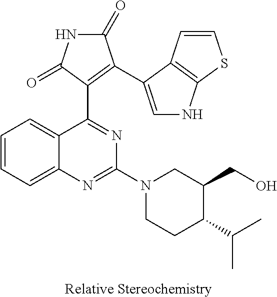 | E.1.37 | 1.69 (c) | 503 |
| 5,6,7,8-Tetrahydro- [1,2,4]triazolo[1,5- a]pyrazine (ArkPharm Inc) | 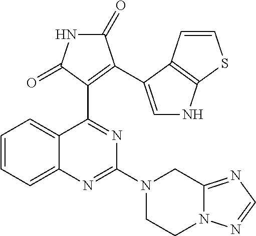 | E.1.38 | 1.92 (c) | 469 |
| 2-Methyloctahydro-1H- pyrrolo-[3,4-c]pyridine (Tyger Scientific) | 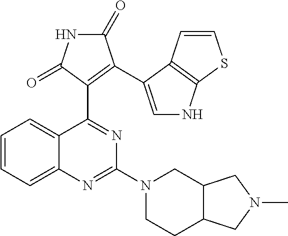 | E.1.39 | 1.66 (c) | 485 |
| 3-Methyl-5,6,7,8- tetrahydro- [1,2,4]triazolo-[4,3- a]pyrazine (ChemFocus LLC) | 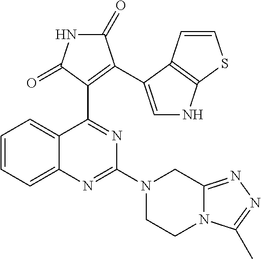 | E.1.40 | 1.72 (c) | 483 |
| N,N,4-Trimethylpiperidin- 4-amine dihydrochloride (prepared using O with palladium hydroxide on carbon, hydrogen, and 1- benzyl-N,N,4- trimethylpiperidin-4-amine (prepared using K with formaldehyde, NaBH₃CN, and 1-benzyl-4- methylpiperidin-4-amine (prepared using V with N- (1-benzyl-4- methylpiperidin-4- yl)acetamide (prepared using U with sulfuric acid and 1-benzyl-4- methylpiperidin-4-ol (prepared using T with 1- benzylpiperidin-4-one and methylmagnesium bromide))))) | 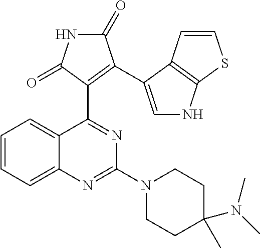 | E.1.41 | 1.67 (c) | 487 |
| N,4-Dimethylpiperidin-4- amine trifluoroacetate (prepared using G with trifluoroacetic acid and tert- butyl 4-(tert- butoxycarbonyl(methyl)amino)- 4-methylpiperidine-1- carboxylate (prepared using I with iodomethane, sodium hydride, and tert-butyl 4- (tert- butoxycarbonylamino)-4- methylpiperidine-1- carboxylate (prepared using R with TEA and tert-butyl 4-methylpiperidin-4- ylcarbamate (prepared using O with palladium hydroxide on carbon, hydrogen, and tert-butyl 1-benzyl-4- methylpiperidin-4- ylcarbamate (prepared using R with TEA and 1-benzyl- 4-methylpiperidin-4-amine (prepared using V with N- (1-benzyl-4- methylpiperidin-4- yl)acetamide (prepared using U with sulfuric acid and 1-benzyl-4- methylpiperidin-4-ol (prepared using T with 1- benzylpiperidin-4-one and methylmagnesium bromide)))))))) | 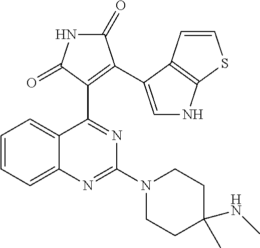 | E.1.42 | 1.70 (c) | 473 |
| 4-Ethylpiperidin-4-amine (prepared using O with palladium hydroxide on carbon, hydrogen, and 1- benzyl-4-ethylpiperidin-4- amine (Preparation #V.1)) | 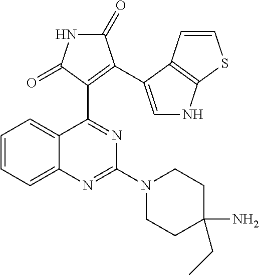 | E.1.43 | 1.80 (c) | 473 |
| (S)-Octahydropyrrolo[1,2- a]pyrazine (CNH Technologies) | 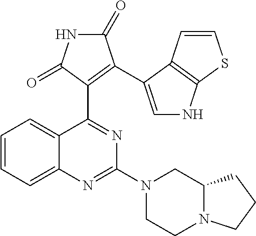 | E.1.44 | 1.70 (c) | 471 |
| (1-Methylpiperazin-2-yl) methanol di-hydrochloride (prepared using G with tert- butyl 3-(hydroxymethyl)-4- methylpiperazine-1- carboxylate (prepared using K with formaldehyde, NaBH₃CN, and tert-butyl 3- (hydroxymethyl)piperazine- 1-carboxylate (AstaTech))) | 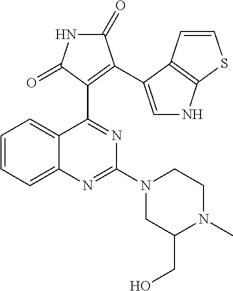 | E.1.45 | 1.52 (c) | 475 |
| 5,6,7,8- Tetrahydroimidazo[1,5]- a]pyrazine (WO 2003076427A1) | 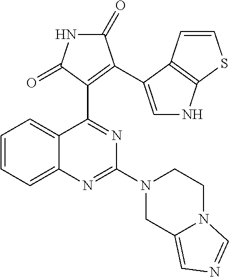 | E.1.46 | 1.64 (c) | 468 |
| 3-Ethyl-5,6,7,8- tetrahydroimidazo[1,5- a]pyrazine (WO 2003076427A1) | 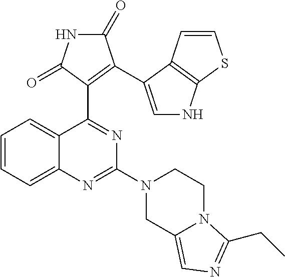 | E.1.47 | 1.70 (c) | 496 |
| (5,6,7,8- Tetrahydroimidazo[1,5- a]pyrazine-3-yl)methanol hydrochloride (WO 2003076427A1) | 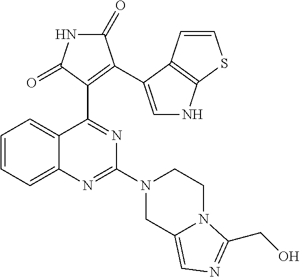 | E.1.48 | 1.65 (c) | 498 |
| 2-(5,6,7,8- Tetrahydroimidazo[1,5- a]pyrazin-3-yl)propan-2-ol (WO 2003076427A1) | 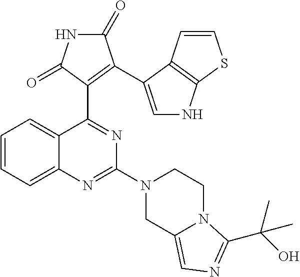 | E.1.49 | 1.69 (c) | 526 |
| (5,6,7,8-Tetrahydroimidazo [1,5-a]pyrazin-1-yl) methanol (prepared by O with Pd/C, ammonium formate, and (7-benzyl- 5,6,7,8-tetrahydroimidazo [1,5-a]pyrazin-1-yl) methanol (prepared by Q with LiAlH₄ and methyl 7- benzyl-5,6,7,8- tetrahydroimidazo[1,5- a]pyrazine-1-carboxylate (WO2008157751A2))) | 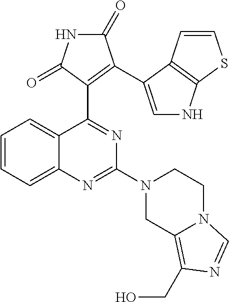 | E.1.50 | 1.65 (c) | 498 |
| N,N-Dimethyl-1-(5,6,7,8- tetrahydroimidazo[1,5-a] pyrazin-3-yl)methanamine dihydrochloride (prepared using O with Pd/C, ammonium formate, and 1- (7-benzyl-5,6,7,8- tetrahydroimidazo[1,5- a]pyrazin-3-yl)-N,N- dimethylmethanamine (prepared using K with dimethylamine, Na(AcO)₃BH, and 7- benzyl-5,6,7,8- tetrahydroimidazo[1,5- a]pyrazine-3-carbaldehyde (WO 2003076427A1))) | 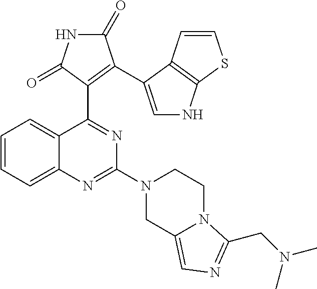 | E.1.51 | 1.65 (c) | 525 |
| 1-(5,6,7,8- Tetrahydroimidazo[1,5- a]pyrazin-3-yl)-ethanol hydrochloride (Preparation #O.1) | 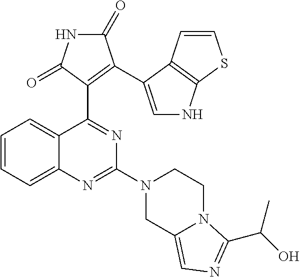 | E.1.52 | 1.62 (c) | 512 |
| 3-Methyl-4,5,6,7- tetrahydro-3H-imidazo[4,5- c]pyridine hydrochloride (prepared using G with tert- butyl 3-methyl-6,7-dihydro- 3H-imidazo[4,5-c]pyridine- 5(4H)-carboxylate (prepared using I with iodomethane, NaH, and tert-butyl 6,7-dihydro-1H- imidaxo[4,5-c]pyridine- 5(4H)-carboxylate (Preparation #4))) | 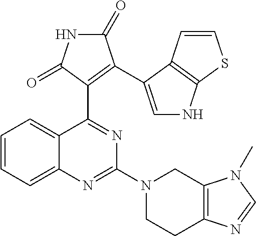 | E.1.53 | 1.72 (c) | 482 |
| 1-Methyl-4,5,6,7- tetrahydro-1H-imidazo[4,5- c]pyridine hydrochloride (prepared using G with tert- butyl 1-methyl-6,7-dihydro- 1H-imidazo[4,5-c]pyridine- 5(4H)-carboxylate (prepared using I with iodomethane, NaH, and tert-butyl 6,7-dihydro-1H- imidazo[4,5-c]pyridine- 5(4H)-carboxylate (Preparation #4))) | 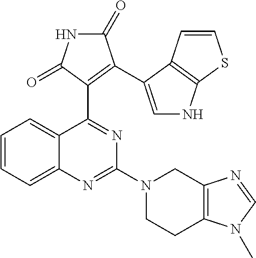 | E.1.54 | 1.73 (c) | 482 |
| 2-Methyl-1-(piperazin-1- yl)propan-2-ol (JP2001302648A) | 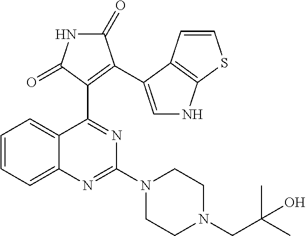 | E.1.55 | 1.77 (c) | 503 |
| 2-Methyl-2-(piperazin-1- yl)propan-1-ol- dihydrochloride (prepared using G with tert butyl 4- (1-hydroxy-2- methylpropan-2- yl)piperazine-1-carboxylate (Preparation #Q.1)) | 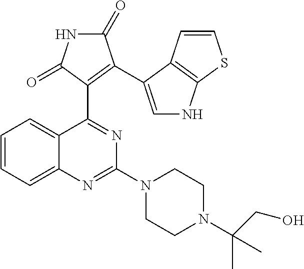 | E.1.56 | 1.68 (c) | 503 |
| (4-Aminopiperidin-4- yl)methanol hydrochloride (prepared using G with tert- butyl 4-(tert- butoxycarbonylamino)-4- (hydroxymethyl)- piperidine-1-carboxylate (prepared using Q with LiAlH₄ and 1-(tert- butoxycarbonyl)-4-(tert- butoxycarbonylamino) piperidine-4-carboxylic acid (Preparation #R.1))) | 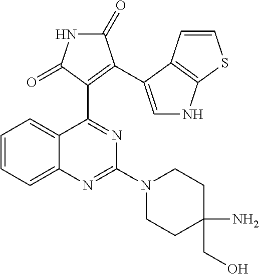 | E.1.57 | 1.62 (c) | 475 |
| 4-Amino-N- cyclopropylpiperidine-4- carboxamide hydrochloride (prepared using G with tert- butyl 4-(tert- butoxycarbonylamino)-4- (cyclopropylcarbamoyl) piperidine-1-carboxylate (prepared using S with HATU, DIEA, cyclopropylamine, and 1- (tert-butoxycarbonyl)-4- (tert-butoxycarbonylamino) piperidine-4-carboxylic acid (Preparation #R.1))) | 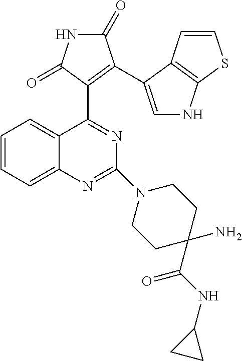 | E.1.58 | 1.69 (c) | 528 |
| 1-Methyloctahydro-1H- pyrrolo[3,2-c]pyridine (Preparation #7) | 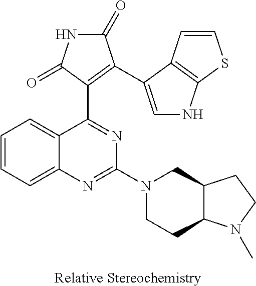 | E.1.59 | 1.66 (c) | 485 |

### Example #E.2.1

### (R)-3-(2-(Hexahydropyrrolo[1,2-a]pyrazin-2(1H)-yl)quinazolin-4-yl)-4-(4H-thieno[3,2-b]pyrrol-6-yl)-1H-pyrrole-2,5-dione

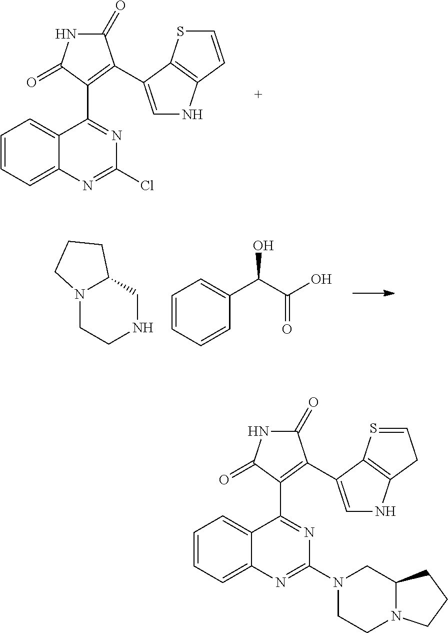

A solution of 3-(2-chloroquinazolin-4-yl)-4-(4H-thieno[3,2-b]pyrrol-6-yl)-1H-pyrrole-2,5-dione (7.50 g, 13.4 mmol, Preparation #F.2), (R)-octahydropyrrolo[1,2-a]pyrazine (R)-2-hydroxy-2-phenylacetate (7.46 g, 26.8 mmol, Preparation #2), TEA (5.60 mL, 40.2 mmol) and DMF (60 mL) was stirred at ambient temperature for about 17 h. Saturated aqueous NaHCO₃ (500 mL) was added and the resulting slurry was filtered. The filter cake was washed with water. The wet filter cake was treated with DCM (500 mL) and the two layers were separated. The organic layer was concentrated and purified by flash column chromatography on silica gel eluting with a gradient of 0-10% MeOH in DCM then 13% (0.67 N NH₃ in MeOH) in DCM. The product containing fractions were concentrated under reduced pressure. The residue was concentrated from EtOH (100 mL) and then dried in a vacuum oven at about 60° C. for about 40 h to give (R)-3-(2-(hexahydropyrrolo[1,2-a]pyrazin-2(1H)-yl)quinazolin-4-yl)-4-(4H-thieno[3,2-b]pyrrol-6-yl)-1H-pyrrole-2,5-dione (3.10 g, 49% yield): LC/MS (Table 1, Method c) Rₜ=1.45 min; MS m/z: 471 (M+H)⁺.

TABLE E.2
Examples prepared from 3-(2-chloroquinazolin-4-yl)-4-(4H-thieno[3,2-b]pyrrol-6-
yl)-1H-pyrrole-2,5-dione (Preparation #F.2) using General Procedure E:

| | | | Rt min | m/z |
|---|---|---|---|---|
| | | Example | (Table 1, | ESI + |
| Amine | Product | # | Method) | (M + H)⁺ |
| 1,4′-Bipiperidine | 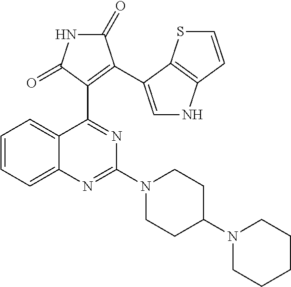 | E.2.2 | 1.82 (c) | 513 |
| 4-Dimethylaminopiperidine | 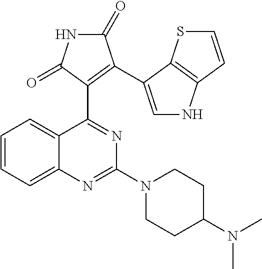 | E.2.3 | 1.60 (c) | 473 |
| (S)-1,4- Diazobicyclo[4.3.0]nonane (CNH Technologies) | 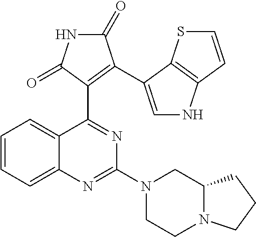 | E.2.4 | 1.67 (c) | 471 |
| 4-Morpholinopiperidine | 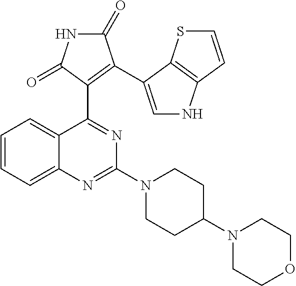 | E.2.5 | 1.73 (c) | 515 |
| 4-(1-Pyrrolidinyl)piperidine | 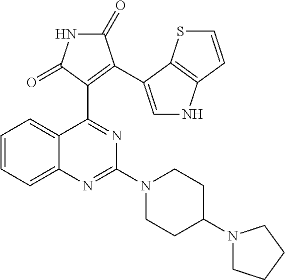 | E.2.6 | 1.68 (a) | 499 |
| 5,6,7,8- Tetrahydroimidazo[1,2- a]pyrazine (Ark Pharm, Inc.) | 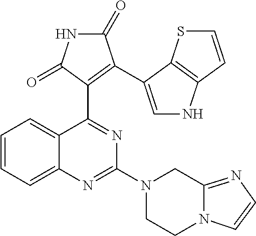 | E.2.7 | 1.66 (c) | 468 |
| 5,6,7,8-Tetrahydro- [1,2,4]triazolo[4,3- a]pyrazine hydrochloride (J & W Pharmlab) | 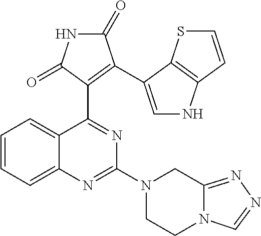 | E.2.8 | 1.78 (c) | 469 |
| Hexahydro-pyrrolo[1,2- a]pyrazin-6-one (AstaTech) | 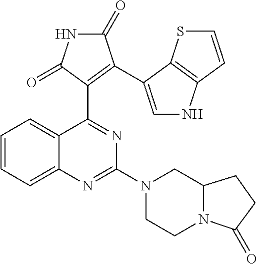 | E.2.9 | 1.90 (c) | 485 |
| 1,4 Diazobicyclo[4.3.0]nonane (CNH Technologies) | 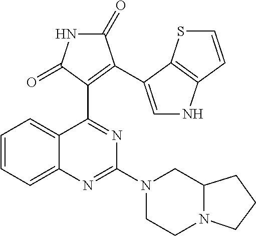 | E.2.10 | 1.61 (c) | 471 |

TABLE E.3
Examples prepared from 3-(2-chlorothieno[2,3-d]pyrimidin-4-yl)-4-(6H-thieno[2,3-b]pyrrol-
4-yl)-1H-pyrrole-2,5-dione (prepared using F with 2-(2-chlorothieno[2,3-d]pyrimidin-4-yl)
acetamide (prepared using D with 2,4-dichlorothieno[2,3-d]pyrimidine (Synthonix)) and tert-
butyl 4-(2-methoxy-2-oxoacetyl)-6H-thieno[2,3-b]pyrrole-6-carboxylate (prepared using C
with tert-butyl 4-(2-methoxy-2-oxoethyl)-6H-thieno[2,3-b]pyrrole-6-carboxylate (prepared
using B with tert-butyl 3-bromothiophen-2-ylcarbamate
(Preparation #A.1), K₂CO₃, methyl 4-bromocrotonate))) using General Procedure E:

| | | | Rₜ min | |
|---|---|---|---|---|
| | | Example | (Table 1, | m/z ESI+ |
| Amine | Product | # | Method) | (M + H)⁺ |
| (S)-Octahydropyrrolo [1,2-a]pyrazine (CNH Technologies) | 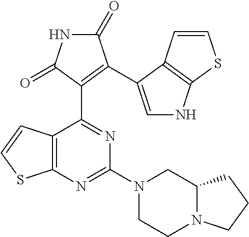 | E.3.1 | 1.71 (c) | 477 |

### General Procedure F

### Formation of a Maleimide

To a mixture of an amide (preferably 1 equiv) and an α-keto ester (preferably 0.8-2 equiv) in an organic solvent such as 1,4-dioxane, Et₂O, DMF, or THF (preferably THF) at about −40-0° C. (preferably −30-10° C.) under a nitrogen atmosphere is added a base such as KOt-Bu (1-10 equiv, preferably 1.9-8 equiv) usually as a solution in an organic solvent such as THF. The reaction mixture is optionally allowed to warm to about −20° C. to reflux (preferably −20-25° C.). After about 0.5-24 h (preferably 1-16 h), the reaction is optionally cooled to −10-0° C. and then quenched with water, aqueous NaHCO₃, or brine. Optionally, an organic solvent such as EtOAc or DCM (preferably EtOAc) is added. The solution pH is optionally adjusted with aqueous HCl to approximately 6-7. The target material is collected by filtration or extracted with an organic solvent such as EtOAc or DCM (preferably EtOAc). The combined organic layers are optionally washed with brine or water, dried over MgSO₄ or Na₂SO₄, filtered, and concentrated under reduced pressure.

### Illustrations of General Procedure F

### Preparation #F.1

### 3-(2-Chloroquinazolin-4-yl)-4-(6H-thieno[2,3-b]pyrrol-4-yl)-1H-pyrrole-2,5-dione

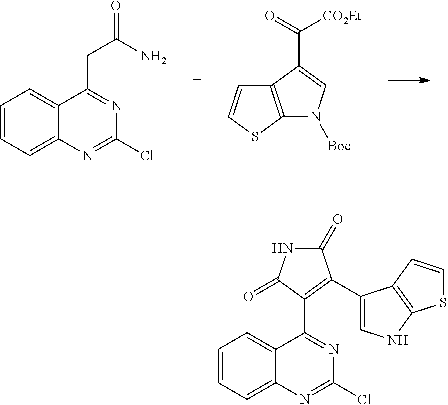

A round-bottomed flask was charged with tert-butyl 4-(2-ethoxy-2-oxoacetyl)-6H-thieno[2,3-b]pyrrole-6-carboxylate (1.00 g, 3.09 mmol, Preparation #C.1), 2-(2-chloroquinazolin-4-yl)acetamide (0.823 g, 3.71 mmol, Preparation #D.1), and THF (20 mL) under a nitrogen atmosphere. The suspension was cooled to about −20° C. and KOt-Bu (1 M solution in THF, 23.2 mL, 23.2 mmol) was added dropwise via syringe over about 30 min. The reaction was stirred at about −20° C. for about 1 h and then at about 0° C. for about 1 h. Brine (50 mL) and EtOAc (150 mL) were added. The pH was adjusted to about 9 with 2 N aqueous HCl (7 mL). The layers were separated and the aqueous layer was extracted with EtOAc (50 mL). The combined organics were dried over MgSO₄, filtered, and concentrated under reduced pressure. The residue was dissolved in 5% MeOH/DCM (20 mL), silica gel (4.3 g) was added, and the volatiles were removed under reduced pressure. The resulting solid was purified by flash column chromatography on silica gel eluting with a gradient of 0% (10% MeOH in DCM)/DCM for 3 min, 0-35% (10% MeOH in DCM)/DCM over 17 min, 35-40% (10% MeOH in DCM)/DCM over 10 min, 40-50% (10% MeOH in DCM)/DCM over 10 min. The product containing fractions were combined and concentrated under reduced pressure to give 3-(2-chloroquinazolin-4-yl)-4-(6H-thieno[2,3-b]pyrrol-4-yl)-1H-pyrrole-2,5-dione (0.740 g, 63% yield): LC/MS (Table 1, Method c) Rₜ=1.95 min; MS m/z: 381 (M+H)⁺.

### Preparation #F.2

### 3-(2-Chloroquinazolin-4-yl)-4-(4H-thieno[3,2-b]pyrrol-6-yl)-1H-pyrrole-2,5-dione

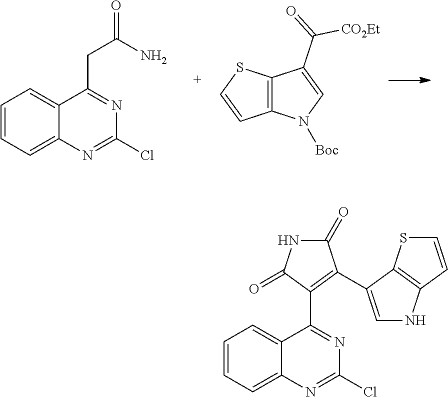

A round bottomed flask was charged with 2-(2-chloroquinazolin-4-yl)acetamide (1.00 g, 4.51 mmol, Preparation #D.1), tert-butyl 6-(2-ethoxy-2-oxoacetyl)-4H-thieno[3,2-b]pyrrole-4-carboxylate (1.46 g, 4.51 mmol, Preparation #C.2) and THF (19.4 mL) under a nitrogen atmosphere. The suspension was cooled to about −20° C. and KOt-Bu (1 M solution in THF, 8.46 mL, 8.46 mmol) was added dropwise and stirred for about 1 h. Water (50 mL) was added and the mixture was warmed to ambient temperature. The solvent was removed under reduced pressure and the aqueous phase was neutralized by adding 2 N aqueous HCl. EtOAc (75 mL) and the layers were separated. The aqueous layer was extracted with EtOAc (2×75 mL). The combined organics were dried over MgSO₄, filtered and concentrated under reduce pressure to give 3-(2-chloroquinazolin-4-yl)-4-(4H-thieno[3,2-b]pyrrol-6-yl)-1H-pyrrole-2,5-dione (1.35 g, 79% yield): LC/MS (Table 1, Method a) Rₜ=1.98 min; MS m/z: 381 (M+H)⁺.

### Example #F.1.1

### 3-(2-(4-Methylpiperazin-1-yl)quinazolin-4-yl)-4-(6H-thieno[2,3-b]pyrrol-4-yl)-1H-pyrrole-2,5-dione

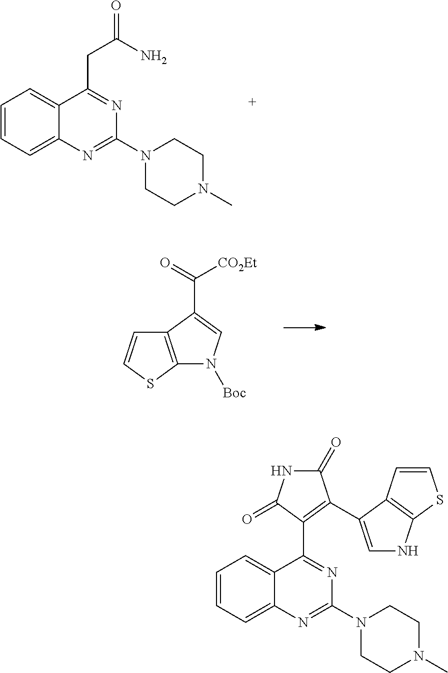

A round bottomed flask was charged with 2-(2-(4-methylpiperazin-1-yl)quinazolin-4-yl)acetamide (0.080 g, 0.28 mmol, Preparation #E.1), tert-butyl 4-(2-ethoxy-2-oxoacetyl)-6H-thieno[2,3-b]pyrrole-6-carboxylate (0.181 g, 0.561 mmol, Preparation #C.1) and THF (2.2 mL) under a nitrogen atmosphere. The suspension was cooled to about −20° C. and KOt-Bu (1 M solution in THF, 2.10 mL, 2.10 mmol) was added dropwise over about 15 min. The reaction mixture was stirred at about 0° C. for about 15 min and then at ambient temperature for about 1.5 h. Water (20 mL) was added and the mixture was extracted with EtOAc (3×50 mL). The combined organics were dried over MgSO₄, filtered, and concentrated under reduced pressure. The residue was purified by flash column chromatography on silica gel eluting with a gradient of 0-60% (2% (2 N NH₃ in EtOH) in 10% MeOH in DCM) in DCM. The product containing fractions were combined and concentrated under reduced pressure. The resulting orange powder was dried in a vacuum oven at about 70° C. to give 3-(2-(4-methylpiperazin-1-yl)quinazolin-4-yl)-4-(6H-thieno[2,3-b]pyrrol-4-yl)-1H-pyrrole-2,5-dione (0.035 g, 28% yield): LC/MS (Table 1, Method c) Rₜ=1.63 min; MS m/z: 445 (M+H); ¹H NMR (400 MHz, DMSO-d₆) δ 12.17-11.90 (m, 1H), 11.36-11.06 (m, 1H), 7.93 (d, J=1.0 Hz, 1H), 7.74-7.65 (m, 2H), 7.57 (d, J=8.3 Hz, 1H), 7.13 (ddd, J=1.2, 7.0, 8.2 Hz, 1H), 6.77 (dd, J=1.1, 5.4 Hz, 1H), 5.31 (d, J=5.4 Hz, 1H), 3.86-3.67 (m, 4H), 2.33-2.19 (m, 4H), 2.17 (s, 3H).

### Example #F.1.2

### 3-(2-(4-Methylpiperazin-1-yl)quinazolin-4-yl)-4-(4H-thieno[3,2-b]pyrrol-6-yl)-1H-pyrrole-2,5-dione

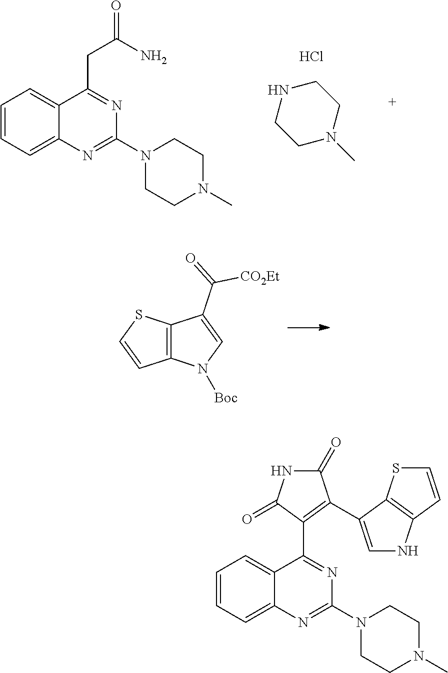

A round bottomed flask was charged with tert-butyl 6-(2-ethoxy-2-oxoacetyl)-4H-thieno[3,2-b]pyrrole-4-carboxylate (0.307 g, 0.948 mmol, Preparation #C.2), 2-(2-(4-methylpiperazin-1-yl)quinazolin-4-yl)acetamide with one equiv 1-methylpiperazine hydrochloride (0.200 g, 0.474 mmol, prepared using E with 2-(2-chloroquinazolin-4-yl)acetamide (Preparation #D.1) and 1-methylpiperazine) and THF (2.0 mL) under a nitrogen atmosphere. The suspension was cooled to about −10° C. and KOt-Bu (1 M solution in THF, 3.55 mL, 3.55 mmol) was added dropwise. The reaction was warmed to ambient temperature and stirred for about 16 h. Water (10 mL) and EtOAc (15 mL) were added and the layers were separated. The aqueous phase was extracted with EtOAc (3×20 mL) and the combined organics were washed with brine (25 mL), dried over MgSO₄, filtered, and concentrated under reduced pressure. The remaining residue was purified by flash column chromatography on silica gel eluting with a gradient of 1-5% MeOH in DCM. The solvent was removed under reduced pressure to give 3-(2-(4-methylpiperazin-1-yl)quinazolin-4-yl)-4-(4H-thieno[3,2-b]pyrrol-6-yl)-1H-pyrrole-2,5-dione (0.050 g, 14% yield): LC/MS (Table 1, Method c) Rₜ=1.60 min; MS m/z: 445 (M+H)⁺; ¹H NMR (400 MHz, DMSO-d₆) δ 12.03 (s, 1H), 11.20 (s, 1H), 8.01 (s, 1H), 7.73-7.67 (m, 1H), 7.65 (d, J=8.2 Hz, 1H), 7.58 (d, J=8.6 Hz, 1H), 7.17-7.09 (m, 2H), 6.94 (dd, J=0.9, 5.3 Hz, 1H), 3.92-3.70 (m, 4H), 2.40-2.25 (m, 4H), 2.18 (s, 3H).

TABLE F.1
Example prepared from 2-(2-(4-methylpiperazin-1-yl)quinazolin-4-yl)acetamide
(Preparation #E.1) using General Procedure F:

| | | | Rₜ min | |
|---|---|---|---|---|
| | | Example | (Table 1, | m/z ESI+ |
| α-Keto ester | Product | # | Method) | (M + H)⁺ |
| tert-Butyl 4-(2-ethoxy-2-oxoacetyl)- 2-methyl-6H-thieno[2,3-b]pyrrole- 6-carboxylate (prepared using C with tert-butyl 4-(2-ethoxy-2- oxoethyl)-2-methyl-6H-thieno[2,3- b]pyrrole-6-carboxylate (prepared using B with ethyl 4- bromocrotonate, K₂CO₃, and tert- butyl 3-bromo-5-methylthiophen-2- ylcarbamate (Preparation #1))) | 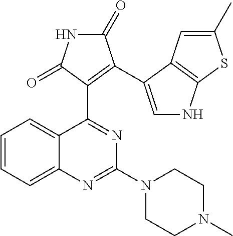 | F.1.3 | 1.92 (c) | 459 |

TABLE F.2
Example prepared from tert-butyl 4-(2-ethoxy-2-oxoacetyl)-6H-thieno[2,3-b]pyrrole-6-carboxylate
(Preparation #C.1) using General Procedure F:

| | | | Rₜ min | |
|---|---|---|---|---|
| | | Example | (Table 1, | m/z ESI+ |
| Acetamide | Product | # | Method) | (M + H)⁺ |
| 2-(2-(Hexahydropyrrolo[1,2- a]pyrazin-2(1H)-yl)quinazolin- 4-yl)acetamide (prepared using E with 2-(2-chloroquinazolin-4- yl)acetamide (Preparation #D.1) and octahydropyrrolo[1,2- a]pyrazine (ChemBridge)) | 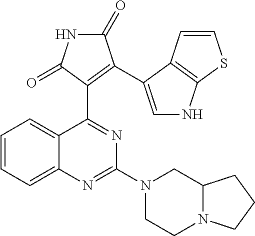 | F.2.1 | 1.14 (b) | 471 |

### Example #F.3.1

### 3-(5-Fluoro-2-(4-methylpiperazin-1-yl)quinazolin-4-yl)-4-(4H-thieno[3,2-b]pyrrol-6-yl)-1H-pyrrole-2,5-dione

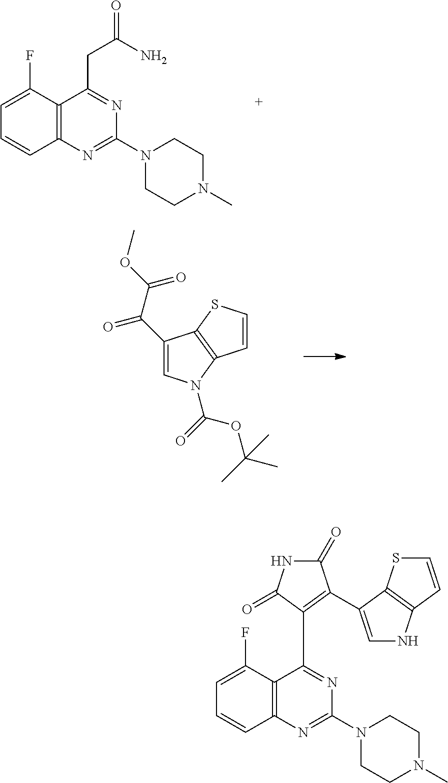

A mixture of 2-(5-fluoro-2-(4-methylpiperazin-1-yl)quinazolin-4-yl)acetamide (0.320 g, 1.06 mmol, Preparation #J.2) and tert-butyl 6-(2-methoxy-2-oxoacetyl)-4H-thieno[3,2-b]pyrrole-4-carboxylate (0.489 g, 1.58 mmol, prepared using C with tert-butyl 6-(2-methoxy-2-oxoethyl)-4H-thieno[3,2-b]pyrrole-4-carboxylate (prepared using B with tert-butyl 2-bromothiophen-3-ylcarbamate (Preparation #A.2), K₂CO₃, methyl 4-bromocrotonate)) in THF (4.53 mL) under a nitrogen atmosphere was cooled to about −30° C. To the reaction mixture was added KOt-Bu (1 M in THF, 4.22 mL, 4.22 mmol) dropwise over about 20 min. The reaction solution was warmed to about 0° C. over about 30 min and then warmed to ambient temperature for about 2 h. The reaction mixture was cooled to about −10° C. prior to addition of water (50 mL) and EtOAc (90 mL). The organic layer was separated, dried over MgSO₄, filtered, and concentrated under reduced pressure. The residue was purified by flash column chromatography on silica gel eluting with a gradient of 1-7% MeOH in DCM. The fractions containing product were set aside to combine with that from the second batch. A mixture of 2-(5-fluoro-2-(4-methylpiperazin-1-yl)quinazolin-4-yl)acetamide (0.269 g, 0.887 mmol, Preparation #J.2) and tert-butyl 6-(2-methoxy-2-oxoacetyl)-4H-thieno[3,2-b]pyrrole-4-carboxylate (0.411 g, 1.33 mmol, prepared using C with tert-butyl 6-(2-methoxy-2-oxoethyl)-4H-thieno[3,2-b]pyrrole-4-carboxylate (prepared using B with tert-butyl 2-bromothiophen-3-ylcarbamate (Preparation #A.2), K₂CO₃, methyl 4-bromocrotonate)) in THF (3.81 mL) under a nitrogen atmosphere was cooled to about −30° C. To the reaction mixture was added KOt-Bu (1 M in THF, 3.55 mL, 3.55 mmol) dropwise over about 20 min. The reaction solution was warmed to about 0° C. over about 1.5 h and then warmed to ambient temperature for about 1 h. The reaction mixture was cooled to about −10° C. prior to addition of water (50 mL) and EtOAc (90 mL). The organic layer was separated, dried over MgSO₄, filtered, and concentrated under reduced pressure. The residue was purified by flash column chromatography on silica gel eluting with a gradient of 1-7% MeOH in DCM. The fractions containing product were combined with the material obtained from the first batch. The material was sonicated with and then concentrated from DCM. The residue was sonicated with and then concentrated from hot EtOH. The resulting solid was dried under vacuum for about 16 h to obtain 3-(5-fluoro-2-(4-methylpiperazin-1-yl)quinazolin-4-yl)-4-(4H-thieno[3,2-b]pyrrol-6-yl)-1H-pyrrole-2,5-dione (0.076 g, 8% yield): LC/MS (Table 1, Method c) Rₜ=1.45 min; MS m/z: 463 (M+H)⁺; ¹H NMR (400 MHz, DMSO-d₆) δ 12.08 (s, 1H), 11.22 (s, 1H), 8.11 (s, 1H), 7.72 (td, J=8.1, 6.3 Hz, 1H), 7.47-7.38 (m, 1H), 7.18 (dd, J=5.3, 1.1, 1H), 7.04-6.91 (m, 2H), 3.98-3.60 (m, 4H), 2.34-2.06 (m, 7H).

### Example #F.3.2

### (R)-3-(5-Fluoro-2-(hexahydropyrrolo[1,2-a]pyrazin-2(1H)-yl)quinazolin-4-yl)-4-(4H-thieno[3,2-b]pyrrol-6-yl)-1H-pyrrole-2,5-dione

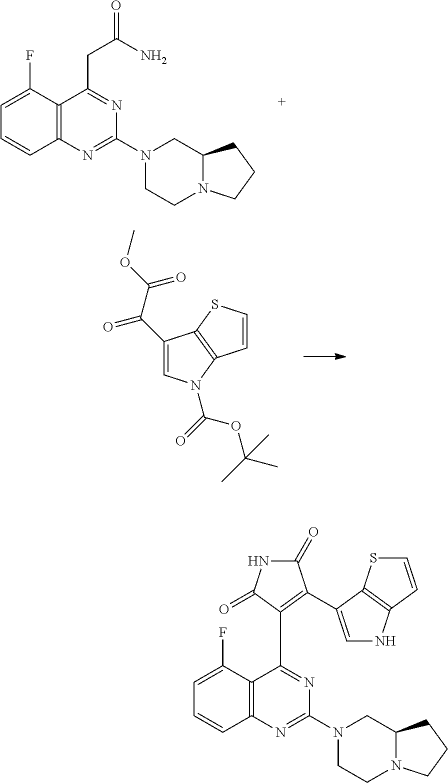

A mixture of (R)-2-(5-fluoro-2-(hexahydropyrrolo[1,2-a]pyrazin-2(1H)-yl)quinazolin-4-yl)acetamide (0.127 g, 0.386 mmol, Preparation #J.3) and tert-butyl 6-(2-methoxy-2-oxoacetyl)-4H-thieno[3,2-b]pyrrole-4-carboxylate (0.179 g, 0.578 mmol, prepared using C with tert-butyl 6-(2-methoxy-2-oxoethyl)-4H-thieno[3,2-b]pyrrole-4-carboxylate (prepared using B with tert-butyl 2-bromothiophen-3-ylcarbamate (Preparation #A.2), K₂CO₃, methyl 4-bromocrotonate)) in THF (1.66 mL) under a nitrogen atmosphere was cooled to about −30° C. To the reaction mixture was added KO-tBu (1 M in THF, 1.54 mL, 1.54 mmol) dropwise over about 20 min. The reaction solution was warmed to about 0° C. over about 1 h and then warmed to ambient temperature for about 16 h. The reaction was cooled to about −10° C. and water (50 mL) and EtOAc (90 mL) were added. The organic layer was dried over MgSO₄, filtered, and concentrated under reduced pressure. The residue was purified by flash column chromatography on silica gel eluting with a gradient of 10-70% (1% (7 N NH₃ in MeOH) in 10% MeOH/DCM) in DCM. The product containing fractions were combined and concentrated under reduced pressure to afford a solid. This material was set aside to combine with the second batch. A mixture of (R)-2-(5-fluoro-2-(hexahydropyrrolo[1,2-a]pyrazin-2(1H)-yl)quinazolin-4-yl)acetamide (0.322 g, 0.978 mmol, Preparation #J.3) and tert-butyl 6-(2-methoxy-2-oxoacetyl)-4H-thieno[3,2-b]pyrrole-4-carboxylate (0.454 g, 1.47 mmol, prepared using C with tert-butyl 6-(2-methoxy-2-oxoethyl)-4H-thieno[3,2-b]pyrrole-4-carboxylate (prepared using B with tert-butyl 2-bromothiophen-3-ylcarbamate (Preparation #A.2), K₂CO₃, methyl 4-bromocrotonate)) in THF (5.2 mL) under a nitrogen atmosphere was cooled to about −30° C. To the reaction mixture was added KO-tBu (1 M in THF, 4.89 mL, 4.89 mmol) dropwise over about 20 min. The reaction solution was warmed to about 0° C. over about 1 h and then warmed to ambient temperature for about 1 h. The reaction was cooled to about −10° C. before water (50 mL) and EtOAc (90 mL) were added. The organic layer was dried over MgSO₄, filtered, and concentrated under reduced pressure. The residue was purified by flash column chromatography on silica gel eluting with a gradient of 20-70% (1% (7 N NH₃ in MeOH) in 10% MeOH/DCM) in DCM. The product containing fractions were combined and concentrated under reduced pressure. The residue was purified by flash column chromatography on silica gel eluting with a gradient of 20-60% (1% (7 N NH₃ in MeOH) in 10% MeOH/DCM) in DCM. The product containing fractions were combined and concentrated under reduced pressure. The material was purified by HPLC (Table 1, Method h) and set aside. The aqueous solution was extracted with EtOAc (2×60 mL). The combined organics were dried over MgSO₄, filtered, and concentrated under reduced pressure. This oil combined with the impure fractions and the material from the first batch was purified by HPLC (Table 1, Method h). The product containing fractions from both HPLC purifications were concentrated under reduced pressure to remove organics, frozen, and placed on a lyophilizer for about 48 h. The residue was suspended in water (30 mL), frozen, and placed on a lyophilizer for about 24 h. The mixture was separated using Varian 218 LC pumps, a Varian CVM 500 with switching valves and heaters for automatic solvent, column and temperature control and a Varian 701 Fraction collector using Method 1 (Table 2) to give the two atropisomers: 10.9 min, negative (−) optical rotation and 14.7 min, positive (+) optical rotation. Each atropisomer was concentrated separately under reduced pressure. NMR and chiral LC/MS indicated that each component went back to mixture of atropisomers. The fractions were combined in one flask. The residue was concentrated from DCM and hot EtOH and then dried in vacuum oven for several hours. The material was triturated with heptane and then dried in a vacuum oven at about 70° C. for about 1 hour. The resulting solid was dissolved in EtOH, and then concentrated under reduced pressure. The residue was placed under house vacuum for about 16 h to give (R)-3-(5-fluoro-2-(hexahydropyrrolo[1,2-a]pyrazin-2(1H)-yl)quinazolin-4-yl)-4-(4H-thieno[3,2-b]pyrrol-6-yl)-1H-pyrrole-2,5-dione (0.636 g, 9% yield): LC/MS (Table 1, Method c) Rₜ=1.70 min; MS m/z: 489 (M+H)⁺; ¹H NMR (400 MHz, DMSO-d₆) δ 12.08 (s, 1H), 11.22 (s, 1H), 8.10 (dd, J=10.2, 0.9 Hz, 1H), 7.78-7.65 (m, 1H), 7.43 (d, J=8.6 Hz, 1H), 7.23-7.11 (m, 1H), 7.04-6.89 (m, 2H), 5.04-4.43 (m, 2H), 3.07-2.79 (m, 3H), 2.71-2.57 (m, 1H), 2.14-1.46 (m, 6H), 1.39-1.19 (m, 1H).

TABLE F.3
Examples prepared from tert-butyl 6-(2-methoxy-2-oxoacetyl)-4H-thieno[3,2-
b]pyrrole-4-carboxylate (prepared using C with tert-butyl 6-(2-methoxy-2-oxoethyl)-4H-
thieno[3,2-b]pyrrole-4-carboxylate (prepared using B with tert-butyl 2-bromothiophen-3-ylcarbamate
(Preparation #A.2), K₂CO₃, methyl 4-bromocrotonate)) using General Procedure F:

| | | | Rₜ min | |
|---|---|---|---|---|
| | | Example | (Table 1, | m/z ESI+ |
| Acetamide | Product | # | Method) | (M + H)⁺ |
| 2-(2-(4-Methylpiperazin-1- yl)quinolin-4-yl)acetamide (prepared using J with methyl 2-(2-(4-methylpiperazin-1- yl)quinolin-4-yl)acetate (prepared using M with tert- butyl 2-(2-(4-methylpiperazin- 1-yl)quinolin-4-yl)acetate (prepared using L with 2-tert- butoxy-2-oxoethyl)zinc(II) chloride (Rieke Metals), Pd(OAc)₂,2- dicyclohexylphosphino-2′,6′- dimethoxybiphenyl, 4-bromo-2- (4-methylpiperazin-1- yl)quinoline (prepared using E with 1-methylpiperazine and 2,4-dibromoquinoline)))) | 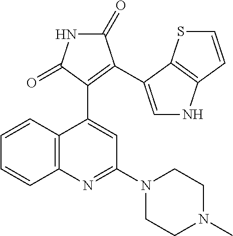 | F.3.3 | 1.43 (c) | 444 |
| 2-(2-(4-Methylpiperazin-1- yl)thieno[2,3-d]pyrimidin-4- yl)acetamide (prepared using E with 1-methylpiperazine and 2- (2-chlorothieno[2,3- d]pyrimidin-4-yl)acetamide (prepared using D with 2,4- dichlorothieno[2,3-d]pyrimidine (ArkPharm Inc))) | 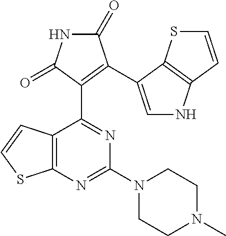 | F.3.4 | 1.53 (c) | 451 |
| 2-(7-Fluoro-2-(4- methylpiperazin-1- yl)quinazolin-4-yl)acetamide (prepared using J with methyl 2-(7-fluoro-2-(4- methylpiperazin-1- yl)quinazolin-4-yl)acetate (prepared using M with tert- butyl 2-(7-fluoro-2-(4- methylpiperazin-1- yl)quinazolin-4-yl)acetate (prepared using E with 1- methylpiperazine and tert-butyl 2-(2-chloro-7-fluoroquinazolin- 4-yl)acetate (prepared using L with 2-tert-butoxy-2- oxoethyl)zinc(II) chloride (Rieke Metals), Pd(OAc)₂, 2- dicyclohexylphosphino-2′,6′- dimethoxybiphenyl, and 2,4- dichloro-7-fluoroquinazoline (WO2005049033A1))))) | 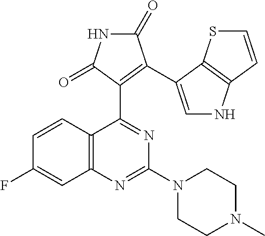 | F.3.5 | 1.67 (c) | 463 |

### Example #F.4.1

### 3-(5-Fluoro-2-(4-methylpiperazin-1-yl)quinazolin-4-yl)-4-(6H-thieno[2,3-b]pyrrol-4-yl)-1H-pyrrole-2,5-dione

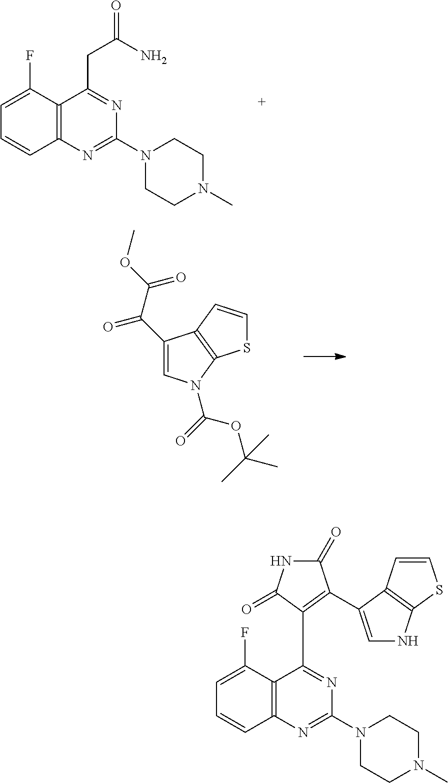

A mixture of 2-(5-fluoro-2-(4-methylpiperazin-1-yl)quinazolin-4-yl)acetamide (0.152 g, 0.501 mmol, Preparation #J.2) and tert-butyl 4-(2-methoxy-2-oxoacetyl)-6H-thieno[2,3-b]pyrrole-6-carboxylate (0.233 g, 0.752 mmol, prepared using C with tert-butyl 4-(2-methoxy-2-oxoethyl)-6H-thieno[2,3-b]pyrrole-6-carboxylate (prepared using B with tert-butyl 3-bromothiophen-2-ylcarbamate (Preparation #A.1), K₂CO₃, methyl 4-bromocrotonate)) in THF (2.15 mL) under a nitrogen atmosphere was cooled to about −30° C. To the reaction mixture was added KOt-Bu (1 M in THF, 2.00 mL, 2.00 mmol) dropwise over about 20 min. The reaction solution was warmed to about 0° C. over about 1.5 h and then warmed to ambient temperature for about 1 h. The reaction mixture was cooled to about −10° C. prior to addition of water (40 mL) and EtOAc (80 mL). The organic layer was separated, dried over MgSO₄, filtered, and concentrated under reduced pressure. The solid was absorbed onto silica gel (8 g) and purified by flash column chromatography on silica gel eluting with a gradient of 0-7% MeOH in DCM. The volatiles were removed under reduced pressure to obtain solid which was set aside to combine with material from the second batch. A mixture of 2-(5-fluoro-2-(4-methylpiperazin-1-yl)quinazolin-4-yl)acetamide (0.153 g, 0.504 mmol, Preparation #J.2) and tert-butyl 4-(2-methoxy-2-oxoacetyl)-6H-thieno[2,3-b]pyrrole-6-carboxylate (0.234 g, 0.757 mmol, prepared using C with tert-butyl 4-(2-methoxy-2-oxoethyl)-6H-thieno[2,3-b]pyrrole-6-carboxylate (prepared using B with tert-butyl 3-bromothiophen-2-ylcarbamate (Preparation #A.1), K₂CO₃, methyl 4-bromocrotonate)) in THF (2.17 mL) under a nitrogen atmosphere was cooled to about −30° C. To the reaction mixture was added KOt-Bu (1 M in THF, 2.00 mL, 2.00 mmol) dropwise over about 20 min. The reaction solution was warmed to about 0° C. over about 1.5 h and then warmed to ambient temperature for about 1 h. The reaction mixture was cooled to about −10° C. prior to addition of water (40 mL) and EtOAc (80 mL). The organic layer was separated, dried over MgSO₄, filtered and concentrated under reduced pressure. The solid was absorbed onto silica gel (8 g) and purified by silica gel chromatography eluting with a gradient of 0-7% MeOH in DCM. The product containing fractions were combined with material from the first batch. The material was sonicated with and then concentrated from DCM. The residue was sonicated with and then concentrated from hot EtOH. The resulting solid was dried in vacuum oven at about 60° C. for about 3 h to obtain 3-(5-fluoro-2-(4-methylpiperazin-1-yl)quinazolin-4-yl)-4-(6H-thieno[2,3-b]pyrrol-4-yl)-1H-pyrrole-2,5-dione (0.061 g, 13% yield): LC/MS (Table 1, Method c) Rₜ=1.48 min; MS m/z: 463 (M+H)⁺; ¹H NMR (400 MHz, DMSO-d₆) δ 12.08 (s, 1H), 11.21 (s, 1H), 7.96 (d, J=0.9 Hz, 1H), 7.74 (td, J=8.1, 6.1 Hz, 1H), 7.51-7.35 (m, 1H), 6.98 (dd, J=11.2, 7.9 Hz, 1H), 6.85 (dd, J=5.4, 1.0 Hz, 1H), 5.43 (d, J=5.4 Hz, 1H), 3.91-3.55 (m, 4H), 2.30-2.07 (m, 7H).

### Example #F.4.2

### 3-(2-(4-Methylpiperazin-1-yl)thieno[2,3-d]pyrimidin-4-yl)-4-(6H-thieno[2,3-b]pyrrol-4-yl)-1H-pyrrole-2,5-dione

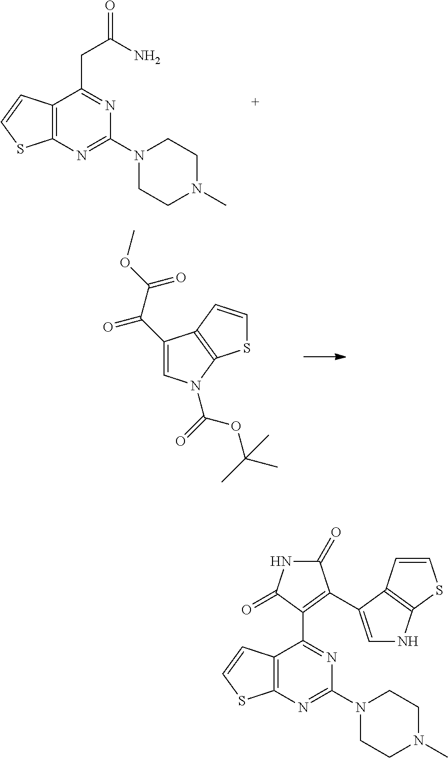

A mixture of 2-(2-(4-methylpiperazin-1-yl)thieno[2,3-d]pyrimidin-4-yl)acetamide (0.345 g, 1.18 mmol, Preparation #E.3) and tert-butyl 4-(2-methoxy-2-oxoacetyl)-6H-thieno[2,3-b]pyrrole-6-carboxylate (0.549 g, 1.78 mmol, prepared using C with tert-butyl 4-(2-methoxy-2-oxoethyl)-6H-thieno[2,3-b]pyrrole-6-carboxylate (prepared using B with tert-butyl 3-bromothiophen-2-ylcarbamate (Preparation #A.1), K₂CO₃, methyl 4-bromocrotonate)) in THF (5.08 mL) under a nitrogen atmosphere was cooled to about −20° C. To the reaction mixture was added KO-tBu (1 M in THF, 8.88 mL, 8.88 mmol) dropwise over about 30 min. The reaction solution was warmed to ambient temperature and stirred for about 2 h. Water (30 mL) was added and the mixture was extracted with EtOAc (3×70 mL). The combined organics were washed with brine, dried over MgSO₄, filtered and concentrated under reduced pressure. The residue was purified by flash column chromatography on silica gel eluting with 70% ((2% 2 N NH₃ in EtOH) in 10% EtOH in DCM) in DCM. The pure fractions were combined and concentrated under reduced pressure. The mixed fractions were combined, concentrated under reduced pressure and purified by flash column chromatography on silica gel eluting with a gradient of 60-70% ((2% 2 N NH₃ in EtOH) in 10% EtOH in DCM) in DCM. The pure fractions were combined with the material from the first column and concentrated under reduced pressure. The residue was dissolved in EtOH (20 mL) and concentrated under reduced pressure. The material was suspended in EtOH (25 mL) and heated at about 45° C. for about 1 h. The volatiles were removed under reduced pressure and the residue was suspended in a solution of 10:1 water/EtOH (20 mL) and heated at about 50° C. for about 2 h. The volatiles were removed under reduced pressure and the mixture was filtered. The solid was dried in a vacuum oven at about 70° C. for about 48 h to afford 3-(2-(4-methylpiperazin-1-yl)thieno[2,3-d]pyrimidin-4-yl)-4-(6H-thieno[2,3-b]pyrrol-4-yl)-1H-pyrrole-2,5-dione (0.082 g, 14% yield): LC/MS (Table 1, method c) Rₜ=1.59 min; MS m/z: 451 (M+H)⁺. ¹H NMR (400 MHz, DMSO-d₆) δ 12.06 (s, 1H), 11.13 (s, 1H), 7.94 (s, 1H), 7.26 (d, J=6.0 Hz, 1H), 7.02 (d, J=6.0 Hz, 1H), 6.84 (d, J=5.3 Hz, 1H), 5.36 (d, J=5.4 Hz, 1H), 3.64 (s, 4H), 2.35-2.06 (m, 7H).

### Example #F.4.3

### (R)-3-(5-Fluoro-2-(hexahydropyrrolo[1,2-a]pyrazin-2(1H)-yl)quinazolin-4-yl)-4-(6H-thieno[2,3-b]pyrrol-4-yl)-1H-pyrrole-2,5-dione

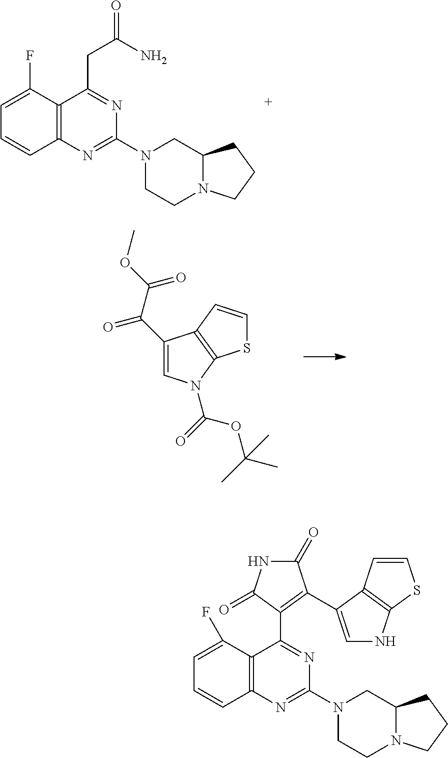

A mixture of (R)-2-(5-fluoro-2-(hexahydropyrrolo[1,2-a]pyrazin-2(1H)-yl)quinazolin-4-yl)acetamide (0.457 g, 1.39 mmol, Preparation #J.3) and tert-butyl 4-(2-methoxy-2-oxoacetyl)-6H-thieno[2,3-b]pyrrole-6-carboxylate (0.644 g, 2.08 mmol, prepared using C with tert-butyl 4-(2-methoxy-2-oxoethyl)-6H-thieno[2,3-b]pyrrole-6-carboxylate (prepared using B with tert-butyl 3-bromothiophen-2-ylcarbamate (Preparation #A.1), K₂CO₃, methyl 4-bromocrotonate)) in THF (6.94 mL) under a nitrogen atmosphere was cooled to about −30° C. To the reaction mixture was added KO-tBu (1.0 M solution in THF, 6.94 mL, 6.94 mmol) dropwise over about 20 min. The reaction solution was warmed to about 0° C. for about 1 h, and then warmed to ambient temperature for about 1 h. The reaction mixture was cooled to about −10° C. and water (30 mL) and EtOAc (90 mL) were added. The organic layer was dried over MgSO₄, filtered, and concentrated under reduced pressure. The material was purified by HPLC (Table 1, Method i). The product containing fractions were combined and the volatiles were removed under reduced pressure. The resulting aqueous solution was frozen and placed on a lyophilizer for about 48 h. The material was suspended in water (50 mL), frozen, and placed on a lyophilizer for about 48 h. The resulting solid was placed in a vacuum oven at about 60° C. for about 1 h to obtain (R)-3-(5-fluoro-2-(hexahydropyrrolo[1,2-a]pyrazin-2(1H)-yl)quinazolin-4-yl)-4-(6H-thieno[2,3-b]pyrrol-4-yl)-1H-pyrrole-2,5-dione (0.077 g, 11% yield): LC/MS (Table 1, Method c) Rₜ=1.53 min; MS m/z: 489 (M+H)⁺; ¹H NMR (400 MHz, DMSO-d₆) δ 7.99-7.88 (m, 1H), 7.76-7.65 (m, 1H), 7.41 (d, J=8.6 Hz, 1H), 7.06-6.88 (m, 1H), 6.80 (d, J=5.3 Hz, 1H), 5.51-5.31 (m, 1H), 5.04-4.34 (m, 2H), 3.02-2.71 (m, 3H), 2.61-2.51 (m, 1H), 2.03-1.88 (m, 2H), 1.84-1.41 (m, 4H), 1.35-1.13 (m, 1H).

TABLE F.4
Examples prepared from tert-butyl 4-(2-methoxy-2-oxoacetyl)-6H-thieno[2,3-b]pyrrole-
6-carboxylate (prepared using C with tert-butyl 4-(2-methoxy-2-oxoethyl)-6H-thieno[2,3-b]
pyrrole-6-carboxylate (prepared using B with tert-butyl 3-bromothiophen-2-ylcarbamate
(Preparation #A.1), K₂CO₃, methyl 4-bromocrotonate)) using General Procedure F:

| | | | Rₜ min | |
|---|---|---|---|---|
| | | Example | (Table 1, | m/z ESI+ |
| Acetamide | Product | # | Method) | (M + H)⁺ |
| 2-(1-((cis-3-Fluoro-1- methylpiperidin-4-yl)methyl)-1H- indazol-3-yl)acetamide (prepared using J with ethyl 2-(1-((cis-3- fluoro-1-methylpiperidin-4- yl)methyl)-1H-indazol-3- yl)acetate (prepared using K with formaldehyde, NaBH₃CN, and ethyl 2-(1-((cis-3-fluoropiperidin- 4-yl)methyl)-1H-indazol-3- yl)acetate hydrochloride (prepared using G with cis-tert-butyl 4-((3- (2-ethoxy-2-oxoethyl)-1H- indazol-1-yl)methyl)-3- fluoropiperidine-1-carboxylate (prepared using I with ethyl 2- (1H-indazol-3-yl)acetate (J. Med. Chem. 1992, 35, 2155-2162.), Cs₂CO₃, and cis-tert-butyl 3- fluoro-4- ((methylsulfonyloxy)methyl) piperidine-1-carboxylate (prepared using H with MsCl, TEA, and cis- tert-butyl 3-fluoro-4- (hydroxymethyl)piperidine-1- carboxylate (prepared using R with Boc₂O, TEA and 3- fluoropiperidin-4-yl)methanol hydrochloride (WO2006069287A1.))))))) | 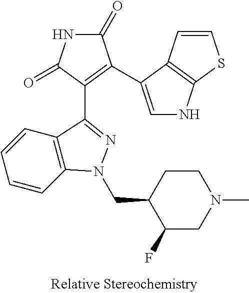 | F.4.4 | 1.60 (c) | 464 |

| | | | | |
|---|---|---|---|---|
| 2-(1-(cis-4- (Dimethylamino)cyclohexyl)-1H- indazol-3-yl)acetamide (prepared using J with ethyl 2-(1-(cis-4- (dimethylamino)cyclohexyl)-1H- indazol-3-yl)acetate (prepared using K with formaldehyde, NaBH₃CN, and ethyl 2-(1-(cis-4- aminocyclohexyl)-1H-indazol-3- yl)acetate hydrochloride (prepared using G with ethyl 2-(1-(cis-4- (tert-butoxycarbonylamino) cyclohexyl)-1H-indazol-3-yl) acetate (prepared using I with ethyl 2-(1H-indazol-3-yl)acetate (J. Med. Chem. 1992, 35, 2155-2162.), diisopropyl azodicarboxylate, triphenylphosphine and trans-4- Boc-aminocyclohexanol (Oakwood))))) | 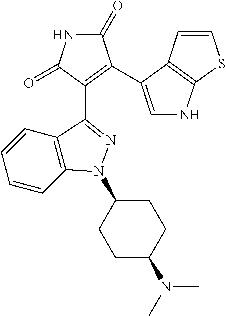 | F.4.5 | 1.85 (c) | 460 |
| 2-(1-((1-Methylpiperidin-4- yl)methyl)-1H-indazol-3- yl)acetamide (prepared using J with ethyl 2-(1-((1- methylpiperidin-4-yl)methyl)-1H- indazol-3-yl)acetate (prepared using K with formaldehyde, NaBH₃CN, and ethyl 2-(1- (piperidin-4-ylmethyl)-1H- indazol-3-yl)acetate hydrochloride (prepared using G with tert-butyl 4-((3-(2-ethoxy-2-oxoethyl)-1H- indazol-1-yl)methyl)piperidine-1- carboxylate (prepared using I with N-Boc-4-(4- toluenesulfonyloxymethyl) piperidine (AstaTech), Cs₂CO₃, and ethyl 2-(1H-indazol-3-yl)acetate (J. Med. Chem. 1992, 35, 2155- 2162.))))) | 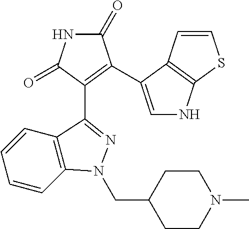 | F.4.6 | 1.61 (c) | 446 |
| 2-(1-(1-Methylpiperidin-4-yl)-1H- indazol-3-yl)acetamide (Preparation #K.1) | 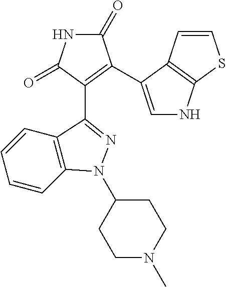 | F.4.7 | 1.62 (c) | 432 |
| 2-(2-(4-Methylpiperazin-1- yl)pyrimidin-4-yl)acetamide (prepared using E with 1- methylpiperazine and 2-(2- chloropyrimidin-4-yl)acetamide (prepared using D with 2,4- dichloropyrimidine)) | 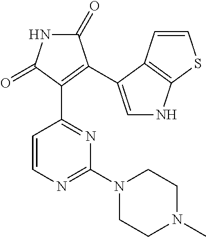 | F.4.8 | 1.35 (c) | 395 |
| 2-(2-(4-Methylpiperazin-1- yl)thieno[3,2-d]pyrimidin-4- yl)acetamide (prepared using E with 1-methylpiperazine and 2-(2- chlorothieno[3,2-d]pyrimidin-4- yl)acetamide (prepared using D with 2,4-dichlorothieno[3,2- d]pyrimidine (Accela ChemBio))) | 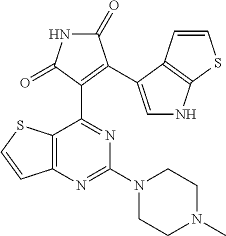 | F.4.9 | 1.52 (c) | 451 |
| 2-(2-(4-Methylpiperazin-1-yl)- 6,7-dihydro-5H-cyclopenta[d] pyrimidin-4-yl)acetamide (prepared using E with 1- methylpiperazine (Sinopharm Chemical Reagent Co. Ltd.) and 2-(2-(methylsulfonyl)-6,7- dihydro-5H-cyclopenta[d] pyrimidin-4-yl)acetamide (prepared using D with 2,4- bis(methylsulfonyl)-6,7-dihydro- 5H-cyclopenta[d]pyrimidine (Tetrahedron, 1996, 7973- 7982.))) | 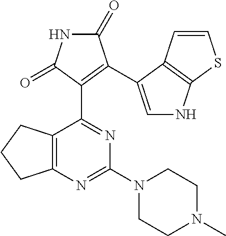 | F.4.10 | 1.52 (c) | 435 |
| 2-(5-Methoxy-2-(4- methylpiperazin-1-yl)quinazolin- 4-yl)acetamide (prepared using J with ethyl 2-(5-methoxy-2-(4- methylpiperazin-1-yl)quinazolin- 4-yl)acetate (prepared using L with 2,4-dichloro-5- methoxyquinazoline (Anichem), 3-oxobutanoate, and 1- methylpiperazine)) | 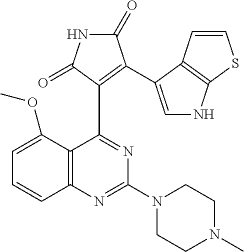 | F.4.11 | 1.60 (c) | 475 |
| 2-(5-Methoxy-2-(4- methylpiperazin-1-yl)quinazolin- 4-yl)acetamide (prepared using J with ethyl 2-(5-methoxy-2-(4- methylpiperazin-1-yl)quinazolin- 4-yl)acetate (prepared using L with 2,4-dichloro-5- methoxyquinazoline (Anichem), 3-oxobutanoate, and 1- methylpiperazine)) | 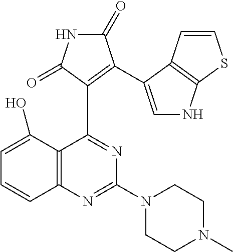 | F.4.12 | 1.42 (c) | 461 |
| 2-(6-Chloro-2-(4- methylpiperazin-1-yl)quinazolin- 4-yl)acetamide (prepared using J with ethyl 2-(6-chloro-2-(4- methylpiperazin-1-yl)quinazolin- 4-yl)acetate (prepared using L with 2,4,6-trichloroquinazoline (ChemImpex), 3-oxobutanoate, and 1-methylpiperazine)) | 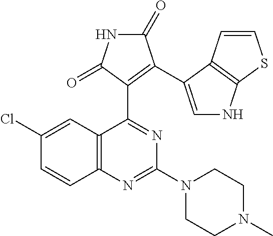 | F.4.13 | 1.82 (c) | 479 |
| 2-(6-Methoxy-2-(4- methylpiperazin-1-yl)quinazolin- 4-yl)acetamide (prepared using J with ethyl 2-(6-methoxy-2-(4- methylpiperazin-1-yl)quinazolin- 4-yl)acetate (prepared using L with 2,4-dichloro-6- methoxyquinazoline (Beta Pharma), 3-oxobutanoate, and 1- methylpiperazine)) | 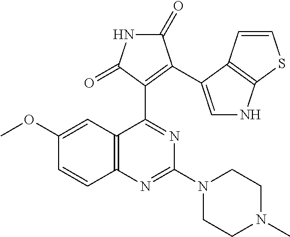 | F.4.14 | 1.59 (c) | 475 |
| 2-(6-Fluoro-2-(4-methylpiperazin- 1-yl)quinazolin-4-yl)acetamide (prepared using J with 2-(6- fluoro-2-(4-methylpiperazin-1- yl)quinazolin-4-yl)acetate (prepared using L with 2,4- dichloro-6-fluoroquinazoline (Beta Pharma), 3-oxobutanoate, and 1-methylpiperazine)) | 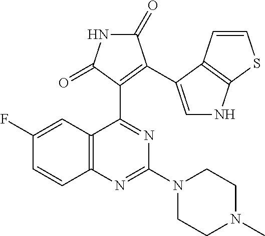 | F.4.15 | 1.66 (c) | 463 |
| 2-(2-(4-Methylpiperazin-l-yl)- 5,6,7,8-tetrahydroquinazolin-4- yl)acetamide (prepared using E with 1-methylpiperazine (Sinopharm Chemical Reagent Co. Ltd.) and 2-(2- (methylsulfonyl)-5,6,7,8- tetrahydroquinazolin-4- yl)acetamide (prepared using D with 2,4-bis(methylsulfonyl)- 5,6,7,8-tetrahydroquinazoline (Tetrahedron, 1996, 7973-7982.))) | 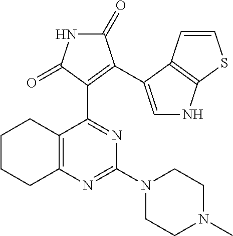 | F.4.16 | 1.73 (c) | 449 |
| 2-(1-((1-Methylpiperidin-4- yl)methyl)-1H-indol-3-yl) acetamide (prepared using J with ethyl 2-(1-((1-methylpiperidin-4- yl)methyl)-1H-indol-3-yl)acetate (prepared using K with formaldehyde, NaBH₃CN, and ethyl 2-(1-(piperidin-4-ylmethyl)- 1H-indol-3-yl)acetate (prepared using G with tert-butyl 4-((3-(2- ethoxy-2-oxoethyl)-1H-indol-1- yl)methyl)piperidine-1- carboxylate (prepared using I with ethyl 2-(1H-indol-3-yl)acetate, Cs₂CO₃, and tert-butyl 4- ((methylsulfonyloxy)methyl) piperidine-1-carboxylate (prepared using H with tert-butyl 4- (hydroxymethyl)piperidine-1- carboxylate, MsCl, and TEA))))) | 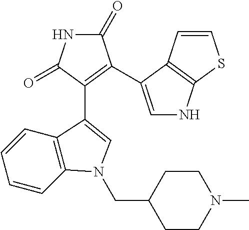 | F.4.17 | 1.68 (c) | 445 |
| 2-(1-((cis-4- (Dimethylamino)cyclohexyl) methyl)-1H-indazol-3-yl)acetamide (prepared using J with ethyl 2-(1- ((cis-4- (dimethylamino)cyclohexyl) methyl)-1H-indazol-3-yl)acetate (prepared using I with ethyl 2- (1H-indazol-3-yl)acetate (J. Med. Chem. 1992, 35, 2155-2162.), Cs₂CO₃, and cis-4- (chloromethyl)-N,N- dimethylcyclohexanamine hydrochloride (prepared using P with SOCl₂ and (cis-4- (dimethylamino)cyclohexyl) methanol (prepared using Q with LiAlH₄ and cis-methyl 4- (dimethylamino) cyclohexanecarboxylate (prepared using K with formaldehyde, NaBH₃CN, and cis-methyl 4- aminocyclohexanecarboxylate hydrochloride (TCI)))))) | 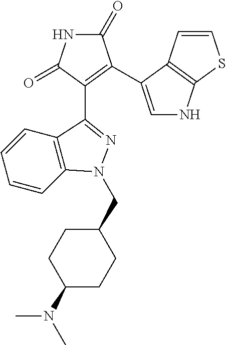 | F.4.18 | 1.67 (c) | 474 |
| 2-(1-((1-Ethylpiperidin-4- yl)methyl)-1H-indazol-3- yl)acetamide (prepared using J with ethyl 2-(1-((1-ethylpiperidin- 4-yl)methyl)-1H-indazol-3- yl)acetate (prepared using I with iodoethane, Cs₂CO₃, and ethyl 2- (1-(piperidin-4-ylmethyl)-1H- indazol-3-yl)acetate hydrochloride (prepared using G with tert-butyl 4-((3-(2-ethoxy-2-oxoethyl)-1H- indazol-1-yl)methyl)piperidine-1- carboxylate (prepared using I with N-Boc-4-(4- toluenesulfonyloxymethyl) piperidine (AstaTech), Cs₂CO₃, and ethyl 2-(1H-indazol-3-yl)acetate (J. Med. Chem. 1992, 35, 2155- 2162.))))) | 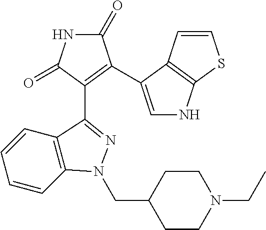 | F.4.19 | 1.58 (c) | 460 |
| 2-(1-((1-(2-fluoroethyl)piperidin- 4-yl)methyl)-1H-indazol-3- yl)acetamide (prepared using J with ethyl 2-(1-((1-(2- fluoroethyl)piperidin-4- yl)methyl)-1H-indazol-3- yl)acetate (prepared using I with 1-bromo-2-fluoroethane, Cs₂CO₃, and ethyl 2-(1-(piperidin-4- ylmethyl)-1H-indazol-3-yl)acetate hydrochloride (prepared using G with tert-butyl 4-((3-(2-ethoxy-2- oxoethyl)-1H-indazol-1- yl)methyl)piperidine-1- carboxylate (prepared using I with N-Boc-4-(4- toluenesulfonyloxymethyl) piperidine (AstaTech), Cs₂CO₃, and ethyl 2-(1H-indazol-3-yl)acetate (J. Med. Chem. 1992, 35, 2155- 2162.))))) | 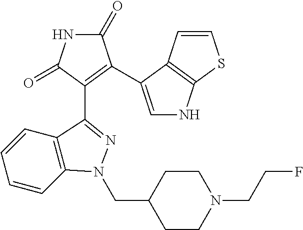 | F.4.20 | 1.66 (c) | 478 |
| 2-(1-((1-(2- Methoxyethyl)piperidin-4- yl)methyl)-1H-indazol-3- yl)acetamide (prepared using J with ethyl 2-(1-((1-(2- methoxyethyl)piperidin-4- yl)methyl)-1H-indazol-3- yl)acetate (prepared using I with 2-bromoethyl methyl ether, Cs₂CO₃, and ethyl 2-(1-(piperidin- 4-ylmethyl)-1H-indazol-3- yl)acetate hydrochloride (prepared using G with tert-butyl 4-((3-(2- ethoxy-2-oxoethyl)-1H-indazol-1- yl)methyl)piperidine-1- carboxylate (prepared using I with N-Boc-4-(4- toluenesulfonyloxymethyl) piperidine (AstaTech), Cs₂CO₃, and ethyl 2-(1H-indazol-3-yl)acetate (J. Med. Chem. 1992, 35, 2155- 2162.))))) | 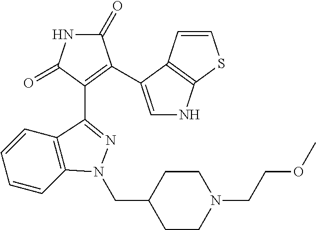 | F.4.21 | 1.68 (c) | 490 |
| 2-(1-((1-Methylazetidin-3- yl)methyl)-1H-indazol-3- yl)acetamide (prepared using J with ethyl 2-(1-((1- methylazetidin-3-yl)methyl)-1H- indazol-3-yl)acetate (prepared using K with formaldehyde, NaBH₃CN, and ethyl 2-(1- (azetidin-3-ylmethyl)-1H-indazol- 3-yl)acetate (prepared using G with tert-butyl 3-((3-(2-ethoxy-2- oxoethyl)-1H-indazol-1- yl)methyl)azetidine-1-carboxylate (prepared using I with ethyl 2- (1H-indazol-3-yl)acetate (J. Med. Chem. 1992, 35, 2155-2162.), Cs₂CO₃, and tert-butyl 3- ((methylsulfonyloxy)methyl) azetidine-1-carboxylate (prepared using H with tert-butyl 3- (hydroxymethyl)azetidine-1- carboxylate (Ace Synthesis), MsCl, and TEA))))) | 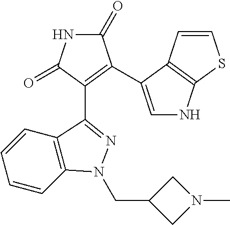 | F.4.22 | 1.55 (c) | 418 |
| 2-(1-((1,4-Dimethylpiperidin-4- yl)methyl)-1H-indazol-3- yl)acetamide (prepared using J with ethyl 2-(1-((1,4- dimethylpiperidin-4-yl)methyl)- 1H-indazol-3-yl)acetate (prepared using K with formaldehyde, NaBH₃CN, and ethyl 2-(1-((4- methylpiperidin-4-yl)methyl)-1H- indazol-3-yl)acetate (prepared using G with tert-butyl 4-((3-(2- ethoxy-2-oxoethyl)-1H-indazol-1- yl)methyl)-4-methylpiperidine-1- carboxylate (prepared using I with ethyl 2-(1H-indazol-3-yl)acetate (J. Med. Chem. 1992, 35, 2155- 2162.), Cs₂CO₃, and tert-butyl 4- methyl-4-((methylsulfonyloxy) methyl)piperidine-1-carboxylate (prepared using H with MsCl, TEA, and tert-butyl 4- (hydroxymethyl)-4- methylpiperidine-1-carboxylate (prepared using Q with LiAlH₄ and 1-tert-butyl 4-ethyl 4- methylpiperidine-1,4- dicarboxylate (Accela Chem Bio))))))) | 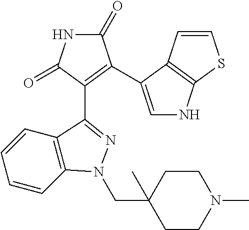 | F.4.23 | 1.75 (c) | 460 |
| 2-(2-(4-Methylpiperazin-1- yl)pyrido[3,4-d]pyrimidin-4- yl)acetamide (prepared using J with ammonium hydroxide (Sinopharm Chemical Reagent Co. Ltd.) and ethyl 2-(2-(4- methylpiperazin-1-yl)pyrido[3,4- d]pyrimidin-4-yl)acetate (prepared using E with 1- methylpiperazine (Sinopharm Chemical Reagent Co. Ltd.) and ethyl 2-(2-chloropyrido[3,4- d]pyrimidin-4-yl)acetate (prepared using L with ethyl acetoacetate (Sinopharm Chemical Reagent Co. Ltd.) and 2,4-dichloro-pyrido[3,4- d]pyrimidine (WO2006090169A1.)))) | 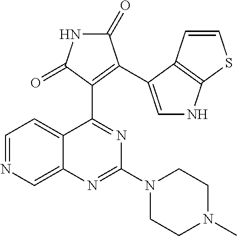 | F.4.24 | 1.47 (c) | 446 |
| 2-(2-(4-Methylpiperazin-1- yl)pyrido[3,2-d]pyrimidin-4- yl)acetamide (prepared using J with ammonium hydroxide (Sinopharm Chemical Reagent Co. Ltd.) and ethyl 2-(2-(4- methylpiperazin-1-yl)pyrido[3,2- d]pyrimidin-4-yl)acetate (prepared using E with 1- methylpiperazine (Sinopharm Chemical Reagent Co. Ltd.) and ethyl 2-(2-chloropyrido[3,2- d]pyrimidin-4-yl)acetate (prepared using L with ethyl acetoacetate (Sinopharm Chemical Reagent Co. Ltd.) and 2,4-dichloropyrido[3,2- d]pyrimidine (Synlett., 2006, 12, 1938-1942.)))) | 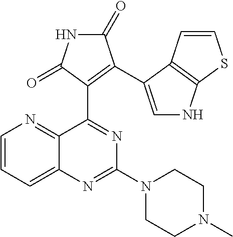 | F.4.25 | 1.46 (c) | 446 |
| 2-(1-((1-(2- Methoxyethyl)pyrrolidin-3- yl)methyl)-1H-indazol-3- yl)acetamide (prepared using J with ethyl 2-(1-((1-(2- methoxyethyl)pyrrolidin-3- yl)methyl)-1H-indazol-3- yl)acetate (prepared using I with ethyl 2-(1H-indazol-3-yl)acetate (J. Med. Chem. 1992, 35, 2155- 2162.) and (1-(2- methoxyethyl)pyrrolidin-3- yl)methyl methanesulfonate (prepared using H with MsCl (Sinopharm Chemical Reagent Co. Ltd.), TEA (Sinopharm Chemical Reagent Co. Ltd.), and (1-(2-methoxyethyl)pyrrolidin-3- yl)methanol (ChemBridge)))) | 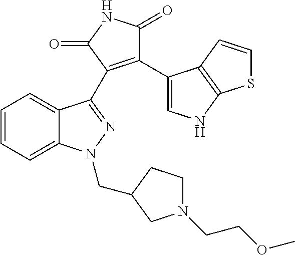 | F.4.26 | 1.64 (c) | 476 |
| 2-(1-(4-(Dimethylamino)butyl)- 1H-indazol-3-yl)acetamide (prepared using J with ethyl 2-(1- (4-(dimethylamino)butyl)-1H- indazol-3-yl)acetate (prepared using I with ethyl 2-(1H-indazol- 3-yl)acetate (J. Med. Chem. 1992, 35, 2155-2162.) and 4-chloro- N,N-dimethylbutan-1-amine hydrochloride (prepared using P with 4-(dimethylamino)butan-1-ol (Sinopharm Chemical Reagent Co. Ltd.)))) | 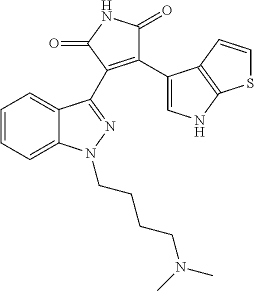 | F.4.27 | 1.62 (c) | 434 |
| 2-(1-((1-Methylpiperidin-3- yl)methyl)-1H-indazol-3- yl)acetamide (prepared using J with ethyl 2-(1-((1- methylpiperidin-3-yl)methyl)-1H- indazol-3-yl)acetate (prepared using I with ethyl 2-(1H-indazol- 3-yl)acetate (J. Med. Chem. 1992, 35, 2155-2162.) and (1- methylpiperidin-3-yl)methyl methanesulfonate (prepared using H with MsCl (Sinopharm Chemical Reagent Co. Ltd.), TEA (Sinopharm Chemical Reagent Co. Ltd.), and (1-methylpiperidin- 3-yl)methanol (Sinopharm Chemical Reagent Co. Ltd.)))) | 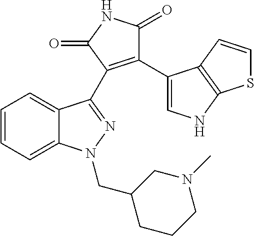 | F.4.28 | 1.58 (c) | 446 |
| 2-(1-(3-(Dimethylamino)propyl)- 1H-indazol-3-yl)acetamide (prepared using J with ethyl 2-(1- (3-(dimethylamino)propyl)-1H- indazol-3-yl)acetate (prepared using I with ethyl 2-(1H-indazol- 3-yl)acetate (J. Med. Chem. 1992, 35, 2155-2162.) and 3-chloro- N,N-dimethylpropan-1-amine (Sinopharm Chemical Reagent Co. Ltd.))) | 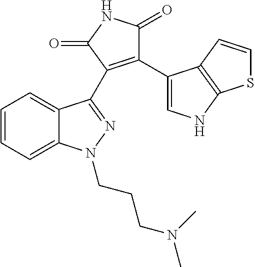 | F.4.29 | 1.54 (c) | 420 |
| 2-(5-Methyl-2-(4- methylpiperazin-1-yl)quinazolin- 4-yl)acetamide (prepared using J with ethyl 2-(5-methyl-2-(4- methylpiperazin-1-yl)quinazolin- 4-yl)acctate (prepared using E with 1-methylpiperazine (Sinopharm Chemical Reagent Co. Ltd.) and ethyl 2-(2-chloro-5- methylquinazolin-4-yl)acetate (prepared using L with ethyl acetoacetate (Sinopharm Chemical Reagent Co. Ltd.) and 2,4-dichloro-5-methylquinazoline (WO2008002596A2)))) | 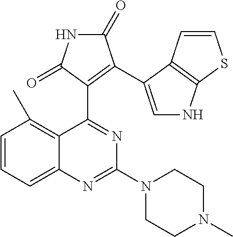 | F.4.30 | 1.70 (c) | 459 |
| 2-(6-Methyl-2-(4- methylpiperazin-1-yl)quinazolin- 4-yl)acetamide (prepared using J with ethyl 2-(6-methyl-2-(4- methylpiperazin-1-yl)quinazolin- 4-yl)acetate (prepared using E with 1-methylpiperazine (Sinopharm Chemical Reagent Co. Ltd.) and ethyl 2-(2-chloro-6- methylquinazolin-4-yl)acetate (prepared using L with ethyl acetoacetate (Sinopharm Chemical Reagent Co. Ltd.) and 2,4-dichloro-6-methylquinazoline (. WO2008002596A2)))) | 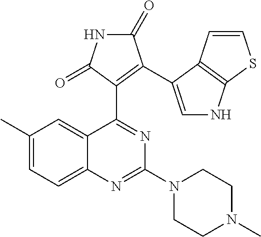 | F.4.31 | 1.70 (c) | 459 |
| 2-(8-Methyl-2-(4- methylpiperazin-1-yl)quinazolin- 4-yl)acetamide (prepared using J with ethyl 2-(8-methyl-2-(4- methylpiperazin-1-yl)quinazolin- 4-yl)acetate (prepared using E with 1-methylpiperazine (Sinopharm Chemical Reagent Co. Ltd.) and ethyl 2-(2-chloro-8- methylquinazolin-4-yl)acetate (prepared using L with ethyl acetoacetate (Sinopharm Chemical Reagent Co. Ltd.) and 2,4-dichloro-8-methylquinazoline (WO2008002596A2)))) | 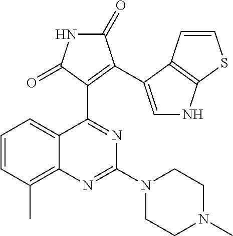 | F.4.32 | 1.78 (c) | 459 |
| 2-(3-((1-Methylpiperidin-4- yl)methyl)imidazo[1,5-a]pyridin- 1-yl)acetamide (prepared using J with ethyl 2-(3-((1- methylpiperidin-4- yl)methyl)imidazo[1,5-a]pyridin- 1-yl)acetate (Preparation #5)) | 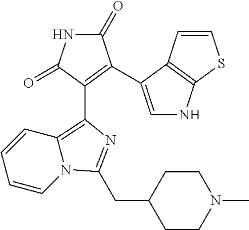 | F.4.33 | 1.53 (c) | 446 |
| 2-(7-Fluoro-3-(4-methylpiperazin- 1-yl)isoquinolin-1-yl)acetamide (Preparation #6) | 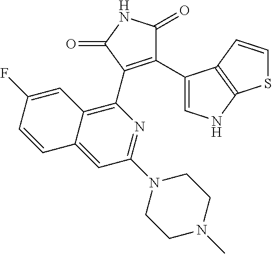 | F.4.34 | 1.70 (c) | 462 |
| 2-(1-(1-(1-Methylpiperidin-4- yl)ethyl)-1H-indazol-3- yl)acetamide (prepared using J with ethyl 2-(1-(1-(1- methylpiperidin-4-yl)ethyl)-1H- indazol-3-yl)acetate (prepared using K with formaldehyde, NaBH₃CN, and ethyl 2-(1-(1- (piperidin-4-yl)ethyl)-1H-indazol- 3-yl)acetate hydrochloride (prepared using G with tert-butyl 4-(1-(3-(2-ethoxy-2-oxoethyl)- 1H-indazol-1-yl)ethyl)piperidine- 1-carboxylate (prepared using I with ethyl 2-(1H-indazol-3- yl)acetate (J. Med. Chem. 1992, 35, 2155-2162.), Cs₂CO₃, and tert-butyl 4-(1- (methylsulfonyloxy)ethyl) piperidine-1-carboxylate (prepared using H with tert-butyl 4-(1- hydroxyethyl)piperidine-1- carboxylate (WO2008070692A2), TEA, and MsCl))))) | 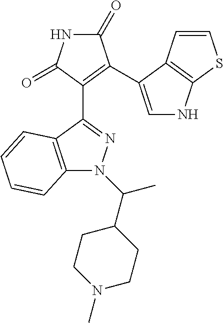 | F.4.35 | 1.69 (c) | 460 |
| 2-(2-(4-Methylpiperazin-1- yl)pyrrolo[1,2-f][1,2,4]triazin-4- yl)acetamide (prepared using E with 1-methylpiperazine and 2-(2- chloropyrrolo[1,2-f][1,2,4]triazin- 4-yl)acetamide (prepared using D with 2,4-dichloropyrrolo[1,2- f][1,2,4]triazine (Synthonix))) | 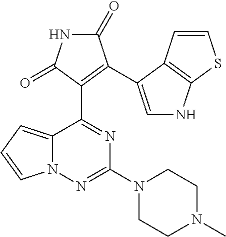 | F.4.36 | 1.60 (c) | 434 |
| 2-(3-((1-Methylpiperidin-4- yl)methyl)-1H-indol-1- yl)acetamide (prepared using K with formaldehyde, NaBH₃CN, and 2-(3-(piperidin-4-ylmethyl)- 1H-indol-1-yl)acetamide (prepared using G with tert-butyl 4-((1-(2-amino-2-oxoethyl)-1H- indol-3-yl)methyl)piperidine-1- carboxylate (Preparation #8))) | 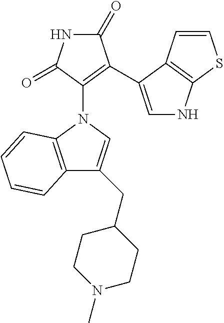 | F.4.37 | 1.75 (c) | 445 |

TABLE F.5
Examples prepared from methyl 2-(1H-indol-3-yl)-2-oxoacetate using General Procedure F:

| | | | Rₜ min | |
|---|---|---|---|---|
| | | Example | (Table 1, | m/z ESI+ |
| Acetamide | Product | # | Method) | (M + H)⁺ |
| 2-(6-((1-Methylpiperidin-4- yl)methyl)-6H-thieno[2,3-b]pyrrol- 4-yl)acetamide (prepared using J with ethyl 2-(6-((1-methylpiperidin- 4-yl)methyl)-6H-thieno[2,3- b]pyrrol-4-yl)acetate (prepared using I with 4-(chloromethyl)-1- methylpiperidine hydrochloride (prepared using P with SOCl₂ and (1-methylpiperidin-4-yl)methanol (AK Scientific, Inc.)), Cs₂CO₃, and ethyl 2-(6H-thieno[2,3-b]pyrrol-4- yl)acetate (prepared using G with tert-butyl 4-(2-ethoxy-2-oxoethyl)- 6H-thieno[2,3-b]pyrrole-6- carboxylate (Preparation #C.l)))) | 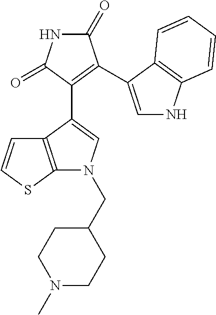 | F.5.1 | 1.65 (c) | 445 |

### General Procedure G

### Boc Cleavage

To a carbamate (1 equiv) is optionally added an organic solvent such as 1,4-dioxane, THF, MeOH, or EtOH (preferably MeOH). An acid such as TFA or HCl (5-30 equiv, preferably HCl, 10-20 equiv), optionally as a solution in an organic solvent such as 1,4-dioxane, THF, or Et₂O (preferably 1,4-dioxane), is added at ambient temperature. The reaction mixture is then stirred for about 1-24 h (preferably 3 to 16 h) at ambient temperature to reflux (preferably ambient temperature). Optionally, water is added. The volatiles are removed under reduced pressure. Optionally, the material is partitioned between an aqueous layer with pH of approximately 6-10 and an appropriate organic solvent such as EtOAc or DCM, dried over MgSO₄ or Na₂SO₄, filtered, and concentrated under reduced pressure.

### Illustration of General Procedure G

### Example #G.1.1

### 3-(2-(piperazin-1-yl)quinazolin-4-yl)-4-(4H-thieno[3,2-b]pyrrol-6-yl)-1H-pyrrole-2,5-dione

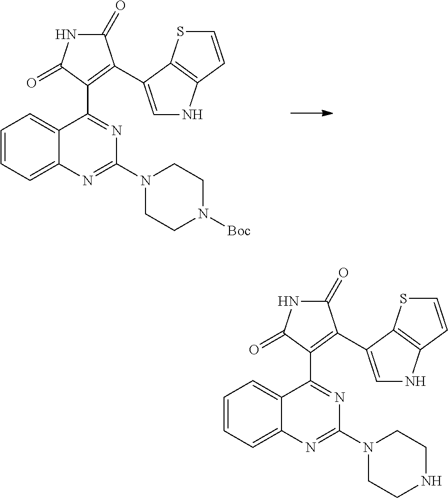

HCl (4.0 M solution in 1,4-dioxane, 1.97 mL, 7.86 mmol) was added to a solution of tert-butyl 4-(4-(2,5-dioxo-4-(4H-thieno[3,2-b]pyrrol-6-yl)-2,5-dihydro-1H-pyrrol-3-yl)quinazolin-2-yl)piperazine-1-carboxylate (0.417 g, 0.786 mmol, prepared using F with tert-butyl 6-(2-ethoxy-2-oxoacetyl)-4H-thieno[3,2-b]pyrrole-4-carboxylate (Preparation #C.2) and tert-butyl 4-(4-(2-amino-2-oxoethyl)quinazolin-2-yl)piperazine-1-carboxylate (prepared using E with 2-(2-chloroquinazolin-4-yl)acetamide (Preparation #D.1) and 1-Boc-piperazine)) and MeOH (7.9 mL). After stirring for about 4 h at ambient temperature, water (30 mL) was added. The volatiles were removed under reduced pressure. The aqueous solution was adjusted to about pH 7 with 2 N aqueous NaOH. The precipitate was collected by filtration and then washed with water. The crude material was dissolved in DMSO and purified by RP-HPLC (Table 1, Method d) to give 3-(2-(piperazin-1-yl)quinazolin-4-yl)-4-(4H-thieno[3,2-b]pyrrol-6-A-1H-pyrrole-2,5-dione (0.176 g, 52% yield): LC/MS (Table 1, Method c) Rₜ=1.63 min; MS m/z: 431 (M+H)⁺.

TABLE G.1
Examples prepared using General Procedure G:

| | | | Rₜ min | |
|---|---|---|---|---|
| | | Example | (Table 1, | m/z ESI+ |
| Carbamate | Product | # | Method) | (M + H)⁺ |
| (1S,4S)-tert-Butyl 5-(4-(2,5- dioxo-4-(6H-thieno[2,3-b]pyrrol- 4-yl)-2,5-dihydro-1H-pyrrol-3- yl)quinazolin-2-yl)-2,5- diazabicyclo[2.2.1]heptane-2- carboxylate (prepared using F from tert-butyl 4-(2-ethoxy-2- oxoacetyl)-6H-thieno[2,3- b]pyrrole-6-carboxylate (Preparation #C.l) and (1S,4S)- tert-butyl 5-(4-(2-amino-2- oxoethyl)quinazolin-2-yl)-2,5- diazabicyclo[2.2.1]heptane-2- carboxylate (prepared using E from 2-(2-chloroquinazolin-4- yl)acetamide (Preparation #D.l) and (1S,4S)-tert-butyl 2,5- diazabicyclo[2.2.1]heptane-2- carboxylate)) | 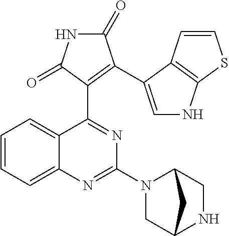 | G.1.2 | 1.38 (b) | 443 |
| tert-Butyl 5-(4-(2,5-dioxo-4-(4H- thieno[3,2-b]pyrrol-6-yl)-2,5- dihydro-1H-pyrrol-3- yl)quinazolin-2- yl)hexahydropyrrolo[3,4- c]pyrrole-2(1H)-carboxylate (prepared using F from tert-butyl 5-(4-(2-amino-2- oxoethyl)quinazolin-2- yl)hexahydropyrrolo[3,4- c]pyrrole-2(1H)-carboxylate (prepared using E with 2-(2- chloroquinazolin-4-yl)acetainide (Preparation #D.l) and 2-Boc- hexahydro-pyrrolo [3,4-c]pyrrole (Astatech)) and tert-butyl 6-(2- ethoxy-2-oxoacetyl)-4H- thieno[3,2-b]pyrrole-4- carboxylate (Preparation #C.2)) | 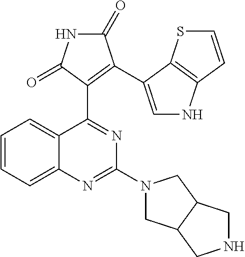 | G.1.3 | 1.52 (a) | 457 |
| tert-Butyl (1-(4-(2,5-dioxo-4-(4H- thieno[3,2-b]pyrrol-6-yl)-2,5- dihydro-1H-pyrrol-3-yl) quinazolin-2-yl)piperidin-4-yl) methylcarbamate (prepared using E with 3-(2-chloroquinazolin-4- yl)-4-(4H-thieno[3,2-b]pyrrol-6- yl)-1H-pyrrole-2,5-dione (Preparation #F.2), TEA, and tert- butyl piperidin-4- ylmethylcarbamate (Astatech)) | 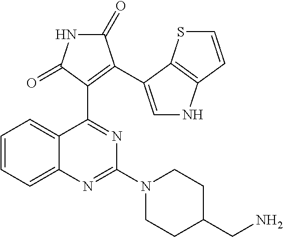 | G.1.4 | 1.61 (c) | 459 |
| tert-Butyl 4-(4-(2,5-dioxo-4-(6H- thieno[2,3-b]pyrrol-4-yl)-2,5- dihydro-1H-pyrrol-3- yl)quinazolin-2-yl)piperazine-1- carboxylate (prepared using F with tert-butyl 4-(2-ethoxy-2- oxoacetyl)-6H-thieno[2,3- b]pyrrole-6-carboxylate (Preparation #C.l) and tert-butyl 4-(4-(2-amino-2- oxoethyl)quinazolin-2- yl)piperazine-1-carboxylate (prepared using E with 2-(2- chloroquinazolin-4-yl)acetamide (Preparation #D.l), TEA, and 1- Boc-piperazine)) | 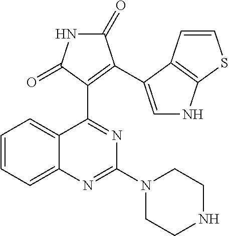 | G.1.5 | 1.60 (c) | 431 |
| tert-Butyl 2-(4-(2,5-dioxo-4-(4H- thieno[3,2-b]pyrrol-6-yl)-2,5- dihydro-1H-pyrrol-3- yl)quinazolin-2-yl)-2,7- diazaspiro[4.5]decane-7- carboxylate (prepared using E with 3-(2-chloroquinazolin-4-yl)- 4-(4H-thieno[3,2-b]pyrrol-6-yl)- 1H-pyrrole-2,5-dione (Preparation #F.2), TEA, and 2,7-diaza- spiro[4.5]decane-7-carboxylic acid tert-butyl ester (Syntech)) | 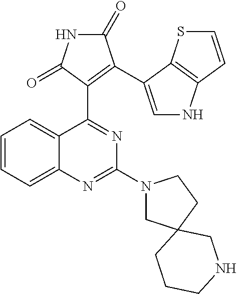 | G.1.6 | 1.66 (c) | 485 |
| tert-Butyl 1-(4-(2,5-dioxo-4-(6H- thieno[2,3-b]pyrrol-4-yl)-2,5- dihydro-1H-pyrrol-3- yl)quinazolin-2-yl)pyrrolidin-3- yl(methyl)carbamate (prepared using E with 3-(2- chloroquinazolin-4-yl)-4-(6H- thieno[2,3-b]pyrrol-4-yl)-1H- pyrrole-2,5-dione (Preparation #F.l), TEA, and 3-(N-tert- butoxycarbonyl-N- methylamino)pyrrolidine (TCI)) | 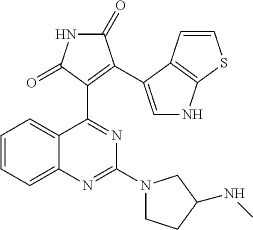 | G.1.7 | 1.49 (c) | 445 |
| tert-Butyl 4-(4-(2,5-dioxo-4-(4H- thieno[3,2-b]pyrrol-6-yl)-2,5- dihydro-1H-pyrrol-3-yl) quinazolin-2-ylamino)piperidine- 1-carboxylate (prepared using E with 3-(2-chloroquinazolin-4-yl)- 4-(4H-thieno[3,2-b]pyrrol-6-yl)- 1H-pyrrole-2,5-dione (Preparation #F.2), TEA, and 4-amino-1-Boc- piperidine) | 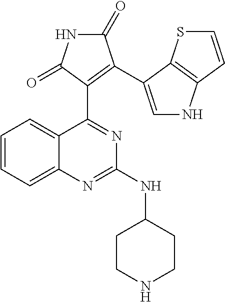 | G.1.8 | 1.52 (c) | 445 |
| tert-Butyl 2-((4-(2,5-dioxo-4-(4H- thieno[3,2-b]pyrrol-6-yl)-2,5- dihydro-1H-pyrrol-3- yl)quinazolin-2- ylamino)methyl)piperidine-1- carboxylate (prepared using E with 3-(2-chloroquinazolin-4-yl)- 4-(4H-thieno[3,2-b]pyrrol-6-yl)- 1H-pyrrole-2,5-dione (Preparation #F.2), TEA, and 1- Boc-2-aminomethylpiperidine) | 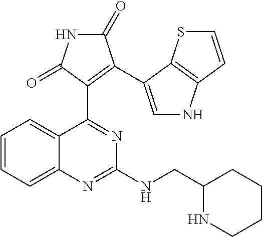 | G.1.9 | 1.70 (c) | 459 |
| tert-Butyl 1-(4-(2,5-dioxo-4-(6H- thieno[2,3-b]pyrrol-4-yl)-2,5- dihydro-1H-pyrrol-3- yl)quinazolin-2-yl)-4- methylpiperidin-4-ylcarbamate (prepared using E with 3-(2- chloroquinazolin-4-yl)-4-(6H- thieno[2,3-b]pyrrol-4-yl)-1H- pyrrole-2,5-dione (Preparation #F.1),TEA, and tert-butyl 4- methylpiperidin-4-ylcarbamate) | 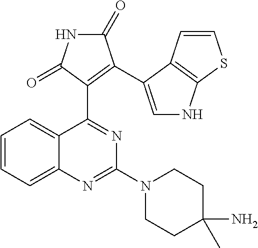 | G.1.10 | 1.74 (c) | 459 |
| tert-Butyl 4-(3-(2,5-dioxo-4-(6H- thieno[2,3-b]pyrrol-4-yl)-2,5- dihydro-1H-pyrrol-3-yl)-1H- indazol-1-yl)piperidine-1- carboxylate (prepared using F with tert-butyl 4-(2-ethoxy-2- oxoacetyl)-6H-thieno[2,3- b]pyrrole-6-carboxylate (Preparation #C.1) and tert-butyl 4-(3-(2-amino-2-oxoethyl)-1H- indazol-1-yl)piperidine-1- carboxylate (Preparation #J.l)) | 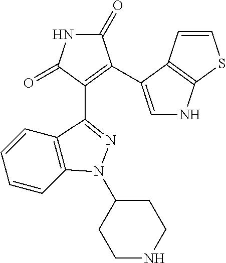 | G.1.11 | 1.71 (c) | 418 |
| tert-Butyl 4-((3-(2,5-dioxo-4-(6H- thieno[2,3-b]pyrrol-4-yl)-2,5- dihydro-1H-pyrrol-3-yl)-1H- indazol-1-yl)methyl)piperidine-1- carboxylate (prepared using F with tert-butyl 4-(2-ethoxy-2- oxoacetyl)-6H-thieno[2,3- b]pyrrole-6-carboxylate (Preparation #C.1) and tert-butyl 4-((3-(2-amino-2-oxoethyl)-1H- indazol-1-yl)methyl)piperidine-1- carboxylate (prepared using J with tert-butyl 4-((3-(2-ethoxy-2- oxoethyl)-1H-indazol-1- yl)methyl)piperidine-1- carboxylate (prepared using with I with N-Boc-4-(4- toluenesulfonyloxymethyl) piperidine (AstaTech), Cs₂CO₃, and ethyl 2-(1H-indazol-3-yl)acetate J. Med. Chem. 1992, 35, 2155- 2162.)))) | 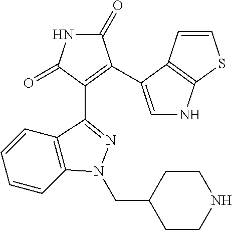 | G.1.12 | 1.63 (c) | 432 |
| tert-Butyl 7-(4-(2,5-dioxo-4-(6H- thieno[2,3-b]pyrrol-4-yl)-2,5- dihydro-1H-pyrrol-3- yl)quinazolin-2-yl)-2,7- diazaspiro[3.5]nonane-2- carboxylate (prepared using E with 3-(2-chloroquinazolin-4-yl)- 4-(6H-thieno[2,3-b]pyrrol-4-yl)- 1H-pyrrole-2,5-dione (Preparation #F.1), TEA, and 2-(tert- butoxycarbonyl)-2,7-diazaspiro [3.5]nonane) | 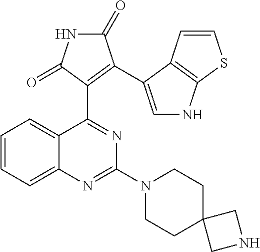 | G.1.13 | 1.64 (c) | 471 |
| tert-Butyl (1-(4-(2,5-dioxo-4-(6H- thieno[2,3-b]pyrrol-4-yl)-2,5- dihydro-1H-pyrrol-3- yl)quinazolin-2-yl)pyrrolidin-3- yl)methyl(methyl)carbamate (prepared using E with 3-(2- chloroquinazolin-4-yl)-4-(6H- thieno[2,3-b]pyrrol-4-yl)-1H- pyrrole-2,5-dione (Preparation #F.l), TEA, and 3-N-Boc-3-N- methyl-3-aminomethyl- pyrrolidine (Atlantic SciTech)) | 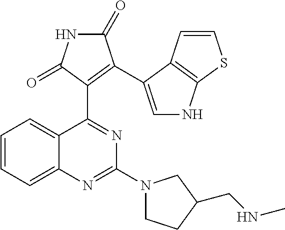 | G.1.14 | 1.63 (c) | 459 |
| tert-Butyl (1-(4-(2,5-dioxo-4-(6H- thieno[2,3-b]pyrrol-4-yl)-2,5- dihydro-1H-pyrrol-3- yl)quinazolin-2-yl)piperidin-4- yl)methyl(methyl)carbamate (prepared using E with 3-(2- chloroquinazolin-4-yl)-4-(6H- thieno[2,3-b]pyrrol-4-yl)-1H- pyrrole-2,5-dione (Preparation #F.l), TEA, and tert-butyl methyl(4- piperidinylmethyl)carbamate (ChemBridge)) | 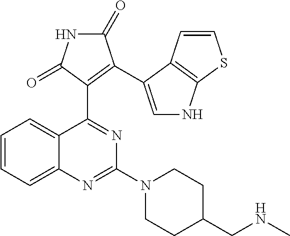 | G.1.15 | 1.63 (c) | 473 |
| tert-Butyl 4-((3-(2,5-dioxo-4-(6H- thieno[2,3-b]pyrrol-4-yl)-2,5- dihydro-1H-pyrrol-3-yl)-2H- indazol-2-yl)methyl)piperidine-1- carboxylate (prepared using F with 3-(2-chloroquinazolin-4-yl)- 4-(6H-thieno[2,3-b]pyrrol-4-yl)- 1H-pyrrole-2,5-dione (Preparation #F.1) and tert-butyl 4-((3-(2- amino-2-oxoethyl)-2H-indazol-2- yl)methyl)piperidine-1- carboxylate (prepared using J with tert-butyl 4-((3-(2-ethoxy-2- oxoethyl)-2H-indazol-2- yl)methyl)piperidine-1- carboxylate (prepared using I with ethyl 2-(1H-indazol-3-yl)acetate (J. Med. Chem. 1992, 35, 2155- 2162.), Cs₂CO₃, and tert-butyl 4- (tosyloxymethyl)piperidine-1- carboxylate (AstaTech)))) | 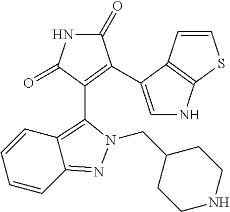 | G.1.16 | 1.68 (c) | 432 |
| cis-tert-Butyl 4-((3-(2,5-dioxo-4- (6H-thieno[2,3-b]pyrrol-4-yl)-2,5- dihydro-1H-pyrrol-3-yl)-1H- indazol-1-yl)methyl)-3- fluoropiperidine-1-carboxylate (prepared using F with tert-butyl 4-(2-methoxy-2-oxoacetyl)-6H- thieno[2,3-b]pyrrole-6- carboxylate (prepared using C with tert-butyl 4-(2-methoxy-2- oxoethyl)-6H-thieno[2,3- b]pyrrole-6-carboxylate (prepared using B with tert-butyl 3- bromothiophen-2-ylcarbamate (Preparation #A.1), K₂CO₃, methyl 4-bromocrotonate)) and cis-tert-butyl 4-((3-(2-amino-2- oxoethyl)-1H-indazol-1- yl)methyl)-3-fluoropiperidine-1- carboxylate (prepared using J with cis-tert-butyl 4-((3-(2- ethoxy-2-oxoethyl)-1H-indazol-1- yl)methyl)-3-fluoropiperidine-1- carboxylate (prepared using I with ethyl 2-(1H-indazol-3-yl)acetate (J. Med. Chem. 1992, 35, 2155- 2162.), Cs₂CO₃, and cis-tert-butyl 3-fluoro-4- ((methylsulfonyloxy)methyl) piperidine-1-carboxylate (prepared using H with MsCl, TEA, and cis- tert-butyl 3-fluoro-4- (hydroxymethyl)piperidine-1- carboxylate (prepared using R with Boc₂O, TEA and (3- fluoropiperidin-4-yl)methanol hydrochloride (WO2006069287A1.)))))) | 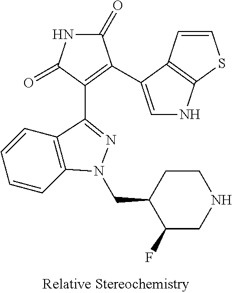 | G.1.17 | 1.58 (c) | 450 |
| (tert-Butyl 4-(4-(2,5-dioxo-4-(6H- thieno[2,3-b]pyrrol-4-yl)-2,5- dihydro-1H-pyrrol-3- yl)thieno[2,3-d]pyrimidin-2- yl)piperazine-1-carboxylate (prepared using F from tert-butyl 4-(2-methoxy-2-oxoacetyl)-6H- thieno[2,3-b]pyrrole-6- carboxylate (prepared using C with tert-butyl 4-(2-methoxy-2- oxoethyl)-6H-thieno[2,3- b]pyrrole-6-carboxylate (prepared using B with tert-butyl 3- bromothiophen-2-ylcarbamate (Preparation #A.1), K₂CO₃, methyl 4-bromocrotonate)) and tert-butyl 4-(4-(2-amino-2- oxoethyl)thieno[2,3-d]pyrimidin- 2-yl)piperazine-1-carboxylate (prepared using E from tert-butyl piperazine-1-carboxylate, TEA, and 2-(2-chlorothieno[2,3- d]pyrimidin-4-yl)acetamide (prepared using D from 2,4- dichlorothieno[2,3-d]pyrimidine (ArkPharm Inc.)))) | 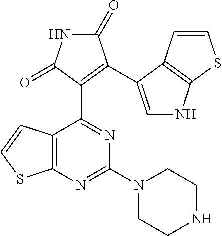 | G.1.18 | 1.54 (c) | 437 |
| tert-Butyl 5-(4-(2,5-dioxo-4-(6H- thieno[2,3-b]pyrrol-4-yl)-2,5- dihydro-1H-pyrrol-3- yl)quinazolin-2-yl)octahydro-1H- pyrrolo[3,2-c]pyridine-1- carboxylate (prepared using E from 3-(2-chloroquinazolin-4-yl)- 4-(6H-thieno[2,3-b]pyrrol-4-yl)- 1H-pyrrole-2,5-dione (Preparation #F.1), TEA, and tert-butyl octahydro-1H-pyrrolo[3,2- c]pyridine-1-carboxylate (Bioorg. Med. Chem. Lett. 2005, 15, 977- 982)) | 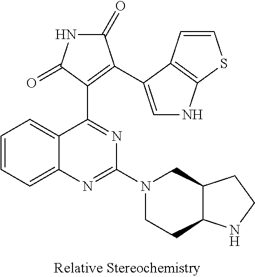 | G.1.19 | 1.65 (c) | 471 |

### General Procedure H

### Formation of a Sulfonate from an Alcohol

To a solution of an alcohol (1 equiv) in an organic solvent such as DCM and optionally a base such as TEA, DIEA, or pyridine (1-5 equiv, preferably TEA, 1-1.5 equiv) under a nitrogen atmosphere is added a sulfonyl halide such as MsCl or p-toluenesulfonyl chloride (1-2 equiv, preferably MsCl, 1-1.3 equiv) at about 0° C. to ambient temperature (preferably 0° C.). The solution is stirred for about 1-24 h (preferably 1-5 h) at ambient temperature. The reaction mixture is optionally washed with water, saturated aqueous NaHCO₃ and/or brine and the organic layer is dried over Na₂SO₄ or MgSO₄ (preferably Na₂SO₄), filtered, and concentrated under reduced pressure.

### Illustration of General Procedure H

### Preparation #H.1

### tert-Butyl 4-(methylsulfonyloxy)piperidine-1-carboxylate

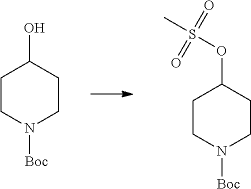

MsCl (2.26 mL, 29.0 mmol) was added dropwise to a solution of tert-butyl 4-hydroxypiperidine-1-carboxylate (5.30 g, 26.3 mmol), TEA (4.77 mL, 34.2 mmol) and DCM (50 mL) under a nitrogen atmosphere at about 0° C. After about 10 min, the ice bath was removed and the reaction mixture was left to stir at ambient temperature for about 4 h. Saturated aqueous NaHCO₃ (50 mL) and water (50 mL) were added. The layers were separated and the organic layer was washed with water (50 mL) and brine (50 mL). The aqueous layers were extracted with DCM (50 mL). The combined organic layers were dried over Na₂SO₄, filtered, and concentrated to afford tert-butyl 4-(methylsulfonyloxy)piperidine-1-carboxylate (7.36 g, 100% yield): ¹H NMR (400 MHz, DMSO-d₆) δ 4.86-4.78 (m, 1H), 3.64-3.55 (m, 2H), 3.20 (s, 3H), 3.20-3.12 (m, 2H), 1.95-1.86 (m, 2H), 1.66-1.55 (m, 2H), 1.40 (s, 9H).

### General Procedure I

### N-Alkylation

To a solution of an optionally substituted indazole, pyrazole, indole, imidazole or amine (1-2 equiv, preferably 1 equiv) in an organic solvent such as THF, 1,4-dioxane, MeCN, DMF, or DMSO (preferably 1,4-dioxane) under a nitrogen atmosphere at about 0° C. to ambient temperature (preferably ambient temperature) is added an alkyl, cycloalkyl, heterocyclyl, aryl, or heteroaryl halide or sulfonate (prepared using General Procedure H, General Procedure P, or commercially available)(1-5 equiv, preferably 3 equiv), optionally as a solution in an appropriate organic solvent such as 1,4-dioxane or THF (preferably 1,4-dioxane). A base such as Cs₂CO₃, K₂CO₃, TEA, DIEA, NaH, or pyridine (0.9-10 equiv, preferably Cs₂CO₃, 3-5 equiv) is added. Alternatively, an alcohol (1-3 equiv, preferably 1 equiv), a trialkyl- or triarylphosphine (1-3.5 equiv, preferably triphenylphosphine, 1.1 equiv) and diisopropyl azodicarboxylate or diethyl azodicarboxylate (1-3.5 equiv, preferably diisopropyl azodicarboxylate, 1.1 equiv) may be added neat or as a solution in an organic solvent such as THF. The reaction mixture is stirred at about 0-120° C. (preferably 80° C.) for about 16-96 h (preferably 16-86 h). The reaction mixture is partitioned between an organic solvent such as EtOAc or DCM and water, saturated aqueous NaHCO₃, and/or brine. The organic layer is dried over Na₂SO₄ or MgSO₄, filtered, and concentrated.

### Illustration of General Procedure I

### Preparation #1.1

### tert-Butyl 4-(3-(2-ethoxy-2-oxoethyl)-1H-indazol-1-yl)piperidine-1-carboxylate

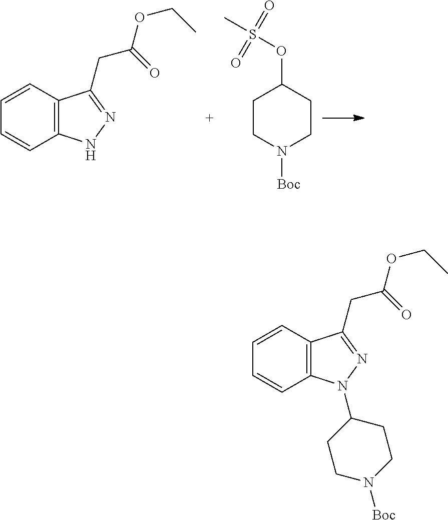

A mixture of ethyl 2-(1H-indazol-3-yl)acetate (1.71 g, 8.37 mmol, J. Med. Chem. 1992, 35, 2155-2162.), tert-butyl 4-(methylsulfonyloxy)piperidine-1-carboxylate (7.02 g, 25.1 mmol, Preparation #H.1), Cs₂CO₃ (8.73 g, 26.8 mmol), and 1,4-dioxane (17 mL) under a nitrogen atmosphere was warmed to about 80° C. After about 86 h, the mixture was allowed to cool to ambient temperature. Water (150 mL) was added and the mixture was extracted with EtOAc (2×75 mL). The combined organic layers were washed with brine (50 mL) and then dried over Na₂SO₄, filtered, and concentrated. The crude material was purified using flash column chromatography on silica gel eluting with a gradient of 0-50% EtOAc in heptane to give tert-butyl 4-(3-(2-ethoxy-2-oxoethyl)-1H-indazol-1-yl)piperidine-1-carboxylate (0.780 g, 24% yield): LC/MS (Table 1, Method a) Rₜ=2.57 min; MS m/z: 388 (M+H)⁺.

### General Procedure J

### Amino-De-Alkoxylation of a Carboxylic Ester with NH₃

Ammonia (20-100 equiv), optionally as a solution in a solvent such as MeOH or EtOH (preferably 7 N NH₃ in MeOH, 20-100 equiv), is added to a carboxylic ester (1 equiv), optionally as a solution in an organic solvent such as MeOH or EtOH. Alternatively, ammonium hydroxide (10-200 equiv) may be used. The solution is stirred at ambient temperature to about 80° C. (preferably 45-70° C.) for about 14-192 h (preferably 16-120 h). Optionally, the solvent is removed under reduced pressure and the residue is resubmitted to the reaction conditions. The volatiles are removed under reduced pressure. Optionally, an organic solvent such as DCM is added to induce precipitation and the material may be collected by filtration. Optionally, the solvent of the mother liquor is removed under reduced pressure and the residue is resubmitted to the reaction conditions. Optionally, an organic solvent such as DCM is added to induce precipitation and the material may be collected by filtration.

### Illustration of General Procedure J

### Preparation #J.1

### tert-Butyl 4-(3-(2-amino-2-oxoethyl)-1H-indazol-1-yl)piperidine-1-carboxylate

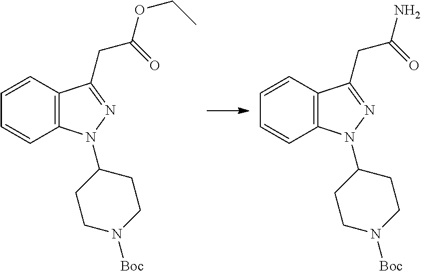

Ammonia (7 N in MeOH, 7.4 mL, 52 mmol) was added to tert-butyl 4-(3-(2-ethoxy-2-oxoethyl)-1H-indazol-1-yl)piperidine-1-carboxylate (0.780 g, 2.01 mmol, Preparation #1.1). The reaction vessel was sealed and the solution was warmed to about 45° C. for about 24 h and then at about 50° C. for about 15 h. After cooling to ambient temperature, the volatiles were removed under reduced pressure. The crude material was purified using flash column chromatography on silica gel eluting with a gradient of 2-10% MeOH in DCM to give tert-butyl 4-(3-(2-amino-2-oxoethyl)-1H-indazol-1-yl)piperidine-1-carboxylate (0.720 g, 100% yield): LC/MS (Table 1, Method a) Rₜ=1.95 min; MS m/z: 359 (M+H)⁺.

### Preparation #J.2

### 2-(5-Fluoro-2-(4-methylpiperazin-1-yl)quinazolin-4-yl)acetamide

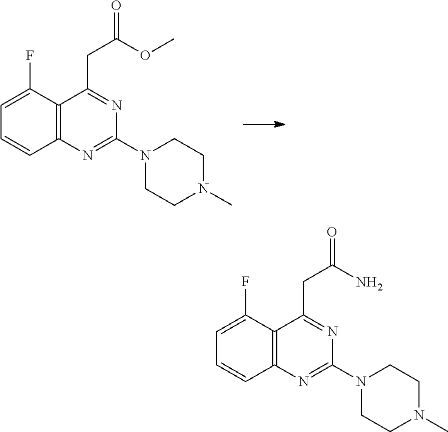

A solution of methyl 2-(5-fluoro-2-(4-methylpiperazin-1-yl)quinazolin-4-yl)acetate (1.26 g, 3.96 mmol, Preparation #M.1) and NH₃ (7 N in MeOH, 56.5 mL, 396 mmol) was divided into 6 sealed 40 mL reaction vials and heated to about 60° C. for about 16 h. The reaction was cooled to ambient temperature, concentrated under reduced pressure, and triturated with DCM. The resulting solid was collected by filtration and dried under vacuum (0.339 g). The mother liquor was concentrated under reduced pressure and the residue was dissolved in NH₃ (7 N in MeOH, 44.0 mL, 308 mmol), divided between four sealed 40 mL reaction vials and heated to about 60° C. for about 16 h. The reaction was cooled to ambient temperature, concentrated under reduced pressure, and triturated with DCM. The resulting solid was collected by filtration and dried under vacuum (0.179 g). The mother liquor was set aside. A solution of methyl 2-(5-fluoro-2-(4-methylpiperazin-1-yl)quinazolin-4-yl)acetate (1.66 g, 5.21 mmol) and NH₃ (7 N in MeOH, 55.9 mL, 391 mmol) was divided into eight sealed 40 mL reaction vials and heated to about 60° C. for about 16 h. The reaction was cooled to ambient temperature, concentrated under reduced pressure, and triturated with DCM. The resulting solid was collected by filtration and dried under vacuum (0.276 g). The mother liquor was concentrated under reduced pressure and the residue was dissolved in NH₃ (7 N in MeOH, 47.0 mL, 329 mmol), divided between five sealed 40 mL reaction vials and heated to about 60° C. for about 48 h. The reaction was cooled to ambient temperature, concentrated under reduced pressure, and triturated with DCM. The resulting solid was collected by filtration and dried under vacuum (0.371 g). The mother liquor was combined with that kept aside earlier and concentrated under reduced pressure. The residue was dissolved in NH₃ (7 N in MeOH, 55.0 mL, 385 mmol), divided into six sealed 40 mL reaction vials and heated to about 58° C. for about 48 h. The reaction was cooled to ambient temperature, concentrated under reduced pressure, and triturated with DCM. The resulting solid was collected by filtration and dried under vacuum (0.284 g). The solids were combined to obtain 2-(5-fluoro-2-(4-methylpiperazin-1-yl)quinazolin-4-yl)acetamide (1.45 g, 52% yield): LC/MS (Table 1, Method a) Rₜ=1.32 min; MS m/z: 304 (M+H)⁺.

### Preparation #J.3

### (R)-2-(5-Fluoro-2-(hexahydropyrrolo[1,2-a]pyrazin-2(1H)-yl)quinazolin-4-yl)acetamide

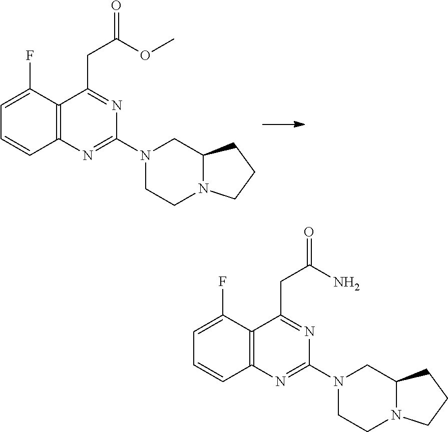

A solution of (R)-methyl 2-(5-fluoro-2-(hexahydropyrrolo[1,2-a]pyrazin-2(1H)-yl)quinazolin-4-yl)acetate (1.11 g, 3.22 mmol, Preparation #M.2) in NH₃ (7 N in MeOH, 36.8 mL, 258 mmol) was divided into four sealed 40 mL reaction vials and heated to about 60° C. for about 72 h. Reaction was then cooled to ambient temperature. The material was combined and then concentrated under reduced pressure. The residue was dissolved in NH₃ (7 N in MeOH, 36.8 mL, 258 mmol) and divided into four sealed 40 mL reaction vials and heated to about 60° C. for about 48 h. Reaction was cooled to ambient temperature and then the combined material concentrated under reduced pressure. The crude material was purified using flash column chromatography on silica gel eluting with a gradient of 30-85% (1% (7 N NH₃ in MeOH) in 10% MeOH/DCM) in DCM. The product containing fractions were combined and concentrated under reduced pressure to afford (R)-2-(5-fluoro-2-(hexahydropyrrolo[1,2-a]pyrazin-2(1H)-yl)quinazolin-4-yl)acetamide (0.470 g, 44% yield): LC/MS (Table 1, Method c) Rₜ=1.40 min; MS m/z: 330 (M+H)⁺.

### General Procedure K

### Reductive Amination of an Aldehyde or Ketone and an Amine

To a solution of an amine (1 equiv) and an aldehyde or a ketone (0.5-20 equiv, preferably 1-5 equiv), optionally treated with titanium tetraisopropoxide (1-1.5 equiv, preferably 1.3 equiv) for about 0.5-3 h (preferably 1 h), in an organic solvent such as MeCN, DCM, THF, 1,4-dioxane, and/or EtOH (preferably MeCN) is added a reducing agent such as Na(AcO)₃BH or NaBH₃CN (1-5 equiv, preferably NaBH₃CN, 1-2.5 equiv). After about 1-72 h (preferably 2-24 h), the reaction mixture is either treated with water, MeOH, or EtOH with subsequent filtration or treated with an aqueous solution such as water, saturated aqueous NaHCO₃, and/or brine and then extracted with an organic solvent such as DCM or EtOAc. The organics were dried over Na₂SO₄ or MgSO₄ (preferably Na₂SO₄), filtered, and concentrated under reduced pressure.

### Illustration of General Procedure K

### Preparation #K.1

### 2-(1-(1-Methylpiperidin-4-yl)-1H-indazol-3-yl)acetamide

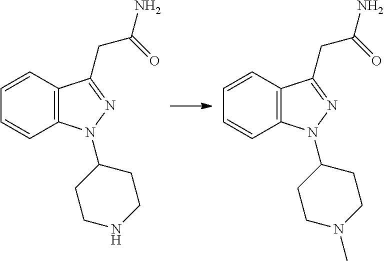

Formaldehyde (37 wt % in water, 0.360 mL, 4.84 mmol) was added to a mixture of 2-(1-(piperidin-4-yl)-1H-indazol-3-yl)acetamide hydrochloride (0.361 g, 0.967 mmol, prepared using G with tert-butyl 4-(3-(2-amino-2-oxoethyl)-1H-indazol-1-yl)piperidine-1-carboxylate (Preparation #J.1)) and MeCN (3.2 mL). The reaction vessel was placed in an ambient temperature water bath and then NaBH₃CN (0.134 g, 2.13 mmol) was added portionwise. After about 2 h, saturated aqueous NaHCO₃ (5 mL) was added and the solution was extracted with DCM (2×20 mL). The combined organics were concentrated under reduced pressure. The residue was purified using silica gel chromatography eluting with a gradient of 0.5% (7 N NH₃ in MeOH) in 5% MeOH in DCM to 1% (7 N NH₃ in MeOH) in 10% MeOH in DCM to give 2-(1-(1-methylpiperidin-4-yl)-1H-indazol-3-yl)acetamide (0.100 g, 38% yield): LC/MS (Table 1, Method a) Rₜ=0.99 min; MS m/z: 273 (M+H)⁺.

### General Procedure L

### Displacement of a Heteroaryl Halide or Heteroaryl Sulfone with an Acetate Equivalent

A solution of diethyl malonate or ethyl acetoacetate (preferably ethyl acetoacetate, 2 equiv) and an organic solvent such as Et₂O, THF, 1,4-dioxane, DCM, DCE, DMF, or toluene (preferably THF) is added dropwise to a mixture of a base such as NaH, NaOt-Bu, or KOt-Bu (1-2 equiv, preferably NaH, 1.2-1.5 equiv) and an organic solvent such as Et₂O, THF, 1,4-dioxane, DCM, DCE, DMF, or toluene (preferably THF) at −10-25° C. (preferably 0° C.) under a nitrogen atmosphere. Alternatively, the order of addition may be reversed. After about 5-120 min, the volatiles are optionally removed under reduced pressure. The heteroaryl halide or heteroaryl sulfone and an organic solvent such as Et₂O, THF, 1,4-dioxane, DCM, DCE, DMF, or toluene (preferably toluene) are added. The resulting slurry is warmed to 40-110° C. (preferably 110° C.) for about 0.5-24 h (preferably 3 h). Alternatively, an acetate anion may be prepared using a base such as LiHMDS, LDA, n-BuLi, or t-BuLi (2.1-5 equiv, preferably LiHMDS, 2.7 equiv) with an appropriate alkyl acetate (2-5 equiv, preferably tert-butyl acetate, 2.5 equiv) in an organic solvent such as Et₂O, THF, 1,4-dioxane, DCM, DCE, DMF, or toluene (preferably toluene) at about −78-0° C. (preferably −10° C.). A palladium source such as Pd₂(dba)₃ or Pd(PPh₃)₄ (1-10 mol %, preferably Pd₂(dba)₃, 3 mol %), is added. A ligand such as (2′-dicyclohexylphosphanyl-biphenyl-2-yl)-dimethylamine (2-20 mol %, preferably 6 mol %) is optionally added. All materials are degassed as described in Degassing Methods. A heteroaryl halide or heteroaryl sulfone (1 equiv) is added. The mixture may be stirred at about −78-110° C. (preferably ambient temperature) for about 0.5-24 h (preferably 0.5-2 h). Alternatively, a heteroaryl halide or heteroaryl sulfone (1 equiv), a palladium(II) source such as Pd(OAc)₂ (2-10 mol %, preferably 5 mol %), and a ligand such as 2-dicyclohexylphosphino-2′,6′-dimethoxybiphenyl (2-10 mol %, preferably 10 mol %) are degassed as described in Degassing Methods and an organozinc reagent (2-10 equiv, preferably 3-5 equiv), either commercially available or generated prior to use using zinc with or without activation and/or sonification with an alkyl α-haloacetate in an organic solvent such as Et₂O, THF, 1,4-dioxane, DCM, DCE, DMF, or toluene (preferably THF). The mixture may be stirred at about 25-110° C. (preferably 45-60° C.) for about 0.5-120 h (preferably 6-40 h). Optionally, the solvent may be removed under reduced pressure, a solvent or mixture of solvents such as DCM, EtOAc, aqueous NaHCO₃, aqueous NH₄Cl, water, and/or brine is added, the mixture is filtered, and/or the mixture is partioned. The organic layer is then optionally dried over Na₂SO₄ or MgSO₄, filtered, and concentrated under reduced pressure. Optionally, the residue may be submitted to General Procedure E.

### Illustration of General Procedure L

### Preparation #L.1

### Ethyl 2-(6-fluoro-2-(4-methylpiperazin-1-yl)quinazolin-4-acetate

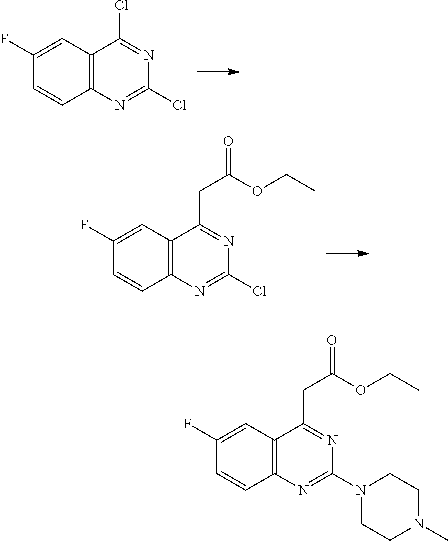

An oven dried flask charged with NaH (60% dispersion in mineral oil, 0.055 g, 1.4 mmol) in THF (1.10 mL) under a nitrogen atmosphere was cooled to about 0° C. To the suspension was added ethyl 3-oxobutanoate (0.233 mL, 1.84 mmol) dropwise. Upon completion of addition, the reaction was stirred for about 5 min at about 0° C. and then concentrated under reduced pressure to give a white solid. To this solid was added toluene (9.17 mL) and 2,4-dichloro-6-fluoroquinazoline (0.200 g, 0.922 mmol, BetaPharma). The reaction mixture was stirred at about reflux for about 3 h and then concentrated under reduced pressure to afford ethyl 2-(2-chloro-6-fluoroquinazolin-4-yl)acetate: LC/MS (Table 1, Method e) Rₜ=0.73 min; MS m/z: 269 (M+H)⁺. To a solution of ethyl 2-(2-chloro-6-fluoroquinazolin-4-yl) acetate (0.248 g, 0.922 mmol) in DMF (1.84 mL) under a nitrogen atmosphere was added 1-methylpiperazine (0.308 mL, 2.77 mmol). The reaction was stirred at ambient temperature for about 16 h. Brine (20 mL) was added and the mixture was extracted with EtOAc (2×40 mL). The combined organic layers were dried over MgSO₄, filtered and concentrated under reduced pressure. The residue was purified by flash column chromatography on silica gel eluting with a gradient of 0-5% MeOH in DCM to afford ethyl 2-(6-fluoro-2-(4-methylpiperazin-1-yl)quinazolin-4-yl)acetate (0.225 g, 71% yield): LC/MS (Table 1, Method c) Rₜ=1.68 min; MS m/z: 333 (M+H)⁺.

### Preparation #L.2

### Preparation of tert-butyl 2-(2-chloro-5-fluoroquinazolin-4-yl)acetate

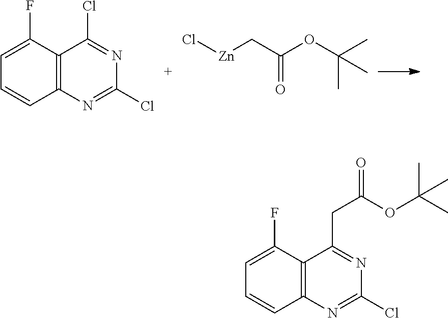

To a solution of 2,4-dichloro-5-fluoroquinazoline (1.88 g, 8.64 mmol, Accela ChemBio) in THF (21.6 mL) under a nitrogen atmosphere was added (2-tert-butoxy-2-oxoethyl)zinc(II) chloride (0.5 M solution in Et₂O, 51.8 mL, 25.9 mmol, Rieke Metals). Nitrogen was bubbled through the solution for about 20 min. Palladium(II) acetate (0.097 g, 0.432 mmol) and 2-dicyclohexylphosphino-2′,6′-dimethoxybiphenyl (0.355 g, 0.864 mmol) were added to the reaction, each in one portion, and nitrogen was bubbled through the solution for about 5 min. The reaction solution was warmed to about 45° C. for about 24 h. After allowing to cool to ambient temperature, (2-tert-butoxy-2-oxoethyl)zinc(II) chloride (0.5 M solution in Et₂O, 20.0 mL, 10.0 mmol, Rieke Metals), Pd(OAc)₂ (0.097 g, 0.432 mmol), and 2-dicyclohexylphosphino-2′,6′-dimethoxybiphenyl (0.355 g, 0.864 mmol) were added respectively. Nitrogen was bubbled through the solution for about 20 min and the reaction was warmed to about 48° C. for about 16 h. After cooling to ambient temperature, saturated aqueous NaHCO₃ (90 mL) and EtOAc (100 mL) were added. The solid was removed by filtration. The layers were separated and the aqueous phase was extracted with EtOAc (100 mL). The combined organics were dried over MgSO₄, filtered, and concentrated under reduced pressure. The residue was purified by flash column chromatography on silica gel eluting with a gradient of 0-30% EtOAc in heptane. The product containing fractions were concentrated under reduced pressure and the residue was triturated with EtOAc. The solid was removed by filtration and the organics were concentrated under reduced pressure to afford tert-butyl 2-(2-chloro-5-fluoroquinazolin-4-yl)acetate (1.14 g, 45% yield): LC/MS (Table 1, method c) Rₜ=2.64 min; MS m/z: 297 (M+H)⁺.

### Preparation #L.3

### tert-Butyl 2-(3-chloro-7-fluoroisoquinolin-1-yl)acetate

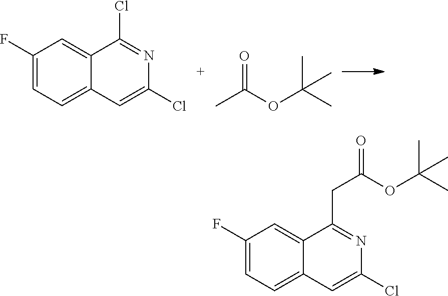

LiHMDS (2 M solution in heptane/THF/ethylbenzene, 12.5 mL, 25.0 mmol) was added to a degassed solution of Pd₂(dba)₃ (0.28 g, 0.30 mmol) and (2′-dicyclohexylphosphanyl-biphenyl-2-yl)-dimethylamine (0.25 g, 0.64 mmol) in toluene (4 mL). The mixture was stirred at ambient temperature for about 10 min. The mixture was cooled to about −10° C. and tert-butyl acetate (3.10 mL, 23.2 mmol) was added. The resulting mixture was stirred for about 10 min at about −10° C. 1,3-Dichloro-7-fluoroisoquinoline (2.00 g, 9.26 mmol, CombiBlocks) was added in one portion and the resulting mixture was allowed to warm to ambient temperature and was then stirred at ambient temperature for about 30 min. The reaction mixture was filtered through a pad of Celite®, washing with 2% MeOH in EtOAc. The filtrate was evaporated under reduced pressure. The resulting dark oil was diluted with DCM. Et₂O was added and the resulting precipitate was filtered off. The filtrate was collected, concentrated under reduced pressure, and purified by flash column chromatography on silica gel eluting with a gradient of 0-50% EtOAc in heptane to give tert-butyl 2-(3-chloro-7-fluoroisoquinolin-1-yl)acetate (0.855 g, 31%): LC/MS (Table 1, method b) Rₜ=1.65 min; MS m/z: 296 (M+H)⁺.

### General Procedure M

### Transesterification of a Carboxylic Ester

To a solution or mixture of a carboxylic ester (1 equiv) and an alcohol such as MeOH or EtOH (preferably MeOH) with an optional co-solvent such as 1,4-dioxane or THF at −10-25° C. (preferably 0-25° C.) is added HCl (3 equiv to saturation, preferably 5 equiv) by bubbling HCl gas through the solution or by adding a solution of HCl in an organic solvent such as 1,4-dioxane or Et₂O (preferably 4 M solution of HCl in 1,4-dioxane). The reaction mixture is stirred at about ambient temperature to 100° C. (preferably ambient temperature) for about 0.5-72 h (preferably 48-72 h). The volatiles are removed under reduced pressure. Optionally, the residue is partitioned between a basic aqueous solution such as aqueous NaHCO₃ and an organic solvent such as DCM or EtOAc (preferably DCM). The aqueous phase is optionally extracted with additional portions of an organic solvent. The combined organics are optionally dried over Na₂SO₄ or MgSO₄, filtered, and concentrated under reduced pressure.

### Illustrations of General Procedure M

### Preparation #M.1

### Preparation of methyl 2-(5-fluoro-2-(4-methylpiperazin-1-yl)quinazolin-4-yl)acetate

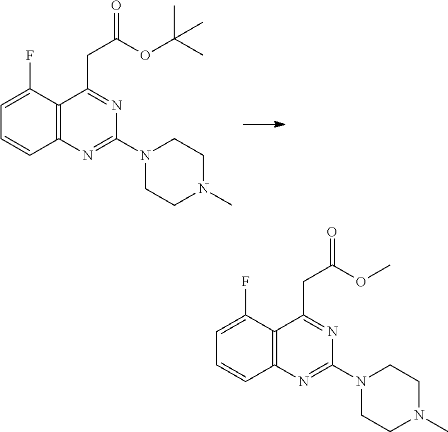

To a solution of tert-butyl 2-(5-fluoro-2-(4-methylpiperazin-1-yl)quinazolin-4-yl)acetate (1.57 g, 4.36 mmol, Preparation #E.2) in MeOH (8.7 mL) under a nitrogen atmosphere at about 0° C. was added HCl (4 M solution in 1,4-dioxane, 5.44 mL, 21.8 mmol). The reaction was stirred at ambient temperature for about 48 h. The solution was concentrated under reduced pressure. Saturated aqueous NaHCO₃ (25 mL) and water (25 mL) were added. The aqueous solution was extracted with DCM (2×70 mL). The combined organics were dried over MgSO₄, filtered, and concentrated under reduced pressure to afford methyl 2-(5-fluoro-2-(4-methylpiperazin-1-yl)quinazolin-4-yl)acetate (1.26 g, 91% yield): LC/MS (Table 1, method c) Rₜ=1.60 min; MS m/z: 319 (M+H)⁺.

### Preparation #M.2

### (R)-Methyl 2-(5-fluoro-2-(hexahydropyrrolo[1,2-a]pyrazin-2(1H)-yl)quinazolin-4-yl)acetate

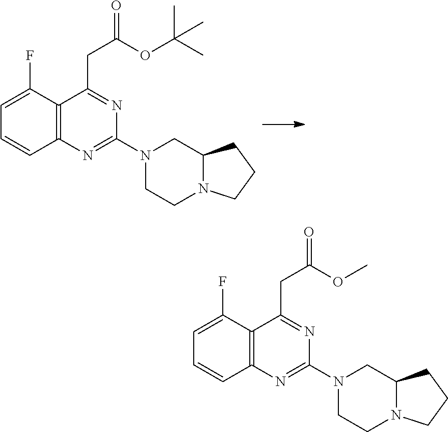

To a solution of (R)-tert-butyl 2-(5-fluoro-2-(hexahydropyrrolo[1,2-a]pyrazin-2(1H)-yl)quinazolin-4-yl)acetate (1.43 g, 3.22 mmol, Preparation #E.4) in MeOH (6.43 mL) under a nitrogen atmosphere at about 0° C. was added HCl (4 M in 1,4-dioxane, 4.02 mL, 16.1 mmol). The reaction was then warmed to ambient temperature and stirred for about 72 h. The reaction solution was concentrated under reduced pressure. Saturated aqueous NaHCO₃ (75 mL) and water (75 mL) were added to the residue. The mixture was extracted from the aqueous layer using DCM (2×200 mL). The combined organics were dried over MgSO₄, filtered and concentrated under reduced pressure to afford (R)-methyl 2-(5-fluoro-2-(hexahydropyrrolo[1,2-a]pyrazin-2(1H)-yl)quinazolin-4-yl)acetate (1.24 g, 100% yield): LC/MS (Table 1, Method c) Rₜ=1.69 min; MS m/z: 345 (M+H)⁺.

### General Procedure N

### Deprotonation of an Imidazole with an Electrophilic Quench

To a substituted imidazole (1 equiv) in an organic solvent such as THF, Et₂O, or 1,4-dioxane (preferably THF) under a nitrogen atmosphere at about −100-0° C. (preferably −78° C.) is added a base such as n-BuLi, t-BuLi, sec-BuLi, LiHMDS, or LDA (0.9-1.5 equiv, preferably n-BuLi, 1.1 equiv). The reaction is optionally allowed to warm to −78° C. to ambient temperature (preferably 0° C.) over 0.25-2 h (preferably 0.5 h) and then re-cooled. An electrophile such as an aldehyde, ketone, alkylhalide, alkylsulfonate, or electrophilic halogen source (1-5 equiv, preferably 3 equiv) is added. The mixture is optionally allowed to warm to about 0° C. to ambient temperature (preferably 0° C.). The reaction mixture is treated with an aqueous quench such as aqueous NaHCO₃, aqueous NH₄Cl, aqueous NaHSO₃, water, or brine (preferably aqueous NaHCO₃). An organic solvent such as EtOAc or DCM (preferably EtOAc) is added. The layers are separated and the aqueous layer is optionally extracted with additional organics. The combined organics are optionally washed with additional aqueous solutions such as water or brine, dried over Na₂SO₄ or MgSO₄, filtered, and concentrated under reduced pressure.

### Illustrations of General Procedure N

### Preparation #N.1

### 1-(7-Benzyl-5,6,7,8-tetrahydroimidazo[1,5-a]pyrazin-3-yl)ethanol

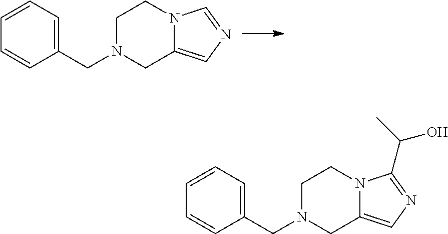

7-Benzyl-5,6,7,8-tetrahydroimidazo[1,5-a]pyrazine (0.650 g, 3.05 mmol, WO 2003076427A1) was added to THF (5 mL) under a nitrogen atmosphere. The solution was cooled to about −78° C. n-BuLi (2.10 mL, 3.35 mmol) was added dropwise via syringe to the solution. The cold bath was allowed to thaw to about 0° C. over about 30 min. The reaction was cooled to about −78° C. A solution of acetaldehyde (0.403 g, 9.14 mmol) in THF (2 mL) was added dropwise via syringe. After completion of addition, the reaction was warmed to about 0° C. and mixed for about 30 min. The reaction was diluted with saturated aqueous NaHCO₃ (30 mL) and EtOAc (50 mL). The layers were separated and the aqueous layer was extracted with EtOAc (50 mL). The combined organics were dried over MgSO₄, filtered, and concentrated under reduced pressure. The crude material was purified by flash column chromatography on silica gel eluting with a gradient of 1-10% MeOH in DCM to afford 1-(7-benzyl-5,6,7,8-tetrahydroimidazo[1,5-a]pyrazin-3-yl)ethanol (0.400 g, 44% yield): LC/MS (Table 1, Method a) Rₜ=1.28 min; MS m/z: 258 (M+H)⁺.

### General Procedure O

### Benzyl Deprotection

To Pd/C or palladium hydroxide on carbon (preferably Pd/C) was added an organic solvent such as MeOH, EtOH, toluene, or THF (preferably MeOH) or solution/mixture of protected amine and an organic solvent. Alternatively, the protected amine, neat or as a solution, may be added to a slurry of the palladium source in an organic solvent Ammonium formate may be added and the mixture warmed to about 30-100° C. (preferably 60° C.) and stirred for (1-48 h, preferably about 18 h). Alternatively, the mixture, optionally with an acid such as acetic acid or HCl, may be stirred or shaken under a hydrogen atmosphere (1 atm-60 psi) for about 1-72 h (preferably 18 h) at ambient temperature to about 50° C. (preferably 50° C.). The reaction mixture is filtered through Celite®. The volatiles are removed under reduced pressure.

### Illustrations of General Procedure O

### Preparation #O.1

### 1-(5,6,7,8-tetrahydroimidazo[1,5-a]pyrazin-3-yl)ethanol hydrochloride

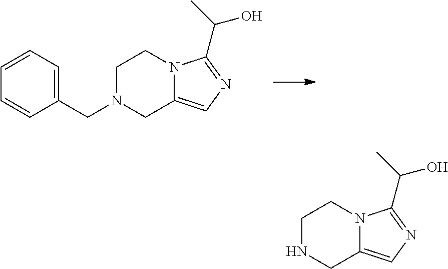

A mixture of 1-(7-benzyl-5,6,7,8-tetrahydroimidazo[1,5-a]pyrazin-3-yl)ethanol (0.400 g, 1.55 mmol, Preparation #N.1), 10% Pd/C (50% wet, 0.331 g, 0.155 mmol), ammonium formate (0.77 mL, 16 mmol) and MeOH (5 mL) were warmed to about 60° C. The reaction mixture was left to stir at about 60° C. for about 18 h. The reaction mixture was cooled to about 20° C. and then filtered through Celite® rinsing with MeOH. The filtrate was concentrated, mostly dissolved in DCM/MeOH, and then filtered to remove the insolubles. The filtrate was concentrated, the residue was dissolved in DCM (5 mL), and 4 N HCl in 1,4-dioxane (0.1 mL) was added. The volatiles were removed under reduced pressure, the residue was triturated with Et₂O, and the sticky solid was dried under high vacuum to afford 1-(5,6,7,8-tetrahydroimidazo[1,5-a]pyrazin-3-yl)ethanol hydrochloride (0.290 g, 92% yield): LC/MS (Table 1, Method c) Rₜ=0.43 min; MS m/z: 168 (M+H)⁺.

### General Procedure P

### Formation of a Halide from an Alcohol

To an alcohol (1 equiv), neat or as a solution in an organic solvent such as DCM, chloroform, or DCE, is added SOCl₂, oxalyl chloride, phosphorous oxychloride, N-chlorosuccinimide and triphenylphosphine or N-bromosuccinimide and triphenylphosphine or iodine and triphenylphosphine or MsCl with TEA then LiBr (preferably SOCl₂, 10-20 equiv). The solution/mixture is optionally warmed to 30-100° C. (preferably 50° C.) for about 1-48 h (preferably 24 h). The volatiles may be removed under reduced pressure or added dropwise into an aqueous solution/mixture such as water or water and ice with subsequent adjustment of the pH to allow for precipitation or extraction with an organic solvent such as DCM or EtOAc. The organics may be dried over Na₂SO₄ or MgSO₄, filtered, and concentrated under reduced pressure.

### Illustrations of General Procedure P

### Preparation #P.1

### cis-4-(Chloromethyl)-N,N-dimethylcyclohexanamine

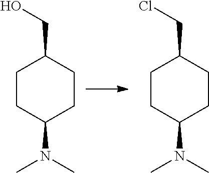

SOCl₂ (6.0 mL, 83 mmol) was added to (cis-4-(dimethylamino)cyclohexyl)methanol (1.00 g, 6.36 mmol). The reaction mixture was left to stir at about 50° C. for about 24 h. The reaction mixture was concentrated, chased with MeCN (20 mL), and dried under reduced pressure to afford cis-4-(chloromethyl)-N—N-dimethylcyclohexanamine (1.06 g, 79% yield): LC/MS (Table 1, Method e) Rₜ=0.32 min; MS m/z: 176 (M+H)⁺.

### General Procedure Q

### Reduction of a Carboxylic Acid, Carboxylic Ester, Aldehyde, or Ketone to an Alcohol

To a solution of the carboxylic acid, carboxylic ester, aldehyde, or ketone (1 equiv) in an organic solvent such as Et₂O, THF, 1,4-dioxane, DCM, toluene, or MeOH (preferably THF) at about −78° C. to ambient temperature (preferably 0° C.) is added, neat or as a solution in an organic solvent such as THF, toluene, cyclohexane, or Et₂O (preferably THF), a reducing agent such as LiAlH₄, LiBH₄, diisobutylaluminum hydride, or sodium borohydride (0.5-15 equiv, preferably LiAlH₄, 1-2 equiv). Alternatively, the order of addition may be reversed. The solution is stirred at −78-100° C. (preferably 0° C. to ambient temperature) for 0.25-72 h (preferably 2-3 h). Optionally, the reaction mixture may be diluted with Et₂O or concentrated. The reaction mixture may be quenched by careful addition of aqueous NaOH, sodium sulfate decahydrate, acetone, EtOAc, aqueous sodium potassium tartrate, water, or saturated aqueous NH₄Cl (preferably sodium sulfate decahydrate or saturated aqueous NH₄Cl). Optionally, the mixture is filtered through Celite® using additional rinses with an organic solvent such as Et₂O as necessary. An organic solvent such as DCM, Et₂O, or EtOAc (preferably DCM) may be added. If an aqueous layer is present, the pH may be adjusted and the layers separated with additional extractions as necessary. The organics are dried over Na₂SO₄ or MgSO₄, filtered, and concentrated under reduced pressure.

### Illustrations of General Procedure Q

### Preparation #Q.1

### tert-Butyl 4-(1-hydroxy-2-methylpropan-2-yl)piperazine-1-carboxylate

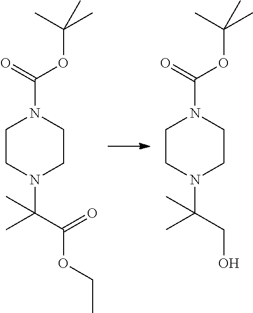

To a solution of tert-butyl 4-(1-ethoxy-2-methyl-1-oxopropan-2-yl)piperazine-1-carboxylate (1.00 g, 3.33 mmol, prepared using I with tert-butyl piperazine-1-carboxylate, K₂CO₃, and ethyl 2-bromo-2-methylpropanoate) in THF (40 mL) at about 0° C. was added LiAlH₄ (1 M solution in THF, 6.66 mL, 6.66 mmol). The reaction mixture was stirred at about 0° C. for about 2 h. The reaction mixture was quenched with saturated aqueous NH₄Cl and the aqueous layer was extracted with DCM. The combined organics were dried over Na₂SO₄, filtered, and concentrated under reduced pressure to give tert-butyl 4-(1-hydroxy-2-methylpropan-2-yl)piperazine-1-carboxylate (0.520 g, 60% yield): LC/MS (Table 1, Method c) Rₜ=1.22 min; MS m/z: 259 (M+H)⁺.

### General Procedure R

### Boc Protection of an Amine

To an amine (1 equiv) in an organic solvent such as DCM, Et₂O, or t-butanol (preferably t-butanol or DCM), optionally with an aqueous layer, is added a base such as TEA, DIEA, NaOH, or K₂CO₃ (1-10 equiv, preferably TEA or 2 M aqueous NaOH, 2-3 equiv) at about 0° C. to ambient temperature (preferably ambient temperature). Boc₂O (1-2 equiv, preferably 1.2 equiv) was added. After about 1-72 h (preferably 18 h), the mixture may be filtered and/or the volatiles removed under reduced pressure. Optionally, an aqueous phase such as water, aqueous NaHCO₃, or aqueous NaOH (preferably water) is added and the pH of the aqueous layer may be adjusted to induce precipitation or to allow extraction into an organic phase such as DCM, EtOAc, or Et₂O (preferably EtOAc). The precipitate may be collected by filtration and then dissolved in an organic solvent such as DCM or EtOAc (preferably EtOAc). The organics are dried over Na₂SO₄ or MgSO₄, filtered, and concentrated under reduced pressure.

### Illustrations of General Procedure R

### Preparation #R.1

### 1-(tert-butoxycarbonyl)-4-(tert-butoxycarbonylamino)piperidine-4-carboxylic acid

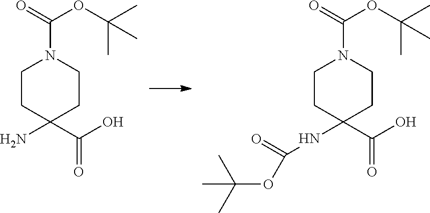

4-Amino-1-(tert-butoxycarbonyl)piperidine-4-carboxylic acid (3.00 g, 12.3 mmol, Alfa Aesar) was dissolved in NaOH (2 N aqueous solution, 14 mL, 28 mmol) and t-butanol (14 mL). Boc₂O (3.21 g, 14.7 mmol) was added in one portion and the reaction mixture was stirred at ambient temperature for about 18 h. The white solid that formed during that time was filtered and washed with Et₂O. The filtrate was collected and extracted with water. The aqueous layer was acidified to about pH 2 with 1 N aqueous HCl which triggered the precipitation of a white solid that was collected by filtration. The filter cake was dissolved in EtOAc and the resulting solution was dried over MgSO₄, filtered, and evaporated under reduced pressure to give 1-(tert-butoxycarbonyl)-4-(tert-butoxycarbonylamino)piperidine-4-carboxylic acid (2.50 g, 59%): LC/MS (Table 1, Method b) Rₜ=1.33 min; MS m/z: 345 (M+H)⁺.

### General Procedure S

### Formation of an Amide from a Carboxylic Acid and an Amine

To a solution or suspension of a carboxylic acid (1-5 equiv, preferably 1 equiv) and an amine or an amine salt (1-5 equiv, preferably 1 equiv) in an organic solvent such as DCM, DCE, THF, DMF, or 1,4-dioxane (preferably DMF) is added a peptide coupling reagent such as BOP—Cl, IBCF, HATU, TBTU, or EDC.HCl (1-10 equiv, preferably TBTU, 1-1.5 equiv), a base such as TEA, DIEA, or pyridine (0-20 equiv, preferably DIEA, 3 equiv). Optionally, HOBt (1-1.3 equiv) is added. The reaction mixture is then stirred at 0-60° C. (preferably ambient temperature) for about 15 min to 72 h (preferably 16 h). The reaction mixture is optionally diluted with an organic solvent such as EtOAc or DCM (preferably EtOAc). The reaction mixture is optionally diluted with water or saturated aqueous NaHCO₃ (preferably water) and then extracted with an organic solvent such as DCM or EtOAc (preferably EtOAc) with additional extractions of the aqueous phase or washes of the organic phase with water, saturated aqueous NaHCO₃ and/or brine. The organic layer is optionally dried over MgSO₄ or Na₂SO₄, filtered, and concentrated under reduced pressure.

### Illustrations of General Procedure S

### Preparation #S.1

### tert-Butyl 4-(2-(3-ethoxy-3-oxo-1-(pyridine-2-yl)propylamino)-2-oxoethyl) piperidine-1-carboxylate

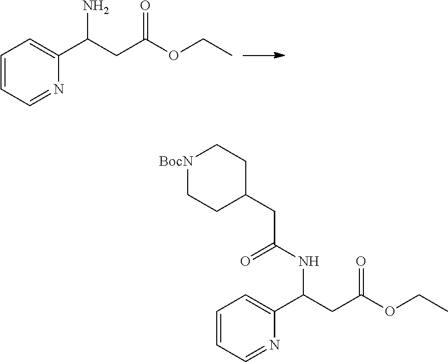

A mixture of ethyl 3-amino-3-(pyridine-2-yl)propanoate 2,2,2-trifluoroacetate (4.40 g, 14.3 mmol, Tetrahedron: Asymmetry 2009, 20, 1771-1777.), DIEA (5.53 g, 42.8 mmol, Sinopharm Chemical Reagent Co. Ltd.), TBTU (4.58 g, 14.3 mmol, Sinopharm Chemical Reagent Co. Ltd.) and 2-(1-(tert-butoxycarbonyl)piperidin-4-yl)acetic acid (3.47 g, 14.3 mmol, Sinopharm Chemical Reagent Co. Ltd.) in DMF (60 mL) was stirred at ambient temperature for about 16 h. EtOAc (400 mL) was added and the mixture was washed with water (3×100 mL). The organic layer was dried over Na₂SO₄, filtered and concentrated under reduced pressure. The residue was purified by Combi-flash (C18, MeOH/water/0.1% TFA) to give tert-butyl 4-(2-(3-ethoxy-3-oxo-1-(pyridine-2-yl)propylamino)-2-oxoethyl)piperidine-1-carboxylate (5.59 g, 93% yield): LC/MS (Table 1, Method t) Rₜ=1.70 min; MS m/z: 420 (M+H)⁺.

### General Procedure T

### Addition of a Grignard or Organolithium Reagent to a Ketone

To a solution of a ketone (1 equiv) in an organic solvent such as Et₂O, THF, 1,4-dioxane, toluene, or DCM (preferably THF) at about −100-25° C. (preferably −20° C.) is added a Grignard or organolithium reagent (0.9-3 equiv, preferably a Grignard reagent, 2 equiv). The mixture is optionally allowed to warm to −78° C. to ambient temperature (preferably ambient temperature) and then left to stir for about 0.25-24 h (preferably 2 h). An aqueous solution such as aqueous NH₄Cl, aqueous NaHCO₃, aqueous HCl or water (preferably aqueous NH₄Cl) is added and the layers are separated. The aqueous phase is optionally extracted with an organic solvent such as DCM or EtOAc and the combined organics may be washed with water, aqueous NH₄Cl, aqueous NaHCO₃ and/or brine. The organic layer is optionally dried over MgSO₄ or Na₂SO₄, filtered, and concentrated under reduced pressure.

### Illustrations of General Procedure T

### Preparation #T.1

### 1-Benzyl-4-ethylpiperidin-4-ol

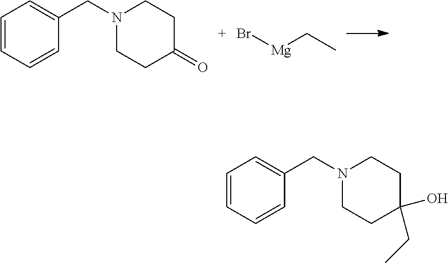

A round bottomed flask was charged with 1-benzylpiperidin-4-one (4.25 mL, 23.8 mmol) and THF (119 mL) under a nitrogen atmosphere. The solution was cooled to about −20° C. and ethyl magnesium bromide (1 M solution in THF, 47.6 mL, 47.6 mmol) was added dropwise and stirred for about 2 h. Saturated aqueous NH₄Cl (60 mL) was added and the layers were separated. The organics were dried over MgSO₄, filtered, and concentrated under reduced pressure. The crude oil was purified by flash column chromatography on silica gel eluting with a gradient of 0-50% (2% (2 N NH₃ in EtOH) in 10% MeOH/DCM)/DCM. The product containing fractions were combined and concentrated under reduced pressure to give 1-benzyl-4-ethylpiperidin-4-ol (1.38 g, 26% yield): LC/MS (Table 1, Method a) Rₜ=0.83 min; MS m/z: 220 (M+H)⁺.

### General Procedure U

### Acetamide Formation from a Tertiary Alcohol

To a solution of an alcohol (1 equiv) and MeCN (10-200 equiv, preferably 30-40 equiv) under a nitrogen atmosphere, optionally using acetic acid as a co-solvent, is added a dehydrating agent such as sulfuric acid, TFA, or trifluoromethanesulfonic anhydride (5-30 equiv, preferably sulfuric acid, 20-25 equiv) at about 0° C. to ambient temperature (preferably 0° C.). After addition, the solution is stirred at ambient temperature for about 1-72 h (preferably 40 h). The reaction solution is optionally poured into ice water. The solution is partitioned between an organic solvent such as EtOAc or DCM (preferably DCM) and an aqueous base such as NaHCO₃, Na₂CO₃ or NaOH (preferably 6 M aqueous NaOH) and the aqueous layer is optionally extracted with additional organics such as EtOAc or DCM. The organic layer is dried over MgSO₄ or Na₂SO₄, filtered, and concentrated under reduced pressure.

### Illustrations of General Procedure U

### Preparation #U.1

### N-(1-Benzyl-4-ethylpiperidin-4-yl)acetamide

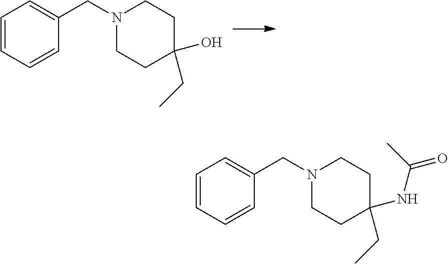

A round bottomed flask was charged with 1-benzyl-4-ethylpiperidin-4-ol (1.38 g, 6.13 mmol, Preparation #T.1) and MeCN (12.4 mL, 237 mmol) under a nitrogen atmosphere. The solution was cooled to about 0° C. and sulfuric acid (6.80 mL, 128 mmol) was added dropwise. After completion of addition, the ice bath was removed and reaction solution was stirred at ambient temperature for about 40 h. The reaction solution was poured into ice water (50 mL) and the pH was adjusted to about 10 using 6 N aqueous NaOH. The aqueous mixture was extracted with DCM (2×100 mL). The combined organics were washed with brine (50 mL), dried over MgSO₄, filtered, and concentrated to give N-(1-benzyl-4-ethylpiperidin-4-yl)acetamide (1.56 g, 83% yield): LC/MS (Table 1, Method c) Rₜ=1.07 min; MS m/z: 261 (M+H)⁺.

### General Procedure V

### Hydrolysis of an Acetyl Protected Amine

To a solution of an N-acetamide (1 equiv), optionally in an organic solvent such as 1,4-dioxane, is added an acid, such as 6 N aqueous HCl (3-100 equiv, preferably 15-40 equiv). The reaction mixture is heated at about 60-150° C. (preferably 100-140° C.) for about 1-200 h (preferably 48-96 h). The reaction mixture is cooled to about 0° C. to ambient temperature before partitioning between an organic solvent such as EtOAc or DCM (preferably DCM) and aqueous base such as NaHCO₃, Na₂CO₃ or NaOH (preferably 2 M aqueous NaOH) and the aqueous layer is optionally extracted with additional organic solvent such as EtOAc or DCM. The organic layer is dried over MgSO₄ or Na₂SO₄, filtered, and concentrated under reduced pressure. The residue may, as necessary, be resubmitted to the reaction conditions.

### Illustrations of General Procedure V

### Preparation #V.1

### 1-Benzyl-4-ethylpiperidin-4-amine

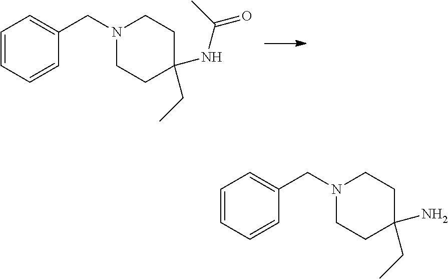

A round bottomed flask was charged with N-(1-benzyl-4-ethylpiperidin-4-yl)acetamide (1.56 g, 5.08 mmol, Preparation #U.1) and 6 N aqueous HCl (13.2 mL, 79 mmol). The mixture was warmed to about 110° C. for about 48 h and then to about 140° C. for about 48 h. After cooling to about 0° C., the pH was adjusted to about 10 with 2 N aqueous NaOH. The aqueous mixture was extracted with DCM (3×100 mL). The combined organics were dried over MgSO₄, filtered, and concentrated under reduced pressure. The crude material was dissolved in 6 N aqueous HCl (13 mL, 79 mmol) and heated to reflux for about 96 h. The reaction was cooled to about 0° C. using an ice bath. The pH was adjusted to about 10 using 2 N aqueous NaOH. The mixture was then extracted with DCM (3×90 mL). The combined organics were dried over MgSO₄, filtered, and concentrated under reduced pressure to afford 1-benzyl-4-ethylpiperidin-4-amine (0.690 g, 62% yield): LC/MS (Table 1, Method c) Rₜ=0.57 min; MS m/z: 219 (M+H)⁺.

TABLE 3
PKCθ enzyme data at 1 mM ATP

| Example# | PKCθ enzyme IC₅₀ |
|---|---|
| Example #1 | B |
| Example #2 | A |
| E.1.1 | A |
| E.1.2 | B |
| E.1.3 | B |
| E.1.4 | C |
| E.1.5 | A |
| E.1.6 | B |
| E.1.7 | C |
| E.1.8 | A |
| E.1.9 | B |
| E.1.10 | C |
| E.1.11 | B |
| E.1.12 | B |
| E.1.13 | A |
| E.1.14 | C |
| E.1.15 | C |
| E.1.16 | B |
| E.1.17 | A |
| E.1.18 | A |
| E.1.19 | A |
| E.1.20 | B |
| E.1.21 | A |
| E.1.22 | A |
| E.1.23 | C |
| E.1.24 | A |
| E.1.25 | B |
| E.1.26 | A |
| E.1.27 | A |
| E.1.28 | B |
| E.1.29 | B |
| E.1.30 | A |
| E.1.31 | A |
| E.1.32 | A |
| E.1.33 | B |
| E.1.34 | A |
| E.1.35 | A |
| E.1.36 | A |
| E.1.37 | A |
| E.1.38 | B |
| E.1.39 | A |
| E.1.40 | A |
| E.1.41 | B |
| E.1.42 | B |
| E.1.43 | A |
| E.1.44 | A |
| E.1.45 | A |
| E.1.46 | A |
| E.1.47 | A |
| E.1.48 | A |
| E.1.49 | B |
| E.1.50 | A |
| E.1.51 | A |
| E.1.52 | A |
| E.1.53 | A |
| E.1.54 | A |
| E.1.55 | A |
| E.1.56 | A |
| E.1.57 | A |
| E.1.58 | A |
| E.1.59 | A |
| E.2.1 | A |
| E.2.2 | A |
| E.2.3 | A |
| E.2.4 | A |
| E.2.5 | B |
| E.2.6 | A |
| E.2.7 | A |
| E.2.8 | B |
| E.2.9 | B |
| E.2.10 | A |
| E.3.1 | A |
| F.1.1 | A |
| F.1.2 | A |
| F.1.3 | B |
| F.2.1 | A |
| F.3.1 | A |
| F.3.2 | A |
| F.3.3 | A |
| F.3.4 | A |
| F.3.5 | A |
| F.4.1 | A |
| F.4.2 | A |
| F.4.3 | A |
| F.4.4 | A |
| F.4.5 | A |
| F.4.6 | A |
| F.4.7 | B |
| F.4.8 | C |
| F.4.9 | A |
| F.4.10 | B |
| F.4.11 | B |
| F.4.12 | C |
| F.4.13 | A |
| F.4.14 | A |
| F.4.15 | A |
| F.4.16 | B |
| F.4.17 | A |
| F.4.18 | B |
| F.4.19 | B |
| F.4.20 | B |
| F.4.21 | B |
| F.4.22 | A |
| F.4.23 | B |
| F.4.24 | A |
| F.4.25 | B |
| F.4.26 | B |
| F.4.27 | C |
| F.4.28 | B |
| F.4.29 | B |
| F.4.30 | A |
| F.4.31 | A |
| F.4.32 | C |
| F.4.33 | C |
| F.4.34 | A |
| F.4.35 | C |
| F.4.36 | A |
| F.4.37 | A |
| F.5.1 | A |
| G.1.1 | A |
| G.1.2 | B |
| G.1.3 | C |
| G.1.4 | B |
| G.1.5 | A |
| G.1.6 | B |
| G.1.7 | B |
| G.1.8 | B |
| G.1.9 | C |
| G.1.10 | A |
| G.1.11 | B |
| G.1.12 | A |
| G.1.13 | B |
| G.1.14 | C |
| G.1.15 | B |
| G.1.16 | B |
| G.1.17 | A |
| G.1.18 | A |
| G.1.19 | A |

Key:
A <0.1 μM
B 0.1-<0.5 μM
C 0.5-1 μM

What is claimed:

1. A compound of Formula (I) 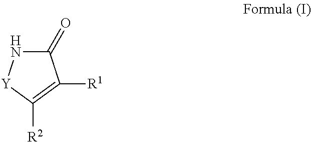
biologically active metabolites, pro-drugs, isomers, stereoisomers, solvates, hydrates and pharmaceutically acceptable salts thereof wherein
Y is —C(R^{c})₂—, —C(═O)—, —C(═S)—, —C(═NR^{e})—, —N(R^{e})—, —O—, —S—, —S(O)—, or —S(O)₂—;
R¹ is 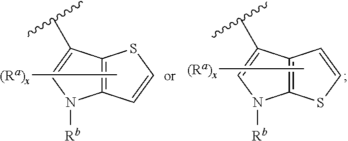
wherein
R^{a} is independently deuterium, halo, —OR^{d}, —CN, —(C₁-C₆)alkoxy, —N(R^{d})₂, —C(O)OR^{d}, —COR^{d}, —N(R^{d})S(O)₂R^{d}, —S(O)₂N(R^{d})₂, —C(O)N(R^{d})₂, —N(R^{d})C(O)R^{d}, —SR^{d}, —S(O)R^{d}, —S(O)₂R^{d}, optionally substituted (C₂-C₆)alkenyl, optionally substituted (C₂-C₆)alkynyl, optionally substituted (C₁-C₆)alkyl, optionally substituted spirocyclic (C₅-C₁₄)cycloalkyl, optionally substituted spirocyclic (C₂-C₁₁)heterocyclyl, optionally substituted (C₃-C₆)cycloalkyl, optionally substituted (C₁-C₁₀)heterocyclyl, optionally substituted (C₆-C₁₀)aryl, optionally substituted (C₁-C₁₀)heteroaryl, optionally substituted bridged (C₅-C₁₂) cycloalkyl, or optionally substituted bridged (C₂-C₁₀)heterocyclyl; or
R^{a} is independently —(C(R^{d})₂)ₓ—B-E-G-J wherein
B is independently a bond, —N(R^{d})—, —O—, —C(O)—, —C(O)O—, —S—, —SO—, —SO₂—, —N(R^{d})S(O)₂—, —S(O)₂N(R^{d})—, —C(O)N(R^{d})—, —N(R^{d})C(O)— or —N(R^{d})C(O)N(R^{d})—;
E is independently a bond, N(R²), optionally substituted (C₁-C₆)alkylene, optionally substituted (C₂-C₆)alkenylene, optionally substituted (C₂-C₆)alkynylene, optionally substituted spirocyclic (C₅-C₁₄)cycloalkylene, optionally substituted spirocyclic (C₃-C₁₁)heterocyclylene, optionally substituted bridged (C₅-C₁₂)cycloalkylene, optionally substituted bridged (C₂-C₁₀)heterocyclylene, optionally substituted (C₃-C₆)cycloalkylene, optionally substituted (C₁-C₁₀)heterocyclylene, optionally substituted (C₆-C₁₀)arylene, or optionally substituted (C₁-C₁₀)heteroarylene;
G is independently a bond, optionally substituted —(C₁-C₆)alkylene-, optionally substituted —(C₂-C₆)alkenylene-, optionally substituted —(C₂-C₆)alkynylene-, —O—, —S—, —S(O)ₚ—, —N(R^{c})—, —N(C(O)OR^{d})—, —N(C(O)R^{d})—, —N(S(O)ₚR^{d})—, —C(R^{d})₂O—, —O—(CR^{d})₂—, —C(R^{d})₂S—, —SC(R^{d})₂—, —C(R^{d})₂N(R^{d})—, —N(R^{d})C(R^{d})₂—, —C(R^{d})₂N(C(O)R^{d})—, —N(C(O)R^{d})C(R^{d})₂—, —C(R^{d})₂N(C(O)OR^{d})—, —N(C(O)OR^{d})C(R^{d})₂—, —C(R^{d})₂N(S(O)ₚR^{d})—, —(N(S(O)ₚR^{d})C(R^{d})₂—, —C(R^{d})(N(R^{d})(OR^{d}))—, —C(R^{d})(ON(R^{d})₂)—, —C(R^{d})(N(R^{d})₂)—, —C(R^{d})(N(R^{d})S(O)ₚR^{d})—, —C(R^{d})(S(O)ₚN(R^{d})₂)—, —C(R^{d})(N(R^{d})C(O)OR^{d})—, —CR^{d}(OC(O)R^{d})—, —CR^{d}(C(O)OR^{d})—, —C(R^{d})(OC(O)N(R^{d})₂—, —C(═NOR^{d})—, —C(O)—, —C(O)O—, —C(R^{d})(OR^{d})—, —C(O)N(R^{d})—, —N(R^{d})C(O)—, —N(R^{d})S(O)ₚ—, —S(O)ₚN(R^{d})—, —N(R^{d})C(O)N(R^{d})—, —N(R^{d})S(O)ₚN(R^{d})—, —OC(O)N(R^{d})—, —N(R^{d})C(O)O—, —ON(R^{d})C(O)—, —C(O)N(R^{d})O—, —N(OR^{d})C(O)—, —C(O)N(OR^{d})—, —N(R^{d})—C(O)—(C(R^{d})₂)ₙ₊₁—N(R^{d})—, —N(R^{d})—(C(R^{d})₂)ₙ₊₁—C(O)—N(R^{d})—, —C(O)—N(R^{d})—(C(R^{d})₂)ₙ₊₂—N(R^{d})—, —N(R^{d})—(C(R^{d})₂)ₙ₊₂—N(R^{d})—C(O)—, —N(R^{d})—(C(R^{d})₂)ₙ₊₁—C(O)—, —C(O)—(C(R^{d})₂)ₙ₊₁—N(R^{d})—, —O—(CR^{d})ₙ₊₁—C(O)—, —C(O)—CR^{d})ₙ₊₁—O—, —O—(C(R^{d})₂)ₙ₊₂—O—, —N(R^{d})—C(O)—(CH₂)ₙ₊₁—O—, —O—(C(R^{d})₂)ₙ₊₁—C(O)—N(R^{d})—, —O—(C(R^{d})₂)ₙ₊₂ N(R^{d})—C(O)—, —C(O)—N(R^{d})—(C(R^{d})₂)ₙ₊₂—O—, —O—(C(R^{d})₂)ₙ₊₂N(R^{d})—, —N(R^{d})—(C(R^{d})₂)ₙ₊₂—O—, —N(R^{d})—(C(R^{d})₂)ₙ₊₂—N(R^{d})—, —C(O)N(R^{d})C(O)—, —S(O)ₚN(R^{d})C(O)—, —C(O)N(R^{d})S(O)ₚ—, —OS(O)ₚN(R^{d})—, —N(R^{d})S(O)ₚO—, —N(R^{d})S(O)ₚC(O)—, —C(O)S(O)ₚN(R^{d})—, —S(O)ₚN(C(O)R^{d})—, —N(C(O)R^{d})S(O)ₚ—, —N(S(O)ₚ(R^{d})C(O)—, —C(O)N(S(O)ₚ(R^{d}))—, —N(R^{d})P(O)(OR^{d})—, —N(R^{d})P(O)(OR^{d})O—, —N(C(O)R^{d})P(O)(OR^{d})—, or —N(C(O)R^{d})P(O)(OR^{d})O—;
wherein
n is 0 to 6;
p is 1 or 2;
J is independently H, N(R^{d})₂, optionally substituted (C₁-C₆)alkyl, optionally substituted (C₂-C₆)alkenyl, optionally substituted (C₂-C₆)alkynyl, optionally substituted spirocyclic (C₅-C₁₄)cycloalkyl, optionally substituted spirocyclic (C₃-C₁₄)heterocyclyl, optionally substituted bridged (C₅-C₁₂)cycloalkyl, optionally substituted bridged (C₂-C₁₀)heterocyclyl, optionally substituted (C₃-C₆)cycloalkyl, optionally substituted (C₁-C₁₀)heterocyclyl, optionally substituted (C₆-C₁₀)aryl, or optionally substituted (C₁-C₁₀)heteroaryl;
provided that —B-E-G-J does not form a three atom combination of oxygen atoms, nitrogen atoms or a combination of oxygen and nitrogen atoms directly bound to one another;
R^{b} is independently H, —C(O)R^{d}, —COOR^{d}, —S(O)₂N(R^{d})₂, —C(O)N(R^{d})₂, —S(O)R^{d}, —S(O)₂R^{d}, optionally substituted (C₁-C₆)alkyl, optionally substituted (C₂-C₆)alkenyl, optionally substituted (C₂-C₆)alkynyl, optionally substituted (C₂-C₆)alkoxy, optionally substituted spirocyclic (C₅-C₁₄)cycloalkyl, optionally substituted spirocyclic (C₂-C₁₀)heterocyclyl; optionally substituted (C₃-C₆)cycloalkyl, optionally substituted (C₁-C₁₀)heterocyclyl, optionally substituted (C₆-C₁₀)aryl, optionally substituted (C₁-C₁₀)heteroaryl, optionally substituted bridged (C₂-C₁₀)heterocyclyl, or optionally substituted bridged (C₂-C₁₀)cycloalkyl; or
R^{b} is independently —(C(R^{d})₂)ₓ—B-E-G-J;
R^{c} is independently H, OH, deuterium, F, —O-optionally substituted (C₃-C₆)cycloalkyl, optionally substituted —OC₁-C₆)alkyl, optionally substituted (C₁-C₆)alkyl or optionally substituted (C₃-C₆)cycloalkyl;
R^{d} is independently H, optionally substituted (C₁-C₆)alkyl, optionally substituted (C₂-C₆)alkenyl, optionally substituted (C₂-C₆)alkynyl, optionally substituted (C₃-C₆)cycloalkyl, optionally substituted (C₆-C₁₀)aryl, optionally substituted (C₁-C₁₀)heteroaryl or optionally substituted (C₁-C₁₀)heterocyclyl;
R^{e} is H, optionally substituted (C₁-C₆)alkyl or optionally substituted (C₃-C₆)cycloalkyl;
R² is optionally substituted (C₆-C₁₀)aryl, optionally substituted (C₃-C₆)cycloalkyl, optionally substituted (C₁-C₁₀)heterocyclyl or optionally substituted (C₁-C₁₀)heteroaryl; and
x is 0 to 3.

2. The compound of claim 1 wherein R² is 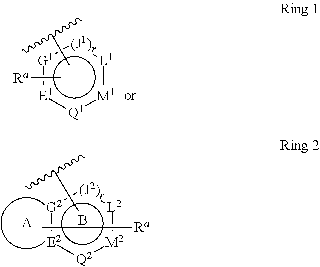
wherein
in Ring 1
r is 1 and E¹, G¹, J¹, L¹, M¹ and Q¹ are each independently C, CR^{a}, N; or
r is 0 and E¹, G¹, L¹, M¹ and Q¹ are each independently C, CR^{a}, NR^{b}, N, S or O; or
in Ring 2
Ring A is a five to seven membered optionally substituted ring selected from aryl, heterocyclyl, heteroaryl and cycloalkyl fused to Ring B;
r is 1 and J², L², M² and Q² are each independently C, CR^{a}, or N; and E², and G² are independently C or N; or
r is 0 and L², M² and Q² are each independently C, CR^{a}, N, NR^{b}, S or O and E² and G², are independently C or N; or
when r is 0, L² and M² together or M² and Q² together optionally form a saturated or unsaturated four to seven membered carbocyclic or heterocylic ring, provided that none of L², M² or Q² is independently O or S and only one of L², M² or Q² is N; or
when r is 1, M² and Q² together optionally form a saturated or unsaturated four to seven membered carbocyclic or heterocylic ring, provided that neither M² or Q² is N; or
when r is 1, L² and M² together optionally form a saturated or unsaturated four to seven membered carbocyclic or heterocylic ring, provided that neither L² or M² is N.

3. The compound of claim 2 wherein R² is Ring 1 and Ring 1 is 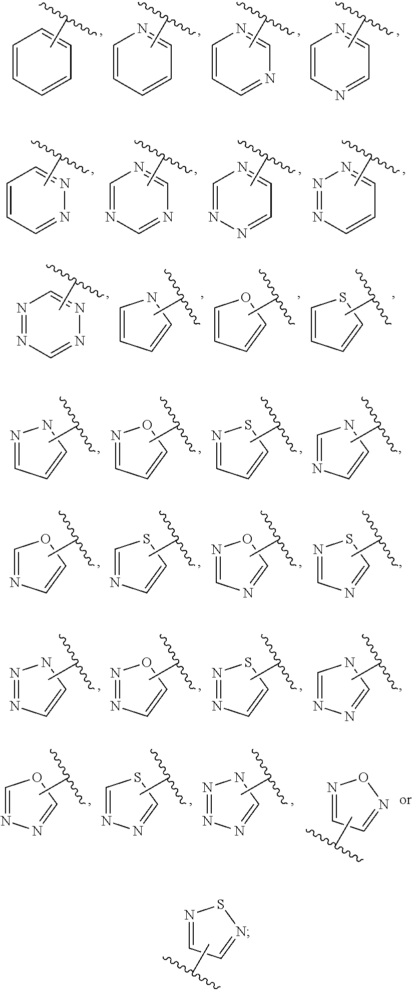
wherein carbon atoms in Ring 1 are optionally substituted by R^{a} and nitrogen atoms are optionally substituted by R^{b};
or R² is Ring 2 and Ring 2 is

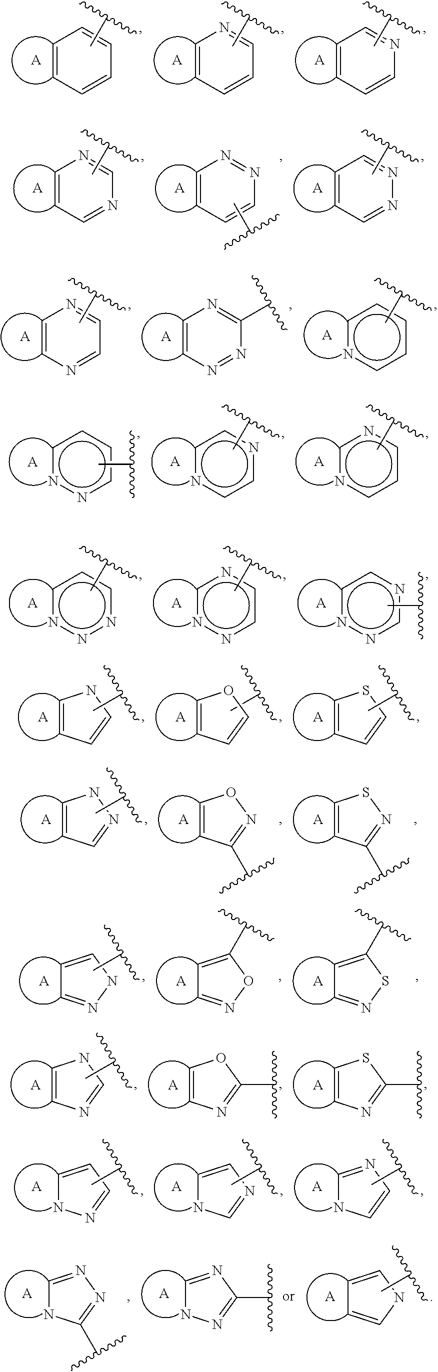

4. The compound of claim 3 wherein Ring A is 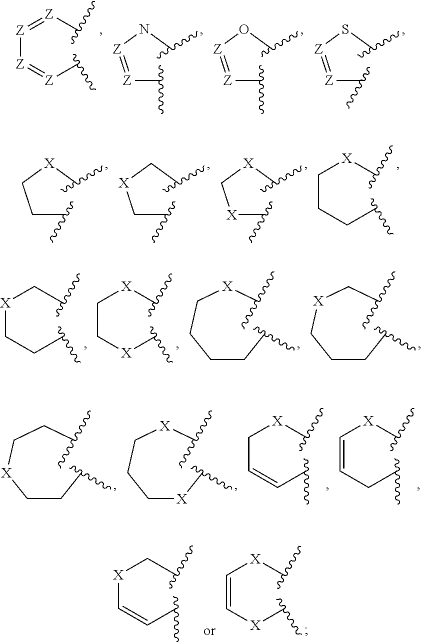
wherein X is independently —S—, —SO—, —SO₂—, —O—, —N(R^{b})—, or —C(R^{a})₂— and when X is N(R^{b}) then an adjacent carbon atom can be optionally substituted with oxo; and
Z is independently C, C(R^{a}) or N;
and carbon atoms in Ring A are optionally substituted by R^{a} and nitrogen atoms are optionally substituted by R^{b}.

5. The compound of claim 4 wherein R² is 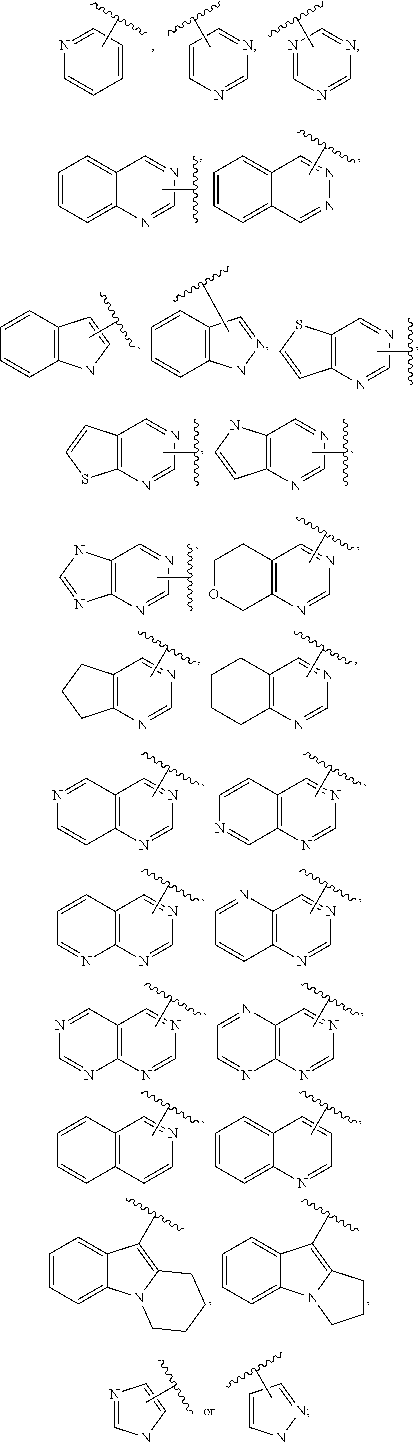
and carbon atoms in R² are independently optionally substituted by R^{a} and nitrogen atoms are optionally substituted by R^{b}.

6. The compound of claim 5 wherein R^{a} is optionally substituted (C₁-C₆)alkyl or 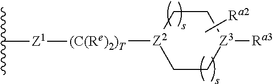
wherein
Z¹ is a bond or —N(R^{e});
Z² is CR^{a1} or N;
Z³ is CR^{a4} or N; or
Z³ is O and R^{a3} is not present;
R^{a1} is H or optionally substituted (C₁-C₆)alkyl;
R^{a2} and R^{a3} are each independently H, —CN, —CF₃, —OH, (C₁-C₆)alkoxy, optionally substituted (C₃-C₆) cycloalkyl, —C(O)—N(R^{e})(R^{f}), F, —N(R^{e})(R^{f}), optionally substituted (C₁-C₆)alkyl; optionally substituted (C₃-C₆)cycloalkyl, —(C(R^{e})₂)ₘ-optionally substituted heterocyclyl, —(C(R^{e})₂)ₘ-optionally substituted heteroaryl;
provided that when Z³ is N then R^{a3} is not —CN or F;
wherein R^{e} and R^{f} are independently H, optionally substituted (C₁-C₆)alkyl or optionally substituted (C₃-C₆)cycloalkyl; or
—N(R^{e})(R^{f}) can form an optionally substituted 4-, 5- or 6-membered saturated or unsaturated heterocyclic ring;
R^{a4} is H, optionally substituted (C₁-C₆)alkyl or optionally substituted (C₃-C₆)cycloalkyl; or
R^{a1} and R^{a2} combine with the atoms to which they are attached to form a 4-, 5- or 6-membered optionally substituted saturated or unsaturated carbocyclic or optionally substituted saturated or unsaturated heterocylic ring; or
R^{a2} and R^{a3} combine with the atoms to which they are attached to form a 4-, 5- or 6-membered saturated or unsaturated carbocyclic or optionally substituted saturated or unsaturated heterocylic ring; or
R^{a3} and R^{a4} form a 4-, 5- or 6-membered optionally substituted saturated carbocyclic or heterocyclic ring to form a spirocyclic moiety;
m is 0, 1 or 2;
s is independently 0, 1, or 2; and
T is 0, 1, 2 or 3.

7. The compound of claim 6 wherein R^{a} is 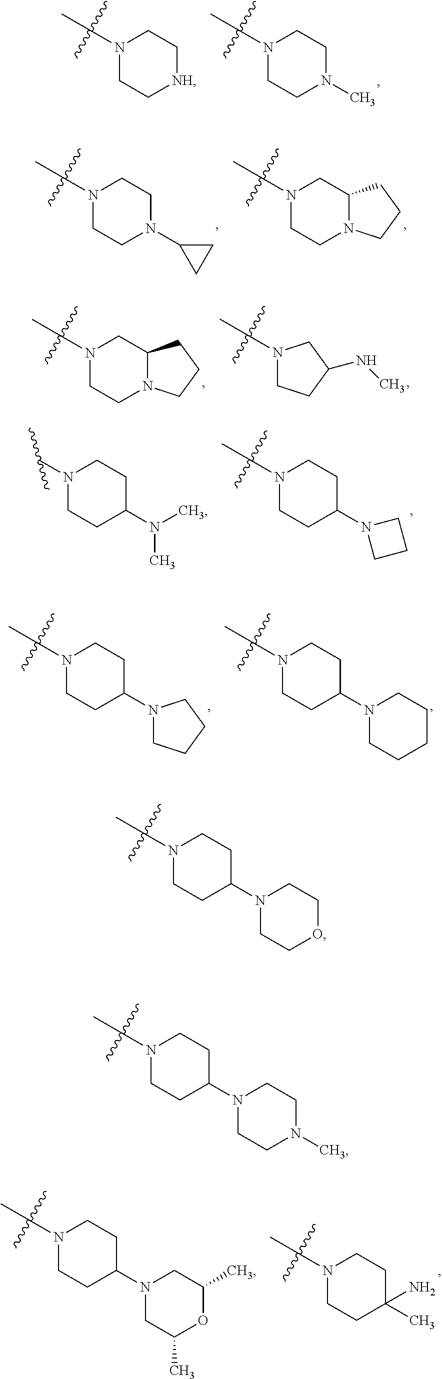 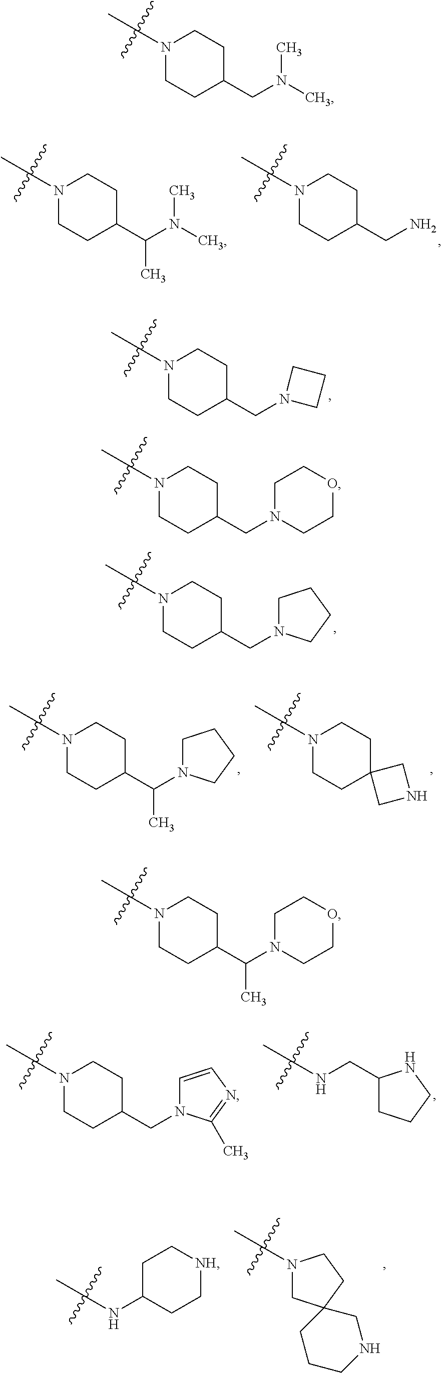 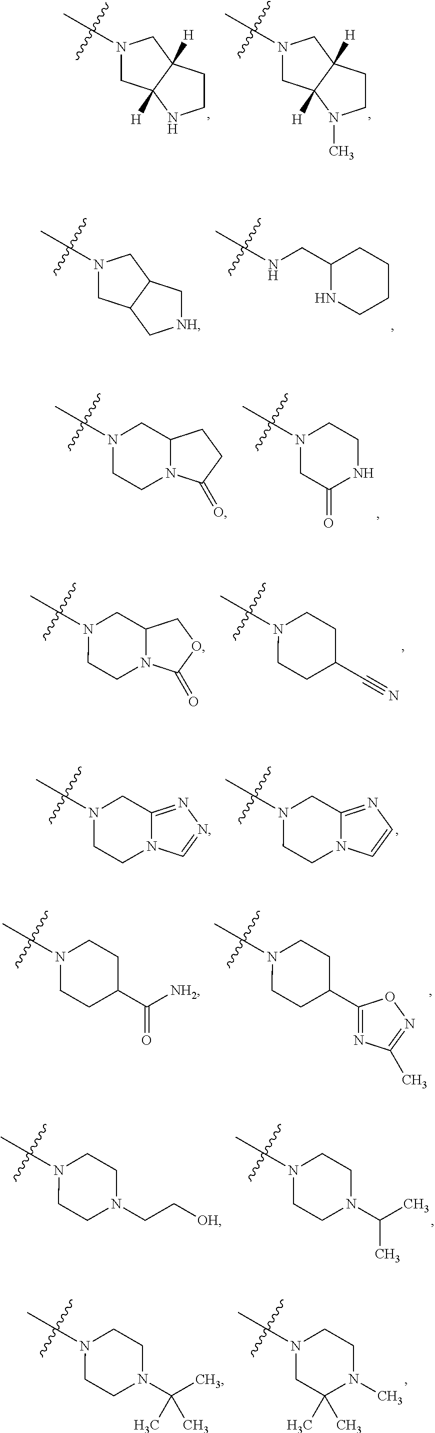 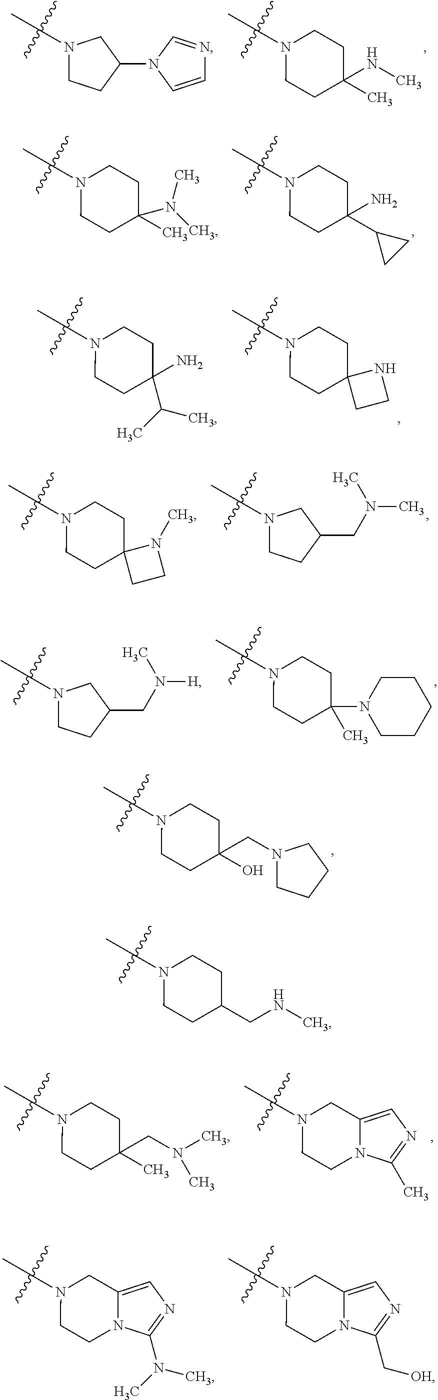 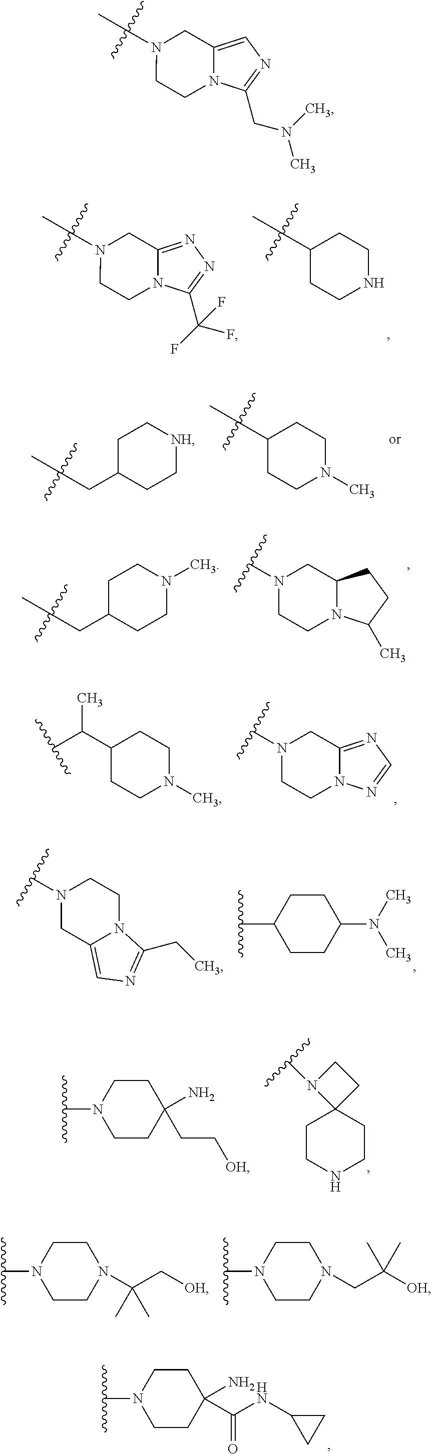 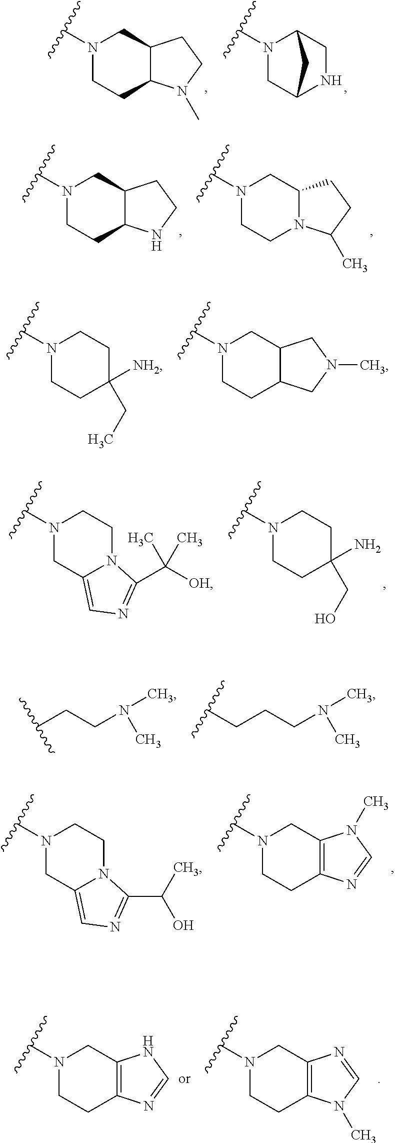
wherein R^{a} is optionally substituted by —(CH₂)_{T}CF₃, —(CH₂)_{T}CHF₂, —(CH₂)_{T}CH₂F, —F, —(CH₂)_{T}OH, —CH₂C(CH₃)₂OH, —C(CH₃)₂CH₂OH, —OCH₃, —OCF₃, —(CH₂)_{T}CH₃, —O(CH₂)_{T}CH₃, —(CH₂)_{T}OCH₃, —(CH₂)_{T}OC(CH₃)₃, —(CH₂)_{T}OCH(CH₃)₂, —(CH₂)_{T}OCH₂CH₃, —(CH₂)_{T}OCF₃, —(CF₂)_{T}CF₃, —(CF₂)_{T}CHF₂, —(CF₂)_{T}CH₂F, —(CHF)_{T}CF₃, —(CHF)_{T}CHF₂, —(CHF)_{T}CH₂F, —(CH₂)_{T}CH₃, —CH(CH₃)₂, —C(CH₃)₃, —(CH₂)_{T}CN, —(CH₂)_{T}NH₂, —(CH₂)_{T}NHCH₃, —(CH₂)_{T}N(CH₃)₂, —(CH₂)_{T}CONH₂, —(CH₂)_{T} CONHCH₃, —CON(CH₃)₂ or optionally substituted (C₃-C₆)cycloalkyl; and
T is 0, 1, 2 or 3

8. The compound of claim 6 wherein R^{b} is 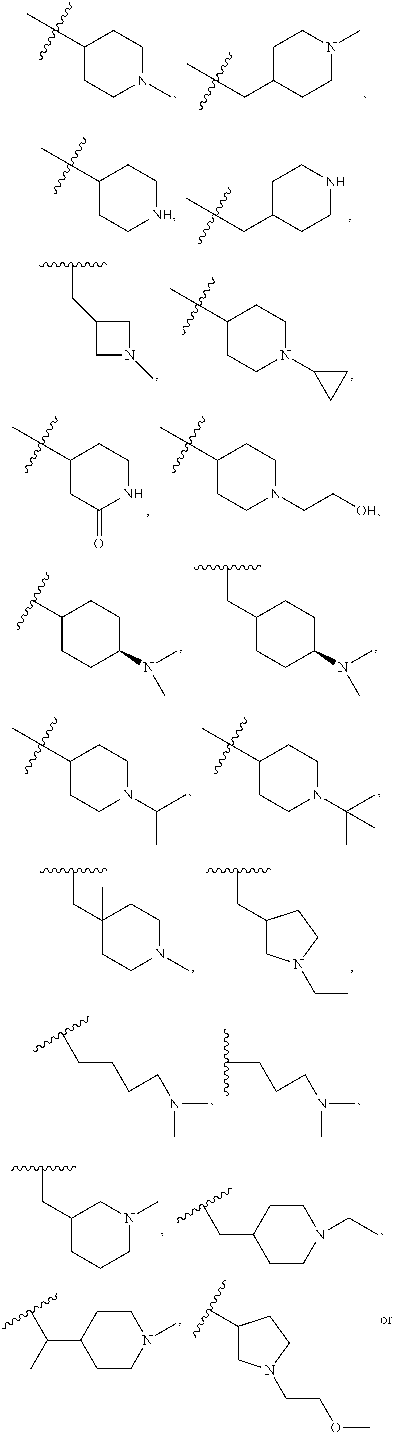 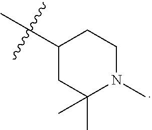
wherein R^{b} is optionally substituted by —(CH₂)_{T}CF₃, —(CH₂)_{T}CHF₂, —(CH₂)_{T}CH₂F, —F, —(CH₂)_{T}OH, —CH₂C(CH₃)₂OH, —C(CH₃)₂CH₂OH, —OCH₃, —OCF₃, —(CH₂)_{T}CH₃, —(CH₂)_{T}OCH₃, —(CH₂)_{T}OC(CH₃)₃, —(CH₂)_{T}OCH(CH₃)₂, —(CH₂)_{T}OCH₂CH₃, —(CH₂)_{T}OCF₃, —(CF₂)_{T}CF₃, —(CF₂)_{T}CHF₂, —(CF₂)_{T}CH₂F, —(CHF)_{T}CF₃, —(CHF)_{T}CHF₂, —(CHF)_{T}CH₂F, —CH(CH₃)₂, —C(CH₃)₃, —(CH₂)_{T}CN, —(CH₂)_{T}NH₂, —(CH₂)_{T}NHCH₃, —(CH₂)_{T}N(CH₃)₂, —(CH₂)_{T}CONH₂, —(CH₂)_{T}CONHCH₃, or —CON(CH₃)_{2 2} or optionally substituted (C₃-C₆)cycloalkyl.

9. The compound of claim 8 wherein Y is —C(R^{c})₂—, —C(═O)—, —C(═S)—, —C(═NR^{e})—, or —S(O)—.

10. A method of inhibiting one or more protein kinase activity in a patient comprising administering a therapeutically effective amount of a compound of claim 1 or a physiologically acceptable salt, pro-drug or biologically active metabolites thereof to said patient.

11. The method of claim 10 wherein said protein kinase is selected from the group consisting of PKC, Jak1, Jak2, Jak3, Tyk2, KDR, Flt-3, ROCK, CDK2, CDK4, TANK, Trk, FAK, Abl, Bcr-Abl, cMet, b-RAF, FGFR3, c-kit, PDGF-R, Syk, or Aurora kinases.

12. A method of treating a condition in a patient comprising administering a therapeutically effective amount of a compound of claim 1 or a physiologically acceptable salt, pro-drug or biologically active metabolites thereof to said patient, wherein said condition is an immunological disorder, an oncological disorder, a diabetic disorder or organ transplant.

13. The method of claim 12 wherein the immunological disorder is rheumatoid arthritis, ankylosing spondylitis, juvenile rheumatoid arthritis, Crohn's Disease, psoriatic arthritis, juvenile idiopathic arthritis, plaque psoriasis, multiple sclerosis, psosiasis, ulcerative colitis or inflammatory bowel disease or uveitis.

14. The method of claim 12 wherein the oncological disorder is cancer, lymphoma, myeloma, leukaemia, malignant ascites, hematopoietic cancers, lung cancer, breast cancer, colon cancer or bladder cancer.

15. The method of claim 12 wherein the diabetic disorder is diabetes, insulin-dependent diabetes mellitus glaucoma, diabetic retinopathy, macular edema, diabetic neuropathy or microangiopathy,

16. The method of claim 12 wherein the organ transplant is liver, heart, lung or kidney transplant.

17. A pharmaceutical composition comprising a compound according to claim 1 and a pharmaceutically acceptable carrier or diluent.
